(19) United States
(12) Patent Application Publication
(10) Pub. No.: US 2025/0197406 A1
(43) Pub. Date: Jun. 19, 2025

(54) INDAZOLES AS HEMATOPOIETIC PROGENITOR KINASE 1 (HPK1) INHIBITORS AND METHODS FOR USING SAME

(71) Applicant: 1ST Biotherapeutics, Inc., Gyeonggi-do (KR)

(72) Inventors: Jinhwa LEE, Gyeonggi-do (KR); Kyung-Hee KIM, Gyeonggi-do (KR); Keonseung LIM, Gyeonggi-do (KR); Changdev Gorakshnath GADHE, Seoul (KR); Yeonguk JEON, Gyeonggi-do (KR); Yejin HWANG, Gyeonggi-do (KR); Eunji JO, Incheon (KR); Boeun GU, Gyeonggi-do (KR); Jae Eun KIM, Gyeonggi-do (KR); Misoon KIM, Gyeonggi-do (KR); Seung Mook LIM, Gyeonggi-do (KR)

(21) Appl. No.: 18/847,802

(22) PCT Filed: Mar. 23, 2023
(86) PCT No.: PCT/IB2023/052861

§ 371 Date: Sep. 17, 2024

Related U.S. Application Data
(60) Provisional application No. 63/323,521, filed on Mar. 25, 2022.

(51) Int. Cl. C07D 487/04; A61K 31/4985; A61K 31/5377; A61K 45/06

(52) U.S. Cl. CPC C07D 487/04; A61K 31/4985; A61K 31/5377; A61K 45/06

(57) ABSTRACT

The present disclosure provides a compound of Formula (I) or pharmaceutically acceptable salts thereof, a composition comprising the compound, methods of using the compound for the treatment of various disorders associated with HPK1, and methods of preparing these compounds.

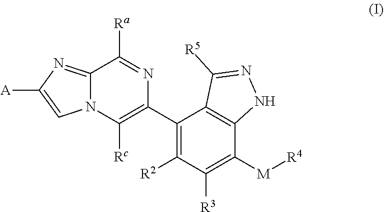

### FIELD

The present disclosure is directed to inhibitors of hematopoietic progenitor kinase 1 (HPK1), pharmaceutical compositions comprising the inhibitors, methods of using the inhibitors for the treatment of various disorders associated with HPK1, and methods of preparing these compounds.

### BACKGROUND

Immunotherapy is treatment that uses the human body's own immune system to help fight cancer and other disorders. This relatively new approach has achieved remarkable clinical successes in the treatment of a variety of tumor types in recent years, especially with the treatment of immune checkpoint inhibitors and chimeric antigen T-cell therapy. The most investigated checkpoint inhibitors including CTLA4, PD-1, or PD-L1 inhibitors have demonstrated significant antitumor activity by overcoming immunosuppressive mechanisms at the tumor site.

Hematopoietic progenitor kinase 1 (HPK1, MAP4K1) is a serine/threonine kinase and a member of MAP4K. HPK1 is prominently expressed in subsets of hematopoietic cell lineages. HPK1 is a newly identified as a critical negative regulator in the activation of T lymphocytes and dendritic cells. It has been recently demonstrated that the important roles for kinase activity of HPK1 in anti-cancer immunity as a new intracellular checkpoint molecule as well as potential advantages of combination therapy with current checkpoint regimens. HPK1 inhibition is expected to have dual functions, 1. prolonged activation of T cells; 2. enhanced APC functions by dendritic cells. This dual targeting may synergistically work together for efficient immune responses in tumor microenvironment. Thus, HPK1 has been validated as a novel target for anticancer immunotherapy. Examples of cancers that are treatable using the compounds of the present disclosure include, but are not limited to, all forms of carcinomas, melanomas, blastomas, sarcomas, lymphomas and leukemias, including without limitation, bladder carcinoma, brain tumors, breast cancer, cervical cancer, colorectal cancer, esophageal cancer, endometrial cancer, hepatocellular carcinoma, laryngeal cancer, lung cancer, osteosarcoma, ovarian cancer, pancreatic cancer, prostate cancer, renal carcinoma and thyroid cancer, acute lymphocytic leukemia, acute myeloid leukemia, ependymoma, Ewing's sarcoma, glioblastoma, medulloblastoma, neuroblastoma, osteosarcoma, rhabdomyosarcoma, rhabdoid cancer, and nephroblastoma (Wilm's tumor).

Inhibition of HPK1 with small molecule inhibitors has the potential for the treatment of cancer and other disorders [Hernandez, S., et. al., (2018) Cell Reports 25, 80-94].

### SUMMARY

The present disclosure provides novel indazole compounds and pharmaceutically acceptable salts as effective HPK1 inhibitors and dual activators of T cell and dendritic cell.

One embodiment of the present disclosure is a compound of Formula (I):

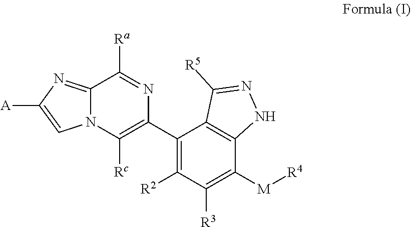

or a pharmaceutically acceptable salt, hydrate, solvate thereof, wherein A, R², R³, R⁴, R⁵, R^{a}, R^{c}, and M are as defined in the detailed descriptions.

In another embodiment, there is provided a pharmaceutical composition comprising a pharmaceutically acceptable carrier or diluent and a compound of Formula (I) or a pharmaceutically acceptable salt thereof.

In yet another embodiment, there is provided a method of treating a subject with a disease or disorder associated with modulation of HPK1 comprising: administering to the subject a therapeutically effective amount of a compound of Formula (I) or a pharmaceutically acceptable salt thereof.

### DETAILED DESCRIPTION

The following description is merely exemplary in nature and is not intended to limit the present disclosure, application, or uses.

### Definitions

The generic terms used in the present disclosure are herein defined for clarity.

This specification uses the terms “substituent”, “radical”, “group”, “moiety”, and “fragment” interchangeably.

As used herein, the term “alkenyl” refers to a straight or branched hydrocarbonyl group with at least one site of unsaturation, i.e., a carbon-carbon, sp2 double bond. In an embodiment, alkenyl has from 2 to 12 carbon atoms. In some embodiments, alkenyl is a C₂-C₁₀ alkenyl group or a C₂-C₆ alkenyl group. Examples of alkenyl group include, but are not limited to, ethylene or vinyl (—CH—CH₂), allyl (—CH₂CH═CH₂), cyclopentenyl (—C₅H₇), and 5-hexenyl (—CH₂CH₂CH₂CH₂CH═CH₂).

As used herein, the term “alkoxy” is RO— where R is alkyl. Non-limiting examples of alkoxy groups include methoxy, ethoxy and propoxy.

As used herein, the term “alkoxyalkyl” refers to an alkyl moiety substituted with an alkoxy group. Examples of alkoxyalkyl groups include methoxymethyl, methoxyethyl, methoxypropyl and ethoxyethyl.

As used herein, the term “alkoxycarbonyl” is ROC(O)—, where R is an alkyl group as defined herein. In various embodiments, R is a C₁-C₁₀ alkyl group or a C₁-C₆ alkyl group.

As used herein, the term “alkyl” refers to a straight or branched chain hydrocarbonyl group. In an embodiment, alkyl has from 1 to 12 carbon atoms. In some embodiments, alkyl is a C₁-C₁₀ alkyl group or a C₁-C₆ alkyl group. Examples of alkyl groups include, but are not limited to, methyl, ethyl, propyl, isopropyl, butyl, isobutyl, t-butyl, pentyl, hexyl, heptyl, octyl, nonyl and decyl. “lower alkyl” means alkyl having from 1 to 4 carbon atoms.

As used herein, if the term “C₁-C₆” is used, it means the number of carbon atoms is from 1 to 6. For example, C₁-C₆ alkyl means an alkyl which carbon number is any integer of from 1 to 6.

As used herein, the term “alkylamino” refers to an amino group substituted with one or more alkyl groups. “N-(alkyl)amino” is RNH— and “N,N-(alkyl)₂amino” is R²N—, where the R groups are alkyl as defined herein and are the same or different. In various embodiments, R is a C₁-C₁₀ alkyl group or a C₁-C₆ alkyl group. Examples of alkylamino groups include methylamino, ethylamino, propylamino, butylamino, dimethylamino, diethylamino, and methylethylamino.

As used herein, the term “alkylaminoalkyl” refers to an alkyl moiety substituted with an alkylamino group, wherein alkylamino is as defined herein. Examples of alkylaminoalkyl groups include methylaminomethyl and ethylaminomethyl.

As used herein, the term “alkynyl” refers to a straight or branched carbon-chain group with at least one site of unsaturation, i.e., a carbon-carbon, sp triple bond. In an embodiment, alkynyl has from 2 to 12 carbon atoms. In some embodiments, alkynyl is a C₂-C₁₀ alkynyl group or a C₂-C₆ alkynyl group. Examples of alkynyl groups include acetylenic (—C≡CH) and propargyl (—CH₂C≡CH).

As used herein, the term “aryl” refers to any monocyclic or bicyclic carbon ring of up to 7 atoms in each ring, wherein at least one ring is aromatic, or an aromatic ring system of 5 to 14 carbon atoms which includes a carbocyclic aromatic group fused with a 5- or 6-membered cycloalkyl group. Representative examples of aryl groups include, but are not limited to, phenyl, tolyl, xylyl, naphthyl, tetrahydronaphthyl, anthracenyl, fluorenyl, indenyl, azulenyl and indanyl. A carbocyclic aromatic group can be unsubstituted or optionally substituted.

As used herein, the term “cycloalkyl” is a hydrocarbyl group containing at least one saturated or partially unsaturated ring structure, and attached via a ring carbon. In various embodiments, it refers to a saturated or a partially unsaturated C₃-C₁₂ cyclic moiety, examples of which include cyclopropyl, cyclobutyl, cyclopentyl, cyclopentenyl, cyclohexyl, cyclohexenyl, cycloheptyl and cyclooctyl. “Cycloalkyloxy” is RO—, where R is cycloalkyl.

As used herein, the terms “halogen” and “halo” refers to chloro (—Cl), bromo (—Br), fluoro (—F) or iodo (—I). “Haloalkoxy” refers to an alkoxy group substituted with one or more halo groups and examples of haloalkoxy groups include, but are not limited to, —OCF₃, —OCHF₂ and —OCH₂F. “Haloalkoxyalkyl” refers to an alkyl moiety substituted with a haloalkoxy group, wherein haloalkoxy is as defined herein. Examples of haloalkoxyalkyl groups include trifluoromethoxymethyl, trifluoroethoxymethyl and trifluoromethoxyethyl. “Haloalkyl” refers to an alkyl moiety substituted with one or more halo groups. Examples of haloalkyl groups include —CF₃, —CHF₂, and —CH₂F.

As used herein, the term “heteroalkyl” refers to a straight- or branched-chain alkyl group having from 2 to 14 carbons (in some embodiments, 2 to 10 carbons) in the chain, one or more of which has been replaced by a heteroatom selected from S, O, P and N. Exemplary heteroalkyls include alkyl ethers, secondary and tertiary alkyl amines, amides, alkyl sulfides, and the like.

As used herein, the term “heterocyclyl” includes the heteroaryls defined below and refers to a saturated or partially unsaturated monocyclic, bicyclic or tricyclic group of 2 to 14 ring-carbon atoms and, in addition to ring-carbon atoms, 1 to 4 heteroatoms selected from P, N, O and S. In various embodiments the heterocyclic group is attached to another moiety through carbon or through a heteroatom, and is optionally substituted on carbon or a heteroatom. Examples of heterocyclyl include azetidinyl, benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrahydrothiopyranyl, tetrahydroisoquinolinyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof. “Heterocyclyloxy” is RO—, where R is heterocyclyl. “Heterocyclylthio” is RS—, where R is heterocyclyl.

As used herein, the term “3- or 4-membered heterocyclyl” refers to a monocyclic ring having 3 or 4 ring atoms wherein at least one ring atom is heteroatom selected from the group consisting of N, O and S. Non-limiting examples of 3- or 4-membered heterocyclyl include aziridinyl, 2H-azirinyl, oxiranyl, thiiranyl, azetidinyl, 2,3-dihyroazetyl, azetyl, 1,3-diazetidinyl, oxetanyl, 2H-oxetyl, thietanyl, and 2H-thietyl.

As used herein, the term “heteroaryl” refers to a monocyclic, bicyclic or tricyclic ring having up to 7 atoms in each ring, wherein at least one ring is aromatic and contains from 1 to 4 heteroatoms in the ring selected from the group consisting of N, O and S. Non-limiting examples of heteroaryl include pyridyl, thienyl, furanyl, pyrimidyl, imidazolyl, pyranyl, pyrazolyl, thiazolyl, thiadiazolyl, isothiazolyl, oxazolyl, isoxazoyl, pyrrolyl, pyridazinyl, pyrazinyl, quinolinyl, isoquinolinyl, benzofuranyl, dibenzofuranyl, dibenzothiophenyl, benzothienyl, indolyl, benzothiazolyl, benzooxazolyl, benzimidazolyl, isoindolyl, benzotriazolyl, purinyl, thianaphthenyl and pyrazinyl. Attachment of heteroaryl can occur via an aromatic ring, or, if heteroaryl is bicyclic or tricyclic and one of the rings is not aromatic or contains no heteroatoms, through a non-aromatic ring or a ring containing no heteroatoms. “Heteroaryl” is also understood to include the N-oxide derivative of any nitrogen containing heteroaryl. “Heteroaryloxy” is RO—, where R is heteroaryl.

As used herein, the term “hydroxyalkoxy” refers to an alkoxy group substituted with a hydroxyl group (—OH), wherein alkoxy is as defined herein. An example of hydroxyalkoxy is hydroxyethoxy.

As used herein, the term “hydroxyalkyl” refers to a linear or branched monovalent C₁-C₁₀ hydrocarbon group substituted with at least one hydroxy group and examples of hydroxyalkyl groups include, but are not limited to, hydroxymethyl, hydroxyethyl, hydroxypropyl and hydroxybutyl.

As used herein, the term “pharmaceutically acceptable” means suitable for use in pharmaceutical preparations, generally considered as safe for such use, officially approved by a regulatory agency of a national or state government for such use, or being listed in the U.S. Pharmacopoeia or other generally recognized pharmacopoeia for use in animals, and more particularly in humans.

As used herein, the term “pharmaceutically acceptable carrier” refers to a diluent, adjuvant, excipient, or carrier, or other ingredient which is pharmaceutically acceptable and with which a compound of the invention is administered.

As used herein, the term “pharmaceutically acceptable salt” refers to a salt which may enhance desired pharmacological activity. Examples of pharmaceutically acceptable salts include acid addition salts formed with inorganic or organic acids, metal salts and amine salts. Examples of acid addition salts formed with inorganic acids include salts with hydrochloric acid, hydrobromic acid, sulfuric acid, nitric acid and phosphoric acid. Examples of acid addition salts formed with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxy-benzoyl)-benzoic acid, cinnamic acid, mandelic acid, methanesulfonic acid, ethanesulfonic acid, 1,2-ethanedisulfonic acid, 2-hydroxyethane-sulfonic acid, benzenesulfonic acid, p chlorobenzenesulfonic acid, 2-naphthalenesulfonic acid, p-toluenesulfonic acid, camphorsulfonic acid, 4-methyl-bicyclo[2.2.2]oct-2-ene1-carboxylic acid, gluco-heptonic acid, 4,4′-methylenebis(3-hydroxy-2-naphthoic) acid, 3-phenylpropionic acid, trimethyl-acetic acid, tertiary butylacetic acid, lauryl sulfuric acid, gluconic acid, glutamic acid, hydroxy-naphthoic acids, salicylic acid, stearic acid and muconic acid. Examples of metal salts include salts with sodium, potassium, calcium, magnesium, aluminum, iron, and zinc ions. Examples of amine salts include salts with ammonia and organic nitrogenous bases strong enough to form salts with carboxylic acids.

As used herein, the term “substituted” means any of above groups (i.e., alkyl, aryl, heteroaryl, heterocycle or cycloalkyl) wherein at least one hydrogen atom of the moiety being substituted is replaced with a substituent. In one embodiment, each carbon atom of the group being substituted is substituted with no more than two substituents. In another embodiment, each carbon atom of the group being substituted is substituted with no more than one substituent. In the case of a keto substituent, two hydrogen atoms are replaced with an oxygen which is attached to the carbon via a double bond. Unless specifically defined, substituents include halo, hydroxyl, (lower) alkyl, haloalkyl, mono- or di-alkylamino, aryl, heterocycle, —NO₂, B(OH)₂, BPin, —NRₐR_{b}, —NRC(═O)R_{b}, —NR₂C(═O)NRₐR_{b}, —NRₐC(═O)OR_{b}, —NRₐSO₂R_{b}, —ORₐ, —CN, —C(═O)Rₐ, —C(═O)ORₐ, —C(═O)NRₐR_{b}, —OC(═O)Rₐ, —OC(═O)ORₐ, —OC(═O)NRₐR_{b}, —NRₐSO₂R_{b}, —PO₃Rₐ, —PO(ORₐ)(OR_{b}), —SO₂Rₐ, —S(O)Rₐ, —SO(N)Rₐ (e.g., sulfoximine), —(Rₐ)S═NR_{b} (e.g., sulfilimine) and —SRₐ, wherein Rₐ and R_{b} are the same or different and independently —H, halo, amino, alkyl, haloalkyl, aryl or heterocycle, or wherein Rₐ and R_{b} taken together with the nitrogen atom to which they are attached form a heterocycle. Rₐ and R_{b} may be in the plural based on atoms which those are attached to.

As used herein, the term “therapeutically effective amount” means when applied to a compound of the invention is intended to denote an amount of the compound that is sufficient to ameliorate, palliate, stabilize, reverse, slow or delay the progression of a disorder or disease state, or of a symptom of the disorder or disease. In an embodiment, the method of the present invention provides for administration of combinations of compounds. In such instances, the “therapeutically effective amount” is the amount of a compound of the present invention in the combination sufficient to cause the intended biological effect.

As used herein, the term “treatment” or “treating” as used herein means ameliorating or reversing the progress or severity of a disease or disorder, or ameliorating or reversing one or more symptoms or side effects of such disease or disorder. “Treatment” or “treating”, as used herein, also means to inhibit or block, as in retard, arrest, restrain, impede or obstruct, the progress of a system, condition or state of a disease or disorder. For purposes of this invention, “treatment” or “treating” further means an approach for obtaining beneficial or desired clinical results, where “beneficial or desired clinical results” include, without limitation, alleviation of a symptom, diminishment of the extent of a disorder or disease, stabilized (i.e., not worsening) disease or disorder state, delay or slowing of a disease or disorder state, amelioration or palliation of a disease or disorder state, and remission of a disease or disorder, whether partial or total.

### Compounds

The present disclosure provides a compound of Formula (I):

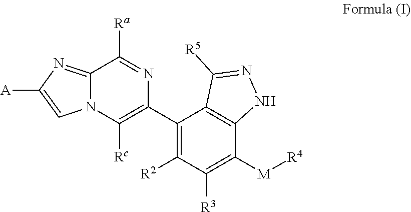

or a pharmaceutically acceptable salt, hydrate, or solvate thereof, wherein:
A is —NH—C(O)-L-R¹, —NH—R¹, or —R¹;
L is a bond, —NH—, —O—, or —CH₂—;
R¹ is —H, -D, —CD₃, alkyl, alkenyl, alkynyl, cycloalkyl, aryl, heteroaryl, heterocyclyl, halo, hydroxyl, —CD₂OH, —CN, —NO₂, haloalkyl, —C(O)R⁹, —C(O)OR⁹, —C(O)NR¹⁰R¹¹, —OR⁹, —OC(O)R⁹, —OC(O)NR¹⁰R¹¹, —SR⁹, —S(O)R⁹, —S(O)₂R⁹, —S(O)(═NH)R¹⁰, —S(O)₂NR¹⁰R¹¹, —NR¹⁰R¹¹, —N(R⁶) NR¹⁰R¹¹, —N(R⁶)OR⁹, —N(R⁶)C(O)R⁹, —N(R⁶)C(O)R⁹, —N(R⁶)C(O)OR⁹, —N(R⁶)C(O)NR¹⁰R¹¹, —N(R⁶)S(O)₂R⁹, —N(R⁶)S(O)₂NR¹⁰R¹¹, or —P(O)R¹²R¹³, wherein the alkyl, alkenyl, alkynyl, cycloalkyl, aryl, heteroaryl, or heterocyclyl is optionally substituted with one or more substituents independently selected from R⁷.
R² is —H, -D, —CD₃, alkyl, alkenyl, alkynyl, cycloalkyl, aryl, heteroaryl, heterocyclyl, halo, hydroxyl, —CD₂OH, —CN, —NO₂, haloalkyl, —C(O)R⁹, —C(O)OR⁹, —C(O)NR¹⁰R¹¹, —OR⁹, —OC(O)R⁹, —OC(O)NR¹⁰R¹¹, —SR⁹, —S(O)R⁹, —S(O)₂R⁹, —S(O)(═NH)R¹⁰, —S(O)₂NR¹⁰R¹¹, —NR¹⁰R¹¹, —N(R⁶) NR¹⁰R¹¹, —N(R⁶) OR⁹, —N(R⁶)C(O)R⁹, —N(R⁶)C(O)R⁹, —N(R⁶)C(O)OR⁹, —N(R⁶)C(O)NR¹⁰R¹¹, —N(R⁶)S(O)₂R⁹, —N(R⁶)S(O)₂NR¹⁰R¹¹, or —P(O)R¹²R¹³, wherein the alkyl, alkenyl, alkynyl, cycloalkyl, aryl, heteroaryl, or heterocyclyl is optionally substituted with one or more groups selected from the group consisting of halo, hydroxyl, alkyl, alkenyl, alkynyl, haloalkyl, hydroxyalkyl, —CN, —NO₂, —NR¹⁰R¹¹, —NR¹⁰C(═O)R⁹, —NR¹⁰C(═O)NR¹⁰R¹¹, —NR¹⁰C(═O)OR⁹, —OR⁹, —C(═O)R⁹, —C(═O)OR⁹, —C(═O)NR¹⁰R¹¹, —OC(═O)R⁹, —OC(═O)OR⁹, and —OC(═O)NR¹⁰R¹¹;
R³ is —H, -D, —CD₃, alkyl, alkenyl, alkynyl, cycloalkyl, aryl, heteroaryl, heterocyclyl, halo, cyano, hydroxy, —CH₂OH, —CD₂OH, —OH, —CN, —NO₂, haloalkyl, —C(O)R⁹, —C(O)OR⁹, —C(O)NR¹⁰R¹¹, —OR⁹, —OC(O)R⁹, —OC(O)NR¹⁰R¹¹, —SR⁹, —S(O)R⁹, —S(O)₂R⁹, —S(O)(═NH)R¹⁰, —S(O)₂NR¹⁰R¹¹, —NR¹⁰R¹¹, —N(R⁶) NR¹⁰R¹¹, —N(R⁶) OR⁹, —N(R⁶)C(O)R⁹, —N(R⁶)C(O)OR⁹, —N(R⁶)C(O)NR¹⁰R¹¹, —N(R⁶)S(O)₂R⁹, N(R⁶)S(O)₂NR¹⁰R¹¹, or —P(O)R¹²R¹³;
M is a bond, —O—, —S—, —CHR⁶—, or —NR⁶—;
R⁴ is —H, -D, alkyl, alkenyl, alkynyl, cycloalkyl, aryl, heteroaryl, heterocyclyl, halo, cyano, hydroxy, —C(O)R⁹, —C(O)OR⁹, —C(O)NR¹⁰R¹¹, —S(O)₂R⁹, —S(O)(═NH)R¹⁰, —S(O)₂NR¹⁰R¹¹, or —P(O)R¹²R¹³, wherein the alkyl, alkenyl, alkynyl, cycloalkyl, aryl, heteroaryl, or heterocyclyl is optionally substituted with one or more groups selected from the group consisting of halo, hydroxyl, alkyl, alkenyl, alkynyl, haloalkyl, hydroxyalkyl, —CN, —CD₃, —NO₂, —NR¹⁰R¹¹, —NR¹⁰C(═O)R⁹, —NR¹⁰C(═O)NR¹⁰R¹¹, —NR¹⁰C(═O)OR⁹, —NR¹⁰S(O)₂R⁹, —OR⁹, —C(═O)R⁹, —C(═O)OR⁹, —C(═O)NR¹⁰R¹¹, —OC(═O)R⁹, —OC(═O)OR⁹, and —OC(═O)NR¹⁰R¹¹;
R⁵ is —H, -D, —CD₃, alkyl, alkenyl, alkynyl, cycloalkyl, halo, hydroxyl, —CH₂OH, —CD₂OH, —CN, or haloalkyl; R⁶ is —H, C₁₋₆ alkyl, or cycloalkyl;
R⁷ is alkyl, alkenyl, alkynyl, cycloalkyl, aryl, heteroaryl, heterocyclyl, halo, oxo, cyano, hydroxy, —C(O)R⁹, —C(O)OR⁹, —C(O)NR¹⁰R¹¹, —OR⁹, —OC(O)R⁹, —OC(O)NR¹⁰R¹¹, —SR⁹, —S(O)R⁹, —S(O)₂R⁹, —S(O)(═NH)R¹⁰, —S(O)₂NR¹⁰R¹¹, —NR¹⁰R¹¹, —N(R⁶) NR¹⁰R¹¹, —N(R⁶) OR⁹, —N(R⁶)C(O)R⁹, —N(R⁶)C(O)OR⁹, —N(R⁶)C(O)NR¹⁰R¹¹, —N(R⁶)S(O)₂R⁹, —N(R⁶)S(O)₂NR¹⁰R¹¹, or —P(O)R¹²R¹³;
R⁹ is —H, alkyl, haloalkyl, alkenyl, alkynyl, cycloalkyl, aryl, heteroaryl, or heterocyclyl;
Each R¹⁰ and R¹¹ is independently —H, alkyl, alkenyl, alkynyl, cycloalkyl, aryl, heteroaryl, or heterocyclyl, or R¹⁰ and R¹¹ are taken together with the nitrogen atom to which they are attached to form a 4- to 12-membered heterocyclyl optionally substituted with one or more groups selected from the group consisting of halo, hydroxyl, alkyl, alkenyl, alkynyl, haloalkyl, hydroxyalkyl, —CN, —NO₂, —NR¹⁰R¹¹, —NR¹⁰C(═O)R⁹, —NR¹⁰C(═O)NR¹⁰R¹¹, —NR¹⁰C(═O)OR⁹, —OR⁹, —C(═O)R⁹, —C(═O)OR⁹, —C(═O)NR¹⁰R¹¹, —OC(═O)R⁹, —OC(═O)OR⁹, and —OC(═O)NR¹⁰R¹¹;
Each R¹² and R₁₃ is independently alkyl, alkoxyl, cycloalkyl, aryl, heteroaryl, or heterocyclyl, or R¹² and R¹³ are taken together with the phosphorus atom to which they are attached to form a 4- to 8-membered heterocyclyl optionally substituted with one or more groups selected from the group consisting of halo, hydroxyl, alkyl, alkenyl, alkynyl, haloalkyl, hydroxyalkyl, —CN, —NO₂, —NR¹⁰R¹¹, —NR¹⁰C(═O)R⁹, —NR¹⁰C(═O)NR¹⁰R¹¹, —NR¹⁰C(═O)OR⁹, —OR⁹, —C(═O)R⁹, —C(═O)OR⁹, —C(═O)NR¹⁰R¹¹, —OC(═O)R⁹, —OC(═O)OR⁹, and —OC(═O)NR¹⁰R¹¹; and
Each of R^{a} and R^{c} is independently —H, -D, halo, haloalkyl, —CN, —OR⁹ or —NR¹⁰R¹¹.

In various embodiments of Formulae (I)-(VI), alkyl is C₁₋₆ alkyl; alkenyl is C₂₋₆ alkenyl; akylnyl is C₂₋₆ alkynyl, and/or cycloalkyl is C₃-8 cycloalkyl.

In some embodiments, each of R² and R³ is independently —H, -D, halo, alkoxy, alkylthio, haloalkyl, alkyl, hydroxyalkyl, —COOH, —C(O) OCH₃, or —CN.

In some embodiments, R² is alkyl, haloalkyl, hydroxyalkyl, alkoxy, halo, —COOH, —C(O) OCH₃, or —CN; and R³ is halo or alkyl.

In some embodiments, R⁵ is —H, halo, or alkyl.

In some embodiments, each of R^{a} and R^{c} is independently —H, halo, or alkyl.

In some embodiments, M is a bond, —O—, —CHR⁶—, or —NR⁶—; and R⁴ is —H, -D, C₁₋₆ alkyl, C₂₋₆ alkenyl, C₂₋₆ alkynyl, cycloalkyl, aryl, heteroaryl, heterocyclyl, halo, cyano, hydroxy, —C(O)R⁹, —NHC(O)R⁹, —C(O)NR¹⁰R¹¹, —S(O)₂R⁹, —S(O)(═NH)R¹⁰, or —S(O)₂NR¹⁰R¹¹, wherein the C₁₋₆ alkyl, C₂₋₆ alkenyl, C₂₋₆ alkynyl, cycloalkyl, aryl, heteroaryl, or heterocyclyl is optionally substituted with one or more groups selected from the group consisting of halo, hydroxy, alkyl, alkenyl, alkynyl, haloalkyl, hydroxyalkyl, —C(O)CH₃, —CN, —CD₃, —NR¹⁰R¹¹, —NR¹⁰S(O)₂R⁹, and —NR¹⁰C(═O)R⁹.

In some embodiments, M-R⁴ is selected from the group consisting of:

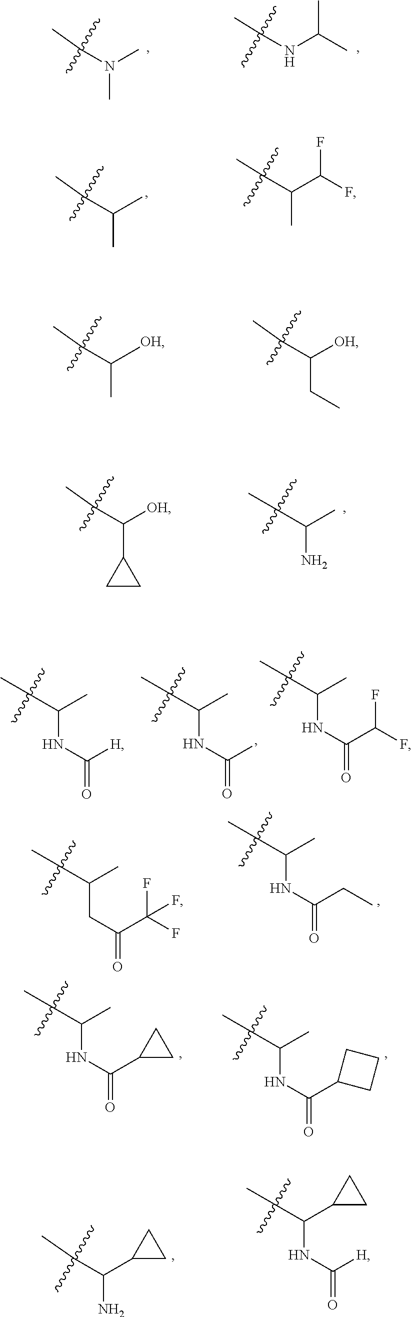

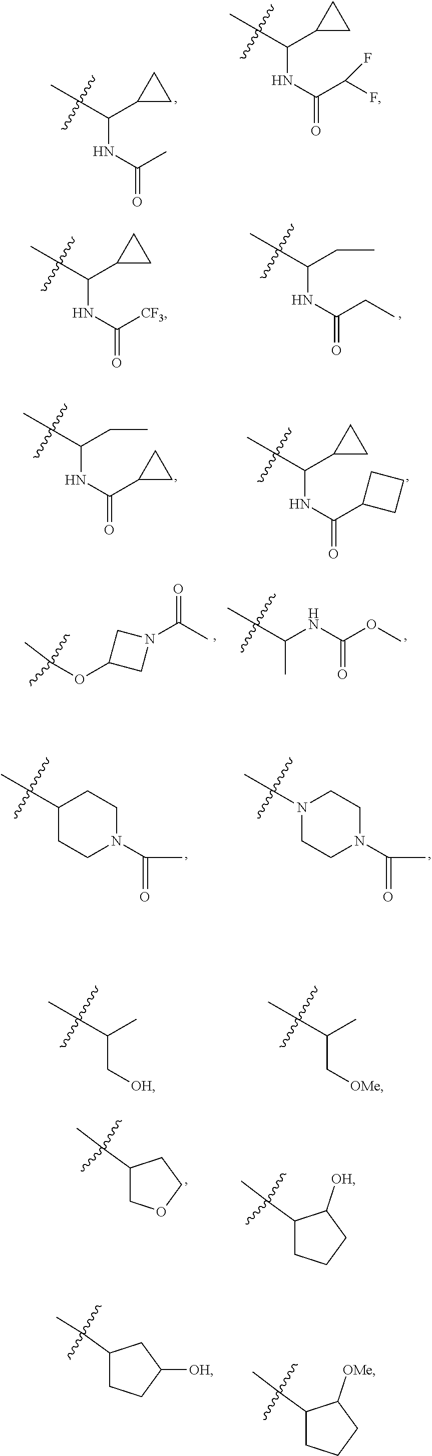

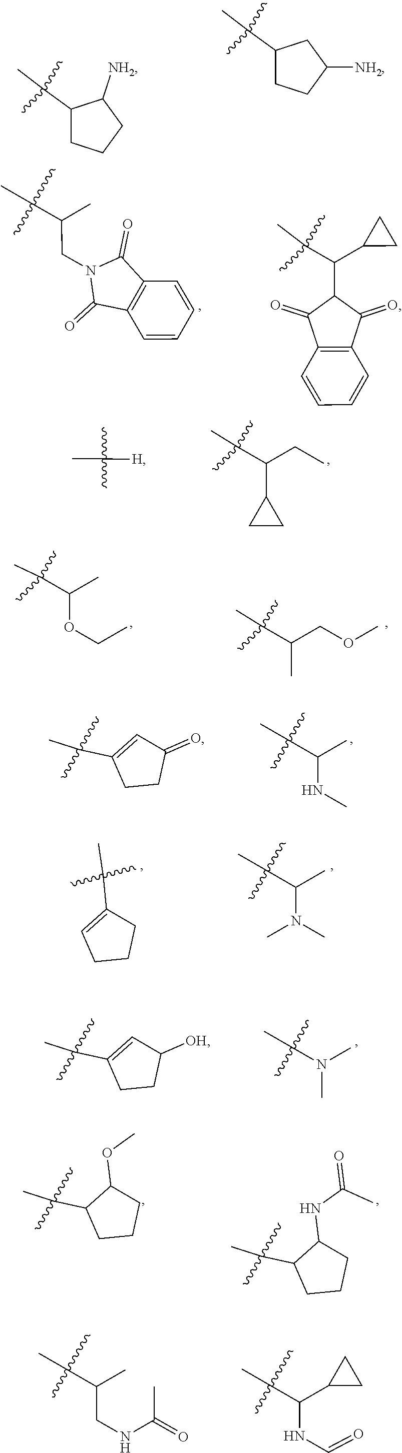

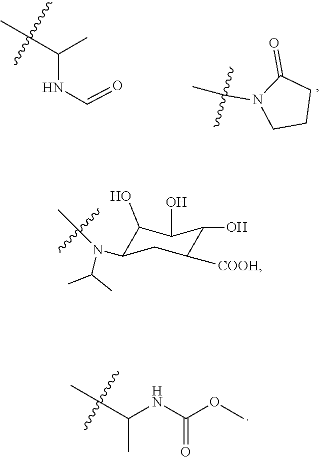

In another embodiment, there is provided a compound of Formula (II):

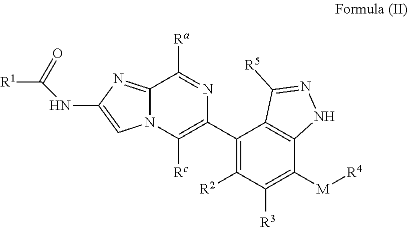

wherein R², R³, R⁴, R⁵, R^{a}, R^{c}, and M are as defined above for Formula (I), and R¹ is cyclopropyl which is optionally substituted with one or two R⁷.

In some embodiments, R¹ is selected from the group consisting of:

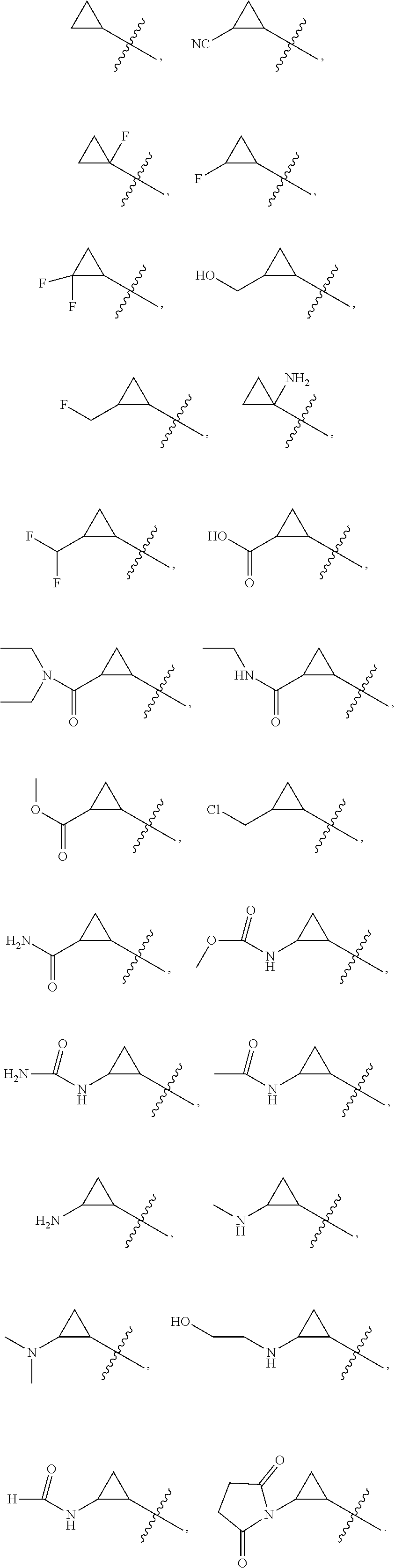

In some embodiments, each of R² and R³ is independently —H, -D, halo, alkoxy, alkylthio, haloalkyl, alkyl, hydroxyalkyl, —COOH, —C(O) OCH₃, or —CN; M is a bond, —O—, —CHR⁶—, or —NR⁶—; R⁴ is —H, -D, alkyl, halo, cyano, hydroxy, dialkylamino, haloalkyl, hydroxyalkyl, amino, cycloalkyl, aryl, heteroaryl, heterocyclyl, —C(O)R⁹, —NHC(O)R⁹, or —C(O)NR¹⁰R¹¹, wherein the cycloalkyl, aryl, heteroaryl, or heterocyclyl is optionally substituted with one or more groups selected from the group consisting of hydroxy, amino, —COOH, and —C(O)CH₃; R⁵ is —H, halo, or alkyl; and each of R^{a} and R^{c} is independently —H, halo, or alkyl.

Non-limiting, exemplary compounds of Formula (II) include the compounds of Examples 1-42, 44-56, 59, 62, 63, 67-73, 75-78, 85-88, 94, 96-99, 104, 107, 115, 118, 127, 128, 132, 133, 136, 137, 141-143, 146, 154, 156, 160, 164, and 165 of Table 1.

In some embodiments, R¹ is

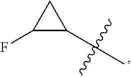 [SMILES: *C1CC1F]

and non-limiting, exemplary compounds include the compounds of Examples 1-19, 21-25, 27, 28, 30, 31, 33-36, 40-42, 44, 45, 48-50, 53-56, 59, 62, 63, 67-73, 75-77, 85-87, 96-99, 107, 115, 127-128, 132, 133, 136, 137, 141-143, 146, and 165 of Table 1.

In some embodiments, R¹ is selected from the group consisting of:

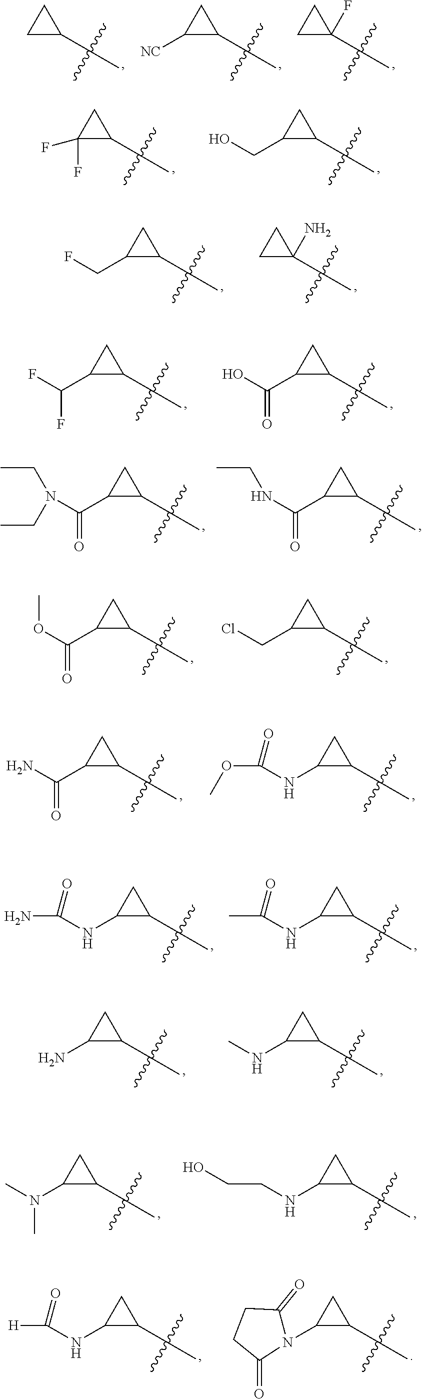

and non-limiting, exemplary compounds include the compounds of Examples 20, 26, 29, 32, 37-39, 46, 47, 51, 52, 78, 94, 104, 118, and 154 of Table 1.

In another embodiment, there is provided a compound of Formula (III):

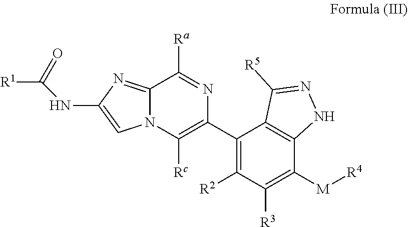

wherein R², R³, R⁴, R⁵, R^{a}, R^{c}, and M are as defined above for Formula (I), and R¹ is alkyl, alkenyl, alkynyl, halo, hydroxyl, —CD₂OH, —CN, —NO₂, haloalkyl, —C(O)R⁹, —C(O)OR⁹, —C(O)NR¹⁰R¹¹, —OR⁹, —OC(O)R⁹, —OC(O)NR¹⁰R¹¹, —SR⁹, —S(O)R⁹, —S(O)₂R⁹, —S(O)(═NH)R¹⁰, —S(O)₂NR¹⁰R¹¹, —NR¹⁰R¹¹, —N(R⁶) NR¹⁰R¹¹, —N(R⁶) OR⁹, —N(R⁶)C(O)R⁹, —N(R⁶)C(O)R⁹, —N(R⁶)C(O)OR⁹, —N(R⁶)C(O)NR¹⁰R¹¹, —N(R⁶)S(O)₂R⁹, —N(R⁶)S(O)₂NR¹⁰R¹¹, or —P(O)R¹²R¹³, wherein the alkyl, alkenyl, or alkynyl is optionally substituted with one or more substituents independently selected from R⁷.

In some embodiments, R¹ is selected from the group consisting of fluoromethyl, difluoromethyl, trifluoromethyl, methoxymethyl, hydroxyethyl, hydroxypropyl, methyl, ethyl, ethyl, butyl, isopropyl, isobutyl, hydroxymethyl, hydroxyethyl, aminomethyl, aminoethyl, dimethylaminomethyl, methiomethyl, methylamino, ethylamino, isopropylamino, methoxy, ethoxy, isopropyloxy,

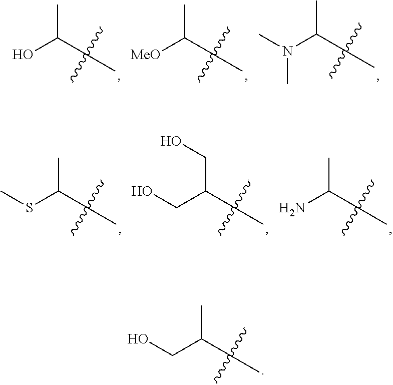

In some embodiments, each of R² and R³ is independently —H, -D, halo, alkoxy, alkylthio, haloalkyl, alkyl, hydroxyalkyl, —COOH, —C(O) OCH₃, or —CN; M is a bond, —O—, —CHR⁶—, or —NR⁶—; R⁴ is —H, -D, alkyl, halo, cyano, hydroxy, dialkylamino, haloalkyl, hydroxyalkyl, amino, cycloalkyl, aryl, heteroaryl, heterocyclyl, —C(O)R⁹, —NHC(O)R⁹, or —C(O)NR¹⁰R¹¹, wherein the cycloalkyl, aryl, heteroaryl, or heterocyclyl is optionally substituted with one or more groups selected from the group consisting of hydroxy, amino, —COOH, and —C(O)CH₃; R⁵ is —H, halo, or alkyl; and each of R^{a} and R^{c} is independently —H, halo, or alkyl.

Non-limiting, exemplary compounds of Formula (III) include the compounds of Examples 43, 61, 64, 66, 79-83, 95, 100, 102, 103, 105, 106, 109, 110, 112-114, 117, 119, 121, 122, 129, 130, 134, 135, 138-140, 145, 147-153, 155, 157-159, and 161-163 of Table 1.

In another embodiment, there is provided a compound of Formula (IV):

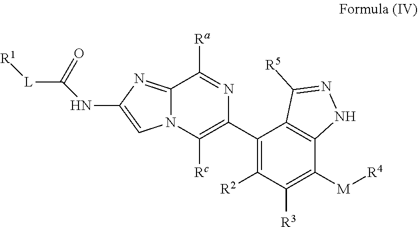

wherein R², R³, R⁴, R⁵, R^{a}, R^{c}, L, and M are as defined above for Formula (I), and R¹ is cycloalkyl, aryl, heteroaryl, or heterocyclyl, wherein the cycloalkyl, aryl, heteroaryl, or heterocyclyl is optionally substituted with one or more substituents independently selected from R⁷.

In some embodiments, R¹ is selected from the group consisting of:

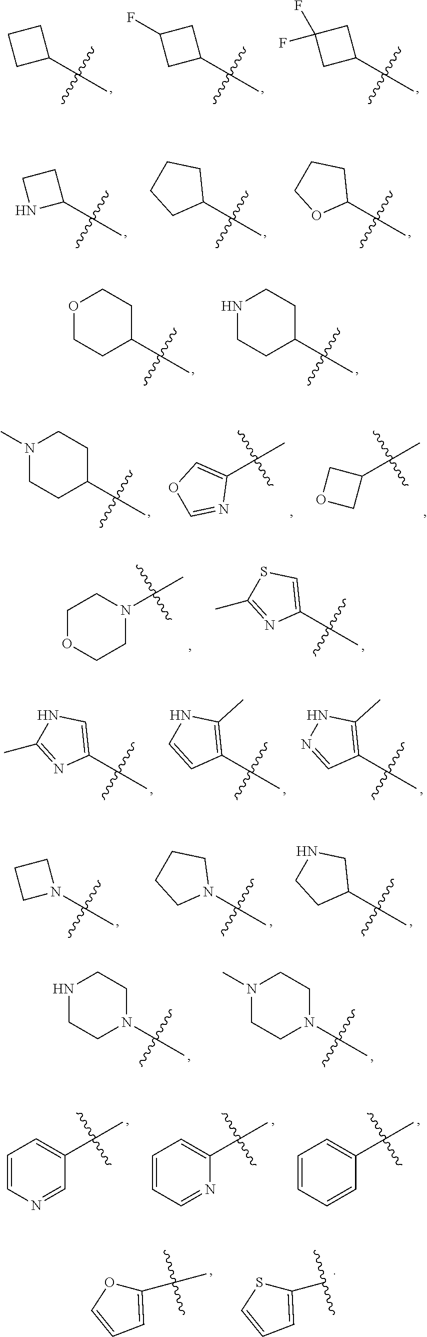

In some embodiments, each of R² and R³ is independently —H, -D, halo, alkoxy, alkylthio, haloalkyl, alkyl, hydroxyalkyl, —COOH, —C(O) OCH₃, or —CN; M is a bond, —O—, —CHR⁶—, or —NR⁶—; R⁴ is —H, -D, alkyl, halo, cyano, hydroxy, dialkylamino, haloalkyl, hydroxyalkyl, amino, cycloalkyl, aryl, heteroaryl, heterocyclyl, —C(O)R⁹, —NHC(O)R⁹, or —C(O)NR¹⁰R¹¹, wherein the cycloalkyl, aryl, heteroaryl, or heterocyclyl is optionally substituted with one or more groups selected from the group consisting of hydroxy, amino, —COOH, and —C(O)CH₃; R⁵ is —H, halo, or alkyl; and each of R^{a} and R^{c} is independently —H, halo, or alkyl.

Non-limiting, exemplary compounds of Formula (IV) include the compounds of Examples 57, 58, 60, 74, 84, 89, 90, 92, 93, 108, 111, 116, 120, 123-126, and 131 of Table 1.

In another embodiment, there is provided a compound of Formula (V):

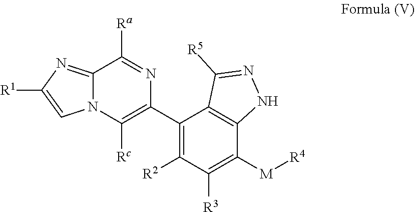

wherein R¹, R², R³, R⁴, R⁵, R^{a}, R^{c}, and M are as defined above for Formula (I).

In some embodiments, R¹ is selected from the group consisting of:

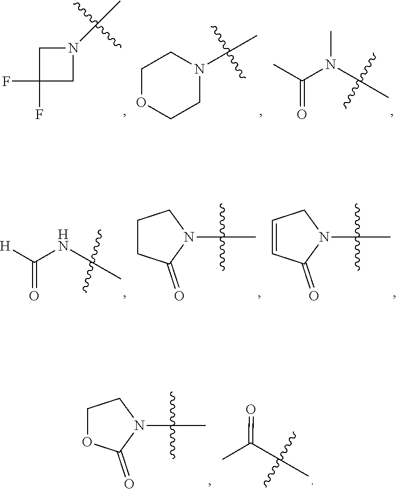

In some embodiments, each of R² and R³ is independently —H, -D, halo, alkoxy, alkylthio, haloalkyl, alkyl, hydroxyalkyl, —COOH, —C(O) OCH₃, or —CN; M is a bond, —O—, —CHR⁶—, or —NR⁶—; R⁴ is —H, -D, alkyl, halo, cyano, hydroxy, dialkylamino, haloalkyl, hydroxyalkyl, amino, cycloalkyl, aryl, heteroaryl, heterocyclyl, —C(O)R′, —NHC(O)R⁹, or —C(O)NR¹⁰R¹¹, wherein the cycloalkyl, aryl, heteroaryl, or heterocyclyl is optionally substituted with one or more groups selected from the group consisting of hydroxy, amino, —COOH, and —C(O)CH₃; R⁵ is —H, halo, or alkyl; and each of R^{a} and R^{c} is independently —H, halo, or alkyl.

Non-limiting, exemplary compounds of Formula (V) include the compounds of Examples 65 and 91 of Table 1.

In another embodiment, there is provided a compound of Formula (VI):

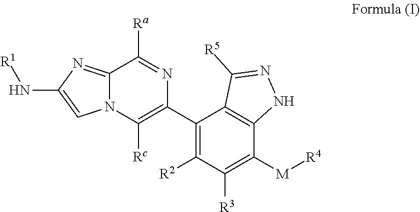

wherein R¹, R², R³, R⁴, R⁵, R^{a}, R^{c}, and M are as defined above for Formula (I).

In some embodiments, R¹ is selected from the group consisting of:

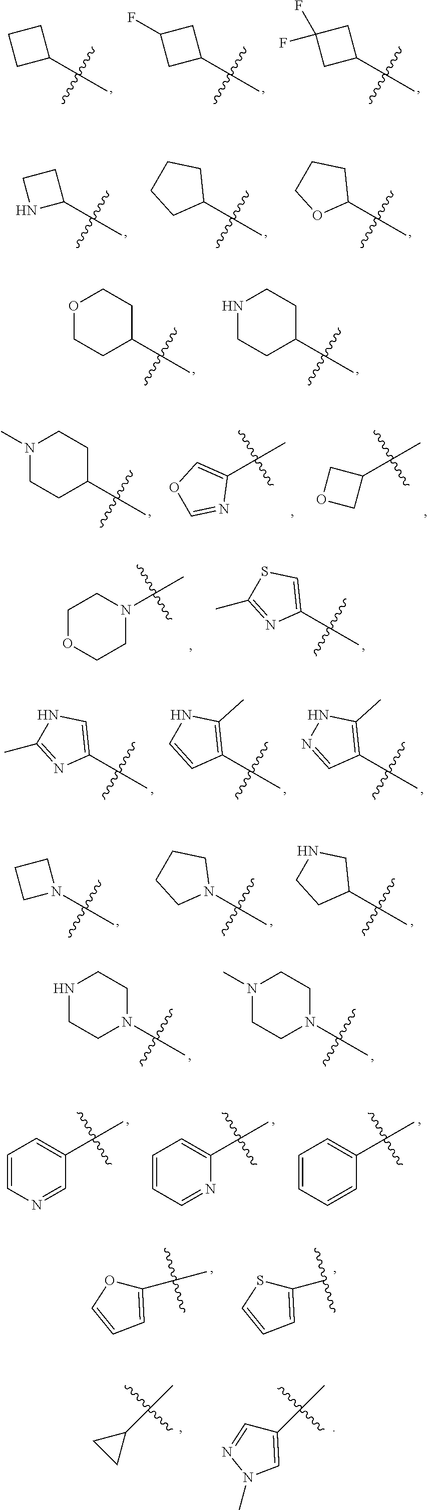

In some embodiments, each of R² and R³ is independently —H, -D, halo, alkoxy, alkylthio, haloalkyl, alkyl, hydroxyalkyl, —COOH, —C(O) OCH₃, or —CN; M is a bond, —O—, —CHR⁶—, or —NR⁶—; R⁴ is —H, -D, alkyl, halo, cyano, hydroxy, dialkylamino, haloalkyl, hydroxyalkyl, amino, cycloalkyl, aryl, heteroaryl, heterocyclyl, —C(O)R⁹, —NHC(O)R⁹, or —C(O)NR¹⁰R¹¹, wherein the cycloalkyl, aryl, heteroaryl, or heterocyclyl is optionally substituted with one or more groups selected from the group consisting of hydroxy, amino, —COOH, and —C(O)CH₃; R⁵ is —H, halo, or alkyl; and each of R^{a} and R^{c} is independently —H, halo, or alkyl.

Non-limiting, exemplary compounds of Formula (VI) include the compounds of Examples 101 of Table 1.

In an embodiment, there is provided a pharmaceutical composition comprising a pharmaceutically acceptable carrier or diluent and a compound of Formulae (I)-(VI) or a pharmaceutically acceptable salt thereof.

### Medical Uses and Methods of Treatment Using the Compounds

The present disclosure provides a method of treating a subject with a disease or disorder associated with modulation of HPK1 comprising: administering to the subject in need thereof a therapeutically effective amount of the compound of claim 1 or a pharmaceutically acceptable salt thereof. In some embodiments, the disease or disorder associated with modulation of HPK1 is cancer, metastasis, inflammation, or auto-immune pathogenesis.

In some other embodiments, the disease is cancer, metastasis, inflammation or auto-immune disease. In particular embodiments, the cancer is selected from the group consisting of carcinomas, melanomas, blastomas, sarcomas, lymphomas and leukemias, including without limitation, bladder carcinoma, brain tumors, breast cancer, cervical cancer, colorectal cancer, esophageal cancer, endometrial cancer, hepatocellular carcinoma, laryngeal cancer, lung cancer, osteosarcoma, ovarian cancer, pancreatic cancer, prostate cancer, renal carcinoma and thyroid cancer, acute lymphocytic leukemia, acute myeloid leukemia, ependymoma, Ewing's sarcoma, glioblastoma, medulloblastoma, neuroblastoma, osteosarcoma, rhabdomyosarcoma, rhabdoid cancer, and nephroblastoma (Wilm's tumor).

In another embodiment, there is provided the use of a compound of Formula (I) or a pharmaceutically acceptable salt thereof for the manufacture of a medicament for inhibiting HPK1 activity in a subject in need of inhibition of HPK1 activity. In some embodiments, the use includes treatment of cancer.

Suitable subjects to be treated according to the present disclosure include mammalian subjects. Mammals according to the present disclosure include, but are not limited to, human, canine, feline, bovine, caprine, equine, ovine, porcine, rodents, lagomorphs, primates, and the like, and encompass mammals in utero. Subjects may be of either gender and at any stage of development. In one embodiment, the suitable subject to be treated according to the present disclosure is human.

The compounds of the present disclosure are generally administered in a therapeutically effective amount. The compounds of the present disclosure can be administered by any suitable route in the form of a pharmaceutical composition adapted to such a route, and in a dose effective for the treatment intended. An effective dosage is typically in the range of about 0.01 to about 1000 mg per kg body weight per day, preferably about 0.01 to about 500 mg/kg/day, in single or divided doses. Depending on age, species and disease or condition being treated, dosage levels below the lower limit of this range may be suitable. In other cases, still larger doses may be used without harmful side effects. Larger doses may also be divided into several smaller doses, for administration throughout the day. Methods for determining suitable doses are well known in the art to which the present disclosure pertains. For example, Remington:

The Science and Practice of Pharmacy, Mack Publishing Co., 20th ed., 2000 can be used.

### Pharmaceutical Compositions, Dosage Forms and Administration Routes

For the treatment of the diseases or conditions referred to above, the compounds described herein or pharmaceutically acceptable salts thereof can be administered as follows:

### Oral Administration

The compounds of the present disclosure may be administered orally, including by swallowing, so that the compound enters the gastrointestinal tract, or absorbed into the blood stream directly from the mouth (e.g., buccal or sublingual administration). Suitable compositions for oral administration include solid, liquid, gel or powder formulations, and have a dosage form such as tablet, lozenge, capsule, granule or powder. Compositions for oral administration may be formulated as immediate or modified release, including delayed or sustained release, optionally with enteric coating. Liquid formulations can include solutions, syrups and suspensions, which can be used in soft or hard capsules. Such formulations may include a pharmaceutically acceptable carrier, for example, water, ethanol, polyethylene glycol, cellulose, or an oil. The formulation may also include one or more emulsifying agents and/or suspending agents.

In a tablet dosage form the amount of drug present may be from about 0.05% to about 95% by weight, more typically from about 2% to about 50% by weight of the dosage form. In addition, tablets may contain a disintegrant, comprising from about 0.5% to about 35% by weight, more typically from about 2% to about 25% of the dosage form. Examples of disintegrants include, but are not limited to, lactose, starch, sodium starch glycolate, crospovidone, croscarmellose sodium, maltodextrin, or mixtures thereof.

Suitable lubricants, for use in a tablet, may be present in amounts from about 0.1% to about 5% by weight, and include, but are not limited to, talc, silicon dioxide, stearic acid, calcium, zinc or magnesium stearate, sodium stearyl fumarate and the like.

Suitable binders, for use in a tablet, include, but are not limited to, gelatin, polyethylene glycol, sugars, gums, starch, polyvinyl pyrrolidone, hydroxypropyl cellulose, hydroxypropylmethyl cellulose and the like. Suitable diluents, for use in a tablet, include, but are not limited to, mannitol, xylitol, lactose, dextrose, sucrose, sorbitol, microcrystalline cellulose and starch.

Suitable solubilizers, for use in a tablet, may be present in amounts from about 0.1% to about 3% by weight, and include, but are not limited to, polysorbates, sodium lauryl sulfate, sodium dodecyl sulfate, propylene carbonate, diethyleneglycol monoethyl ether, dimethyl isosorbide, polyethylene glycol (natural or hydrogenated) castor oil, HCOR™ (Nikkol), oleyl ester, Gelucire™, caprylic/caprylic acid mono/diglyceride, sorbitan fatty acid esters, and Solutol HS™.

### Parenteral Administration

Compounds of the present disclosure may be administered directly into the blood stream, muscle, or internal organs. Suitable means for parenteral administration include intravenous, intra-muscular, subcutaneous intraarterial, intraperitoneal, intrathecal, intracranial, and the like. Suitable devices for parenteral administration include injectors (including needle and needle-free injectors) and infusion methods.

Compositions for parenteral administration may be formulated as immediate or modified release, including delayed or sustained release. Most parenteral formulations are aqueous solutions containing excipients, including salts, buffering agents and isotonic agents. Parenteral formulations may also be prepared in a dehydrated form (e.g., by lyophilization) or as sterile non-aqueous solutions. These formulations can be used with a suitable vehicle, such as sterile water. Solubility-enhancing agents may also be used in preparation of parenteral solutions.

### Transdermal Administration

Compounds of the present disclosure may be administered topically to the skin or transdermally. Formulations for this topical administration can include lotions, solutions, creams, gels, hydrogels, ointments, foams, implants, patches and the like. Pharmaceutically acceptable carriers for topical administration formulations can include water, alcohol, mineral oil, glycerin, polyethylene glycol and the like. Topical or transdermal administration can also be performed by electroporation, iontophoresis, phonophoresis and the like. Compositions for topical administration may be formulated as immediate or modified release, including delayed or sustained release.

### Combination Therapy

A pharmaceutical composition according to the present disclosure may contain one or more additional therapeutic agents, for example, to increase the efficacy or decrease the side effects. In some embodiments, accordingly, a pharmaceutical composition further contains one or more additional therapeutic agents selected from active ingredients useful to treat or inhibit diseases mediated directly or indirectly by HPK1. Examples of such active ingredients are, without limitation, agents to treat cancer, metastasis, inflammation, or auto-immune pathogenesis. In some embodiments, the compound of Formula (I) is administered with anti-PD-1 agent, anti-PD-L1 agent, or anti-CTLA4 agent.

### References for Preparing Pharmaceutical Compositions

Methods for preparing pharmaceutical compositions for treating or preventing a disease or condition are well known in the art to which the present disclosure pertains. For example, based on Handbook of Pharmaceutical Excipients (7^{th} ed.), Remington: The Science and Practice of Pharmacy (20^{th} ed.), Encyclopedia of Pharmaceutical Technology (3^{rd} ed.), or Sustained and Controlled Release Drug Delivery Systems (1978), pharmaceutically acceptable excipients, carriers, additives and so on can be selected and then mixed with the compounds of the present disclosure for making the pharmaceutical compositions.

The present disclosure provides a compound having various pharmacological effects by inhibiting HPK1 activity, a pharmaceutical composition having the compound as an effective agent, a medical use, particularly for treating a disease or disorder modulated by HPK1, of the compound, and a method of treatment or prevention comprising administering the compound to a subject in need of such treatment or prevention. The compounds of the present disclosure and pharmaceutically acceptable salts thereof have good safety and high selectivity for HPK1, and thus exhibit superior property as a drug.

### Compound Preparation

The following Preparative Examples illustrate the preparation of intermediate compounds that are useful for preparing compounds of formula (I). The novel intermediate compounds described herein, as well as the synthetic processes useful for preparing the intermediate compounds represent embodiments of the current invention.

### Intermediate 1A. 4-bromo-5-chloro-6-fluoro-N-isopropyl-2-(tetrahydro-2H-pyran-2-yl)-2H-indazol-7-amine

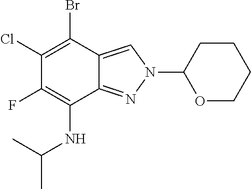 [SMILES: CC(C)Nc1c(F)c(Cl)c(Br)c2cn(C3CCCCO3)nc12]

### Step 1) 4-bromo-5-chloro-6-fluoro-7-iodo-1H-indazole

To a solution of 4-bromo-5-chloro-6-fluoro-1H-indazole (2 g, 8.02 mmol) in sulfuric acid (1.7 mL) was added N-iodosuccinimide (2.7 g, 12.03 mmol) portionwise. The mixture was stirred at 0° C. for 3 h. After the reaction completed, the mixture was poured into ice water and quenched by solid NaOH and then extracted with dichloromethane. The combined organic residue was concentrated in vacuo (2.99 g, crude).

¹H NMR (400 MHZ, DMSO-d₆) δ 13.91 (s, 1H), 8.27 (d, J=1.6 Hz, 1H).

### Step 2) 4-bromo-5-chloro-6-fluoro-7-iodo-2-(tetrahydro-2H-pyran-2-yl)-2H-indazole

To a solution of 4-bromo-5-chloro-6-fluoro-7-iodo-1H-indazole (2.99 g, 7.97 mmol, 1 eq) in THF (40 mL) was added 3,4-dihydro-2H-pyran (2.18 ml, 23.9 mmol, 3 eq) and p-toluenesulfonic acid monohydrate (300 mg, 1.59 mmol, 0.2 eq). The reaction mixture was stirred 60° C. for 16 hr. The reaction mixture was extracted with EtOAc and dried over MgSO₄. The organic residue was purified by column chromatography (silica gel, hexanes:EtOAc=1:0 to 4:1). 4-bromo-5-chloro-6-fluoro-7-iodo-2-(tetrahydro-2H-pyran-2-yl)-2H-indazole (1.64 g, 7.97 mmol, 60.7% yield) was obtained.

¹H NMR (400 MHZ, DMSO-d₆) δ 8.84 (s, 1H), 5.80 (dd, J=9.9, 2.7 Hz, 1H), 5.66 (s, 1H), 4.02 (t, J=6.6 Hz, 1H), 3.85-3.70 (m, 1H), 2.33-2.21 (m, 1H), 2.08-1.91 (m, 2H), 1.79-1.45 (m, 4H).

### Step 3) 4-bromo-5-chloro-6-fluoro-N-isopropyl-2-(tetrahydro-2H-pyran-2-yl)-2H-indazol-7-amine

To a solution of 4-bromo-5-chloro-6-fluoro-7-iodo-2-(tetrahydro-2H-pyran-2-yl)-2H-indazole (100 mg, 0.218 mmol, 1 eq) in 2-methyl-2-butanol (1.09 mL) was added Xantphos Pd G3 (21 mg, 21.8 μmol, 0.1 eq) and Cs₂CO₃ (142 mg, 0.436 mmol, 2.0 eq). The mixture was degassed and purged with N₂ for 3 times, and then propan-2-amine (0.19 mL, 2.18 mmol, 10 eq) was added. The mixture was stirred at 90° C. for 3 hr in sealed tube. The reaction mixture was diluted with H₂O (40 mL), and then the mixture was extracted with DCM (50 mL*3). The combined organic layers were dried over Na₂SO₄, filtered and the filtrate was concentrated in vacuum to give a residue. The residue was purified by silica gel chromatography (product came out at hexanes/EtOAc=10/1) to afford Intermediate 1A (47 mg, 0.120 mmol, 55% yield) as beige color solid.

¹H NMR (400 MHZ, DMSO-d₆) δ 8.43 (s, 1H), 5.74 (dd, J=9.6, 2.5 Hz, 1H), 5.29 (dd, J=9.9, 3.3 Hz, 1H), 4.63-4.57 (m, 1H), 3.99 (d, J=11.0 Hz, 1H), 3.74-3.68 (m, 1H), 2.23-2.17 (m, 1H), 2.05-1.95 (m, 2H), 1.74-1.57 (m, 3H), 1.23-1.18 (m, 6H).

### Intermediate 1B. 5-chloro-6-fluoro-N-isopropyl-2-(tetrahydro-2H-pyran-2-yl)-4-(4,4,5,5-tetramethyl-1,3,2-dioxaborolan-2-yl)-2H-indazol-7-amine

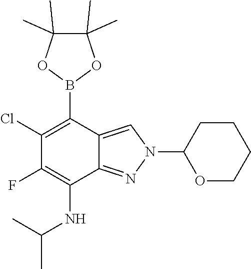 [SMILES: CC(C)Nc1c(F)c(Cl)c(B2OC(C)(C)C(C)(C)O2)c2cn(C3CCCCO3)nc12]

To a solution of Intermediate 1A (210 mg, 537.53 μmol, 1 eq) and BPD (409.50 mg, 1.61 mmol, 3 eq) in Tol. (5 mL) was added Pd(OAc)₂ (12.07 mg, 53.75 μmol, 0.1 eq), PPh₂t-Bu (26.05 mg, 107.51 μmol, 0.2 eq) and KOAc (158.26 mg, 1.61 mmol, 3 eq). The mixture was stirred at 110° C. for 4 hr under N₂ atmosphere. The reaction mixture was concentrated under reduced pressure to give a residue. The residue was diluted with water (20 mL) and extracted with EtOAc (20 mL*2). The combined organic layers were washed with brine (30 mL*2), dried over anhydrous Na₂SO₄, filtered and the filtrate was concentrated under reduced pressure to give a residue. The residue was purified by flash reverse phase (0.5% FA condition, 91%˜100% ACN). Intermediate 1B (115 mg, 262.71 μmol, 48.87% yield) was obtained as a yellow solid.

¹H NMR (400 MHZ, DMSO-d₆) δ 8.36 (s, 1H), 5.76 (dd, J=2.5, 9.4 Hz, 1H), 5.59 (dd, J=3.2, 9.5 Hz, 1H), 4.84-4.69 (m, 1H), 4.05-3.92 (m, 1H), 3.79-3.66 (m, 1H), 2.21-2.10 (m, 1H), 2.08-1.94 (m, 2H), 1.78-1.68 (m, 1H), 1.64-1.55 (m, 2H), 1.32 (s, 12H), 1.22 (dd, J=2.1, 6.4 Hz, 6H).

### Intermediate 1C. 4-bromo-5-(difluoromethyl)-6-fluoro-1H-indazole

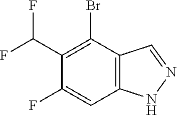 [SMILES: Fc1cc2[nH]ncc2c(Br)c1C(F)F]

### Step 1) 4-bromo-6-fluoro-1H-indazole

To a solution of 3-bromo-5-fluoro-2-methylaniline (25 g, 123 mmol, 1.0 eq) in AcOH (2371 mL) and H₂O (79 mL) was added NaNO₂ (10.14 g, 147 mmol, 1.2 eq). The reaction mixture was stirred at RT for 16 hr. Checked LCMS, the reaction mixture was concentrated and residue solidified with ice water. The reaction mixture was filtered then washed with water and vacuum dried to afford 4-bromo-6-fluoro-1H-indazole (26.3 g, crude) as an orange color solid.

### Step 2) 4-bromo-6-fluoro-1-trityl-1H-indazole

To a solution of 4-bromo-6-fluoro-1H-indazole (26.3 g, 122 mmol, 1.0 eq) in THF (306 mL) was added NaH (6.36 g, 159 mmol, 1.3 eq) at 0° C. After 30 min, Trityl chloride (37.5 g, 135 mmol, 1.1 eq) was added at 0° C. The reaction mixture was stirred at RT for 16 hr. The mixture was added ice/EtOAc and stirred at RT for 30 min. The mixture was extracted with EtOAc/water. The organic layers were dried over Na₂SO₄, filtered, and concentrated. The residue was solidified with DCM/Hexane to afford 4-bromo-6-fluoro-1-trityl-1H-indazole (42.7 g, 93 mmol, 76% yield) as a yellow color solid.

### Step 3) 4-bromo-6-fluoro-1-trityl-1H-indazole-5-carbaldehyde

A solution of 4-bromo-6-fluoro-1-trityl-1H-indazol (500 mg, 1.093 mmol, 1.0 eq) in THF (5.5 mL) was added LDA (0.82 mL, 1.640 mmol, 1.5 eq) at −78° C. After 2 hr methyl formate (0.101 mL, 1.640 mmol, 1.5 eq) was added at −78° C. The reaction mixture was warmed to RT and stirred at RT for 3 hr. The mixture was quenched by sat. NH₄Cl, extracted with EtOAc (200 mL*3), and then dried over Na₂SO₄, and concentrated. The crude 4-bromo-6-fluoro-1-trityl-1H-indazole-5-carbaldehyde was used into next step.

### Step 4) 4-bromo-5-(difluoromethyl)-6-fluoro-1-trityl-1H-indazole

To a solution of 4-bromo-6-fluoro-1-trityl-1H-indazole-5-carbaldehyde (1062 mg, 2.188 mmol, 1.0 eq) in DCM (44 mL) was added DAST (1.445 mL, 17.63 mmol, 5.0 eq) at 0° C. The mixture was stirred at 25° C. for 16 hr under nitrogen atmosphere. The reaction mixture was diluted with DCM, and then the resulting organic phase was washed with saturated NaHCO₃ and brine, dried over Na₂SO₄, filtered and the filtrate was concentrated in vacuum to give a residue. The crude 4-bromo-5-(difluoromethyl)-6-fluoro-1-trityl-1H-indazole was used into next step directly.

### Step 5) 4-bromo-5-(difluoromethyl)-6-fluoro-1H-indazole, Intermediate 1C

To a solution of 4-bromo-5-(difluoromethyl)-6-fluoro-1-trityl-1H-indazole (1110 mg, 2.188 mmol, 1.0 eq) in DCM (15 mL) was added TFA (5.1 mL, 65.6 mmol, 30 eq) at 0° C. The reaction mixture was stirred at RT for 5 hr. The reaction was quenched with ice and saturated NaHCO₃. The mixture was extracted with DCM and dried over Na₂SO₄, filtered, concentrated. The residue was purified by silica gel chromatography (product came out at EtOAc/hexanes=30/100) to afford 4-bromo-5-(difluoromethyl)-6-fluoro-1H-indazole (418 mg, 1.577 mmol, 72%) as a yellow color solid.

### Intermediate 1D. 4-bromo-5-(difluoromethyl)-6-fluoro-1H-indazol-7-amine

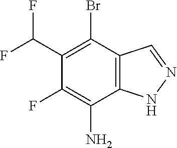 [SMILES: Nc1c(F)c(C(F)F)c(Br)c2cn[nH]c12]

### Step 1) 4-bromo-5-(difluoromethyl)-6-fluoro-7-nitro-1H-indazole

4-bromo-5-(difluoromethyl)-6-fluoro-1H-indazole, intermediate 1C (133 mg, 0.502 mmol, 1.0 eq) was dissolved in H₂SO₄ (0.627 mL) at 0° C. HNO₃ (0.112 mL, 2.509 mmol, 5.0 eq) was added drop-wise at 0° C. The reaction mixture was stirred at 0° C. for 2 hr. Reaction was checked by TLC. Mixture was poured into ice water and precipitated solid was filtered. 4-bromo-5-(difluoromethyl)-6-fluoro-7-nitro-1H-indazole was obtained as an ivory color solid

### Step 2) 4-bromo-5-(difluoromethyl)-6-fluoro-1H-indazol-7-amine, Intermediate 1D

To a solution of 4-bromo-5-(difluoromethyl)-6-fluoro-7-nitro-1H-indazole (150 mg, 0.503 mmol, 1.0 eq) in EtOH/H₂O (1.6 mL/0.915 mL) was added iron (140 mg, 2.52 mmol, 5.0 eq) and NH₄Cl (135 mg, 2.52 mmol, 5.0 eq). The reaction mixture was stirred at 80° C. for 3 hr. The mixture was celite filtered to remove iron. Filtrate was concentrated and extracted with EtOAc/saturated NaHCO₃. The organic layers were dried over Na₂SO₄, filtered, and concentrated. The residue was purified by silica gel chromatography (product came out at EtOAc/hexanes=45/100) to afford 4-bromo-5-(difluoromethyl)-6-fluoro-1H-indazol-7-amine (79 mg, 0.282 mmol, 56%) as a brown color solid.

### Intermediate 1E. 4-bromo-5-(difluoromethyl)-6-fluoro-N,N-dimethyl-1H-indazol-7-amine

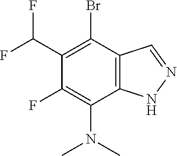 [SMILES: CN(C)c1c(F)c(C(F)F)c(Br)c2cn[nH]c12]

To a solution of Intermediate 1D, 4-bromo-5-(difluoromethyl)-6-fluoro-1H-indazol-7-amine (79 mg, 0.282 mmol, 1.0 eq) in MeOH (1.41 mL) was added formaldehyde (0.105 mL, 1.410 mmol, 5.0 eq), acetic acid (0.017 mL, 0.282 mmol, 1.0 eq) and sodium cyanoborohydride (54 mg, 0.846 mmol, 3.0 eq). The reaction mixture was stirred at RT for 16 hr. The reaction was quenched by water and concentrated. Extracted with EtOAc and dried over Na₂SO₄, filtered, concentrated. The residue was purified by silica gel chromatography to afford 4-bromo-5-(difluoromethyl)-6-fluoro-N,N-dimethyl-1H-indazol-7-amine (35 mg, 0.114 mmol, 40%) as a yellow color solid.

### Intermediate 1F. (4-bromo-5-(difluoromethyl)-6-fluoro-1-(tetrahydro-2H-pyran-2-yl)-1H-indazol-7-yl)(cyclopropyl)methanol

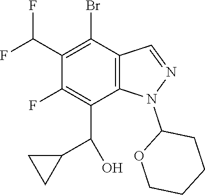 [SMILES: OC(c1c(F)c(C(F)F)c(Br)c2cnn(C3CCCCO3)c12)C1CC1]

### Step 1) 4-bromo-5-(difluoromethyl)-6-fluoro-1-(tetrahydro-2H-pyran-2-yl)-1H-indazole

To a solution of 4-bromo-5-(difluoromethyl)-6-fluoro-1H-indazole (418 mg, 1.577 mmol, 1.0 eq) in THF (7.89 mL) was added DHP (0.433 mL, 4.73 mmol, 3.0 eq) and PPTS (79 mg, 0.315 mmol, 0.2 eq). The reaction mixture was stirred at 75° C. for 16 hr. The reaction mixture was extracted with EtOAc (50 mL*3). The combined organic layers were dried over Na₂SO₄, filtered and the filtrate was concentrated in vacuum. The crude product was purified by silica gel chromatography to afford 4-bromo-5-(difluoromethyl)-6-fluoro-1-(tetrahydro-2H-pyran-2-yl)-1H-indazole (437 mg, 1.252 mmol, 79%) as a white color solid.

### Step 2) (4-bromo-5-(difluoromethyl)-6-fluoro-1-(tetrahydro-2H-pyran-2-yl)-1H-indazol-7-yl)(cyclopropyl)methanol, Intermediate 1F

To a solution of 4-bromo-5-(difluoromethyl)-6-fluoro-1-(tetrahydro-2H-pyran-2-yl)-1H-indazole (437 mg, 1.252 mmol, 1.0 eq) in THF (25.0 mL) was added LDA (1.88 mL, 3.75 mmol, 3.0 eq) at −78° C. The mixture was stirred at −78° C. for 2 hr and cyclopropanecarbaldehyde (0.187 mL, 2.503 mmol, 2.0 eq) was added at −78° C. The mixture was stirred at 25° C. for 16 hr. The reaction mixture was quenched by saturated NH₄Cl at 0° C. and diluted with H₂O. The mixture was extracted with EtOAc (150 mL*3) and washed with brine. The combined organic layers were dried over Na₂SO₄, filtered and the filtrate was concentrated in vacuum. The crude product was purified by silica gel chromatography (product came out at EtOAc/hexanes=30/100) to afford (4-bromo-5-(difluoromethyl)-6-fluoro-1-(tetrahydro-2H-pyran-2-yl)-1H-indazol-7-yl)(cyclopropyl)methanol (275 mg, 0.656 mmol, 52%) as a yellow color solid.

### Intermediate 1G. 4-bromo-5-(difluoromethyl)-6-fluoro-N-isopropyl-2-(tetrahydro-2H-pyran-2-yl)-2H-indazol-7-amine

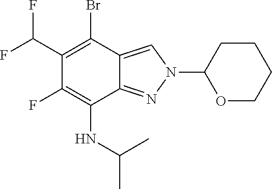 [SMILES: CC(C)Nc1c(F)c(C(F)F)c(Br)c2cn(C3CCCCO3)nc12]

### Step 1) 4-bromo-5-(difluoromethyl)-6-fluoro-N-isopropyl-1H-indazol-7-amine

To a solution of 4-bromo-5-(difluoromethyl)-6-fluoro-1H-indazol-7-amine (164 mg, 0.586 mmol, 1.0 eq) in DCM (2.93 mL) was added acetone (0.43 mL, 5.86 mmol, 10.0 eq) and acetic acid (0.335 mL, 5.86 mmol, 10.0 eq). The mixture was stirred at 25° C. for 10 min. Sodium cyanoborohydride (74 mg, 1.171 mmol, 2.0 eq) was added and then the mixture was stirred at 25° C. for 1 hr. The reaction was quenched by water and concentrated. Extracted with DCM and dried over Na₂SO₄, filtered, and concentrated. The residue was purified by silica gel chromatography (product came out at EtOAc/Hexane=20/100) to afford 4-bromo-5-(difluoromethyl)-6-fluoro-N-isopropyl-1H-indazol-7-amine (154 mg, 0.478 mmol, 82%) as a yellow color solid.

### Step 2) 4-bromo-5-(difluoromethyl)-6-fluoro-N-isopropyl-2-(tetrahydro-2H-pyran-2-yl)-2H-indazol-7-amine, Intermediate 1G

To a solution of 4-bromo-5-(difluoromethyl)-6-fluoro-N-isopropyl-1H-indazol-7-amine (154 mg, 0.478 mmol, 1.0 eq) in THF (2.39 mL) was added DHP (0.131 mL, 1.434 mmol, 3.0 eq) and PPTS (24 mg, 0.096 mmol, 0.2 eq). The reaction mixture was stirred at 65° C. for 16 hr. The reaction mixture was extracted with EtOAc (50 mL*3). The combined organic layers were dried over Na₂SO₄, filtered and the filtrate was concentrated in vacuum. The crude product was purified by silica gel chromatography (product came out at EtOAc/hexanes=10/100) to afford 4-bromo-5-(difluoromethyl)-6-fluoro-N-isopropyl-2-(tetrahydro-2H-pyran-2-yl)-2H-indazol-7-amine (137 mg, 0.337 mmol, 71%) as a yellow color oil.

### Intermediate 1H. (1S,2S)-2-fluoro-N-(6-(tributylstannyl)imidazo[1,2-a]pyrazin-2-yl)cyclopropane-1-carboxamide

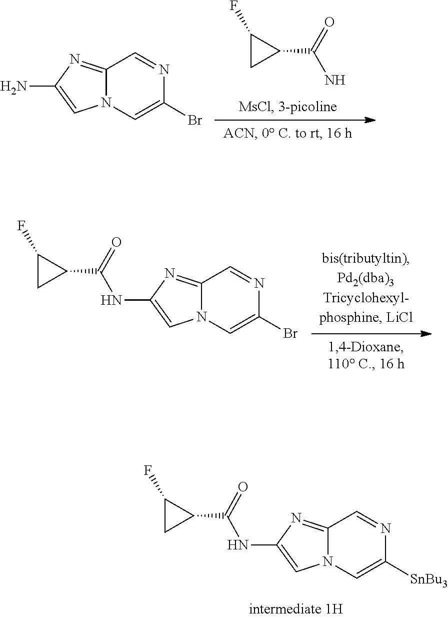

### Step 1) (1S,2S)—N-(6-bromoimidazo[1,2-a]pyrazin-2-yl)-2-fluorocyclopropane-1-carboxamide

To a solution of 6-bromoimidazo[1,2-a]pyrazin-2-amine (5 g, 23.47 mmol, 1.0 eq) and (1S,2S)-2-fluorocyclopropane-1-carboxylic acid (2.93 g, 28.2 mmol, 1.2 eq) in ACN (117 mL) was added MsCl (4.57 mL, 58.7 mmol, 2.5 eq) and 3-picoline (11.38 mL, 117 mmol, 5.0 eq) at 0° C. The reaction mixture was stirred at 25° C. for 16 hr. The mixture was poured into ice-water, a yellow solid precipitated. The mixture was filtered and the filter cake was washed with little water, dried under vacuum to obtain (1S,2S)—N-(6-bromoimidazo[1,2-a]pyrazin-2-yl)-2-fluorocyclopropane-1-carboxamide (5.81 g, 19.42 mmol, 83%) as a yellow color solid.

### Step 2) (1S,2S)-2-fluoro-N-(6-(tributylstannyl)imidazo[1,2-a]pyrazin-2-yl)cyclopropane-1-carboxamide

To a solution of (1S,2S)—N-(6-bromoimidazo[1,2-a]pyrazin-2-yl)-2-fluorocyclopropane-1-carboxamide (5.81 g, 19.42 mmol, 1.0 eq) in 1,4-dioxane (97 mL) was added bis(tributylyin) (24.5 mL, 48.6 mmol, 2.5 eq), Pd₂ (dba) 3 (1779 mg, 1.942 mmol, 0.1 eq), tricyclohexylphosphine (1089 mg, 3.88 mmol, 0.2 eq) and LiCl (4.94 g, 117 mmol, 6.0 eq). The reaction mixture was degassed and purged with N₂ for 3 times, and then stirred at 110° C. for 16 hr. The mixture was poured into water, extracted with EtOAc. The combined organic phase was washed with brine, dried over Na₂SO₄, filtered and the filtrate was concentrated to give the crude. The residue was purified by silica gel chromatography to afford intermediate 1H, (1S,2S)-2-fluoro-N-(6-(tributylstannyl)imidazo[1,2-a]pyrazin-2-yl)cyclopropane-1-carboxamide (2.81 g, 5.52 mmol, 28%) as an ivory color solid.

### Synthesis of Formula (I) Compounds

Synthetic methods A to J were used to prepare the compounds of the following. Below, the illustrating synthetic examples of some compounds of the present disclosure are described, and other compounds can be prepared by the similar method to the one described below with different starting or reacting materials.

### Synthetic Method A

### Example 14. 6-((5-chloro-6-fluoro-4-(2-((1S,2S)-2-fluorocyclopropane-1-carboxamido)imidazo[1,2-a]pyrazin-6-yl)-1H-indazol-7-yl)(isopropyl)amino)-3,4,5-trihydroxytetrahydro-2H-pyran-2-carboxylic acid; Example 18. 2-((5-chloro-6-fluoro-4-(2-((1S,2S)-2-fluorocyclopropane-1-carboxamido)imidazo[1,2-a]pyrazin-6-yl)-1H-indazol-7-yl)(isopropyl)amino)-6-(methoxycarbonyl)tetrahydro-2H-pyran-3,4,5-triyl triacetate

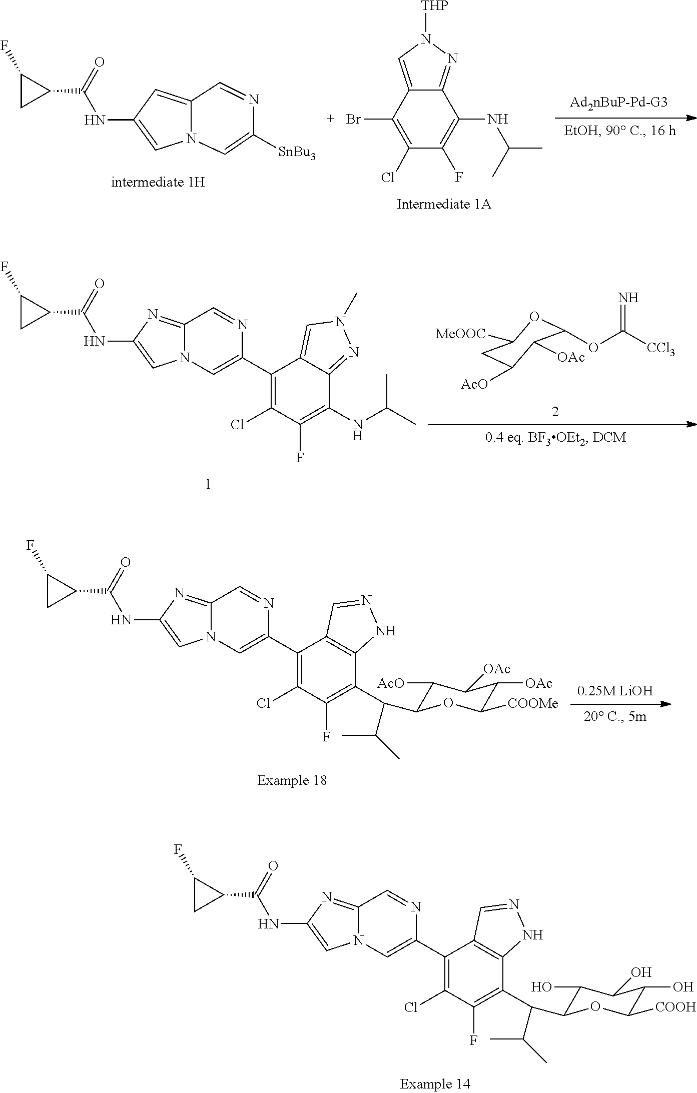

### Step 1) (1S,2S)—N-(6-(5-chloro-6-fluoro-7-(isopropylamino)-2-(tetrahydro-2H-pyran-2-yl)-2H-indazol-4-yl)imidazo[1,2-a]pyrazin-2-yl)-2-fluorocyclopropane-1-carboxamide

To a solution of Intermediate 1H (644 mg, 1.26 mmol, 1.3 eq) and Intermediate 1A (380 mg, 0.973 mmol, 1.0 eq) in EtOH (4.86 mL) was added Ad₂nBuP-Pd-G3 (71 mg, 0.097 mmol, 0.1 eq). The mixture was degassed and purged with N₂ for 3 times, and then stirred at 90° C. for 16 hr under N₂ atmosphere. The reaction mixture was concentrated in vacuum. The crude product was purified by silica gel chromatography to afford Compound 1 (400 mg, 0.754 mmol, 77.5% yield) as yellow color solid.

### Step 2) (2R,3R,4S,5S,6S)-2-((5-chloro-6-fluoro-4-(2-((1S,2S)-2-fluorocyclopropane-1-carboxamido)imidazo[1,2-a]pyrazin-6-yl)-1H-indazol-7-yl)(isopropyl)amino)-6-(methoxycarbonyl)tetrahydro-2H-pyran-3,4,5-triyl triacetate

To a solution of Compound 1 (0.4 g, 754.76 μmol, 1 eq) in DCM (40 mL) was added Compound 2 (541.91 mg, 1.13 mmol, 1.5 eq) and BF₃·Et₂O (214.25 mg, 1.51 mmol, 186.30 μL, 2 eq). The mixture was stirred at 20° C. for 2 hr. LC-MS found the desired MS. The mixture was poured into saturated NaHCO₃ (30 mL) and extracted with DCM (30 mL). The organic layer was concentrated. The residue was purified by prep-TLC (SiO₂, DCM:MeOH=20:1). Example 18 (200 mg, 244.06 μmol, 10.78% yield, 93% purity) was obtained as white solid.

¹H NMR (400 MHZ, CDCl₃-d) δ 12.21 (br s, 1H), 9.11 (br s, 1H), 8.97 (br s, 1H), 8.51 (s, 1H), 8.34 (br s, 1H), 8.04 (s, 1H), 5.43-5.30 (m, 1H), 5.10 (d, J=9.5 Hz, 1H), 5.00-4.77 (m, 2H), 4.57 (br t, J=9.4 Hz, 1H), 4.25 (d, J=9.9 Hz, 1H), 3.90 (s, 3H), 3.62 (td, J=6.6, 13.2 Hz, 1H), 2.09 (s, 3H), 2.04 (s, 3H), 2.01-1.94 (m, 2H), 1.92 (s, 3H), 1.40-1.26 (m, 7H); LCMS(electrospray) m/z 762.6 (M+H)⁺.

### Step 3) (2S,3S,4S,5R,6R)-6-((5-chloro-6-fluoro-4-(2-((1S,2S)-2-fluorocyclopropane-1-carboxamido)imidazo[1,2-a]pyrazin-6-yl)-1H-indazol-7-yl)(isopropyl)amino)-3,4,5-trihydroxytetrahydro-2H-pyran-2-carboxylic acid

To a solution of Example 18 (150 mg, 196.82 μmol, 1 eq) in THF (2 mL) was added MeOH (2 mL)/H₂O (1 mL) and LiOH—H₂O (104.91 mg, 2.5 mmol, 12.70 eq). The mixture was stirred at 0° C. for 5 min. The mixture was neutralized with 1 M HCl to pH 7.0 and concentrated. The mixture was purified by prep-HPLC: column: Phenomenex C18 150*25 mm*10 um; mobile phase: [water (NH₄HCO₃)-ACN]; B %: 5%-35%, 8 min. After lyophilization we got the desired product. Example 14 (50 mg, 79.59 μmol, 40.44% yield, 99% purity) was obtained as white solid.

¹H NMR (400 MHZ, DMSO-d₆) δ 13.62 (br s, 1H), 11.61-11.16 (m, 1H), 9.07 (s, 1H), 9.00 (s, 1H), 8.37 (s, 1H), 7.96 (br s, 1H), 5.05 (br s, 1H), 4.94-4.74 (m, 1H), 4.62 (br d, J=8.6 Hz, 1H), 4.49 (br s, 1H), 3.69-3.63 (m, 1H), 3.61-3.54 (m, 1H), 3.24 (br d, J=9.0 Hz, 2H), 2.90 (br t, J=8.8 Hz, 1H), 2.62 (br s, 1H), 2.20 (br s, 1H), 1.77-1.64 (m, 1H), 1.29-1.10 (m, 8H); LCMS(electrospray) m/z 622.2 (M+H)⁺.

### Synthetic Method B

### Example 20. (1S,2R)-2-((6-(5-chloro-6-fluoro-7-(isopropylamino)-1H-indazol-4-yl)imidazo[1,2-a]pyrazin-2-yl)carbamoyl)cyclopropane-1-carboxylic acid

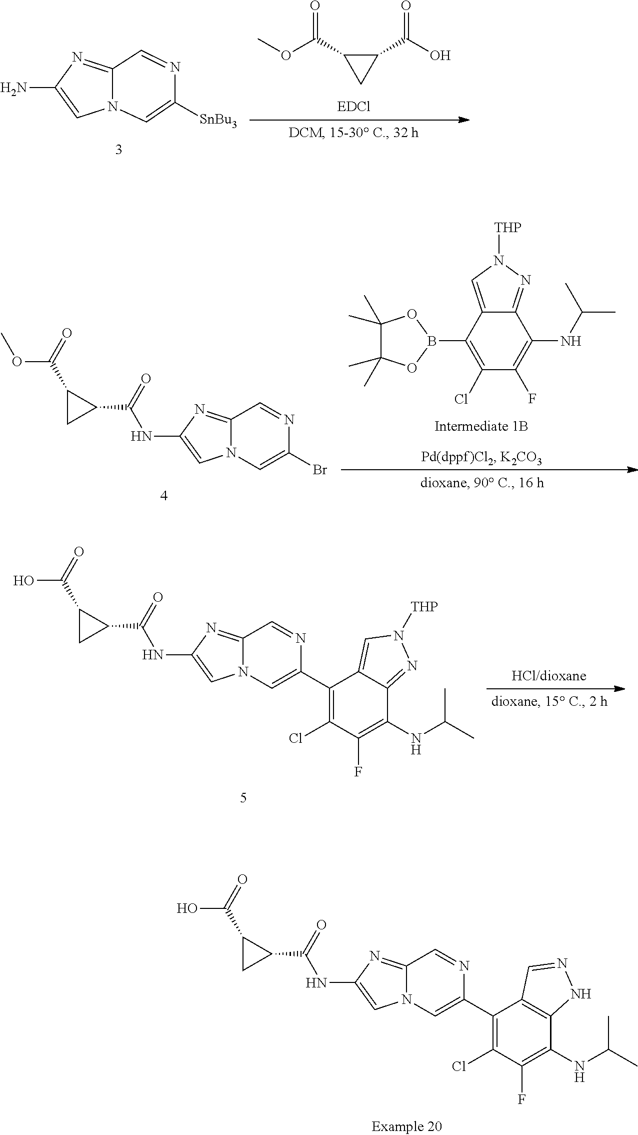

### Step 1) methyl (1S,2R)-2-((6-bromoimidazo[1,2-a]pyrazin-2-yl)carbamoyl)cyclopropane-1-carboxylate

To a solution of Compound 3 (430 mg, 2.02 mmol, 1 eq) and (1R,2S)-2-(methoxycarbonyl)cyclopropane-1-carboxylic acid (436.36 mg, 3.03 mmol, 1.5 eq) in DCM (5 mL) was added EDCI (580.41 mg, 3.03 mmol, 1.5 eq). The mixture was stirred at 15° C. for 16 hr. Then the mixture was warmed to 30° C. and stirred for 16 hr. The reaction mixture was concentrated under reduced pressure to remove solvent. The residue was purified by flash reverse phase (0.5% FA condition, 25%˜33% ACN). Compound 4 (50 mg, 147.43 μmol, 7.30% yield) was obtained as a white solid.

¹H NMR (400 MHz, DMSO-d₆) δ 11.40 (s, 1H), 8.90 (d, J=1.3 Hz, 1H), 8.75 (s, 1H), 8.26 (s, 1H), 3.53 (s, 3H), 2.34 (dt, J=6.4, 8.6 Hz, 1H), 2.21 (dt, J=6.9, 8.7 Hz, 1H), 1.45 (dt, J=4.3, 6.5 Hz, 1H), 1.28-1.20 (m, 1H).

### Step 2) (1S,2R)-2-((6-(5-chloro-6-fluoro-7-(isopropylamino)-2-(tetrahydro-2H-pyran-2-yl)-2H-indazol-4-yl)imidazo[1,2-a]pyrazin-2-yl)carbamoyl)cyclopropane-1-carboxylic acid

To a solution of Compound 4 (50 mg, 147.43 μmol, 1 eq) and Intermediate 1B (70.99 mg, 162.17 μmol, 1.1 eq) in dioxane (2 mL) was added Pd(dppf)Cl₂ (10.79 mg, 14.74 μmol, 0.1 eq) and K₂CO₃ (61.13 mg, 442.29 μmol, 3 eq). The mixture was degassed and purged with N₂ for 3 times, and then the mixture was stirred at 90° C. for 16 hr under N₂ atmosphere. The reaction mixture was concentrated under reduced pressure to give a residue. The residue was purified by flash reverse phase (0.5% FA condition, 53%˜59% ACN). Compound 5 (20 mg, 35.97 μmol, 24.40% yield) was obtained as a yellow solid.

### Step 3) (1S,2R)-2-((6-(5-chloro-6-fluoro-7-(isopropylamino)-1H-indazol-4-yl)imidazo[1,2-a]pyrazin-2-yl)carbamoyl)cyclopropane-1-carboxylic acid

To a solution of Compound 5 (20 mg, 35.97 μmol, 1 eq) in dioxane (1 mL) was added HCl/dioxane (4 M, 1 mL). The mixture was stirred at 15° C. for 2 hr. The reaction mixture was concentrated under reduced pressure to give a residue. The residue was purified by prep-HPLC (column: Phenomenex luna C18 150*25 mm*10 um; mobile phase: [water (TFA)-ACN]; B %: 25%-55%, 11 min) and lyophilized to give a product. Example 20 (5 mg, 10.60 μmol, 29.46% yield, 100% purity) was obtained as a white solid.

¹H NMR (400 MHZ, DMSO-d₆) δ 13.22-13.11 (m, 1H), 12.21-12.07 (m, 1H), 11.49-11.33 (m, 1H), 9.01 (s, 1H), 8.88 (s, 1H), 8.31 (s, 1H), 7.94 (br s, 1H), 5.19 (br d, J=8.3 Hz, 1H), 4.12-3.95 (m, 1H), 2.31-2.24 (m, 1H), 2.12-2.02 (m, 1H), 1.50-1.41 (m, 1H), 1.23 (d, J=6.2 Hz, 6H), 1.21-1.17 (m, 1H); LCMS(electrospray) m/z 472.2 (M+H)⁺.

### Synthetic Method C

### Example 32. methyl (1S,2R)-2-((6-(5-chloro-6-fluoro-7-(isopropylamino)-1H-indazol-4-yl)imidazo[1,2-a]pyrazin-2-yl)carbamoyl)cyclopropane-1-carboxylate

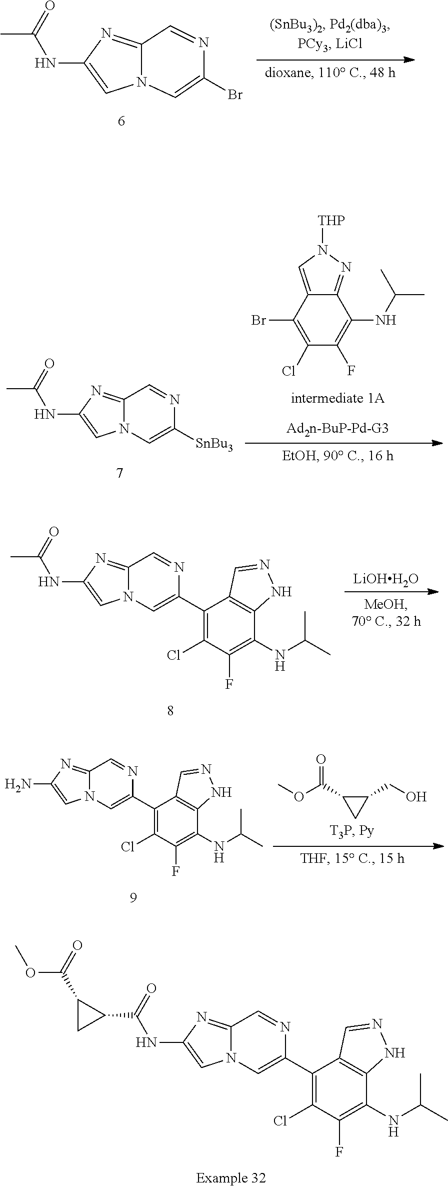

### Step 1) N-(6-(tributylstannyl)imidazo[1,2-a]pyrazin-2-yl) acetamide

To a solution of Compound 6 (18.9 g, 74.10 mmol, 1 eq) in dioxane (200 mL) was added Pd₂(dba)₃ (1.36 g, 1.48 mmol, 0.02 eq), PCy₃ (831.15 mg, 2.96 mmol, 960.87 μL, 0.04 eq) and LiCl (18.85 g, 444.58 mmol, 9.11 mL, 6 eq). The mixture was degassed and purged with N₂ for 3 times. The mixture was heated to 110° C. and then (SnBu₃)₂ (92.54 g, 159.53 mmol, 79.78 mL, 2.15 eq) was dropwised to the mixture. The mixture was stirred at 110° C. for 48 hr under N₂ atmosphere. The reaction mixture was filtered, and the filtrate was concentrated under reduced pressure to give a residue. The residue was purified by column chromatography on silical gel (petroleum ether/EtOAc=10/1 to 1/2). Compound 7 (7.9 g, 16.98 mmol, 22.92% yield) was obtained as a yellow solid.

¹H NMR (400 MHZ, DMSO-d₆) δ 10.89 (s, 1H), 8.97 (s, 1H), 8.53-8.45 (m, 1H), 8.20 (s, 1H), 2.09 (s, 3H), 1.65-1.49 (m, 6H), 1.38-1.24 (m, 6H), 1.15-1.06 (m, 6H), 0.93-0.80 (m, 9H).

### Step 2) N-(6-(5-chloro-6-fluoro-7-(isopropylamino)-1H-indazol-4-yl)imidazo[1,2-a]pyrazin-2-yl) acetamide

To a solution of Compound 7 (1 g, 2.15 mmol, 1 eq) and Intermediate 1A (839.77 mg, 2.15 mmol, 1 eq) in EtOH (10 mL) was added Ad₂n-BuP-Pd-G3 (156.54 mg, 214.95 μmol, 0.1 eq). The mixture was stirred at 90° C. for 16 hr under N₂ atmosphere. The reaction mixture was quenched by addition with saturated KF aqueous (20 mL), the mixture was stirred at 15° C. for 1 hr. The mixture was added with EtOAc (50 mL), and then filtered. The filtrate was extracted with EtOAc (30 mL*2). The combined organic layers were washed with brine (80 mL*2), dried over anhydrous Na₂SO₄, filtered and the filtrate was concentrated under reduced pressure to give a residue. The residue was purified by column chromatography on silical gel (petroleum ether/EtOAc=10/1 to 1/5). Compound 8 (250 mg, 622.16 μmol, 28.94% yield) was obtained as a yellow solid.

### Step 3) 6-(5-chloro-6-fluoro-7-(isopropylamino)-1H-indazol-4-yl)imidazo[1,2-a]pyrazin-2-amine

To a solution of Compound 8 (250 mg, 622.16 μmol, 1 eq) in MeOH (10 mL) was added LiOH—H₂O (130.54 mg, 3.11 mmol, 5 eq). The mixture was stirred at 70° C. for 16 hr. Then the mixture was stirred at 70° C. for another 16 hr. The reaction mixture was concentrated under reduced pressure to give a residue. The residue was purified by flash reverse phase (0.5% FA in H₂O/ACN condition, 21%˜27% ACN). Compound 9 (90 mg, 250.15 μmol, 40.21% yield) was obtained as a yellow solid.

### Step 4) methyl (1S,2R)-2-((6-(5-chloro-6-fluoro-7-(isopropylamino)-1H-indazol-4-yl)imidazo[1,2-a]pyrazin-2-yl)carbamoyl)cyclopropane-1-carboxylate

To a solution of Compound 9 (20 mg, 55.59 μmol, 1 eq) and (1R,2S)-2-(methoxycarbonyl)cyclopropane-1-carboxylic acid (9.61 mg, 66.71 μmol, 1.2 eq) in THF (2 mL) was added T3P (53.06 mg, 83.38 μmol, 49.59 μL, 50% purity, 1.5 eq) and Py (21.99 mg, 277.94 μmol, 22.43 μL, 5 eq). The mixture was stirred at 15° C. for 16 hr. The reaction mixture was concentrated under reduced pressure to give a residue. The residue was purified by prep-HPLC (column: Waters Xbridge 150*25 mm*5 um; mobile phase: [water (NH₄HCO₃)-ACN]; B %: 31%-61%, 8 min) and lyophilized to give a product. Example 32 (5.7 mg, 11.44 μmol, 20.58% yield, 97.5% purity) was obtained as a yellow solid.

¹H NMR (400 MHZ, DMSO-d₆) δ 13.28-13.07 (m, 1H), 11.36 (s, 1H), 9.01 (s, 1H), 8.87 (s, 1H), 8.31 (s, 1H), 7.97 (br s, 1H), 5.20 (br dd, J=1.9, 9.3 Hz, 1H), 4.19-3.99 (m, 1H), 3.54 (s, 3H), 2.40-2.33 (m, 1H), 2.26-2.17 (m, 1H), 1.51-1.44 (m, 1H), 1.27 (br d, J=4.3 Hz, 1H), 1.23 (d, J=6.3 Hz, 6H); LCMS(electrospray) m/z 486.1 (M+H)⁺.

### Synthetic Method D

### Example 17. (1S,2S)—N-(6-(5-(difluoromethyl)-7-(dimethylamino)-6-fluoro-1H-indazol-4-yl)imidazo[1,2-a]pyrazin-2-yl)-2-fluorocyclopropane-1-carboxamide

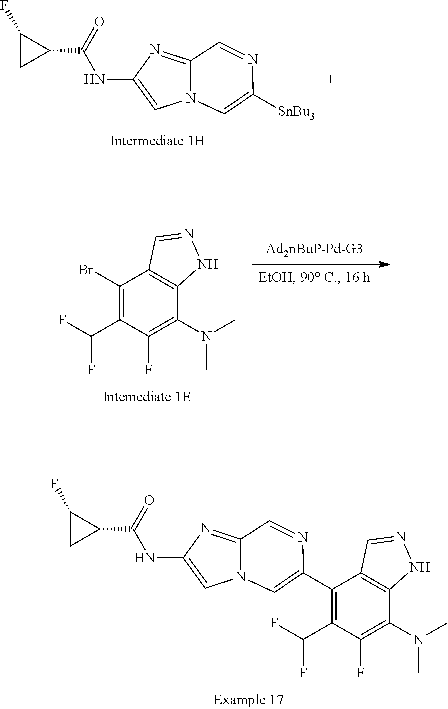

To a solution of Intermediate 1E (35 mg, 0.114 mmol, 1.0 eq) and Intermediate 1H (70 mg, 0.136 mmol, 1.2 eq) in EtOH (0.568 mL) was added Ad₂nBuP-Pd-G3 (9 mg, 0.011 mmol, 0.1 eq). The mixture was degassed and purged with N₂ for 3 times, and then stirred at 90° C. for 16 hr under N₂ atmosphere. The reaction mixture was concentrated in vacuum. The crude product was purified by prep-TLC (DCM/MeOH=10/1, DCM/MeOH=15/1) to afford Example 17 (0.87 mg, 0.001945 mmol, 2%) as a yellow color solid.

¹H NMR (400 MHz, DMSO-d₆) δ 13.58 (s, 1H), 11.38 (s, 1H), 9.02 (s, 1H), 8.92 (s, 1H), 8.38 (s, 1H), 8.16 (s, 1H), 7.08 (t, J=52.5 Hz, 1H), 5.09-4.86 (m, 1H), 3.00-2.91 (m, 6H), 2.22-2.17 (m, 1H), 1.72-1.64 (m, 2H), 1.20-1.20 (m, 1H); LCMS(electrospray) m/z 448.10 (M+H)⁺.

### Synthetic Method E

### Example 19. (1S,2S)—N-(6-(7-(cyclopropyl(hydroxy)methyl)-5-(difluoromethyl)-6-fluoro-1H-indazol-4-yl)imidazo[1,2-a]pyrazin-2-yl)-2-fluorocyclopropane-1-carboxamide

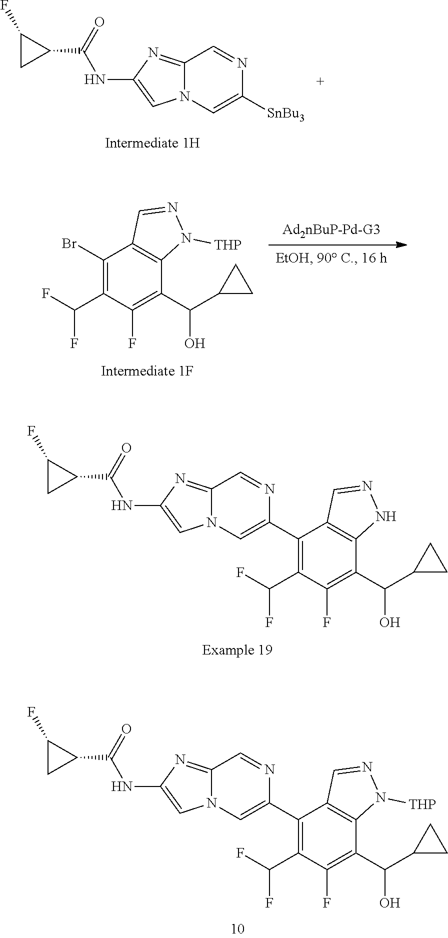

To a solution of Intermediate 1F (275 mg, 0.656 mmol, 1.0 eq) and Intermediate 1H (401 mg, 0.787 mmol, 1.2 eq) in EtOH (3.28 mL) was added Ad₂nBuP-Pd-G3 (48 mg, 0.066 mmol, 0.1 eq). The mixture was degassed and purged with N₂ for 3 times, and then stirred at 90° C. for 16 hr under N₂ atmosphere. The reaction mixture was concentrated in vacuum. The crude product was purified by silica gel chromatography (product came out at MeOH/DCM=10/100) to afford Example 19 (47.7 mg, 0.101 mmol, 15%) as a yellow color solid and Compound 10 (25 mg, 0.045 mmol, 7%).

¹H NMR (400 MHZ, DMSO-d₆) δ 13.33 (s, 1H), 11.39 (s, 1H), 9.04 (s, 1H), 8.97 (d, J=1.6 Hz, 1H), 8.39 (s, 1H), 8.15 (s, 1H), 7.10 (t, J=52.5 Hz, 1H), 5.83 (d, J=3.8 Hz, 1H), 5.07-4.86 (m, 1H), 4.65 (q, J=3.8 Hz, 1H), 2.23-2.16 (m, 1H), 1.75-1.65 (m, 1H), 1.46-1.41 (m, 1H), 1.22-1.19 (m, 1H), 0.62-0.54 (m, 2H), 0.44-0.31 (m, 2H); LCMS(electrospray) m/z 475.10 (M+H)⁺.

### Synthetic Method F

### Example 28. (1S,2S)—N-(6-(5-(difluoromethyl)-6-fluoro-7-(isopropylamino)-1H-indazol-4-yl)imidazo[1,2-a]pyrazin-2-yl)-2-fluorocyclopropane-1-carboxamide

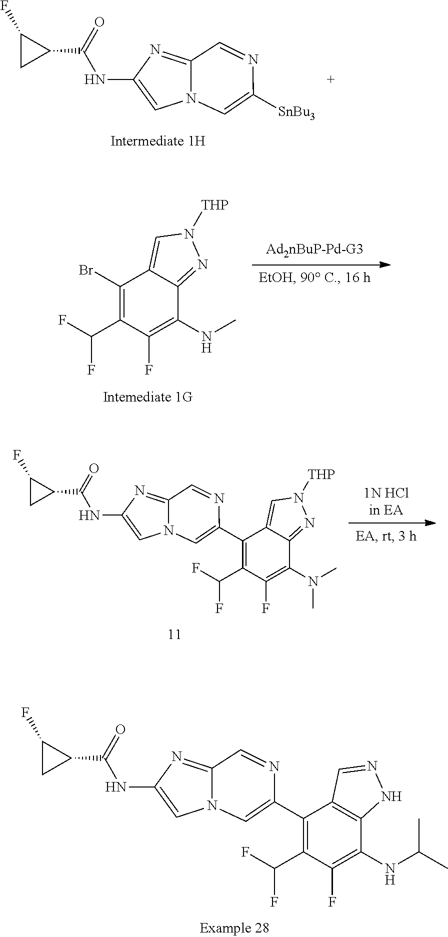

### Step 1) (1S,2S)—N-(6-(5-(difluoromethyl)-6-fluoro-7-(isopropylamino)-2-(tetrahydro-2H-pyran-2-yl)-2H-indazol-4-yl)imidazo[1,2-a]pyrazin-2-yl)-2-fluorocyclopropane-1-carboxamide

To a solution of Intermediate 1G (137 mg, 0.337 mmol, 1.0 eq) and Intermediate 1H (189 mg, 0.371 mmol, 1.1 eq) in EtOH (1.69 mL) was added Ad₂nBuP-Pd-G3 (25 mg, 0.034 mmol, 0.1 eq). The mixture was degassed and purged with N₂ for 3 times, and then stirred at 90° C. for 16 hr under N₂ atmosphere. The reaction mixture was concentrated in vacuum. The crude product was purified by silica gel chromatography to afford Compound 11 (19 mg, 0.035 mmol, 10%) as a yellow color solid.

### Step 2) (1S,2S)—N-(6-(5-(difluoromethyl)-6-fluoro-7-(isopropylamino)-1H-indazol-4-yl)imidazo[1,2-a]pyrazin-2-yl)-2-fluorocyclopropane-1-carboxamide

To a solution of Compound 11 (19 mg, 0.035 mmol, 1.0 eq) in EtOAc (0.17 mL) was added 1N HCl in EtOAc solution (0.871 mL, 0.871 mmol, 25.0 eq). The mixture was stirred at 25° C. for 3 hr. The reaction mixture was quenched by saturated NaHCO₃ and diluted with EtOAc (20 mL). Organic layers were washed with saturated NaHCO₃ (15 mL*2). The combined organic layers were dried over Na₂SO₄, filtered and the filtrate was concentrated in vacuum. The crude product was purified prep-TLC (MC:MeOH=20:1, 10:1) twice to afford Example 28 as a yellow color solid.

¹H NMR (400 MHZ, DMSO-d₆) δ 13.54-13.09 (s, 1H), 11.52-11.23 (s, 1H), 9.07-8.91 (s, 1H), 8.91-8.80 (s, 1H), 8.49-8.29 (s, 1H), 7.38-6.84 (t, 1H), 5.19-4.74 (m, 2H), 4.26-3.87 (m, 1H), 2.28-2.10 (m, 1H), 1.80-1.59 (m, 1H), 1.29-1.20 (m, 7H); LCMS(electrospray) m/z 462.10 (M+H)⁺

### Synthetic Method G

### Example 146. (1S,2S)—N-(6-(5-chloro-6-fluoro-7-((S)-1-(2,2,2-trifluoroacetamido)ethyl)-1H-indazol-4-yl)imidazo[1,2-a]pyrazin-2-yl)-2-fluorocyclopropane-1-carboxamide

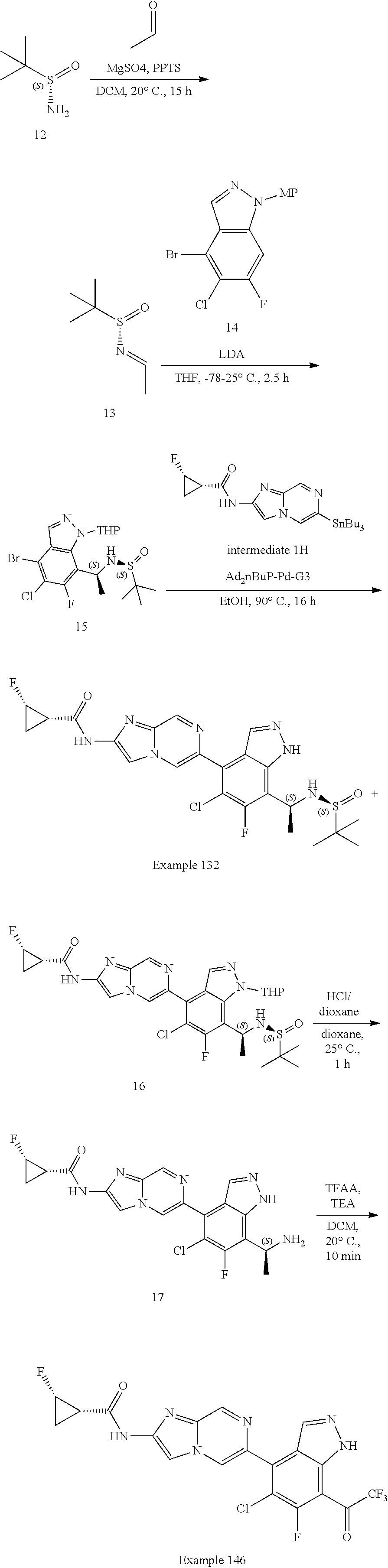

### Step 1) (S,E)-N-ethylidene-2-methylpropane-2-sulfinamide

To a solution of(S)-2-methylpropane-2-sulfinamide (3.00 g, 24.75 mmol, 1 eq) and acetaldehyde (9.81 g, 222.77 mmol, 12.50 mL, 9 eq) in DCM (30 mL) was added PPTS (622.03 mg, 2.48 mmol, 0.1 eq) and MgSO₄ (8.94 g, 74.26 mmol, 3 eq). The mixture was stirred at 25° C. for 16 hr. The reaction mixture was filtered, the filtrate was concentrated under reduced pressure to give a residue. The residue was purified by column chromatography on silical gel (petroleum ether:EtOAc=1:0 to 10:1). Compound 13 (2.9 g, 19.70 mmol, 79.57% yield) was obtained as a colorless liquid.

### Step 2) (S)—N-((1S)-1-(4-bromo-5-chloro-6-fluoro-1-(tetrahydro-2H-pyran-2-yl)-1H-indazol-7-yl)ethyl)-2-methylpropane-2-sulfinamide

To a solution of Compound 14 (1 g, 3.00 mmol, 1 eq) in THF (20 mL) was added LDA (2 M, 2.25 mL, 1.5 eq) at −78° C. under argon atmosphere. The reaction was stirred at −78° C. for 30 min, and then Compound 13 (662.57 mg, 4.50 mmol, 62.60 μL, 1.5 eq) in THF (3 mL) was added. The reaction was allowed to warm to 20° C. for 2 h. An aqueous solution of NH₄Cl (50 mL) was added dropwise into the reaction mixture. The aqueous phase was extracted with EtOAc (50 mL*3). The combined organic phase was washed with brine (20 mL*2), dried with anhydrous Na₂SO₄, filtered and concentrated in vacuum. The residue was purified by silica gel chromatography (petroleum ether:EtOAc=100:1 to 10:1) to afford product. TLC (petroleum ether:EtOAc=2:1). Compound 15 (1.1 g, 2.29 mmol, 76.26% yield) was obtained as a yellow oil.

### Step 3) (1S,2S)—N-(6-(7-((S)-1-(((S)-tert-butylsulfinyl)amino)ethyl)-5-chloro-6-fluoro-1H-indazol-4-yl)imidazo[1,2-a]pyrazin-2-yl)-2-fluorocyclopropane-1-carboxamide

To a solution of Compound 15 (550.00 mg, 1.14 mmol, 1 eq) in EtOH (20 mL) was added Intermediate 1H (582.51 mg, 1.14 mmol, 1 eq) and Ad₂nBuP-Pd-G3 (83.31 mg, 114.39 μmol, 0.1 eq). The suspension was degassed and purged with N₂ for 1 min, then the reaction mixture was stirred at 90° C. for 15 hr under N₂. 5 mL saturation KF aqueous solution was added to quench the reaction mixture. The mixture was quenched by water (20 mL) and extracted with EtOAc (20 mL*2). The combined organic layers were washed with brine 20 mL, dried over Na₂SO₄, filtered and concentrated under reduced pressure to give a residue. The residue was purified by reverse flash (MeCN/H₂O, 0.05% FA, 55%˜60%) to give product. A mixture of Compound 16 and Example 132 (350 mg, 564.42 μmol, 49.34% yield) was obtained as a yellow solid.

Take 150 mg of the mixture to purify by prep-HPLC (column: Unisil 3-100 C18 Ultra 150*50 mm*3 um; mobile phase: [water (FA)-ACN]; B %: 31%-61%, 7 min) to give product. Example 132 (10.6 mg, 19.46 μmol, 8.04% yield, 98.4% purity) was obtained as a light yellow solid. Compound 16 (60 mg, 96.76 μmol, 40.00% yield) was obtained as a light yellow solid.

¹H NMR (400 MHZ, DMSO-d₆) δ 13.40 (br s, 1H), 11.47-11.35 (m, 1H), 9.07 (br s, 1H), 9.02 (s, 1H), 8.44-8.36 (m, 1H), 8.12-8.02 (m, 1H), 5.62-5.47 (m, 1H), 5.24-5.12 (m, 1H), 5.10-4.85 (m, 1H), 2.21 (br d, J=4.3 Hz, 1H), 1.78-1.74 (m, 1H), 1.71 (br d, J=6.7 Hz, 3H), 1.24-1.20 (m, 1H), 1.06 (br s, 9H); LCMS(electrospray) m/z 536.1 (M+H)⁺.

### Step 4) (1S,2S)—N-(6-(7-((S)-1-aminoethyl)-5-chloro-6-fluoro-1H-indazol-4-yl)imidazo[1,2-a]pyrazin-2-yl)-2-fluorocyclopropane-1-carboxamide

To a solution of Compound 16 and Example 132 (130 mg, 209.64 μmol, 1 eq) in dioxane (1 mL) was added HCl/dioxane (4 M, 52.41 μL, 1 eq). The reaction was stirred at 20° C. for 1 hr. The reaction mixture was concentrated. The residue was purified by reverse flash (MeCN/H₂O, 0.05% TFA, 25%˜30%) to give product. Compound 17 (83 mg, 152.06 μmol, 72.53% yield, TFA) was obtained as a light yellow solid.

### Step 5) (1S,2S)—N-(6-(5-chloro-6-fluoro-7-((S)-1-(2,2,2-trifluoroacetamido)ethyl)-1H-indazol-4-yl)imidazo[1,2-a]pyrazin-2-yl)-2-fluorocyclopropane-1-carboxamide

To a solution of Compound 17 (83 mg, 177.24 μmol, 1 eq, HCl) and TEA (53.81 mg, 531.73 μmol, 74.01 μL, 3 eq) in DCM (1 mL) was added TFAA (55.84 mg, 265.86 μmol, 36.98 μL, 1.5 eq). The reaction mixture was stirred at 20° C. for 10 min. The reaction mixture was concentrated. The residue was purified by prep-HPLC (column: Phenomenex luna C18 150*25 mm*10 um; mobile phase: [water (FA)-ACN]; B %: 5%-35%, 10 min) to give product. Example 146 (50.2 mg, 95.11 μmol, 53.66% yield, 100% purity) was obtained as a white solid.

¹H NMR (400 MHZ, DMSO-d₆) δ 13.52 (s, 1H), 11.41 (s, 1H), 9.98 (br d, J=6.2 Hz, 1H), 9.08 (d, J=0.6 Hz, 1H), 9.02 (d, J=1.3 Hz, 1H), 8.39 (s, 1H), 8.10 (s, 1H), 5.64-5.52 (m, 1H), 5.09-4.86 (m, 1H), 2.25-2.15 (m, 1H), 1.70 (d, J=7.2 Hz, 3H), 1.69-1.64 (m, 1H), 1.26-1.16 (m, 1H); LCMS (electrospray) m/z 528.2 (M+H)⁺.

### Synthetic Method H

### Example 91. 5-chloro-6-fluoro-N-isopropyl-4-(2-morpholinoimidazo[1,2-a]pyrazin-6-yl)-1H-indazol-7-amine

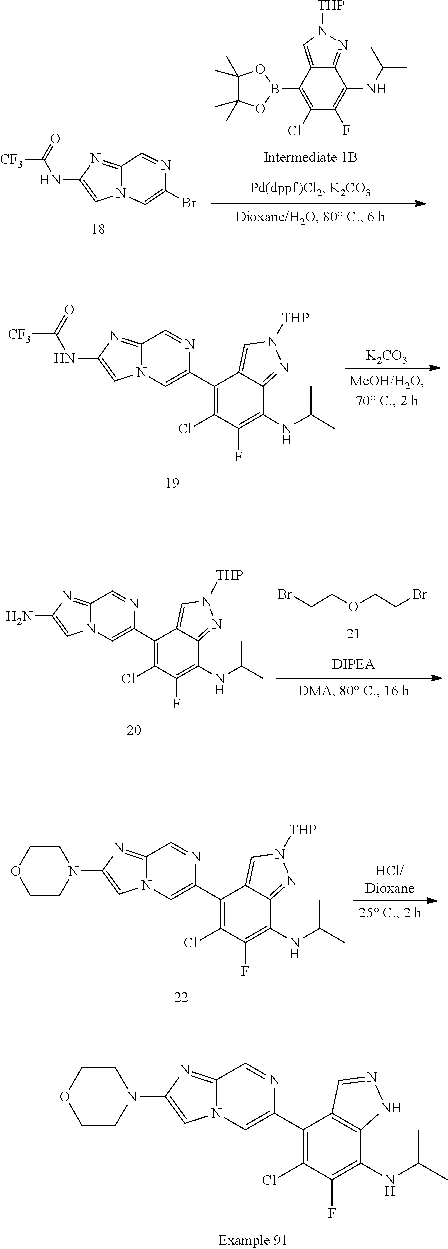

### Step 1) N-[6-[5-chloro-6-fluoro-7-(isopropylamino)-2-tetrahydropyran-2-yl-indazol-4-yl]imidazo[1,2-a]pyrazin-2-yl]-2,2,2-trifluoro-acetamide

To a solution of Intermediate 1B (3.5 g, 8.00 mmol, 1 eq) and Compound 18 (2.72 g, 8.80 mmol, 1.1 eq) in dioxane (50 mL) and H₂O (5 mL) was added Pd(dppf)Cl₂ (585.04 mg, 799.56 μmol, 0.1 eq) and K₃PO₄ (5.09 g, 23.99 mmol, 3 eq). The mixture was stirred at 80° C. for 6 hr under N₂ atmosphere.

The reaction mixture was concentrated under reduced pressure to give a residue. The residue was diluted with water (80 mL) and extracted with EtOAc (100 mL*2). The combined organic layers were washed with brine (100 mL*2), dried over anhydrous Na₂SO₄, filtered and the filtrate was concentrated under reduced pressure to give a residue. The residue was purified by column chromatography on silical gel (Petroleum ether/EtOAc=10/1 to 1/1. Compound 19 (2 g, 3.70 mmol, 46.33% yield) was obtained as a red solid.

### Step 2) 6-[5-chloro-6-fluoro-7-(isopropylamino)-2-tetrahydropyran-2-yl-indazol-4-yl]imidazo[1,2-a]pyrazin-2-amine

To a solution of Compound 19 (2.1 g, 3.89 mmol, 1 eq) in MeOH (20 mL) and H₂O (6 mL) was added K₂CO₃ (2.69 g, 19.45 mmol, 5 eq). The mixture was stirred at 70° C. for 2 hr. The reaction mixture was concentrated under reduced pressure to give a residue. The residue was purified by flash reverse phase [water (0.5% FA)/ACN condition, 51%˜57% ACN]. Compound 20 (1.2 g, 2.70 mmol, 69.50% yield) was obtained as a yellow solid.

### Step 3) 5-chloro-6-fluoro-N-isopropyl-4-(2-morpholinoimidazo[1,2-a]pyrazin-6-yl)-2-tetrahydropyran-2-yl-indazol-7-amine

To a solution of Compound 20 (100.00 mg, 225.27 μmol, 1 eq) in DMA (1 mL) were added DIPEA (58.23 mg, 450.55 μmol, 78.48 μL, 2 eq) and Compound 21 (78.37 mg, 337.91 μmol, 42.36 μL, 1.5 eq). The reaction mixture was stirred at 80° C. for 16 hr. The reaction mixture was concentrated under reduced pressure to give residue. The crude product was purified by reverse flash (ACN, Water (FA 0.05%) to give Compound 22 (30 mg, 58.37 μmol, 25.91% yield) as a yellow solid. LCMS(electrospray) m/z 514.1 (M+H)⁺.

### Step 4) 5-chloro-6-fluoro-N-isopropyl-4-(2-morpholinoimidazo[1,2-a]pyrazin-6-yl)-1H-indazol-7-amine

The Compound 22 (30 mg, 58.37 μmol, 1 eq) was dissolved in HCl/dioxane (4 M, 2 mL, 137.06 eq). The reaction mixture was stirred at 25° C. for 2 hr. The reaction mixture was concentrated under reduced pressure to give residue. The crude product was purified by prep-HPLC (column: Unisil 3-100 C18 Ultra 150*50 mm*3 um; mobile phase: [water (FA)-ACN]; B %: 33%-63%, 7 min) and lyophilized to give Example 91 (11.2 mg, 25.98 μmol, 44.52% yield) as a yellow solid.

¹H NMR (400 MHZ, DMSO-d₆) δ 13.15 (s, 1H), 8.85 (s, 1H), 8.72 (s, 1H), 7.92 (s, 1H), 7.57 (s, 1H), 5.21-5.10 (m, 1H), 4.10-3.98 (m, 1H), 3.80-3.72 (m, 4H), 3.31-3.24 (m, 4H), 1.23 (d, J=6.3 Hz, 6H); LCMS(electrospray) m/z 430.1 (M+H)⁺.

### Synthetic Method I

### Example 101. 6-(5-chloro-6-fluoro-7-(isopropylamino)-1H-indazol-4-yl)-N-(tetrahydrofuran-3-yl)imidazo[1,2-a]pyrazin-2-amine

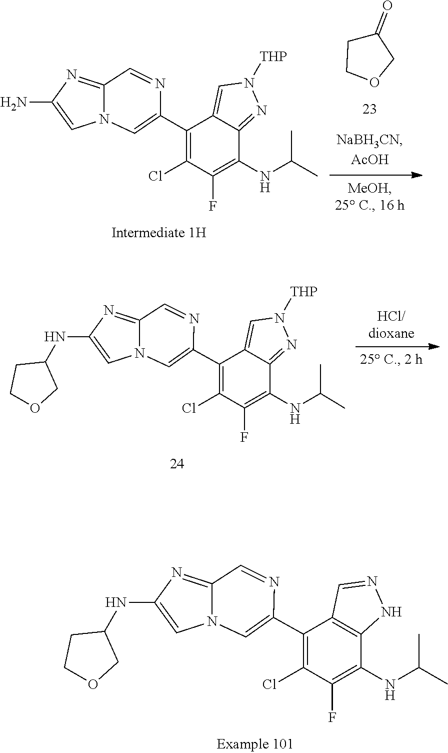

### Step 1) 6-[5-chloro-6-fluoro-7-(isopropylamino)-2-tetrahydropyran-2-yl-indazol-4-yl]-N-tetrahydrofuran-3-yl-imidazo[1,2-a]pyrazin-2-amine

The Compound 23 (38.79 mg, 450.55 μmol, 2 eq) and Compound 20 (100 mg, 225.27 μmol, 1 eq) and AcOH (1.35 mg, 22.53 μmol, 1.29 μL, 0.1 eq) were dissolved in MeOH (2 mL). After 0.5 h, NaBH₃CN (28.31 mg, 450.55 μmol, 2 eq) was added to the mixture. The reaction mixture was stirred at 25° C. for 15.5 h. The reaction mixture was quenched with water 5 mL and extracted with EtOAc (10 mL*3). The combined organic phase was washed with saturated brine (10 mL*2), dried over Na₂SO₄, filtered and the filtrate was concentrated under reduced pressure to give Compound 24 (100 mg, 194.55 μmol, 86.36% yield) as a yellow solid.

### Step 2) 6-(5-chloro-6-fluoro-7-(isopropylamino)-1H-indazol-4-yl)-N-(tetrahydrofuran-3-yl)imidazo[1,2-a]pyrazin-2-amine

The Compound 24 (100 mg, 194.55 μmol, 1 eq) was dissolved in HCl/dioxane (4 M, 2 mL, 41.12 eq). The reaction mixture was stirred at 25° C. for 2 hr. The reaction mixture was concentrated under reduced pressure to give residue. The crude product was purified by prep-HPLC (column: Unisil 3-100 C18 Ultra 150*50 mm*3 um; mobile phase: [water (FA)-ACN]; B %: 27%-57%, 7 min) to give Example 101 (23.0 mg, 53.26 μmol, 27.38% yield) as a light yellow solid.

¹H NMR (400 MHz, DMSO-d₆) δ 13.29-12.99 (m, 1H), 8.72 (s, 1H), 8.64 (s, 1H), 7.93 (br d, J=1.9 Hz, 1H), 7.33 (s, 1H), 6.47 (d, J=6.6 Hz, 1H), 5.15 (br d, J=9.0 Hz, 1H), 4.21-4.13 (m, 1H), 3.93-3.81 (m, 2H), 3.78-3.70 (m, 1H), 3.60 (dd, J=3.8, 8.7 Hz, 1H), 2.24-2.14 (m, 1H), 1.91-1.80 (m, 1H), 1.23 (d, J=6.3 Hz, 6H); LCMS(electrospray) m/z 430.2 (M+H)⁺.

### Synthetic Method J

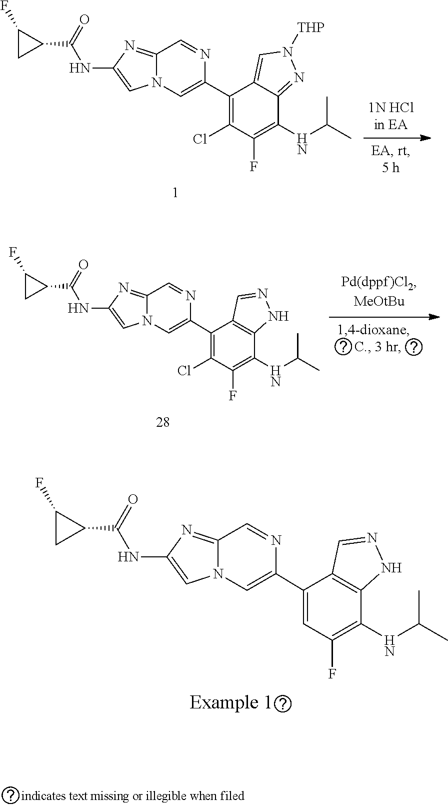

Step 1) To a solution of Compound 1 (184 g, 347 mmol, 1.00 eq) in MeOH (1.00 L) was added HCl/MeOH (4.00 M, 86.8 mL, 1.00 eq). The mixture was stirred at 15° C. for 6 hr. The reactant was concentrated in vacuum. The residue was purified by prep-HPLC column: Phenomenex Luna C18 (250*80 mm*15 um); mobile phase: [water (0.05% HCl)-CAN]; B %: 35%-60%, 35 min. The elution was basified with sat. aq NaHCO₃ solution, The mixture was concentrated in vacuum. the mixture was extracted with EtOAc (3.00 L*2). The combined organic layer was separated, washed with brine (5.00 L), dried over Na₂SO₄. To a solution of the product (160 g, 358 mmol, 1.00 eq) in MeOH (1000 mL) was added HCl/MeOH (4.00 M, 800 mL, 8.92 eq). The mixture was stirred at 25° C. for 1 hr. The reactant mixture was concentrated in vacuum to give Compound 25 (152 g, 311 mmol, 86.6% yield, 98.7% purity, HCl) as yellow solid.

¹H NMR (400 MHZ, DMSO-d₆) δ 11.58 (s, 1H), 9.21 (s, 1H), 9.02 (s, 1H), 8.50 (s, 1H), 8.00 (s, 2H), 4.98 (dtd, J=3.7, 6.8, 66.0 Hz, 1H), 4.14 (dq, J=4.0, 6.4 Hz, 1H), 2.23-2.20 (m, 1H), 1.72-1.68 (m, 1H), 1.26 (d, J=6.4 Hz, 6H), 1.23-1.21 (m, 1H); LCMS(electrospray) m/z=446.1 (M+H)⁺.

To a solution of Compound 25 (120 mg, 0.269 mmol, 1.0 eq) in 1,4-dioxane (1.35 mL) was added Pd(dppf)Cl₂ (98 mg, 0.135 mmol, 0.5 eq) and sodium tert-butoxide (38.8 mg, 0.404 mmol, 1.5 eq) at 25° C. The reaction mixture was degassed under N₂ condition and was heated to 160° C. in microwave reactor for 3 hr. After cooling down, the reaction mixture was diluted with sat. NH₄Cl, extracted with EtOAc (20 mL+3), and then dried over MgSO₄, concentrated. The residue was purified by silica gel chromatography (DCM/Methanol=20/1) to afford Example 165 (4.8 mg, 0.012 mmol, 4.33%) as a yellow solid.

¹H NMR (500 MHZ, DMSO-d₆) δ 13.07 (s, 1H), 11.33 (s, 1H), 9.17 (s, 1H), 9.01 (s, 1H), 8.61 (s, 1H), 8.35 (s, 1H), 7.55 (d, J=14.2 Hz, 2H), 5.11-4.76 (m, 2H), 4.00 (d, J=11.3 Hz, 1H), 2.19 (t, J=7.2 Hz, 1H), 1.70 (dd, J=23.2, 3.8 Hz, 1H), 1.22 (d, J=6.3 Hz, 7H); LCMS(electrospray) m/z 412.15 (M+H)⁺.

Table 1 below shows the compounds of Examples along with general synthetic methods used to make the compound and characterization data.

TABLE 1
Compounds of Examples

| Ex | | | |
|---|---|---|---|
| # | Structure/Name | ¹H NMR/MS (M + 1) | Method |
| 1 | 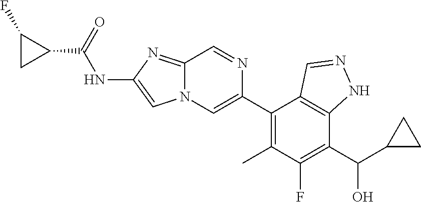 [SMILES: Cc1c(F)c(C(O)C2CC2)c2[nH]ncc2c1-c1cn2cc(NC(=O)[C@@H]3C[C@@H]3F)nc2cn1] | ¹H NMR (400 MHz, DMSO-d₆) δ 12.93 (s, 1H), 11.39 (s, 1H), 9.05 (d, J = 1.6 Hz, 1H), 8.85 (d, J = 1.6 Hz, 1H), 8.34 (s, 1H), 7.90 (d, J = 1.1 Hz, 1H), 5.70 (d, J = 3.8 Hz, 1H), 5.07-4.88 (m, 1H), 4.60 (q, J = 3.8 Hz, 1H), 2.26 (d, J = 2.7 Hz, 3H), 2.19 (t, J = 6.9 Hz, 1H), 1.72-1.65 (m, 1H), 1.44 (d, J = 7.7 Hz, 1H), 1.21-1.18 (m, 1H), 0.55 (t, J = 8.0 Hz, 2H), 0.38-0.30 (m, 2H); LCMS (electrospray) m/z 439.10 (M + H)⁺. | E |
| | (1S,2S)-N-(6-(7- | | |
| | (cyclopropyl(hydroxy)methyl)-6-fluoro- | | |
| | 5-methyl-1H-indazol-4-yl)imidazo[1,2- | | |
| | a]pyrazin-2-yl)-2-fluorocyclopropane-1- | | |
| | carboxamide | | |
| 2 | 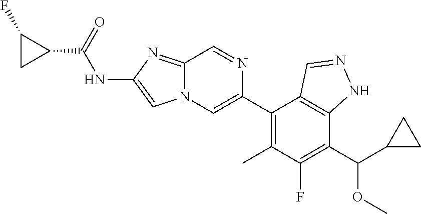 [SMILES: COC(c1c(F)c(C)c(-c2cn3cc(NC(=O)[C@@H]4C[C@@H]4F)nc3cn2)c2cn[nH]c12)C1CC1] | ¹H NMR (400 MHz, DMSO-d₆) δ 13.08 (s, 1H), 11.39 (s, 1H), 9.05 (s, 1H), 8.87 (d, J = 1.9 Hz, 1H), 8.34 (s, 1H), 7.94 (s, 1H), 5.07-4.86 (m, 1H), 4.23 (d, J = 8.8 Hz, 1H), 3.24 (s, 3H), 2.27 (d, J = 2.7 Hz, 3H), 2.22-2.15 (m, 1H), 1.74-1.64 (m, 1H), 1.50-1.48 (m, 1H), 1.21-1.16 (m, 1H), 0.70-0.57 (m, 2H), 0.43-0.36 (m, 1H), 0.28-0.24 (m, 1H); LCMS (electrospray) m/z 453.10 (M + H)⁺. | E |
| | (1S,2S)-N-(6-(7- | | |
| | (cyclopropyl(methoxy)methyl)-6-fluoro- | | |
| | 5-methyl-1H-indazol-4-yl)imidazo[1,2- | | |
| | a]pyrazin-2-yl)-2-fluorocyclopropane-1- | | |
| | carboxamide | | |
| 3 | 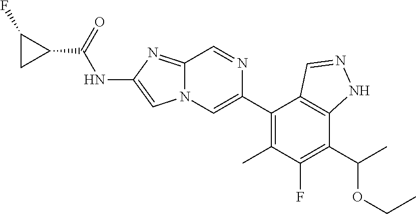 [SMILES: CCOC(C)c1c(F)c(C)c(-c2cn3cc(NC(=O)[C@@H]4C[C@@H]4F)nc3cn2)c2cn[nH]c12] | ¹H NMR (400 MHz, DMSO-d₆) δ 12.95 (s, 1H), 11.35 (s, 1H), 9.01 (s, 1H), 8.82 (d, J = 1.4 Hz, 1H), 8.30 (s, 1H), 7.90 (s, 1H), 5.09 (q, J = 6.6 Hz, 1H), 5.03-4.82 (m, 1H), 3.39 (dt, J = 16.5, 7.1 Hz, 1H), 3.25-3.20 (m, 1H), 2.23 (d, J = 2.7 Hz, 3H), 2.14 (q, J = 7.0 Hz, 1H), 1.68-1.61 (m, 1H), 1.53 (d, J = 6.6 Hz, 3H), 1.17- 1.14 (m, 1H), 1.10 (t, J = 6.9 Hz, 3H); LCMS (electrospray) m/z 441.10 (M + H)⁺. | E |
| | (1S,2S)-N-(6-(7-(1-ethoxyethyl)-6- | | |
| | fluoro-5-methyl-1H-indazol-4- | | |
| | yl)imidazo[1,2-a]pyrazin-2-yl)-2- | | |
| | fluorocyclopropane-1-carboxamide | | |
| 4 | 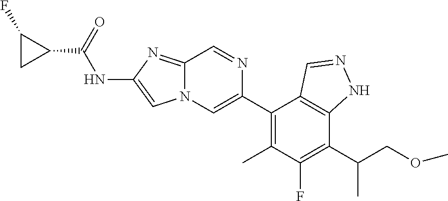 [SMILES: COCC(C)c1c(F)c(C)c(-c2cn3cc(NC(=O)[C@@H]4C[C@@H]4F)nc3cn2)c2cn[nH]c12] | ¹H NMR (400 MHz, DMSO-d₆) δ 13.20 (s, 1H), 11.38 (s, 1H), 9.03 (d, J = 3.3 Hz, 1H), 8.82 (d, J = 1.6 Hz, 1H), 8.34 (s, 1H), 7.92 (s, 1H), 5.09-4.83 (m, 1H), 3.79-3.65 (m, 3H), 3.25 (d, J = 9.3 Hz, 3H), 2.24 (d, J = 3.3 Hz, 3H), 2.22-2.14 (m, 1H), 1.75- 1.62 (m, 1H), 1.38 (d, J = 6.0 Hz, 3H), 1.24-1.15 (m, 1H); LCMS (electrospray) m/z 441.10 (M + H)⁺. | F |
| | (1S,2S)-2-fluoro-N-(6-(6-fluoro-7-(1- | | |
| | methoxypropan-2-yl)-5-methyl-1H- | | |
| | indazol-4-yl)imidazo[1,2-a]pyrazin-2- | | |
| | yl)cyclopropane-1-carboxamide | | |
| 5 | 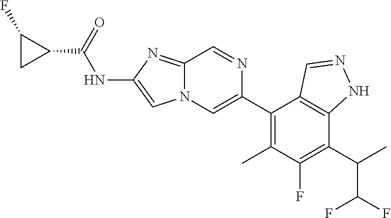 [SMILES: Cc1c(F)c(C(C)C(F)F)c2[nH]ncc2c1-c1cn2cc(NC(=O)[C@@H]3C[C@@H]3F)nc2cn1] | ¹H NMR (400 MHz, DMSO-d₆) δ 13.32 (s, 1H), 11.39 (s, 1H), 9.05 (s, 1H), 8.86 (s, 1H), 8.34 (s, 1H), 7.98 (s, 1H), 6.45 (td, J = 56.8, 6.4 Hz, 1H), 5.08-4.82 (m, 1H), 3.99-3.85 (m, 1H), 2.26 (d, 3H), 2.23-2.13 (m, 1H), 1.69 (dtd, J = 23.5, 6.9, 3.5 Hz, 1H), 1.53 (d, J = 7.1 Hz, 3H), 1.24-1.15 (m, 1H); LCMS (electrospray) m/z 447.10 (M + H)⁺. | F |
| | (1S,2S)-N-(6-(7-(1,1-difluoropropan-2- | | |
| | yl)-6-fluoro-5-methyl-1H-indazol-4- | | |
| | yl)imidazo[1,2-a]pyrazin-2-yl)-2- | | |
| | fluorocyclopropane-1-carboxamide | | |
| 6 | 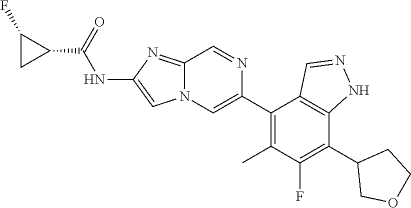 [SMILES: Cc1c(F)c(C2CCOC2)c2[nH]ncc2c1-c1cn2cc(NC(=O)[C@@H]3C[C@@H]3F)nc2cn1] | ¹H NMR (400 MHz, DMSO-d₆) δ 13.16 (s, 1H), 11.38 (s, 1H), 9.04 (s, 1H), 8.82 (d, J = 1.9 Hz, 1H), 8.34 (s, 1H), 7.96 (s, 1H), 5.11-4.80 (m, 1H), 4.14-4.03 (m, 2H), 4.03-3.80 (m, 3H), 2.38-2.22 (m, 5H), 2.22-2.14 (m, 1H), 1.75-1.60 (m, 1H), 1.24-1.14 (m, 1H); LCMS (electrospray) m/z 439.10 (M + H)⁺. | F |
| | (1S,2S)-2-fluoro-N-(6-(6-fluoro-5- | | |
| | methyl-7-(tetrahydrofuran-3-yl)-1H- | | |
| | indazol-4-yl)imidazo[1,2-a]pyrazin-2- | | |
| | yl)cyclopropane-1-carboxamide | | |
| 7 | 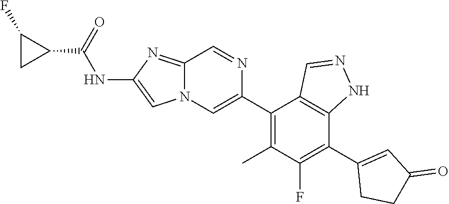 [SMILES: Cc1c(F)c(C2=CC(=O)CC2)c2[nH]ncc2c1-c1cn2cc(NC(=O)[C@@H]3C[C@@H]3F)nc2cn1] | ¹H NMR (400 MHz, DMSO-d₆) δ 13.35 (s, 1H), 11.40 (s, 1H), 9.07 (s, 1H), 8.91 (s, 1H), 8.37 (s, 1H), 8.15 (s, 1H), 6.81 (s, 1H), 5.07-4.86 (m, 1H), 3.31 (d, J = 3.3 Hz, 2H), 2.54-2.52 (m, 2H), 2.32 (d, J = 3.3 Hz, 3H), 2.23-2.16 (m, 1H), 1.74-1.64 (m, 1H), 1.25-1.16 (m, 1H); LCMS (electrospray) m/z 449.10 (M + H)⁺. | F |
| | (1S,2S)-2-fluoro-N-(6-(6-fluoro-5- | | |
| | methyl-7-(3-oxocyclopent-1-en-1-yl)-1H- | | |
| | indazol-4-yl)imidazo[1,2-a]pyrazin-2- | | |
| | yl)cyclopropane-1-carboxamide | | |
| 8 | 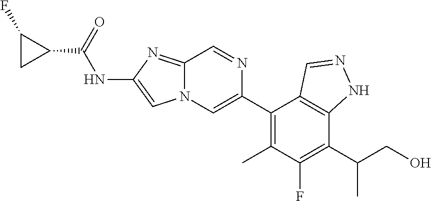 [SMILES: Cc1c(F)c(C(C)CO)c2[nH]ncc2c1-c1cn2cc(NC(=O)[C@@H]3C[C@@H]3F)nc2cn1] | ¹H NMR (400 MHz, DMSO-d₆) δ 13.12 (s, 1H), 11.37 (s, 1H), 9.04 (d, J = 2.2 Hz, 1H), 8.81 (d, J = 1.6 Hz, 1H), 8.33 (s, 1H), 7.92 (s, 1H), 5.08-5.00 (m, 1H), 4.96-4.84 (m, 2H), 3.78 (q, J = 5.3 Hz, 3H), 2.24 (d, J = 2.7 Hz, 3H), 2.21-2.14 (m, 1H), 1.76- 1.61 (m, 1H), 1.37 (d, J = 7.1 Hz, 3H), 1.24-1.14 (m, 1H); LCMS (electrospray) m/z 427.10 (M + H)⁺. | F |
| | (1S,2S)-2-fluoro-N-(6-(6-fluoro-7-(1- | | |
| | hydroxypropan-2-yl)-5-methyl-1H- | | |
| | indazol-4-yl)imidazo[1,2-a]pyrazin-2- | | |
| | yl)cyclopropane-1-carboxamide | | |
| 9 | 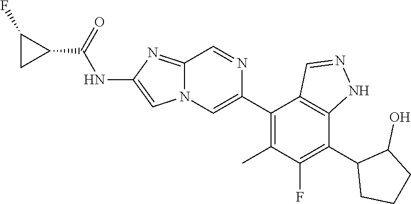 [SMILES: Cc1c(F)c(C2CCCC2O)c2[nH]ncc2c1-c1cn2cc(NC(=O)[C@@H]3C[C@@H]3F)nc2cn1] | ¹H NMR (400 MHz, DMSO-d₆) δ 13.15 (s, 1H), 11.37 (s, 1H), 9.04 (s, 1H), 8.80 (d, J = 1.6 Hz, 1H), 8.35 (s, 1H), 7.92 (s, 1H), 4.96 (dd, J = 62.7, 4.4 Hz, 2H), 4.55- 4.46 (m, 1H), 2.25 (d, J = 3.3 Hz, 3H), 2.23-2.17 (m, 1H), 2.17-1.99 (m, 3H), 1.98-1.74 (m, 3H), 1.74-1.57 (m, 2H), 1.26-1.18 (m, 1H); LCMS (electrospray) m/z 453.20 (M + H)⁺. | F |
| | (1S,2S)-2-fluoro-N-(6-(6-fluoro-7-(2- | | |
| | hydroxycyclopentyl)-5-methyl-1H- | | |
| | indazol-4-yl)imidazo[1,2-a]pyrazin-2- | | |
| | yl)cyclopropane-1-carboxamide | | |
| 10 | 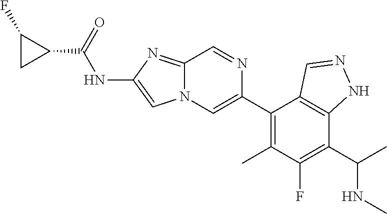 [SMILES: CNC(C)c1c(F)c(C)c(-c2cn3cc(NC(=O)[C@@H]4C[C@@H]4F)nc3cn2)c2cn[nH]c12] | ¹H NMR (400 MHz, DMSO-d₆) δ 12.66 (s, 1H), 11.37 (s, 1H), 9.03 (s, 1H), 8.78 (d, J = 1.6 Hz, 1H), 8.33 (s, 1H), 7.80 (d, J = 1.1 Hz, 1H), 5.48 (d, J = 3.0 Hz, 1H), 5.40 (q, J = 3.3 Hz, 1H), 5.06-4.86 (m, 1H), 3.76 (s, 3H), 2.23 (d, J = 8.2 Hz, 3H), 2.20-2.15 (m, 1H), 1.72-1.65 (m, 1H), 1.53 (d, J = 6.6 Hz, 3H), 1.21-1.15 (m, 1H); LCMS (electrospray) m/z 426.15 (M + H)⁺. | E |
| | (1S,2S)-2-fluoro-N-(6-(6-fluoro-5- | | |
| | methyl-7-(1-(methylamino)ethyl)-1H- | | |
| | indazol-4-yl)imidazo[1,2-a]pyrazin-2- | | |
| | yl)cyclopropane-1-carboxamide | | |
| 11 | 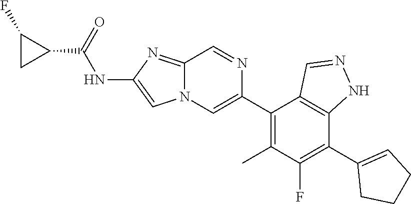 [SMILES: Cc1c(F)c(C2=CCCC2)c2[nH]ncc2c1-c1cn2cc(NC(=O)[C@@H]3C[C@@H]3F)nc2cn1] | ¹H NMR (400 MHz, DMSO-d₆) δ 13.05 (s, 1H), 11.38 (s, 1H), 9.05 (s, 1H), 8.84 (d, J = 1.6 Hz, 1H), 8.35 (s, 1H), 7.97 (s, 1H), 6.33 (s, 1H), 5.11-4.83 (m, 1H), 2.86 (s, 2H), 2.59 (s, 2H), 2.26 (d, J = 3.3 Hz, 3H), 2.22-2.13 (m, 1H), 2.08-1.96 (m, 2H), 1.75-1.62 (m, 1H), 1.25-1.15 (m, 1H); LCMS (electrospray) m/z 435.10 (M + H)⁺. | F |
| | (1S,2S)-N-(6-(7-(cyclopent-1-en-1-yl)-6- | | |
| | fluoro-5-methyl-1H-indazol-4- | | |
| | yl)imidazo[1,2-a]pyrazin-2-yl)-2- | | |
| | fluorocyclopropane-1-carboxamide | | |
| 12 | 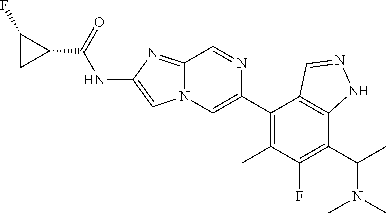 [SMILES: Cc1c(F)c(C(C)N(C)C)c2[nH]ncc2c1-c1cn2cc(NC(=O)[C@@H]3C[C@@H]3F)nc2cn1] | ¹H NMR (400 MHz, DMSO-d₆) δ 12.81 (s, 1H), 11.38 (s, 1H), 9.04 (s, 1H), 8.85 (d, J = 1.6 Hz, 1H), 8.33 (s, 1H), 7.91 (s, 1H), 5.04-4.87 (m, 1H), 3.84 (q, 1H), 2.26 (d, J = 2.7 Hz, 3H), 2.20-2.20 (m, 7H), 1.72-1.66 (m, 1H), 1.47 (d, J = 6.6 Hz, 3H), 1.20 (s, 1H); LCMS (electrospray) m/z 440.20 (M + H)⁺. | D |
| | (1S,2S)-N-(6-(7-(1- | | |
| | (dimethylamino)ethyl)-6-fluoro-5- | | |
| | methyl-1H-indazol-4-yl)imidazo[1,2- | | |
| | a]pyrazin-2-yl)-2-fluorocyclopropane-1- | | |
| | carboxamide | | |
| 13 | 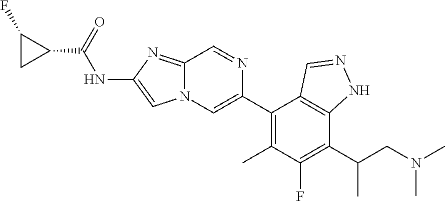 [SMILES: Cc1c(F)c(C(C)CN(C)C)c2[nH]ncc2c1-c1cn2cc(NC(=O)[C@@H]3C[C@@H]3F)nc2cn1] | ¹H NMR (400 MHz, DMSO-d₆) δ 13.20 (s, 1H), 11.38 (s, 1H), 9.04 (s, 1H), 8.83 (d, J = 1.6 Hz, 1H), 8.34 (s, 1H), 7.91 (s, 1H), 5.07-4.82 (m, 1H), 3.66-3.55 (m, 1H), 2.86-2.76 (m, 1H), 2.56-2.51 (m, 1H), 2.24 (d, J = 3.3 Hz, 3H), 2.18 (t, J = 7.1 Hz, 1H), 2.13 (s, 6H), 1.75-1.62 (m, 1H), 1.36 (d, J = 6.6 Hz, 3H), 1.24-1.15 (m, 1H); LCMS (electrospray) m/z 454.20 (M + H)⁺. | F |
| | (1S,2S)-N-(6-(7-(1- | | |
| | (dimethylamino)propan-2-yl)-6-fluoro-5- | | |
| | methyl-1H-indazol-4-yl)imidazo[1,2- | | |
| | a]pyrazin-2-yl)-2-fluorocyclopropane-1- | | |
| | carboxamide | | |
| 14 | 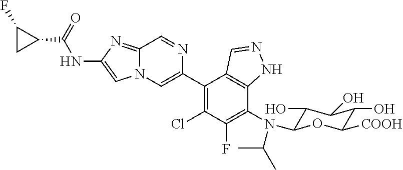 [SMILES: CC(C)N(c1c(F)c(Cl)c(-c2cn3cc(NC(=O)[C@@H]4C[C@@H]4F)nc3cn2)c2cn[nH]c12)C1OC(C(=O)O)C(O)C(O)C1O] | ¹H NMR (400 MHz, DMSO-d₆) δ 13.62 (br s, 1H), 11.61 − 11.16 (m, 1H), 9.07 (s, 1H), 9.00 (s, 1H), 8.37 (s, 1H), 7.96 (br s, 1H), 5.05 (br s, 1H), 4.94 − 4.74 (m, 1H), 4.62 (br d, J = 8.6 Hz, 1H), 4.49 (br s, 1H), 3.69 − 3.63 (m, 1H), 3.61 − 3.54 (m, 1H), 3.24 (br d, J = 9.0 Hz, 2H), 2.90 (br t, J = 8.8 Hz, 1H), 2.62 (br s, 1H), 2.20 (br s, 1H), 1.77 − 1.64 (m, 1H), 1.29 − 1.10 (m, 8H); LCMS (electrospray) m/z = 622.2 (M + H)⁺. | A |
| | 6-((5-chloro-6-fluoro-4-(2-((1S,2S)-2- | | |
| | fluorocyclopropane-1- | | |
| | carboxamido)imidazo[1,2-a]pyrazin-6- | | |
| | yl)-1H-indazol-7-yl)(isopropyl)amino)- | | |
| | 3,4,5-trihydroxytetrahydro-2H-pyran-2- | | |
| | carboxylic acid | | |
| 15 | 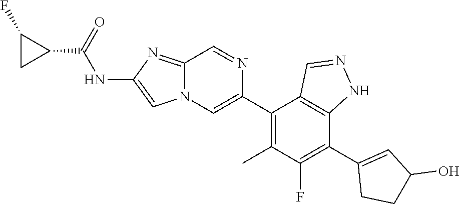 [SMILES: Cc1c(F)c(C2=CC(O)CC2)c2[nH]ncc2c1-c1cn2cc(NC(=O)[C@@H]3C[C@@H]3F)nc2cn1] | ¹H NMR (400 MHz, DMSO-d₆) δ 13.02 (s, 1H), 11.39 (s, 1H), 9.05 (s, 1H), 8.86 (d, J = 1.1 Hz, 1H), 8.35 (s, 1H), 7.99 (s, 1H), 6.30 (s, 1H), 5.05-4.86 (m, 3H), 3.03-3.03 (m, 1H), 2.75-2.75 (m, 1H), 2.32 (d, J = 3.3 Hz, 1H), 2.28 (d, J = 2.7 Hz, 3H), 2.22-2.15 (m, 1H), 1.79-1.64 (m, 2H), 1.21-1.18 (m, 1H); LCMS (electrospray) m/z 451.15 (M + H)⁺. | F |
| | (1S,2S)-2-fluoro-N-(6-(6-fluoro-7-(3- | | |
| | hydroxycyclopent-1-en-1-yl)-5-methyl- | | |
| | 1H-indazol-4-yl)imidazo[1,2-a]pyrazin-2- | | |
| | yl)cyclopropane-1-carboxamide | | |
| 16 | 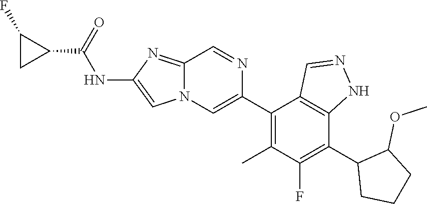 [SMILES: COC1CCCC1c1c(F)c(C)c(-c2cn3cc(NC(=O)[C@@H]4C[C@@H]4F)nc3cn2)c2cn[nH]c12] | ¹H NMR (400 MHz, DMSO-d₆) δ 13.27 (s, 1H), 11.38 (s, 1H), 9.04 (s, 1H), 8.83 (d, J = 1.1 Hz, 1H), 8.34 (s, 1H), 7.93 (s, 1H), 5.08-4.85 (m, 1H), 4.19 (d, J = 6.6 Hz, 1H), 3.60-3.51 (m, 1H), 3.10 (s, 3H), 2.26 (d, J = 3.3 Hz, 3H), 2.22-2.12 (m, 2H), 2.08 (m, 1H), 1.94-1.71 (m, 4H), 1.71-1.61 (m, 1H), 1.24-1.13 (m, 1H); LCMS (electrospray) m/z 467.20 (M + H)+ | F |
| | (1S,2S)-2-fluoro-N-(6-(6-fluoro-7-(2- | | |
| | methoxycyclopentyl)-5-methyl-1H- | | |
| | indazol-4-yl)imidazo[1,2-a]pyrazin-2- | | |
| | yl)cyclopropane-1-carboxamide | | |
| 17 | 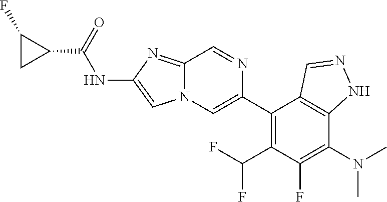 [SMILES: CN(C)c1c(F)c(C(F)F)c(-c2cn3cc(NC(=O)[C@@H]4C[C@@H]4F)nc3cn2)c2cn[nH]c12] | ¹H NMR (400 MHz, DMSO-d₆) δ 13.58 (s, 1H), 11.38 (s, 1H), 9.02 (s, 1H), 8.92 (s, 1H), 8.38 (s, 1H), 8.16 (s, 1H), 7.08 (t, J = 52.5 Hz, 1H), 5.09-4.86 (m, 1H), 3.00-2.91 (m, 6H), 2.22-2.17 (m, 1H), 1.72-1.64 (m, 2H), 1.20-1.20 (m, 1H); LCMS (electrospray) m/z 448.10 (M + H)⁺. | D |
| | (1S,2S)-N-(6-(5-(difluoromethyl)-7- | | |
| | (dimethylamino)-6-fluoro-1H-indazol-4- | | |
| | yl)imidazo[1,2-a]pyrazin-2-yl)-2- | | |
| | fluorocyclopropane-1-carboxamide | | |
| 18 | 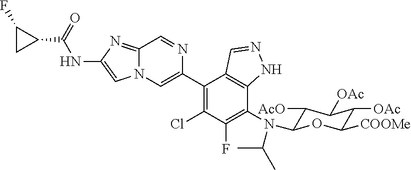 [SMILES: COC(=O)C1OC(N(c2c(F)c(Cl)c(-c3cn4cc(NC(=O)[C@@H]5C[C@@H]5F)nc4cn3)c3cn[nH]c23)C(C)C)C(OC(C)=O)C(OC(C)=O)C1OC(C)=O] | ¹H NMR (400 MHz, CDCl₃-d) δ 12.21 (br s, 1H), 9.11 (br s, 1H), 8.97 (br s, 1H), 8.51 (s, 1H), 8.34 (br s, 1H), 8.04 (s, 1H), 5.43 − 5.30 (m, 1H), 5.10 (d, J = 9.5 Hz, 1H), 5.00 − 4.77 (m, 2H), 4.57 (br t, J = 9.4 Hz, 1H), 4.25 (d, J = 9.9 Hz, 1H), 3.90 (s, 3H), 3.62 (td, J = 6.6, 13.2 Hz, 1H), 2.09 (s, 3H), 2.04 (s, 3H), 2.01 − 1.94 (m, 2H), 1.92 (s, 3H), 1.40 − 1.26 (m, 7H); LCMS (electrospray), m/z = 762.6 (M + H)⁺. | A |
| | 2-((5-chloro-6-fluoro-4-(2-((1S,2S)-2- | | |
| | fluorocyclopropane-1- | | |
| | carboxamido)imidazo[1,2-a]pyrazin-6- | | |
| | yl)-1H-indazol-7-yl)(isopropyl)amino)-6- | | |
| | (methoxycarbonyl)tetrahydro-2H-pyran- | | |
| | 3,4,5-triyl triacetate | | |
| 19 | 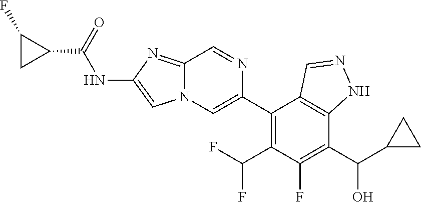 [SMILES: O=C(Nc1cn2cc(-c3c(C(F)F)c(F)c(C(O)C4CC4)c4[nH]ncc34)ncc2n1)[C@@H]1C[C@@H]1F] | ¹H NMR (400 MHz, DMSO-d₆) δ 13.33 (s, 1H), 11.39 (s, 1H), 9.04 (s, 1H), 8.97 (d, J = 1.6 Hz, 1H), 8.39 (s, 1H), 8.15 (s, 1H), 7.10 (t, J = 52.5 Hz, 1H), 5.83 (d, J = 3.8 Hz, 1H), 5.07-4.86 (m, 1H), 4.65 (q, J = 3.8 Hz, 1H), 2.23-2.16 (m, 1H), 1.75- 1.65 (m, 1H), 1.46-1.41 (m, 1H), 1.22- 1.19 (m, 1H), 0.62-0.54 (m, 2H), 0.44- 0.31 (m, 2H); LCMS (electrospray) m/z 475.10 (M + H)⁺. | E |
| | (1S,2S)-N-(6-(7- | | |
| | (cyclopropyl(hydroxy)methyl)-5- | | |
| | (difluoromethyl)-6-fluoro-1H-indazol-4- | | |
| | yl)imidazo[1,2-a]pyrazin-2-yl)-2- | | |
| | fluorocyclopropane-1-carboxamide | | |
| 20 | 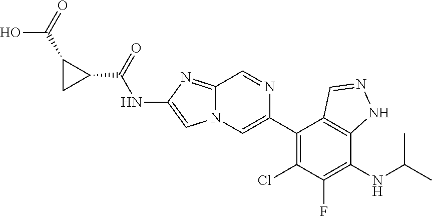 [SMILES: CC(C)Nc1c(F)c(Cl)c(-c2cn3cc(NC(=O)[C@@H]4C[C@@H]4C(=O)O)nc3cn2)c2cn[nH]c12] | ¹H NMR (400 MHz, DMSO-d₆) δ 13.22 − 13.11 (m, 1H), 12.21 − 12.07 (m, 1H), 11.49 − 11.33 (m, 1H), 9.01 (s, 1H), 8.88 (s, 1H), 8.31 (s, 1H), 7.94 (br s, 1H), 5.19 (br d, J = 8.3 Hz, 1H), 4.12 − 3.95 (m, 1H), 2.31 − 2.24 (m, 1H), 2.12 − 2.02 (m, 1H), 1.50 − 1.41 (m, 1H), 1.23 (d, J = 6.2 Hz, 6H), 1.21 − 1.17 (m, 1H); LCMS (electrospray) m/z = 472.2 (M + H)⁺. | B |
| | (1S,2R)-2-((6-(5-chloro-6-fluoro-7- | | |
| | (isopropylamino)-1H-indazol-4- | | |
| | yl)imidazo[1,2-a]pyrazin-2- | | |
| | yl)carbamoyl)cyclopropane-1-carboxylic | | |
| | acid | | |
| 21 | 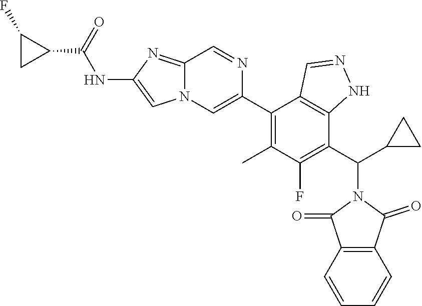 [SMILES: Cc1c(F)c(C(C2CC2)N2C(=O)c3ccccc3C2=O)c2[nH]ncc2c1-c1cn2cc(NC(=O)[C@@H]3C[C@@H]3F)nc2cn1] | ¹H NMR (400 MHz, DMSO-d₆) δ 13.11 (s, 1H), 11.37 (s, 1H), 9.03 (s, 1H), 8.84 (d, J = 1.1 Hz, 1H), 8.33 (s, 1H), 7.99 (s, 1H), 7.87 (s, 4H), 5.08-4.81 (m, 2H), 2.18 (d, J = 2.7 Hz, 4H), 1.76-1.62 (m, 1H), 1.27-1.13 (m, 2H), 0.71 (dt, J = 22.4, 9.1 Hz, 2H), 0.50 (s, 2H); LCMS (electrospray) m/z 568.20 (M + H)⁺. | E |
| | (1S,2S)-N-(6-(7-(cyclopropyl(1,3- | | |
| | dioxoisoindolin-2-yl)methyl)-6-fluoro-5- | | |
| | methyl-1H-indazol-4-yl)imidazo[1,2- | | |
| | a]pyrazin-2-yl)-2-fluorocyclopropane-1- | | |
| | carboxamid | | |
| 22 | 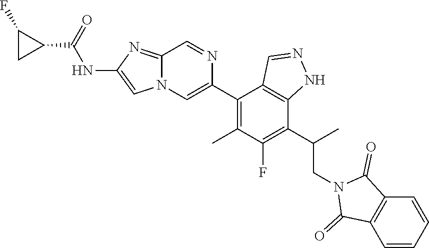 [SMILES: Cc1c(F)c(C(C)CN2C(=O)c3ccccc3C2=O)c2[nH]ncc2c1-c1cn2cc(NC(=O)[C@@H]3C[C@@H]3F)nc2cn1] | ¹H NMR (400 MHz, DMSO-d₆) δ 13.25 (s, 1H), 11.36 (s, 1H), 9.04-8.96 (1H), 8.82-8.78 (1H), 8.33 (s, 1H), 7.91 (s, 1H), 7.82 (dd, J = 6.6, 4.4 Hz, 4H), 5.07-4.83 (m, 1H), 4.18 (dd, J = 13.7, 8.2 Hz, 1H), 4.01 (q, J = 7.0 Hz, 1H), 3.98-3.84 (m, 1H), 2.21-2.13 (4H), 1.77-1.62 (m, 1H), 1.48 (d, J = 7.1 Hz, 3H), 1.24-1.16 (m, 1H); LCMS (electrospray) m/z 556.15 (M + H)⁺. | F |
| | (1S,2S)-N-(6-(7-(1-(1,3-dioxoisoindolin- | | |
| | 2-yl)propan-2-yl)-6-fluoro-5-methyl-1H- | | |
| | indazol-4-yl)imidazo[1,2-a]pyrazin-2-yl)- | | |
| | 2-fluorocyclopropane-1-carboxamide | | |
| 23 | 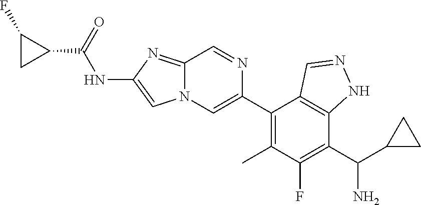 [SMILES: Cc1c(F)c(C(N)C2CC2)c2[nH]ncc2c1-c1cn2cc(NC(=O)[C@@H]3C[C@@H]3F)nc2cn1] | ¹H NMR (400 MHz, DMSO-d₆) δ 11.35 (s, 1H), 9.01 (s, 1H), 8.79 (d, J = 1.1 Hz, 1H), 8.30 (s, 1H), 7.88 (s, 1H), 4.93 (ddd, J = 66.2, 9.9, 6.3 Hz, 1H), 3.74 (d, J = 8.8 Hz, 1H), 2.22 (d, J = 2.7 Hz, 3H), 2.19- 2.09 (m, 1H), 1.71-1.58 (m, 1H), 1.38- 1.27 (m, 1H), 1.20-1.10 (m, 1H), 0.58- 0.37 (m, 2H), 0.34-0.12 (m, 2H); LCMS (electrospray) m/z 438.20 (M + H)⁺. | E |
| | (1S,2S)-N-(6-(7- | | |
| | (amino(cyclopropyl)methyl)-6-fluoro-5- | | |
| | methyl-1H-indazol-4-yl)imidazo[1,2- | | |
| | a]pyrazin-2-yl)-2-fluorocyclopropane-1- | | |
| | carboxamide | | |
| 24 | 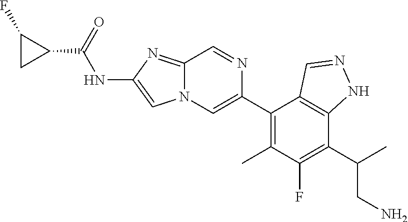 [SMILES: Cc1c(F)c(C(C)CN)c2[nH]ncc2c1-c1cn2cc(NC(=O)[C@@H]3C[C@@H]3F)nc2cn1] | ¹H NMR (400 MHz, DMSO-d₆) δ 11.36 (s, 1H), 9.04 (s, 1H), 8.82 (s, 1H), 8.34 (s, 1H), 7.92 (s, 1H), 5.14-4.82 (1H), 3.47- 3.38 (2H), 3.05-2.92 (m, 1H), 2.25 (d, J = 2.7 Hz, 3H), 2.22-2.12 (m, 1H), 1.77-1.59 (m, 1H), 1.38 (d, J = 7.1 Hz, 3H), 1.24- 1.15 (m, 1H); LCMS (electrospray) m/z 426.15 (M + H)⁺. | F |
| | (1S,2S)-N-(6-(7-(1-aminopropan-2-yl)-6- | | |
| | fluoro-5-methyl-1H-indazol-4- | | |
| | yl)imidazo[1,2-a]pyrazin-2-yl)-2- | | |
| | fluorocyclopropane-1-carboxamide | | |
| 25 | 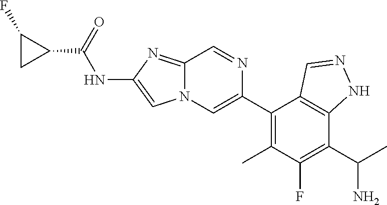 [SMILES: Cc1c(F)c(C(C)N)c2[nH]ncc2c1-c1cn2cc(NC(=O)[C@@H]3C[C@@H]3F)nc2cn1] | ¹H NMR (400 MHz, DMSO-d₆) δ 11.37 (s, 1H), 9.04 (s, 1H), 8.89-8.75 (1H), 8.34 (s, 1H), 7.92 (s, 1H), 4.96 (ddd, J = 66.2, 9.9, 6.3 Hz, 1H), 4.68 (q, J = 6.6 Hz, 1H), 2.34-2.24 (m, 3H), 2.23-2.13 (m, 1H), 1.75-1.60 (m, 1H), 1.48 (dd, J = 21.2, 6.9 Hz, 3H), 1.25-1.12 (m, 1H); LCMS (electrospray) m/z 412.10 (M + H)⁺. | E |
| | (1S,2S)-N-(6-(7-(1-aminoethyl)-6-fluoro- | | |
| | 5-methyl-1H-indazol-4-yl)imidazo[1,2- | | |
| | a]pyrazin-2-yl)-2-fluorocyclopropane-1- | | |
| | carboxamide | | |
| 26 | 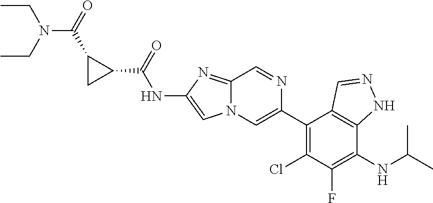 [SMILES: CCN(CC)C(=O)[C@H]1C[C@H]1C(=O)Nc1cn2cc(-c3c(Cl)c(F)c(NC(C)C)c4[nH]ncc34)ncc2n1] | ¹H NMR (400 MHz, DMSO-d₆) δ 13.35 − 13.00 (m, 1H), 11.22 (s, 1H), 9.00 (s, 1H), 8.89 (d, J = 1.2 Hz, 1H), 8.27 (s, 1H), 8.10 − 7.85 (m, 1H), 5.28 − 5.11 (m, 1H), 4.24 − 3.87 (m, 1H), 3.55 (qd, J = 7.0, 14.4 Hz, 1H), 3.29 − 3.19 (m, 2H), 3.18 − 3.08 (m, 1H), 2.37 − 2.30 (m, 2H), 2.25 − 2.18 (m, 1H), 1.51 (dt, J = 4.1, 6.4 Hz, 1H), 1.23 (d, J = 6.4 Hz, 7H), 1.16 (dt, J = 3.9, 8.2 Hz, 2H), 1.06 (t, J = 7.1 Hz, 3H), 0.90 (t, J = 7.0 Hz, 3H); LCMS (electrospray) m/z = 527.3 (M + H)⁺. | C |
| | (1R,2S)-N1-(6-(5-chloro-6-fluoro-7- | | |
| | (isopropylamino)-1H-indazol-4- | | |
| | yl)imidazo[1,2-a]pyrazin-2-yl)-N2,N2- | | |
| | diethylcyclopropane-1,2-dicarboxamide | | |
| 27 | 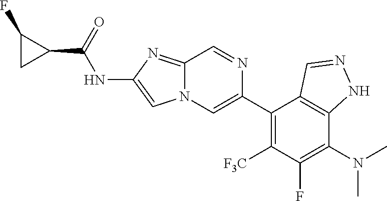 [SMILES: CN(C)c1c(F)c(C(F)(F)F)c(-c2cn3cc(NC(=O)[C@H]4C[C@H]4F)nc3cn2)c2cn[nH]c12] | ¹H NMR (400 MHz, DMSO-d₆) δ 13.71 (br s, 1H), 11.40 (s, 1H), 8.99 (s, 1H), 8.82 (s, 1H), 8.34 (s, 1H), 7.98 (s, 1H), 5.11 − 4.81 (m, 1H), 2.22 − 2.15 (m, 1H), 1.74 − 1.64 (m, 1H), 1.23 − 1.16 (m, 1H); LCMS (electrospray m/z = 466.1 (M + H)⁺. | C |
| | (1R,2R)-N-(6-(7-(dimethylamino)-6- | | |
| | fluoro-5-(trifluoromethyl)-1H-indazol-4- | | |
| | yl)imidazo[1,2-a]pyrazin-2-yl)-2- | | |
| | fluorocyclopropane-1-carboxamide | | |
| 28 | 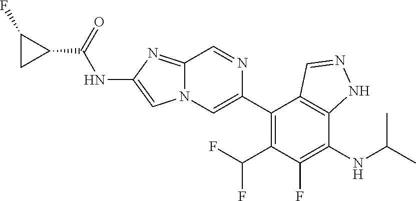 [SMILES: CC(C)Nc1c(F)c(C(F)F)c(-c2cn3cc(NC(=O)[C@@H]4C[C@@H]4F)nc3cn2)c2cn[nH]c12] | ¹H NMR (400 MHz, DMSO-d₆) δ 13.54 − 13.09 (s, 1H), 11.52 − 11.23 (s, 1H), 9.07 − 8.91 (s, 1H), 8.91 − 8.80 (s, 1H), 8.49 − 8.29 (s, 1H), 7.38 − 6.84 (t, 1H), 5.19 − 4.74 (m, 2H), 4.26 − 3.87 (m, 1H), 2.28 − 2.10 (m, 1H), 1.80 − 1.59 (m, 1H), 1.29 − 1.20 (m, 7H); LCMS (electrospray) m/z 462.10 (M + H)⁺. | F |
| | (1S,2S)-N-(6-(5-(difluoromethyl)-6- | | |
| | fluoro-7-(isopropylamino)-1H-indazol-4- | | |
| | yl)imidazo[1,2-a]pyrazin-2-yl)-2- | | |
| | fluorocyclopropane-1-carboxamide | | |
| 29 | 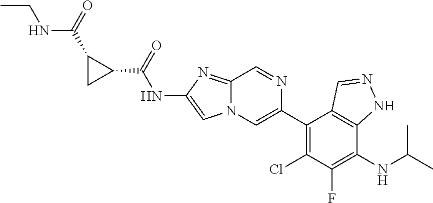 [SMILES: CCNC(=O)[C@H]1C[C@H]1C(=O)Nc1cn2cc(-c3c(Cl)c(F)c(NC(C)C)c4[nH]ncc34)ncc2n1] | ¹H NMR (400 MHz, DMSO-d₆) δ 13.25 − 13.10 (m, 1H), 11.13 (s, 1H), 9.00 (s, 1H), 8.89 (s, 1H), 8.31 (s, 1H), 8.03 (br t, J = 5.6 Hz, 1H), 7.96 (br s, 1H), 5.20 (br dd, J = 2.0, 9.7 Hz, 1H), 4.16 − 3.94 (m, 1H), 3.10 − 3.00 (m, 2H), 2.21 − 2.12 (m, 1H), 2.04 − 1.94 (m, 1H), 1.56 − 1.47 (m, 1H), 1.24 (d, J = 6.4 Hz, 8H), 1.17 (dt, J = 4.2, 8.4 Hz, 1H), 0.99 (t, J = 7.2 Hz, 3H); LCMS (electrospray) m/z = 499.2 (M + H)⁺. | C |
| | (1R,2S)-N1-(6-(5-chloro-6-fluoro-7- | | |
| | (isopropylamino)-1H-indazol-4- | | |
| | yl)imidazo[1,2-a]pyrazin-2-yl)-N2- | | |
| | ethylcyclopropane-1,2-dicarboxamide | | |
| 30 | 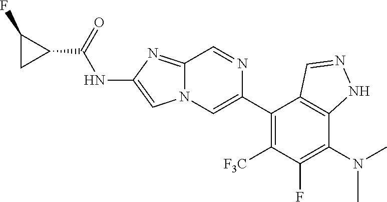 [SMILES: CN(C)c1c(F)c(C(F)(F)F)c(-c2cn3cc(NC(=O)[C@@H]4C[C@H]4F)nc3cn2)c2cn[nH]c12] | ¹H NMR (400 MHz, DMSO-d₆) δ 13.89 − 13.45 (m, 1H), 11.50 (s, 1H), 9.00 (s, 1H), 8.82 (d, J = 1.2 Hz, 1H), 8.29 (s, 1H), 8.09 − 7.91 (m, 1H), 5.06 − 4.80 (m, 1H), 3.04 (d, J = 2.0 Hz, 6H), 1.64 − 1.52 (m, 1H), 1.34 − 1.24 (m, 1H); LCMS (electrospray) m/z= 466.3 (M + H)⁺. | C |
| | (1S,2R)-N-(6-(7-(dimethylamino)-6- | | |
| | fluoro-5-(trifluoromethyl)-1H-indazol-4- | | |
| | yl)imidazo[1,2-a]pyrazin-2-yl)-2- | | |
| | fluorocyclopropane-1-carboxamide | | |
| 31 | 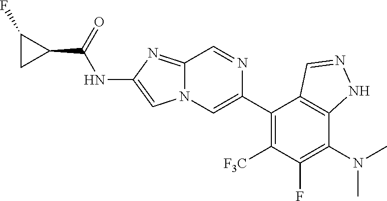 [SMILES: CN(C)c1c(F)c(C(F)(F)F)c(-c2cn3cc(NC(=O)[C@H]4C[C@@H]4F)nc3cn2)c2cn[nH]c12] | ¹H NMR (400 MHz, DMSO-d₆) δ 13.86 − 13.60 (m, 1H), 11.50 (s, 1H), 9.00 (s, 1H), 8.82 (d, J = 1.1 Hz, 1H), 8.29 (s, 1H), 8.00 (s, 1H), 5.05 − 4.82 (m, 1H), 3.04 (d, J = 2.0 Hz, 6H), 1.64 − 1.51 (m, 1H), 1.28 (td, J = 6.9, 13.5 Hz, 1H); LCMS (electrospray) m/z = 466.3 (M + H)⁺. | C |
| | (1R,2S)-N-(6-(7-(dimethylamino)-6- | | |
| | fluoro-5-(trifluoromethyl)-1H-indazol-4- | | |
| | yl)imidazo[1,2-a]pyrazin-2-yl)-2- | | |
| | fluorocyclopropane-1-carboxamide | | |
| 32 | 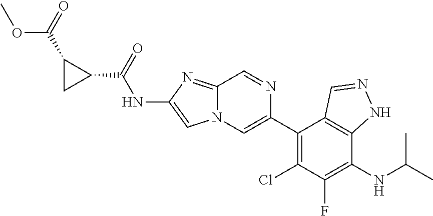 [SMILES: COC(=O)[C@H]1C[C@H]1C(=O)Nc1cn2cc(-c3c(Cl)c(F)c(NC(C)C)c4[nH]ncc34)ncc2n1] | ¹H NMR (400 MHz, DMSO-d₆) δ 13.28 − 13.07 (m, 1H), 11.36 (s, 1H), 9.01 (s, 1H), 8.87 (s, 1H), 8.31 (s, 1H), 7.97 (br s, 1H), 5.20 (br dd, J = 1.9, 9.3 Hz, 1H), 4.19 − 3.99 (m, 1H), 3.54 (s, 3H), 2.40 − 2.33 (m, 1H), 2.26 − 2.17 (m, 1H), 1.51 − 1.44 (m, 1H), 1.27 (br d, J = 4.3 Hz, 1H), 1.23 (d, J = 6.3 Hz, 6H); LCMS (electrospray) m/z= 486.1 (M +H)⁺. | C |
| | methyl (1S,2R)-2-((6-(5-chloro-6-fluoro- | | |
| | 7-(isopropylamino)- 1H-indazol-4- | | |
| | yl)imidazo[1,2-a]pyrazin-2- | | |
| | yl)carbamoyl)cyclopropane-1- | | |
| | carboxylate | | |
| 33 | 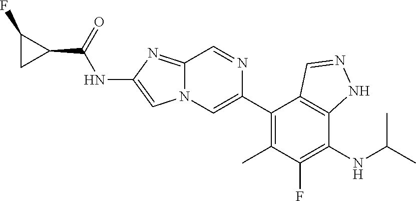 [SMILES: Cc1c(F)c(NC(C)C)c2[nH]ncc2c1-c1cn2cc(NC(=O)[C@H]3C[C@H]3F)nc2cn1] | ¹H NMR (400 MHz, DMSO-d₆) δ 12.99 − 12.86 (m, 1H), 11.33 (s, 1H), 9.00 (s, 1H), 8.75 (s, 1H), 8.31 (s, 1H), 7.87 (br s, 1H), 5.08 − 4.83 (m, 1H), 4.79 − 4.67 (m, 1H), 4.09 − 3.92 (m, 1H), 2.26 (br d, J = 2.9 Hz, 3H), 2.22 − 2.12 (m, 1H), 1.78 − 1.61 (m, 1H), 1.20 (br d, J = 6.2 Hz, 7H); LCMS (electrospray) m/z = 425.9 (M + H)⁺. | C |
| | 1R,2R)-2-fluoro-N-(6-(6-fluoro-7- | | |
| | (isopropylamino)-5-methyl-1H-indazol- | | |
| | 4-yl)imidazo[1,2-a]pyrazin-2- | | |
| | yl)cyclopropane-1-carboxamide | | |
| 34 | 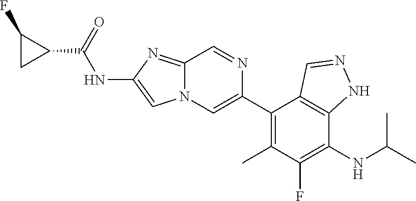 [SMILES: Cc1c(F)c(NC(C)C)c2[nH]ncc2c1-c1cn2cc(NC(=O)[C@@H]3C[C@H]3F)nc2cn1] | ¹H NMR (400 MHz, DMSO-d₆) δ 13.04 − 12.83 (m, 1H), 11.46 (s, 1H), 9.01 (s, 1H), 8.75 (s, 1H), 8.27 (s, 1H), 7.97 − 7.83 (m, 1H), 5.06 − 4.81 (m, 1H), 4.79 − 4.69 (m, 1H), 4.11 − 3.92 (m, 1H), 2.25 (d, J = 3.2 Hz, 3H), 1.63 − 1.51 (m, 1H), 1.33 − 1.24 (m, 1H), 1.20 (d, J = 6.2 Hz, 7H); LCMS (electrospray) m/z = 426.2 (M + H)⁺. | C |
| | (1S,2R)-2-fluoro-N-(6-(6-fluoro-7- | | |
| | (isopropylamino)-5-methyl-1H-indazol- | | |
| | 4-yl)imidazo[1,2-a]pyrazin-2- | | |
| | yl)cyclopropane-1-carboxamide | | |
| 35 | 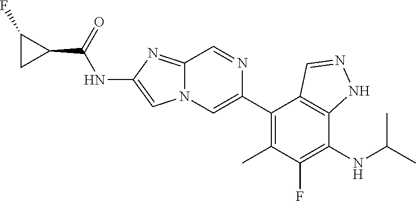 [SMILES: Cc1c(F)c(NC(C)C)c2[nH]ncc2c1-c1cn2cc(NC(=O)[C@H]3C[C@@H]3F)nc2cn1] | ¹H NMR (400 MHz, DMSO-d₆) δ 12.91 (br s, 1H), 11.44 (s, 1H), 9.01 (s, 1H), 8.75 (s, 1H), 8.26 (s, 1H), 7.87 (br s, 1H), 5.03 − 4.81 (m, 1H), 4.79 − 4.68 (m, 1H), 4.08 − 3.91 (m, 1H), 2.25 (d, J = 3.2 Hz, 3H), 1.62 − 1.50 (m, 1H), 1.32 − 1.24 (m, 2H), 1.20 (d, J = 6.2 Hz, 7H); LCMS (electrospray) m/z = 426.3 (M + H)⁺. | C |
| | (1R,2S)-2-fluoro-N-(6-(6-fluoro-7- | | |
| | (isopropylamino)-5-methyl-1H-indazol- | | |
| | 4-yl)imidazo[1,2-a]pyrazin-2- | | |
| | yl)cyclopropane-1-carboxamide | | |
| 36 | 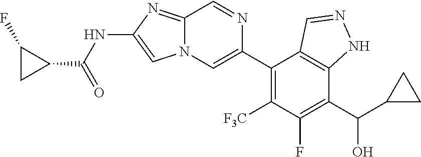 [SMILES: O=C(Nc1cn2cc(-c3c(C(F)(F)F)c(F)c(C(O)C4CC4)c4[nH]ncc34)ncc2n1)[C@@H]1C[C@@H]1F] (1S,2S)-N-(6-(7- | ¹H NMR (400 MHz, DMSO-d₆) δ 13.68 − 13.33 (m, 1H), 11.41 (s, 1H), 9.01 (s, 1H), 8.90 (d, J = 1.2 Hz, 1H), 8.35 (s, 1H), 8.05 − 7.99 (m, 1H), 5.92 (br d, J = 3.3 Hz, 1H), 5.08 − 4.86 (m, 1H), 4.68 (dd, J = 3.9, 8.0 Hz, 1H), 2.23 − 2.17 (m, 1H), 1.75 − 1.66 (m, 1H), 1.46 (br dd, J = 5.6, 13.0 Hz, 1H), 1.23 − 1.16 (m, 1H), 0.65 − 0.56 (m, 2H), 0.46 − 0.33 (m, 2H); LCMS (electrospray) m/z = 493.1 (M + H)⁺. | F |
| | (cyclopropyl(hydroxy)methyl)-6-fluoro- | | |
| | 5-(trifluoromethyl)-1H-indazol-4- | | |
| | yl)imidazo[1,2-a]pyrazin-2-yl)-2- | | |
| | fluorocyclopropane-1-carboxamide | | |
| 37 | 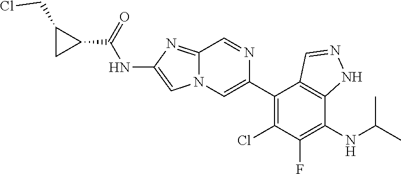 [SMILES: CC(C)Nc1c(F)c(Cl)c(-c2cn3cc(NC(=O)[C@@H]4C[C@@H]4CCl)nc3cn2)c2cn[nH]c12] | ¹H NMR (400 MHz, DMSO-d₆) δ 13.29 − 13.05 (m, 1H), 11.42 (s, 1H), 9.03 (s, 1H), 8.89 (s, 1H), 8.37 (s, 1H), 8.03 − 7.94 (m, 1H), 5.38 − 5.11 (m, 1H), 4.04 (dd, J = 5.8, 10.8 Hz, 2H), 3.83 (t, J = 10.1 Hz, 1H), 2.30 − 2.22 (m, 1H), 1.86 − 1.75 (m, 1H), 1.24 (d, J = 6.1 Hz, 7H), 1.12 (q, J = 5.3 Hz, 1H); LCMS (electrospray) m/z = 476.1 (M + H)⁺. | C |
| | (1R,2S)-N-(6-(5-chloro-6-fluoro-7- | | |
| | (isopropylamino)-1H-indazol-4- | | |
| | yl)imidazo[1,2-a]pyrazin-2-yl)-2- | | |
| | (chloromethyl)cyclopropane-1- | | |
| | carboxamide | | |
| 38 | 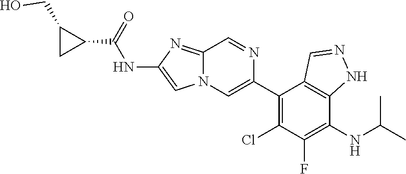 [SMILES: CC(C)Nc1c(F)c(Cl)c(-c2cn3cc(NC(=O)[C@@H]4C[C@@H]4CO)nc3cn2)c2cn[nH]c12] | ¹H NMR (400 MHz, DMSO-d₆) δ 13.25 − 13.14 (m, 1H), 11.23 (s, 1H), 9.00 (s, 1H), 8.88 (s, 1H), 8.34 (s, 1H), 8.02 − 7.90 (m, 1H), 5.21 (dd, J = 2.3, 9.4 Hz, 1H), 4.48 (t, J = 5.1 Hz, 1H), 4.11 − 3.99 (m, 1H), 3.71 − 3.61 (m, 1H), 3.56 − 3.45 (m, 1H), 2.10 − 2.01 (m, 1H), 1.48 (br dd, J = 7.3, 14.6 Hz, 1H), 1.23 (d, J = 6.4 Hz, 6H), 1.02 (dt, J = 3.9, 8.0 Hz, 1H), 0.98 − 0.91 (m, 1H); LCMS (electrospray) m/z = 458.3 (M + H)⁺. | C |
| | (IR,2S)-N-(6-(5-chloro-6-fluoro-7- | | |
| | (isopropylamino)-1H-indazol-4- | | |
| | yl)imidazo[1,2-a]pyrazin-2-yl)-2- | | |
| | (hydroxymethyl)cyclopropane-1- | | |
| | carboxamide | | |
| 39 | 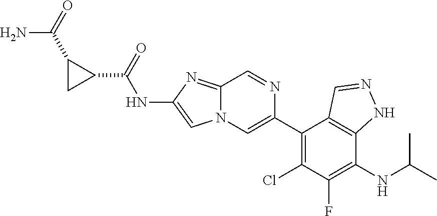 [SMILES: CC(C)Nc1c(F)c(Cl)c(-c2cn3cc(NC(=O)[C@@H]4C[C@@H]4C(N)=O)nc3cn2)c2cn[nH]c12] | ¹H NMR (400 MHz, DMSO-d₆) δ 11.27 − 11.09 (m, 1H), 9.05 − 8.85 (m, 2H), 8.31 (s, 1H), 7.97 (s, 1H), 7.57 − 7.48 (m, 1H), 7.01 − 6.88 (m, 1H), 5.26 − 5.17 (m, 1H), 4.24 − 4.03 (m, 1H), 2.23 − 2.11 (m, 1H), 2.03 − 1.94 (m, 1H), 1.52 − 1.45 (m, 1H), 1.23 (d, J = 6.2 Hz, 7H), 1.17 (dt, J = 4.4, 8.3 Hz, 1H); LCMS (electrospray) m/z = 471.2 (M + H)⁺. | C |
| | (1R,2S)-N1-(6-(5-chloro-6-fluoro-7- | | |
| | (isopropylamino)-1H-indazol-4- | | |
| | yl)imidazo[1,2-a]pyrazin-2- | | |
| | yl)cyclopropane-1,2-dicarboxamide | | |
| 40 | 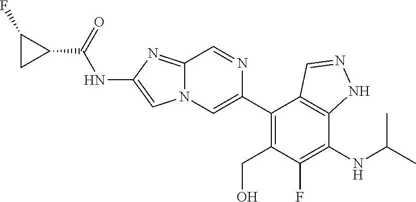 [SMILES: CC(C)Nc1c(F)c(CO)c(-c2cn3cc(NC(=O)[C@@H]4C[C@@H]4F)nc3cn2)c2cn[nH]c12] | ¹H NMR (400 MHz, DMSO-d₆) δ 12.99 (s, 1H), 11.34 (s, 1H), 9.02 (s, 1H), 8.86 (s, 1H), 8.33 (s, 1H), 8.00 (s, 1H), 5.06 − 4.88 (m, 2H), 4.79 − 4.78 (d, 1H), 4.52 (s, 2H), 4.02 (m, 1H), 2.21 − 2.19 (m, 1H), 1.72 − 1.65 (m, 1H), 1.23 − 1.16 (m, 7H); LCMS (electrospray) m/z 442.10 (M + H)⁺. | F |
| | (1S,2S)-2-fluoro-N-(6-(6-fluoro-5- | | |
| | (hydroxymethyl)-7-(isopropylamino)-1H- | | |
| | indazol-4-yl)imidazo[1,2-a]pyrazin-2- | | |
| | yl)cyclopropane-1-carboxamide | | |
| 41 | 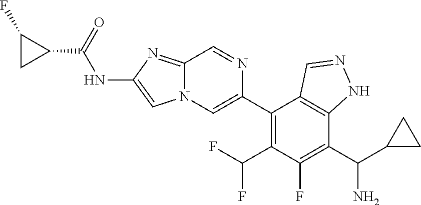 [SMILES: NC(c1c(F)c(C(F)F)c(-c2cn3cc(NC(=O)[C@@H]4C[C@@H]4F)nc3cn2)c2cn[nH]c12)C1CC1] | ¹H NMR (400 MHz, DMSO-d₆) δ 11.43 − 11.32 (s, 1H), 9.06 − 8.99 (s, 1H), 8.99 − 8.91 (d, J = 1.5 Hz, 1H), 8.42 − 8.31 (s, 1H), 8.20 − 8.08 (d, J = 16.3 Hz, 1H), 7.23 − 6.91 (t, J = 52.4 Hz, 1H), 5.09 − 4.81 (m, 1H), 3.89 − 3.77 (d, J = 8.9 Hz, 1H), 2.23 − 2.12 (m, 1H), 1.75 − 1.60 (m, 1H), 1.42 − 1.34 (m, 1H), 1.19 − 1.16 (m, 1H), 0.61 − 0.44 (m, 2H), 0.39 − 0.18 (m, 2H); LCMS (electrospray) m/z 474.15 (M + H)⁺. | E |
| | (1S,2S)-N-(6-(7- | | |
| | (amino(cyclopropyl)methyl)-5- | | |
| | (difluoromethyl)-6-fluoro-1H-indazol-4- | | |
| | yl)imidazo[1,2-a]pyrazin-2-yl)-2- | | |
| | fluorocyclopropane-1-carboxamide | | |
| 42 | 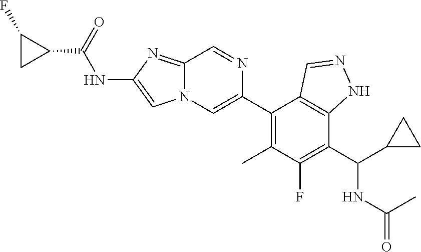 [SMILES: CC(=O)NC(c1c(F)c(C)c(-c2cn3cc(NC(=O)[C@@H]4C[C@@H]4F)nc3cn2)c2cn[nH]c12)C1CC1] | ¹H NMR (400 MHz, DMSO-d₆) δ 13.10 (s, 1H), 11.36 (s, 1H), 9.03 (s, 1H), 8.82 (d, J = 1.5 Hz, 1H), 8.50 (d, J = 7.3 Hz, 1H), 8.31 (s, 1H), 8.05 − 7.80 (m, 1H), 5.09 − 4.80 (m, 1H), 4.65 (t, J = 8.5 Hz, 1H), 2.24 (d, J = 2.8 Hz, 3H), 2.17 (s, 1H), 1.84 (s, 3H), 1.67 (d, J = 21.3 Hz, 1H), 1.49 (s, 1H), 1.17 (d, J = 11.9 Hz, 1H), 0.61 (s, 1H), 0.48 − 0.29 (m, 3H); LCMS (electrospray) m/z 480.20 (M + H)⁺. | E |
| | (1S,2S)-N-(6-(7- | | |
| | (acetamido(cyclopropyl)methyl)-6- | | |
| | fluoro-5-methyl-1H-indazol-4- | | |
| | yl)imidazo[1,2-a]pyrazin-2-yl)-2- | | |
| | fluorocyclopropane-1-carboxamide | | |
| 43 | 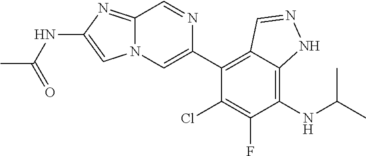 [SMILES: CC(=O)Nc1cn2cc(-c3c(Cl)c(F)c(NC(C)C)c4[nH]ncc34)ncc2n1] | ¹H NMR (400 MHz, DMSO-d₆) δ 13.17 (s, 1H), 11.03 (s, 1H), 9.01 (s, 1H), 8.90 (s, 1H), 8.36 (s, 1H), 7.95 (s, 1H), 5.20 (d, J = 9.6 Hz, 1H), 4.10 − 3.97 (m, 1H), 2.13 (s, 3H), 1.23 (d, J = 6.3 Hz, 7H); LCMS (electrospray) m/z 403.10 (M + H)⁺. | C |
| | N-(6-(5-chloro-6-fluoro-7- | | |
| | (isopropylamino)-1H-indazol-4- | | |
| | yl)imidazo[1,2-a]pyrazin-2-yl)acetamide | | |
| 44 | 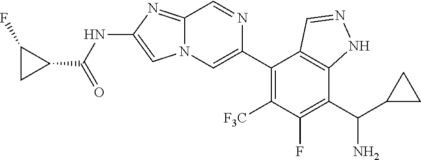 [SMILES: NC(c1c(F)c(C(F)(F)F)c(-c2cn3cc(NC(=O)[C@@H]4C[C@@H]4F)nc3cn2)c2cn[nH]c12)C1CC1] | ¹H NMR (400 MHz, DMSO-d₆) δ 11.40 (s, 1H), 9.00 (s, 1H), 8.88 (s, 1H), 8.34 (s, 1H), 8.22 (s, 1H), 8.03 (s, 1H), 5.10 − 4.78 (m, 1H), 3.94 − 3.82 (m, 1H), 2.27 − 2.12 (m, 1H), 1.75 − 1.62 (m, 1H), 1.47 − 1.34 (m, 1H), 1.27 − 1.12 (m, 1H), 0.65 − 0.49 (m, 2H), 0.40 − 0.23 (m, 2H); LCMS (electrospray) 492.1 (M + H)⁺. | F |
| | (1S,2S)-N-(6-(7- | | |
| | (amino(cyclopropyl)methyl)-6-fluoro-5- | | |
| | (trifluoromethyl)-1H-indazol-4- | | |
| | yl)imidazo[1,2-a]pyrazin-2-yl)-2- | | |
| | fluorocyclopropane-1-carboxamide | | |
| 45 | 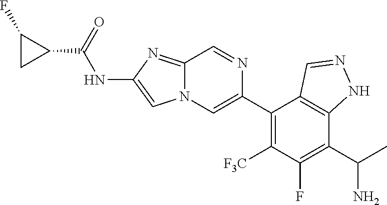 [SMILES: CC(N)c1c(F)c(C(F)(F)F)c(-c2cn3cc(NC(=O)[C@@H]4C[C@@H]4F)nc3cn2)c2cn[nH]c12] | ¹H NMR (400 MHz, DMSO-d₆) δ 13.56 − 13.32 (m, 1H), 11.39 (s, 1H), 9.19 (s, 1H), 9.05 (s, 1H), 8.94 (s, 1H), 8.42 (s, 1H), 8.32 − 8.25 (m, 1H), 5.09 − 4.87 (m, 1H), 4.83 − 4.74 (m, 1H), 2.23 − 2.15 (m, 1H), 1.77 − 1.62 (m, 1H), 1.51 (d, J = 6.6 Hz, 3H), 1.21 (tdd, J = 6.3, 8.9, 12.6 Hz, 1H); LCMS (electrospray) 466.1 (M + H)⁺. | F |
| | (1S,2S)-N-(6-(7-(1-aminoethyl)-6-fluoro- | | |
| | 5-(trifluoromethyl)-1H-indazol-4- | | |
| | yl)imidazo[1,2-a]pyrazin-2-yl)-2- | | |
| | fluorocyclopropane-1-carboxamide | | |
| 46 | 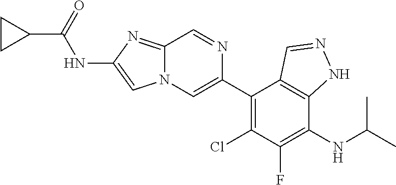 [SMILES: CC(C)Nc1c(F)c(Cl)c(-c2cn3cc(NC(=O)C4CC4)nc3cn2)c2cn[nH]c12] | ¹H NMR (400 MHz, DMSO-d₆) δ 13.16 (s, 1H), 11.31 (s, 1H), 9.01 (s, 1H), 8.90 (d, J = 1.5 Hz, 1H), 8.32 (s, 1H), 7.94 (d, J = 1.5 Hz, 1H), 5.19 (d, J = 9.9 Hz, 1H), 4.04 (s, 1H), 2.02 − 1.94 (m, 1H), 1.23 (d, J = 6.3 Hz, 6H), 0.85 (d, J = 5.2 Hz, 4H); LCMS (electrospray) 428.10 (M + H)⁺. | C |
| | N-(6-(5-chloro-6-fluoro-7- | | |
| | (isopropylamino)-1H-indazol-4- | | |
| | yl)imidazo[1,2-a]pyrazin-2- | | |
| | yl)cyclopropanecarboxamide | | |
| 47 | 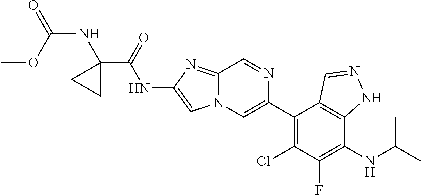 [SMILES: COC(=O)NC1(C(=O)Nc2cn3cc(-c4c(Cl)c(F)c(NC(C)C)c5[nH]ncc45)ncc3n2)CC1] | ¹H NMR (400 MHz, DMSO-d₆) δ 13.17 (s, 1H), 10.62 (s, 1H), 9.03 (s, 1H), 8.91 (s, 1H), 8.37 (s, 1H), 7.95 (s, 1H), 7.81 (s, 1H), 5.21 (d, J = 9.0 Hz, 1H), 3.96-4.12 (1H), 3.59 (s, 3H), 1.43 (d, J = 3.3 Hz, 2H), 1.23 (d, J = 6.3 Hz, 6H), 1.08 (s, 2H); LCMS (electrospray) 501.1 (M + H)⁺. | C |
| | methyl (1-((6-(5-chloro-6-fluoro-7- | | |
| | (isopropylamino)-1H-indazol-4- | | |
| | yl)imidazo[1,2-a]pyrazin-2- | | |
| | yl)carbamoyl)cyclopropyl)carbamate | | |
| 48 | 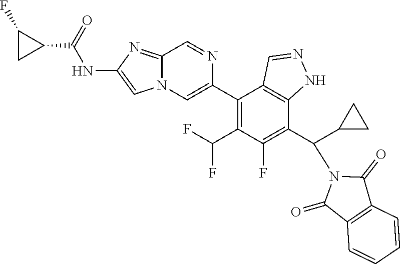 [SMILES: O=C(Nc1cn2cc(-c3c(C(F)F)c(F)c(C(C4CC4)N4C(=O)c5ccccc5C4=O)c4[nH]ncc34)ncc2n1)[C@@H]1C[C@@H]1F] | ¹H NMR (400 MHz, DMSO-d₆) δ 13.61 − 13.36 (s, 1H), 11.50 − 11.28 (s, 1H), 9.10 − 8.85 (d, J = 17.4 Hz, 2H), 8.45 − 8.31 (s, 1H), 8.31 − 8.15 (s, 1H), 8.05 − 7.72 (m, 4H), 7.25 − 6.86 (t, 1H), 5.11 − 4.76 (m, 2H), 2.28 − 2.08 (s, 1H), 1.80 − 1.57 (m, 1H), 1.38 − 1.09 (m, 2H), 0.81 − 0.62 (m, 2H), 0.62 − 0.47 (s, 2H); LCMS (electrospray) 604.20 (M + H)⁺. | E |
| | (1S,2S)-N-(6-(7-(cyclopropyl(1,3- | | |
| | dioxoisoindolin-2-yl)methyl)-5- | | |
| | (difluoromethyl)-6-fluoro-1H-indazol-4- | | |
| | yl)imidazo[1,2-a]pyrazin-2-yl)-2- | | |
| | fluorocyclopropane-1-carboxamide | | |
| 49 | 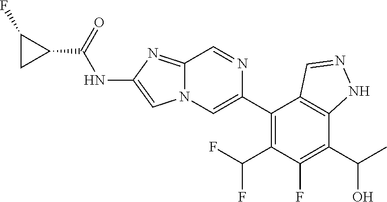 [SMILES: CC(O)c1c(F)c(C(F)F)c(-c2cn3cc(NC(=O)[C@@H]4C[C@@H]4F)nc3cn2)c2cn[nH]c12] | ¹H NMR (400 MHz, DMSO-d₆) δ 13.42 − 13.22 (s, 1H), 11.45 − 11.29 (s, 1H), 9.12 − 8.99 (s, 1H), 8.99 − 8.89 (d, J = 1.5 Hz, 1H), 8.48 − 8.31 (s, 1H), 8.23 − 8.05 (s, 1H), 7.30 − 6.93 (t, J = 52.5 Hz, 1H), 5.91 − 5.68 (s, 1H), 5.56 − 5.30 (m, 1H), 5.16 − 4.84 (m, 1H), 2.25 − 2.11 (m, 1H), 1.78 − 1.62 (m, 1H), 1.62 − 1.43 (d, J = 6.5 Hz, 3H), 1.21 − 1.12 (m, 1H); LCMS (electrospray) m/z 449.10 (M + H)⁺. | E |
| | (1S,2S)-N-(6-(5-(difluoromethyl)-6- | | |
| | fluoro-7-(1-hydroxyethyl)-1H-indazol-4- | | |
| | yl)imidazo[1,2-a]pyrazin-2-yl)-2- | | |
| | fluorocyclopropane-1-carboxamide | | |
| 50 | 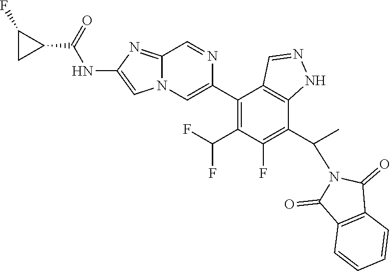 [SMILES: CC(c1c(F)c(C(F)F)c(-c2cn3cc(NC(=O)[C@@H]4C[C@@H]4F)nc3cn2)c2cn[nH]c12)N1C(=O)c2ccccc2C1=O] | ¹H NMR (400 MHz, DMSO-d₆) δ 13.72 − 13.45 (s, 1H), 11.53 − 11.28 (s, 1H), 9.13 − 9.00 (s, 1H), 9.00 − 8.86 (s, 1H), 8.46 − 8.30 (s, 1H), 8.30 − 8.12 (s, 1H), 7.95 − 7.72 (s, 4H), 7.30 − 6.80 (t, J = 52.6 Hz, 1H), 6.17 − 5.86 (d, J = 7.4 Hz, 1H), 5.12 − 4.74 (m, 1H), 2.26 − 2.15 (m, 1H), 2.15 − 1.99 (d, J = 7.4 Hz, 3H), 1.78 − 1.59 (m, 1H), 1.22 − 1.14 (m, 1H); LCMS (electrospray) m/z 578.10 (M + H)⁺. | E |
| | (1S,2S)-N-(6-(5-(difluoromethyl)-7-(1- | | |
| | (1,3-dioxoisoindolin-2-yl)ethyl)-6-fluoro- | | |
| | 1H-indazol-4-yl)imidazo[1,2-a]pyrazin-2- | | |
| | yl)-2-fluorocyclopropane-1-carboxamide | | |
| 51 | 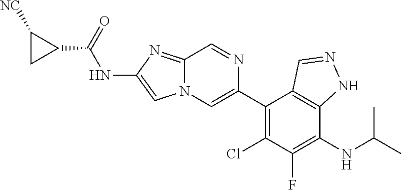 | ¹H NMR (400 MHz, DMSO-d₆) δ 13.17 (s, 1H), 11.65 (s, 1H), 9.05 (s, 1H), 8.91 (s, 1H), 8.41 (s, 1H), 7.96 (s, 1H), 5.20 (d, J = 8.6 Hz, 1H), 4.04 (s, 1H), 2.30 (dd, J = 15.5, 8.4 Hz, 1H), 1.49 (m, 2H), 1.23 (d, J = 6.3 Hz, 7H); LCMS (electrospray) 453.1 (M + H)⁺. | C |
| | (1R,2S)-N-(6-(5-chloro-6-fluoro-7- | | |
| | (isopropylamino)-1H-indazol-4- | | |
| | yl)imidazo[1,2-a]pyrazin-2-yl)-2- | | |
| | cyanocyclopropane-1-carboxamide | | |
| 52 | 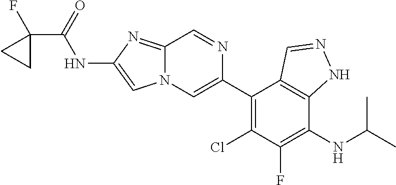 | ¹H NMR (400 MHz, DMSO-d₆) δ 13.17 (s, 1H), 11.34 (s, 1H), 9.07 (s, 1H), 8.94 (s, 1H), 8.42 (s, 1H), 7.96 (s, 1H), 5.20 (d, J = 8.2 Hz, 1H), 4.04 (s, 1H), 1.48 (s, 1H), 1.43 (s, 1H), 1.38 (m, 2H), 1.24 (d, J = 6.3 Hz, 6H); LCMS (electrospray) 466.1 (M + H)+ | C |
| | N-(6-(5-chloro-6-fluoro-7- | | |
| | (isopropylamino)-1H-indazol-4- | | |
| | yl)imidazo[1,2-a]pyrazin-2-yl)-1- | | |
| | fluorocyclopropane-1-carboxamide | | |
| 53 | 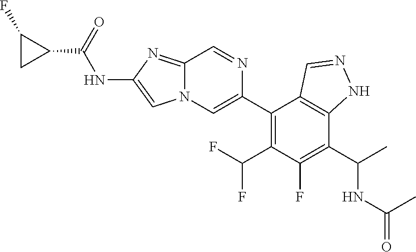 [SMILES: CC(=O)NC(C)c1c(F)c(C(F)F)c(-c2cn3cc(NC(=O)[C@@H]4C[C@@H]4F)nc3cn2)c2cn[nH]c12] | ¹H NMR (400 MHz, DMSO-d₆) δ 13.67 − 13.40 (s, 1H), 11.50 − 11.30 (s, 1H), 9.10 − 9.00 (s, 1H), 9.00 − 8.90 (d, J = 1.5 Hz, 1H), 8.49 − 8.29 (m, 2H), 8.29 − 8.12 (s, 1H), 7.31 − 6.87 (t, J = 52.6 Hz, 1H), 5.59 − 5.37 (m, 1H), 5.20 − 4.78 (m, 1H), 2.26 − 2.13 (m, 1H), 1.92 − 1.81 (s, 3H), 1.78 − 1.62 (m, 1H), 1.61 − 1.51 (d, J = 7.2 Hz, 3H), 1.21 − 1.17 (m, 1H); LCMS (electrospray) 490.15 (M + H)⁺. | E |
| | (1S,2S)-N-(6-(7-(1-acetamidoethyl)-5- | | |
| | (difluoromethyl)-6-fluoro-1H-indazol-4- | | |
| | yl)imidazo[1,2-a]pyrazin-2-yl)-2- | | |
| | fluorocyclopropane-1-carboxamide | | |
| 54 | 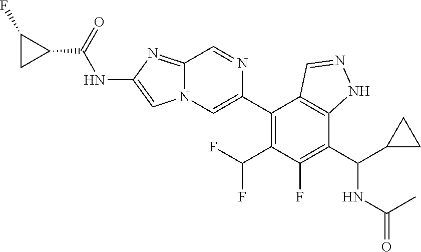 [SMILES: CC(=O)NC(c1c(F)c(C(F)F)c(-c2cn3cc(NC(=O)[C@@H]4C[C@@H]4F)nc3cn2)c2cn[nH]c12)C1CC1] | ¹H NMR (400 MHz, DMSO-d₆) δ 13.68 − 13.49 (s, 1H), 11.47 − 11.32 (s, 1H), 9.10 − 9.02 (s, 1H), 9.02 − 8.92 (d, J = 1.5 Hz, 1H), 8.73 − 8.55 (d, J = 6.9 Hz, 1H), 8.44 − 8.32 (s, 1H), 8.27 − 8.11 (s, 1H), 7.26 − 6.91 (t, J = 52.5 Hz, 1H), 5.14 − 4.82 (m, 1H), 4.72 − 4.63 (m, 1H), 2.27 − 2.14 (m, 1H), 1.92 − 1.84 (s, 3H), 1.76 − 1.64 (m, 1H), 1.56 − 1.48 (m, 1H), 1.20 − 1.17 (m, 1H), 0.94 − 0.63 (m, 2H), 0.47 − 0.33 (d, J = 33.0 Hz, 2H); LCMS (electrospray) 516.10 (M + H)⁺. | E |
| | (1S,2S)-N-(6-(7- | | |
| | (acetamido(cyclopropyl)methyl)-5- | | |
| | (difluoromethyl)-6-fluoro-1H-indazol-4- | | |
| | yl)imidazo[1,2-a]pyrazin-2-yl)-2- | | |
| | fluorocyclopropane-1-carboxamide | | |
| 55 | 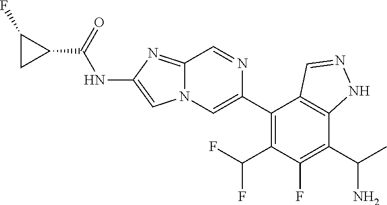 [SMILES: CC(N)c1c(F)c(C(F)F)c(-c2cn3cc(NC(=O)[C@@H]4C[C@@H]4F)nc3cn2)c2cn[nH]c12] | ¹H NMR (400 MHz, DMSO-d₆) δ 11.48 − 11.28 (s, 1H), 9.09 − 8.99 (s, 1H), 8.99 − 8.88 (s, 1H), 8.47 − 8.30 (s, 1H), 8.26 − 8.07 (s, 1H), 7.26 − 6.90 (t, J = 52.5 Hz, 1H), 5.14 − 4.81 (m, 1H), 4.78 − 4.66 (m, 1H), 2.26 − 2.13 (m, 1H), 1.74 − 1.69 (m, 1H), 1.52 − 1.44 (d, J = 6.8 Hz, 3H), 1.20 − 1.16 (m, 1H); LCMS (electrospray) 448.10 (M + H)⁺. | E |
| | (1S,2S)-N-(6-(7-(1-aminoethyl)-5- | | |
| | (difluoromethyl)-6-fluoro-1H-indazol-4- | | |
| | yl)imidazo[1,2-a]pyrazin-2-yl)-2- | | |
| | fluorocyclopropane-1-carboxamide | | |
| 56 | 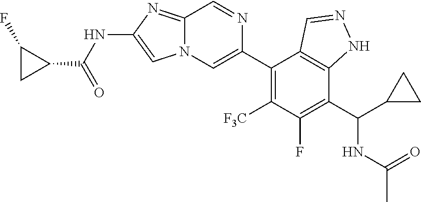 [SMILES: CC(=O)NC(c1c(F)c(C(F)(F)F)c(-c2cn3cc(NC(=O)[C@@H]4C[C@@H]4F)nc3cn2)c2cn[nH]c12)C1CC1] | ¹H NMR (400 MHz, DMSO-d₆) δ 13.98 − 13.58 (m, 1H), 11.42 (s, 1H), 9.01 (s, 1H), 8.93 − 8.85 (m, 1H), 8.67 (br d, J = 6.5 Hz, 1H), 8.36 − 8.27 (m, 1H), 8.06 (s, 1H), 5.09 − 4.84 (m, 1H), 4.66 (br dd, J = 7.4, 10.0 Hz, 1H), 2.21 − 2.15 (m, 1H), 1.87 (s, 3H), 1.73 − 1.62 (m, 1H), 1.57 − 1.44 (m, 1H), 1.31 − 1.12 (m, 1H), 0.73 − 0.62 (m, 1H), 0.54 − 0.42 (m, 2H), 0.41 − 0.32 (m, 1H); LCMS (electrospray) 534.2 (M + H)⁺. | F |
| | (1S,2S)-N-(6-(7- | | |
| | (acetamido(cyclopropyl)methyl)-6- | | |
| | fluoro-5-(trifluoromethyl)-1H-indazol-4- | | |
| | yl)imidazo[1,2-a]pyrazin-2-yl)-2- | | |
| | fluorocyclopropane-1-carboxamide | | |
| | formate | | |
| 57 | 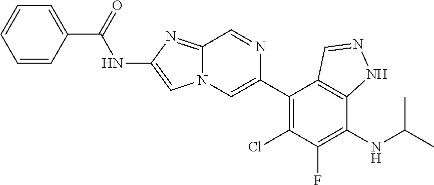 [SMILES: CC(C)Nc1c(F)c(Cl)c(-c2cn3cc(NC(=O)c4ccccc4)nc3cn2)c2cn[nH]c12] | ¹H NMR (400 MHz, DMSO-d₆) δ 13.20 (s, 1H), 11.56 (s, 1H), 9.08 (s, 1H), 8.97 (s, 1H), 8.59 (s, 1H), 8.11 (d, J = 7.4 Hz, 2H), 7.98 (s, 1H), 7.62 (m, 1H), 7.54 (m, 2H), 5.23 (d, J = 9.8 Hz, 1H), 4.04 (m, 1H), 1.24 (d, J = 6.3 Hz, 6H); LCMS (electrospray) 464.1 (M + H)⁺. | C |
| | N-(6-(5-chloro-6-fluoro-7- | | |
| | (isopropylamino)-1H-indazol-4- | | |
| | yl)imidazo[1,2-a]pyrazin-2-yl)benzamide | | |
| 58 | 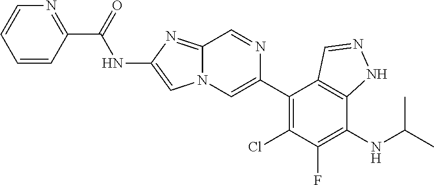 [SMILES: CC(C)Nc1c(F)c(Cl)c(-c2cn3cc(NC(=O)c4ccccn4)nc3cn2)c2cn[nH]c12] | ¹H NMR (400 MHz, DMSO-d₆) δ 13.18 (s, 1H), 10.91 (s, 1H), 9.11 (s, 1H), 8.99 (s, 1H), 8.79 (d, J = 4.7 Hz, 1H), 8.60 (s, 1H), 8.22 (d, J = 7.8 Hz, 1H), 8.12 (t, J = 7.4 Hz, 1H), 7.98 (s, 1H), 7.75-7.72 (m, 1H), 5.24 (s, 1H), 4.04 (d, J = 7.0 Hz, 1H), 1.24 (d, J = 6.7 Hz, 6H); LCMS (electrospray) 465.1 (M + H)⁺. | C |
| | N-(6-(5-chloro-6-fluoro-7- | | |
| | (isopropylamino)-1H-indazol-4- | | |
| | yl)imidazo[1,2-a]pyrazin-2- | | |
| | yl)picolinamide | | |
| 59 | 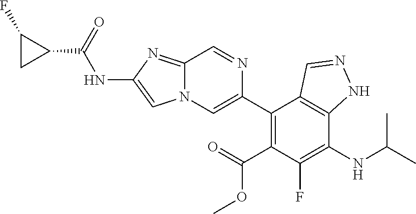 [SMILES: COC(=O)c1c(F)c(NC(C)C)c2[nH]ncc2c1-c1cn2cc(NC(=O)[C@@H]3C[C@@H]3F)nc2cn1] | ¹H NMR (400 MHz, DMSO-d₆) δ 13.26 (s, 1H), 11.35 (s, 1H), 9.00 (s, 1H), 8.93 (s, 1H), 8.39 (d, J = 3.5 Hz, 2H), 5.10 (d, J = 9.2 Hz, 1H), 4.96 (d, J = 66.8 Hz, 1H), 4.04 − 3.95 (m, 1H), 3.66 (s, 3H), 2.21 − 2.16 (m, 1H), 1.69 (d, J = 22.7 Hz, 1H), 1.22 (d, J = 6.4 Hz, 7H); LCMS (electrospray) 470.15 (M + H)⁺. | F |
| | methyl 6-fluoro-4-(2-((1S,2S)-2- | | |
| | fluorocyclopropane-1- | | |
| | carboxamido)imidazo[1,2-a]pyrazin-6- | | |
| | yl)-7-(isopropylamino)-1H-indazole-5- | | |
| | carboxylate | | |
| 60 | 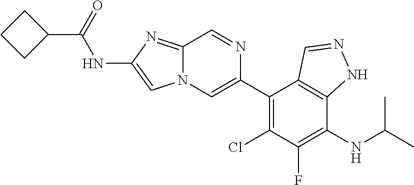 [SMILES: CC(C)Nc1c(F)c(Cl)c(-c2cn3cc(NC(=O)C4CCC4)nc3cn2)c2cn[nH]c12] | ¹H NMR (400 MHz, DMSO-d₆) δ 13.16 (s, 1H), 10.88 (s, 1H), 9.00 (s, 1H), 8.91 (d, J = 1.5 Hz, 1H), 8.39 (s, 1H), 7.94 (d, J = 1.6 Hz, 1H), 5.19 (d, J = 9.8 Hz, 1H), 4.12 − 3.96 (m, 1H), 3.38 (q, J = 8.5 Hz, 1H), 2.24 (p, J = 9.1 Hz, 2H), 2.18 − 2.08 (m, 2H), 2.01 − 1.91 (m, 1H), 1.88 − 1.77 (m, 1H), 1.23 (d, J = 6.3 Hz, 6H); LCMS (electrospray) 442.10 (M + H)⁺. | C |
| | N-(6-(5-chloro-6-fluoro-7- | | |
| | (isopropylamino)-1H-indazol-4- | | |
| | yl)imidazo[1,2-a]pyrazin-2- | | |
| | yl)cyclobutanecarboxamide | | |
| 61 | 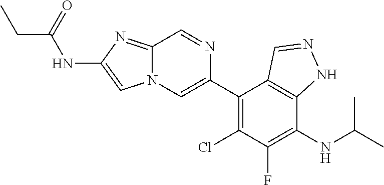 [SMILES: CCC(=O)Nc1cn2cc(-c3c(Cl)c(F)c(NC(C)C)c4[nH]ncc34)ncc2n1] | ¹H NMR (400 MHz, DMSO-d₆) δ 13.17 (s, 1H), 10.98 (s, 1H), 9.01 (s, 1H), 8.91 (s, 1H), 8.37 (s, 1H), 7.95 (s, 1H), 5.20 (d, J = 9.5 Hz, 1H), 4.13 − 3.96 (m, 1H), 2.42 (q, J = 7.6 Hz, 2H), 1.23 (d, J = 6.2 Hz, 6H), 1.10 (t, J = 7.5 Hz, 3H); LCMS (electrospray) 416.10 (M + H)⁺. | C |
| | N-(6-(5-chloro-6-fluoro-7- | | |
| | (isopropylamino)-1H-indazol-4- | | |
| | yl)imidazo[1,2-a]pyrazin-2- | | |
| | yl)propionamide | | |
| 62 | 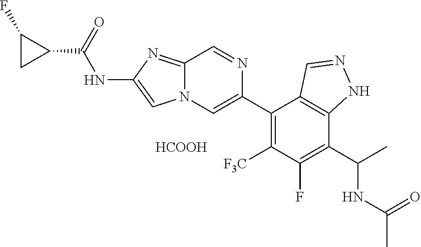 [SMILES: CC(=O)NC(C)c1c(F)c(C(F)(F)F)c(-c2cn3cc(NC(=O)[C@@H]4C[C@@H]4F)nc3cn2)c2cn[nH]c12 ; "HCOOH"] | ¹H NMR (400 MHz, DMSO-ds) δ 13.55 (s, 1H), 11.39 (s, 1H), 9.21 (s, 1H), 9.06 (s, 1H), 8.88 (br d, J = 5.0 Hz, 1H), 8.78 (s, 1H), 8.43 (s, 1H), 5.47 (br dd, J = 5.1, 7.0 Hz, 1H), 5.10 − 4.86 (m, 1H), 4.35 (t, J = 5.1 Hz, 1H), 2.23 − 2.18 (m, 1H), 1.81 (s, 3H), 1.75 − 1.71 (m, 1H), 1.60 (br d, J = 7.2 Hz, 3H), 1.25 − 1.16 (m, 1H); LCMS (electrospray) 508.1 (M + H)⁺. | F |
| | (1S,2S)-N-(6-(7-(1-acetamidoethyl)-6- | | |
| | fluoro-5-(trifluoromethyl)-1H-indazol-4- | | |
| | yl)imidazo[1,2-a]pyrazin-2-yl)-2- | | |
| | fluorocyclopropanecarboxamide formate | | |
| 63 | 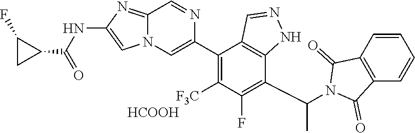 [SMILES: CC(c1c(F)c(C(F)(F)F)c(-c2cn3cc(NC(=O)[C@@H]4C[C@@H]4F)nc3cn2)c2cn[nH]c12)N1C(=O)c2ccccc2C1=O ; "HCOOH"] | ¹H NMR (400 MHz, DMSO-d₆) δ 11.39 ( d, J = 1.8 Hz, 1H), 8.99 ( s, 1H), 8.88 ( d, J = 0.7 Hz, 1H), 8.33 ( s, 1H), 8.23 − 8.08 (m, 1H), 7.95 − 7.80 (m, 4H), 7.51 − 7.34 (m, 1H), 6.11 − 5.96 (m, 1H), 5.14 − 4.81 (m, 1H), 2.24 − 2.13 (m, 1H), 2.09 − 1.99 (m, 3H), 1.76 − 1.61 (m, 1H), 1.26 − 1.13 (m, 1H); LCMS (electrospray) 596.1 (M + H)⁺. | F |
| | (1S,2S)-N-(6-(7-(1-(1,3-dioxoisoindolin- | | |
| | 2-yl)ethyl)-6-fluoro-5-(trifluoromethyl)- | | |
| | 1H-indazol-4-yl)imidazo[1,2-a]pyrazin-2- | | |
| | yl)-2-fluorocyclopropanecarboxamide | | |
| | formate | | |
| 64 | 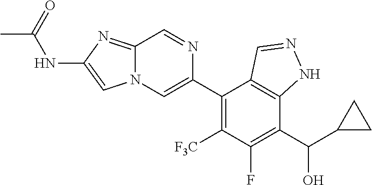 [SMILES: CC(=O)Nc1cn2cc(-c3c(C(F)(F)F)c(F)c(C(O)C4CC4)c4[nH]ncc34)ncc2n1] | ¹H NMR (400 MHz, DMSO-d₆) δ 13.54 (s, 1H), 11.09 (s, 1H), 9.00 (s, 1H), 8.89 (s, 1H), 8.36 (s, 1H), 8.02 (s, 1H), 6.13 − 5.81 (m, 1H), 4.67 (d, J = 7.9 Hz, 1H), 2.14 (s, 3H), 1.56 − 1.38 (m, 1H), 0.66 − 0.55 (m, 2H), 0.47 − 0.31 (m, 2H); LCMS (electrospray) 449.1 (M + H)⁺. | C |
| | N-(6-(7-(cyclopropyl(hydroxy)methyl)-6- | | |
| | fluoro-5-(trifluoromethyl)-2H-indazol-4- | | |
| | yl)imidazo[1,2-a]pyrazin-2-yl)acetamide | | |
| 65 | 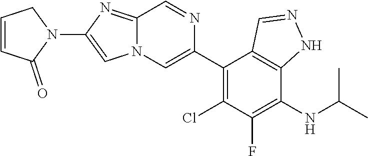 [SMILES: CC(C)Nc1c(F)c(Cl)c(-c2cn3cc(N4CC=CC4=O)nc3cn2)c2cn[nH]c12] | ¹H NMR (400 MHz, DMSO-d₆) δ 13.20 (br d, J = 4.9 Hz, 1H), 9.11 (d, J = 3.9 Hz, 1H), 8.99 − 8.91 (m, 1H), 8.51 (d, J = 5.4 Hz, 1H), 8.03 − 7.90 (m, 1H), 6.46 (d, J = 6.4 Hz, 1H), 6.12 (t, J = 5.8 Hz, 1H), 5.43 − 5.35 (m, 1H), 5.24 (br d, J = 7.7 Hz, 1H), 4.17 − 3.95 (m, 1H), 2.84 − 2.70 (m, 2H), 2.03 − 1.91 (m, 1H), 1.90 − 1.78 (m, 1H), 1.23 (d, J = 6.2 Hz, 6H); LCMS (electrospray) 426.1 (M + H)⁺. | C |
| | 1-(6-(5-chloro-6-fluoro-7- | | |
| | (isopropylamino)-1H-indazol-4- | | |
| | yl)imidazo[1,2-a]pyrazin-2-yl)-1,5- | | |
| | dihydro-2H-pyrrol-2-one | | |
| 66 | 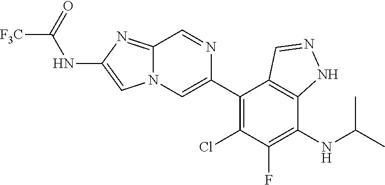 [SMILES: CC(C)Nc1c(F)c(Cl)c(-c2cn3cc(NC(=O)C(F)(F)F)nc3cn2)c2cn[nH]c12] | ¹H NMR (400 MHz, DMSO-d₆) δ 13.18 (s, 1H), 12.81 (br s, 1H), 9.15 (s, 1H), 8.98 (d, J = 1.0 Hz, 1H), 8.51 (s, 1H), 7.97 (d, J = 1.0 Hz, 1H), 5.23 (br dd, J = 2.1, 9.1 Hz, 1H), 4.11 − 4.00 (m, 1H), 1.24 (d, J = 6.4 Hz, 6H); LCMS (electrospray) 456.2 (M + H)⁺. | B |
| | N-(6-(5-chloro-6-fluoro-7- | | |
| | (isopropylamino)-1H-indazol-4- | | |
| | yl)imidazo[1,2-a]pyrazin-2-yl)-2,2,2- | | |
| | trifluoroacetamide | | |
| 67 | 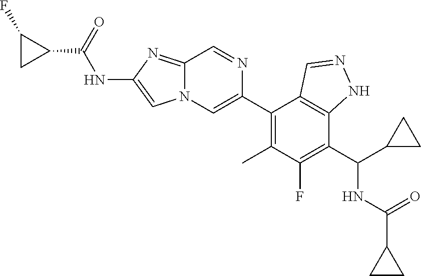 [SMILES: Cc1c(F)c(C(NC(=O)C2CC2)C2CC2)c2[nH]ncc2c1-c1cn2cc(NC(=O)[C@@H]3C[C@@H]3F)nc2cn1] | ¹H NMR (400 MHz, DMSO-D6) δ 13.13 (s, 1H), 11.36 (s, 1H), 9.03 (s, 1H), 8.83 (d, J = 1.5 Hz, 1H), 8.74 (d, J = 7.3 Hz, 1H), 8.32 (s, 1H), 7.92 (s, 1H), 5.11 − 4.76 (m, 1H), 4.66 (t, J = 8.6 Hz, 1H), 2.24 (d, J = 2.8 Hz, 3H), 2.17 (m, 1H), 1.73 − 1.43 (m, 3H), 1.16 (m, 1H), 0.83 (d, J = 7.0 Hz, 1H), 0.73 − 0.27 (m, 7H); LCMS (electrospray) m/z 506.20 (M + H)⁺. | E |
| | (1S,2S)-N-(6-(7- | | |
| | (cyclopropanecarboxamido(cyclopropyl) | | |
| | methyl)-6-fluoro-5-methyl-1H-indazol-4- | | |
| | yl)imidazo[1,2-a]pyrazin-2-yl)-2- | | |
| | fluorocyclopropane-1-carboxamide | | |
| 68 | 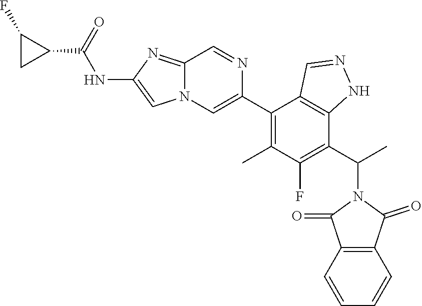 [SMILES: Cc1c(F)c(C(C)N2C(=O)c3ccccc3C2=O)c2[nH]ncc2c1-c1cn2cc(NC(=O)[C@@H]3C[C@@H]3F)nc2cn1] | ¹H NMR (400 MHz, DMSO-D6) δ 13.13 (s, 1H), 11.35 (s, 1H), 9.02 (s, 1H), 8.81 (s, 1H), 8.32 (s, 1H), 7.97 (s, 1H), 7.84 (s, 4H), 5.97 (d, J = 7.4 Hz, 1H), 4.94 (m, J = 66.3 Hz, 1H), 2.17 (d, J = 2.8 Hz, 4H), 2.04 (d, J = 7.4 Hz, 3H), 1.67 (m, J = 24.2 Hz, 1H), 1.15 (m, 1H); LCMS (electrospray) m/z 542.15 (M + H)⁺. | E |
| | (1S,2S)-N-(6-(7-(1-(1,3-dioxoisoindolin- | | |
| | 2-yl)ethyl)-6-fluoro-5-methyl-1H- | | |
| | indazol-4-yl)imidazo[1,2-a]pyrazin-2-yl)- | | |
| | 2-fluorocyclopropane-1-carboxamide | | |
| 69 | 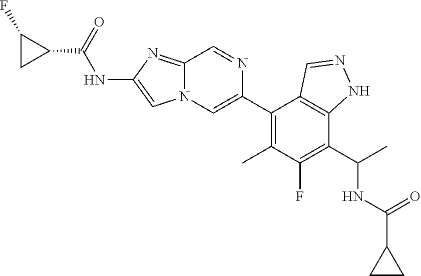 [SMILES: Cc1c(F)c(C(C)NC(=O)C2CC2)c2[nH]ncc2c1-c1cn2cc(NC(=O)[C@@H]3C[C@@H]3F)nc2cn1] | ¹H NMR (400 MHz, DMSO-D6) δ 13.09 (s, 1H), 11.35 (s, 1H), 9.02 (s, 1H), 8.81 (d, J = 1.4 Hz, 1H), 8.52 (d, J = 7.1 Hz, 1H), 8.32 (s, 1H), 7.92 (s, 1H), 5.62 − 5.41 (m, 1H), 4.94 (m, J = 66.1 Hz, 1H), 2.33 − 2.06 (m, 4H), 1.78 − 1.39 (m, 5H), 1.21 (m, 1H), 0.83 (m, 1H), 0.71 − 0.48 (m, 3H); LCMS (electrospray) m/z 480.20 (M + H)⁺. | E |
| | (1S,2S)-N-(6-(7-(1- | | |
| | (cyclopropanecarboxamido)ethyl)-6- | | |
| | fluoro-5-methyl-1H-indazol-4- | | |
| | yl)imidazo[1,2-a]pyrazin-2-yl)-2- | | |
| | fluorocyclopropane-1-carboxamide | | |
| 70 | 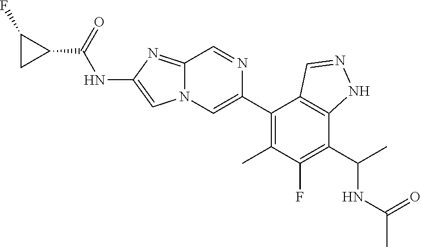 [SMILES: CC(=O)NC(C)c1c(F)c(C)c(-c2cn3cc(NC(=O)[C@@H]4C[C@@H]4F)nc3cn2)c2cn[nH]c12] | ¹H NMR (400 MHz, DMSO-D6) δ 13.05 (s, 1H), 11.35 (s, 1H), 9.02 (s, 1H), 8.80 (d, J = 1.5 Hz, 1H), 8.32 (s, 1H), 8.28 (d, J = 7.1 Hz, 1H), 7.92 (s, 1H), 5.51 − 5.38 (m, 1H), 4.94 (m, J = 66.3 Hz, 1H), 2.26 − 2.13 (m, 4H), 1.83 (s, 3H), 1.64 (m, 1H), 1.51 (d, J = 7.1 Hz, 3H), 1.21 (m, 1H); LCMS (electrospray) m/z 454.10 (M + H)⁺. | E |
| | (1S,2S)-N-(6-(7-(1-acetamidoethyl)-6- | | |
| | fluoro-5-methyl-1H-indazol-4- | | |
| | yl)imidazo[1,2-a]pyrazin-2-yl)-2- | | |
| | fluorocyclopropane-1-carboxamide | | |
| 71 | 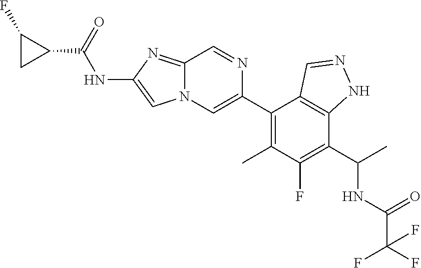 [SMILES: Cc1c(F)c(C(C)NC(=O)C(F)(F)F)c2[nH]ncc2c1-c1cn2cc(NC(=O)[C@@H]3C[C@@H]3F)nc2cn1] | ¹H NMR (400 MHz, DMSO-D6) δ 13.16 (s, 1H), 11.36 (s, 1H), 9.85 (s, 1H), 9.03 (s, 1H), 8.83 (d, J = 1.4 Hz, 1H), 8.32 (s, 1H), 7.96 (s, 1H), 5.54 (m, 1H), 2.25 (d, J = 2.8 Hz, 3H), 2.17 (m, 1H), 1.70 (m, 1H), 1.65 (d, J = 7.2 Hz, 3H), 1.16 (m, 1H); LCMS (electrospray) m/z 508.10 (M + H)⁺. | E |
| | (1S,2S)-2-fluoro-N-(6-(6-fluoro-5- | | |
| | methyl-7-(1-(2,2,2- | | |
| | trifluoroacetamido)ethyl)-1H-indazol-4- | | |
| | yl)imidazo[1,2-a]pyrazin-2- | | |
| | yl)cyclopropane-1-carboxamide | | |
| 72 | 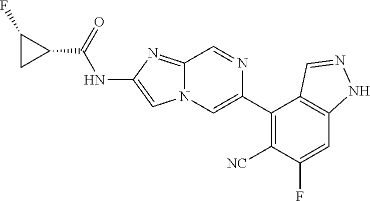 [SMILES: N#Cc1c(F)cc2[nH]ncc2c1-c1cn2cc(NC(=O)[C@@H]3C[C@@H]3F)nc2cn1] | ¹H NMR (400 MHz, DMSO-d₆) δ 13.84 (s, 1H), 11.45 (s, 1H), 9.27 (d, J = 1.4 Hz, 1H), 9.13 (s, 1H), 8.47 (d, J = 8.9 Hz, 2H), 7.75 (d, J = 9.5 Hz, 1H), 4.97 (d, J = 66.4 Hz, 1H), 2.22 − 2.17 (m, 1H), 1.70 (d, J = 23.0 Hz, 1H), 1.24 − 1.19 (m, 1H); LCMS (electrospray) m/z 380.10 (M + H)⁺. | E |
| | (1S,2S)-N-(6-(5-cyano-6-fluoro-1H- | | |
| | indazol-4-yl)imidazo[1,2-a]pyrazin-2-yl)- | | |
| | 2-fluorocyclopropane-1-carboxamide | | |
| 73 | 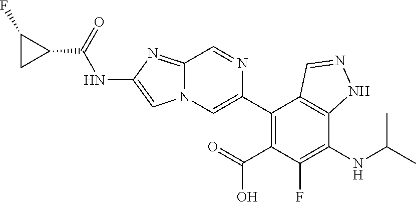 [SMILES: CC(C)Nc1c(F)c(C(=O)O)c(-c2cn3cc(NC(=O)[C@@H]4C[C@@H]4F)nc3cn2)c2cn[nH]c12] | ¹H NMR (400 MHz, DMSO-d₆) δ 13.54 (s, 1H), 12.68 (s, 1H), 11.26 (s, 1H), 9.04 (s, 1H), 8.94 (s, 1H), 8.12 (s, 1H), 8.06 (s, 1H), 5.92 (d, 1H), 4.57 − 4.51 (m, 1H), 3.93 (m, 1H), 2.18 − 2.13 (m, 1H), 1.64 (m, 1H), 1.17 (d, J = 6.3 Hz, 7H); LCMS (electrospray) m/z 456.10 (M + H)⁺. | F |
| | 6-fluoro-4-(2-((1S,2S)-2- | | |
| | fluorocyclopropane-1- | | |
| | carboxamido)imidazo[1,2-a]pyrazin-6- | | |
| | yl)-7-(isopropylamino)-1H-indazole-5- | | |
| | carboxylic acid | | |
| 74 | 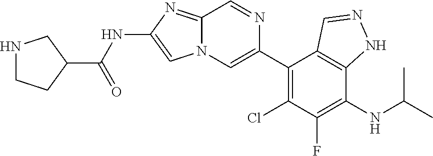 [SMILES: CC(C)Nc1c(F)c(Cl)c(-c2cn3cc(NC(=O)C4CCNC4)nc3cn2)c2cn[nH]c12] | ¹H NMR (400 MHz, DMSO-d₆) δ 13.44 (s, 1H), 11.05 (s, 1H), 9.00 (s, 1H), 8.90 (s, 1H), 8.36 (s, 1H), 7.97 (s, 1H), 5.31 (d, J = 9.3 Hz, 1H), 4.14 − 4.10 (m, 1H), 3.08 − 3.02 (m, 2H), 2.87 − 2.66 (m, 4H), 1.91 − 1.86 (m, 1H), 1.23 (d, J = 6.3 Hz, 7H); LCMS (electrospray) m/z 457.10 (M + H)⁺. | C |
| | N-(6-(5-chloro-6-fluoro-7- | | |
| | (isopropylamino)-1H-indazol-4- | | |
| | yl)imidazo[1,2-a]pyrazin-2- | | |
| | yl)pyrrolidine-3-carboxamide | | |
| 75 | 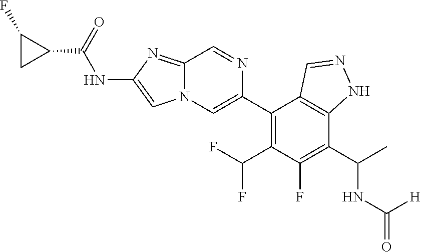 [SMILES: CC(NC=O)c1c(F)c(C(F)F)c(-c2cn3cc(NC(=O)[C@@H]4C[C@@H]4F)nc3cn2)c2cn[nH]c12] | ¹H NMR (400 MHz, DMSO-d₆) δ 13.62 − 13.55 (s, 1H), 11.44 − 11.36 (s, 1H), 9.07 − 9.01 (s, 1H), 8.99 − 8.92 (s, 1H), 8.71 − 8.60 (d, J = 7.2 Hz, 1H), 8.42 − 8.33 (s, 1H), 8.25 − 8.16 (s, 1H), 8.09 − 8.03 (s, 1H), 7.26 − 6.93 (t, J = 52.4 Hz, 1H), 5.66 − 5.50 (m, 1H), 5.12 − 4.82 (m, 1H), 2.22 − 2.15 (m, 1H), 1.72 − 1.65 (m, 1H), 1.62 − 1.55 (d, J = 7.1 Hz, 3H), 1.18 − 1.16 (m, 1H); LCMS (electrospray) m/z 476.10 (M + H)⁺. | E |
| | (1S,2S)-N-(6-(5-(difluoromethyl)-6- | | |
| | fluoro-7-(1-formamidoethyl)-1H-indazol- | | |
| | 4-yl)imidazo[1,2-a]pyrazin-2-yl)-2- | | |
| | fluorocyclopropane-1-carboxamide | | |
| 76 | 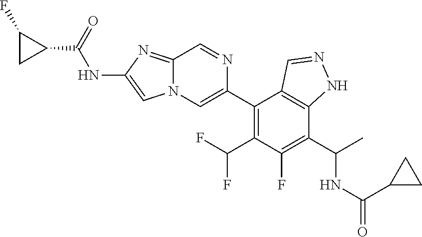 [SMILES: CC(NC(=O)C1CC1)c1c(F)c(C(F)F)c(-c2cn3cc(NC(=O)[C@@H]4C[C@@H]4F)nc3cn2)c2cn[nH]c12] | ¹H NMR (400 MHz, DMSO-d₆) δ 13.62 − 13.52 (s, 1H), 11.43 − 11.37 (s, 1H), 9.07 − 8.99 (s, 1H), 8.99 − 8.93 (d, J = 1.5 Hz, 1H), 8.67 − 8.61 (d, J = 7.0 Hz, 1H), 8.42 − 8.33 (s, 1H), 8.24 − 8.16 (s, 1H), 7.24 − 6.94 (t, J = 52.5 Hz, 1H), 5.58 − 5.47 (m, 1H), 5.09 − 4.84 (m, 1H), 2.24 − 2.15 (m, 1H), 1.73 − 1.66 (d, 3H), 1.74 − 1.63 (m, 2H), 1.60 − 1.57 (d, J = 7.2 Hz, 3H), 1.24 − 1.22 (m, 1H), 0.73 − 0.58 (m, 4H); LCMS (electrospray) m/z 516.10 (M + H)⁺. | E |
| | (1S,2S)-N-(6-(7-(1- | | |
| | (cyclopropanecarboxamido)ethyl)-5- | | |
| | (difluoromethyl)-6-fluoro-1H-indazol-4- | | |
| | yl)imidazo[1,2-a]pyrazin-2-yl)-2- | | |
| | fluorocyclopropane-1-carboxamide | | |
| 77 | 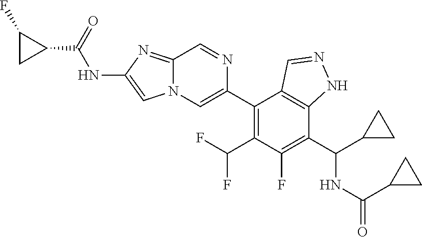 [SMILES: O=C(NC(c1c(F)c(C(F)F)c(-c2cn3cc(NC(=O)[C@@H]4C[C@@H]4F)nc3cn2)c2cn[nH]c12)C1CC1)C1CC1] | ¹H NMR (400 MHz, DMSO-d₆) δ 13.62 − 13.48 (s, 1H), 11.48 − 11.29 (s, 1H), 9.11 − 9.01 (s, 1H), 9.01 − 8.94 (s, 1H), 8.92 − 8.82 (d, J = 6.9 Hz, 1H), 8.43 − 8.29 (s, 1H), 7.27 − 6.89 (t, J = 52.4 Hz, 1H), 5.11 − 4.82 (m, 1H), 4.74 − 4.63 (m, 1H), 2.25 − 2.10 (m, 1H), 1.78 − 1.62 (m, 2H), 1.61 − 1.51 (m, 1H), 0.71 − 0.34 (m, 8H); LCMS (electrospray) m/z 542.20 (M + H)⁺. | E |
| | (1S,2S)-N-(6-(7- | | |
| | (cyclopropanecarboxamido(cyclopropyl) | | |
| | methyl)-5-(difluoromethyl)-6-fluoro-1H- | | |
| | indazol-4-yl)imidazo[1,2-a]pyrazin-2-yl)- | | |
| | 2-fluorocyclopropane-1-carboxamide | | |
| 78 | 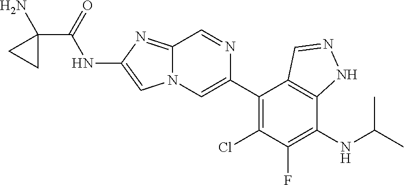 [SMILES: CC(C)Nc1c(F)c(Cl)c(-c2cn3cc(NC(=O)C4(N)CC4)nc3cn2)c2cn[nH]c12] | ¹H NMR (400 MHz, DMSO-d₆) δ 13.17 (s, 1H), 10.88-11.11 (OH), 9.04 (s, 1H), 8.92 (s, 1H), 8.36 (s, 1H), 7.95 (s, 1H), 5.20 (d, J = 8.2 Hz, 1H), 4.03 (s, 1H), 1.23 (d, J = 6.3 Hz, 8H), 0.95 (d, J = 3.1 Hz, 2H); LCMS (electrospray) 443.10 (M + H)⁺. | C |
| | 1-amino-N-(6-(5-chloro-6-fluoro-7- | | |
| | (isopropylamino)-1H-indazol-4- | | |
| | yl)imidazo[1,2-a]pyrazin-2- | | |
| | yl)cyclopropane-1-carboxamide | | |
| 79 | 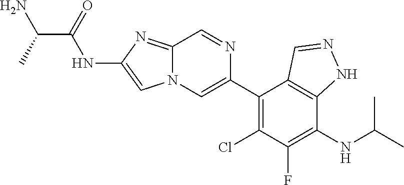 [SMILES: CC(C)Nc1c(F)c(Cl)c(-c2cn3cc(NC(=O)[C@H](C)N)nc3cn2)c2cn[nH]c12] | ¹H NMR (400 MHz, DMSO-d₆) δ 13.28 (s, 1H), 9.02 (s, 1H), 8.92 (s, 1H), 8.39 (s, 1H), 7.98 (s, 1H), 5.23 (d, J = 9.8 Hz, 1H), 4.11 (s, 1H), 3.55 (q, J = 6.8 Hz, 1H), 1.23 (d, J = 6.7 Hz, 9H); LCMS (electrospray) 431.15 (M + H)⁺. | C |
| | (S)-2-amino-N-(6-(5-chloro-6-fluoro-7- | | |
| | (isopropylamino)-1H-indazol-4- | | |
| | yl)imidazo[1,2-a]pyrazin-2- | | |
| | yl)propanamide | | |
| 80 | 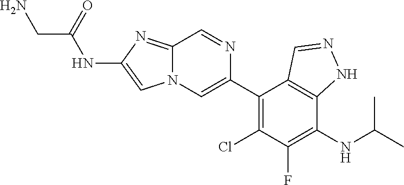 [SMILES: CC(C)Nc1c(F)c(Cl)c(-c2cn3cc(NC(=O)CN)nc3cn2)c2cn[nH]c12] | ¹H NMR (400 MHz, DMSO-d₆) δ 13.47 (s, 1H), 11.88 (s, OH), 9.02 (s, 1H), 8.92 (s, 1H), 8.40 (s, 1H), 7.97 (s, 1H), 5.34 (d, J = 9.0 Hz, 1H), 4.07-4.29 (1H), 3.36 (s, 2H), 1.24 (d, J = 6.3 Hz, 6H); LCMS (electrospray) 417.10 (M + H)⁺. | C |
| | 2-amino-N-(6-(5-chloro-6-fluoro-7- | | |
| | (isopropylamino)-1H-indazol-4- | | |
| | yl)imidazo[1,2-a]pyrazin-2-yl)acetamide | | |
| 81 | 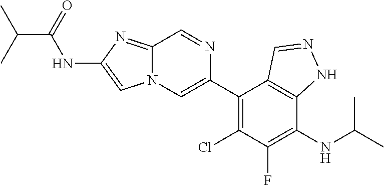 [SMILES: CC(C)Nc1c(F)c(Cl)c(-c2cn3cc(NC(=O)C(C)C)nc3cn2)c2cn[nH]c12] | ¹H NMR (400 MHz, DMSO-d₆) δ 13.17 (s, 1H), 10.97 (s, 1H), 9.01 (s, 1H), 8.91 (s, 1H), 8.37 (s, 1H), 7.94 (s, 1H), 5.20 (d, J = 9.4 Hz, 1H), 4.03 (d, J = 6.7 Hz, 1H), 2.79-2.73 (m, 1H), 1.23 (d, J = 6.3 Hz, 6H), 1.15-1.11 (m, 6H); LCMS (electrospray) 430.10 (M + H)⁺. | C |
| | N-(6-(5-chloro-6-fluoro-7- | | |
| | (isopropylamino)-1H-indazol-4- | | |
| | yl)imidazo[1,2-a]pyrazin-2- | | |
| | yl)isobutyramide | | |
| 82 | 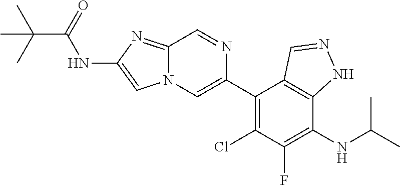 [SMILES: CC(C)Nc1c(F)c(Cl)c(-c2cn3cc(NC(=O)C(C)(C)C)nc3cn2)c2cn[nH]c12] | ¹H NMR (400 MHz, DMSO-d₆) δ 13.18 (s, 1H), 10.60 (s, 1H), 9.01 (s, 1H), 8.91 (s, 1H), 8.38 (s, 1H), 7.94 (s, 1H), 5.20 (d, J = 9.4 Hz, 1H), 4.05 (s, 1H), 1.26 (s, 9H), 1.23 (d, J = 6.3 Hz, 6H); LCMS (electrospray) 444.15 (M + H)⁺. | C |
| | N-(6-(5-chloro-6-fluoro-7- | | |
| | (isopropylamino)-1H-indazol-4- | | |
| | yl)imidazo[1,2-a]pyrazin-2-yl)pivalamide | | |
| 83 | 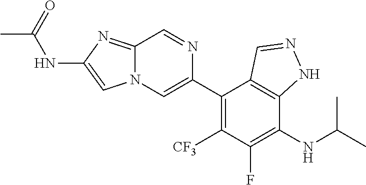 [SMILES: CC(=O)Nc1cn2cc(-c3c(C(F)(F)F)c(F)c(NC(C)C)c4[nH]ncc34)ncc2n1] | ¹H NMR (400 MHz, DMSO-d₆) δ 13.43 (br s, 1H), 11.04 (s, 1H), 8.95 (s, 1H), 8.78 (s, 1H), 8.32 (s, 1H), 7.92 (br s, 1H), 5.32 − 5.16 (m, 1H), 2.13 (s, 3H), 1.25 (br d, J = 6.0 Hz, 6H); LCMS (electrospray) m/z 436.1 (M + H)⁺. | C |
| | N-(6-(6-fluoro-7-(isopropylamino)-5- | | |
| | (trifluoromethyl)-1H-indazol-4- | | |
| | yl)imidazo[1,2-a]pyrazin-2-yl)acetamide | | |
| 84 | 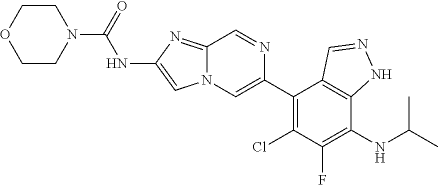 [SMILES: CC(C)Nc1c(F)c(Cl)c(-c2cn3cc(NC(=O)N4CCOCC4)nc3cn2)c2cn[nH]c12] | ¹H NMR (400 MHz, DMSO-d₆) δ 13.27 − 13.08 (m, 1H), 9.80 (s, 1H), 8.94 (s, 1H), 8.87 (s, 1H), 8.21 (s, 1H), 7.94 (br s, 1H), 5.24 − 5.12 (m, 1H), 4.14 − 3.91 (m, 1H), 3.64 − 3.57 (m, 4H), 3.53 − 3.47 (m, 4H), 1.23 (d, J = 6.2 Hz, 6H); LCMS (electrospray) m/z 473.2 (M + H)⁺. | C |
| | N-(6-(5-chloro-6-fluoro-7- | | |
| | (isopropylamino)-1H-indazol-4- | | |
| | yl)imidazo[1,2-a]pyrazin-2- | | |
| | yl)morpholine-4-carboxamide | | |
| 85 | 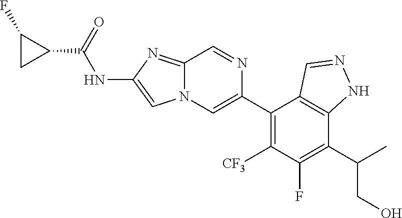 [SMILES: CC(CO)c1c(F)c(C(F)(F)F)c(-c2cn3cc(NC(=O)[C@@H]4C[C@@H]4F)nc3cn2)c2cn[nH]c12] | ¹H NMR (400 MHz, DMSO-d₆) δ 13.72 (br s, 1H), 11.41 (s, 1H), 9.01 (s, 1H), 8.86 (s, 1H), 8.35 (s, 1H), 8.04 (s, 1H), 5.08 − 4.84 (m, 3H), 3.85 (br d, J = 8.7 Hz, 1H), 3.63 (br d, J = 7.9 Hz, 1H), 1.79 (br d, J = 3.2 Hz, 1H), 1.41 (br d, J = 7.1 Hz, 3H), 1.38 − 1.32 (m, 1H), 1.22 (br dd, J = 3.1, 5.8 Hz, 1H); LCMS (electrospray) m/z 481.1 (M + H)⁺. | E |
| | (1S,2S)-2-fluoro-N-(6-(6-fluoro-7-(1- | | |
| | hydroxypropan-2-yl)-5-(trifluoromethyl)- | | |
| | 1H-indazol-4-yl)imidazo[1,2-a]pyrazin-2- | | |
| | yl)cyclopropane-1-carboxamide | | |
| 86 | 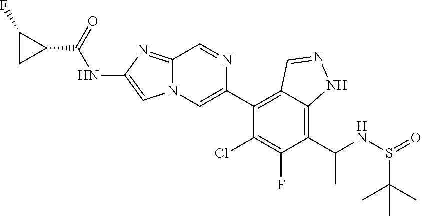 [SMILES: CC(NS(=O)C(C)(C)C)c1c(F)c(Cl)c(-c2cn3cc(NC(=O)[C@@H]4C[C@@H]4F)nc3cn2)c2cn[nH]c12] | ¹H NMR (400 MHz, DMSO-d₆) δ 13.40 (br s, 1H), 11.42 (s, 1H), 9.08 (s, 1H), 9.03 (s, 1H), 8.39 (s, 1H), 8.06 (s, 1H), 5.50 (d, J = 3.2 Hz, 1H), 5.16 (br dd, J = 3.1, 6.7 Hz, 1H), 5.09 − 4.86 (m, 1H), 2.20 (td, J = 7.0, 13.7 Hz, 1H), 1.71 (d, J = 6.8 Hz, 3H), 1.68 − 1.64 (m, 1H), 1.26 − 1.19 (m, 1H), 1.06 (s, 9H); LCMS (electrospray) m/z 536.1 (M + H)⁺. | G |
| | (1S,2S)-N-(6-(7-(1-((tert- | | |
| | butylsulfinyl)amino)ethyl)-5-chloro-6- | | |
| | fluoro-1H-indazol-4-yl)imidazo[1,2- | | |
| | a]pyrazin-2-yl)-2-fluorocyclopropane-1- | | |
| | carboxamide | | |
| 87 | 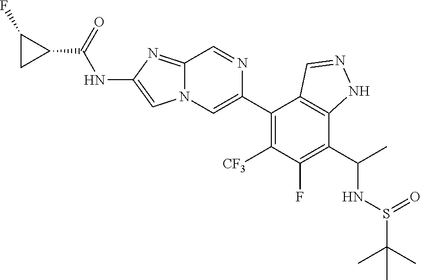 [SMILES: CC(NS(=O)C(C)(C)C)c1c(F)c(C(F)(F)F)c(-c2cn3cc(NC(=O)[C@@H]4C[C@@H]4F)nc3cn2)c2cn[nH]c12] | ¹H NMR (400 MHz, DMSO-d₆) δ 13.65 (br s, 1H), 11.42 (s, 1H), 9.01 (s, 1H), 8.92 (s, 1H), 8.34 (s, 1H), 8.08 (s, 1H), 5.54 (br d, J = 2.8 Hz, 1H), 5.25 − 5.16 (m, 1H), 5.09 − 4.85 (m, 1H), 2.21 − 2.17 (m, 1H), 1.72 (br d, J = 6.6 Hz, 3H), 1.67 (br d, J = 4.6 Hz, 1H), 1.22 (br dd, J = 3.4, 6.9 Hz, 1H), 1.06 (s, 9H); LCMS (electrospray) m/z 570.1 (M + H)⁺. | G |
| | (1S,2S)-N-(6-(7-(1-((tert- | | |
| | butylsulfinyl)amino)ethyl)-6-fluoro-5- | | |
| | (trifluoromethyl)-1H-indazol-4- | | |
| | yl)imidazo[1,2-a]pyrazin-2-yl)-2- | | |
| | fluorocyclopropane-1-carboxamide | | |
| 88 | 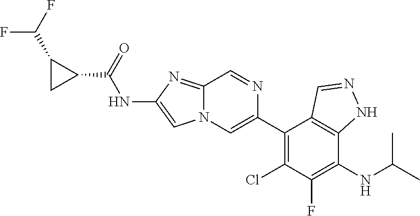 [SMILES: CC(C)Nc1c(F)c(Cl)c(-c2cn3cc(NC(=O)[C@@H]4C[C@@H]4C(F)F)nc3cn2)c2cn[nH]c12] | ¹H NMR (400 MHz, DMSO-d₆) δ 13.17 (s, 1H), 11.59 (s, 1H), 9.04 (s, 1H), 8.88 (s, 1H), 8.35 (s, 1H), 7.95 (s, 1H), 6.30 − 5.81 (m, 1H), 5.35 − 5.04 (m, 1H), 4.18 − 3.90 (m, 1H), 2.34 − 2.30 (q, J = 7.1 Hz, 1H), 2.01 − 1.83 (m, 1H), 1.44 − 1.28 (m, 2H), 1.23 (br d, J = 6.1 Hz, 6H); LCMS (electrospray) m/z 478.1 (M + H)⁺. | C |
| | (1R,2S)-N-(6-(5-chloro-6-fluoro-7- | | |
| | (isopropylamino)-1H-indazol-4- | | |
| | yl)imidazo[1,2-a]pyrazin-2-yl)-2- | | |
| | (difluoromethyl)cyclopropane-1- | | |
| | carboxamide | | |
| 89 | 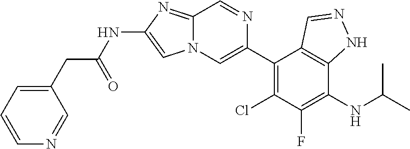 [SMILES: CC(C)Nc1c(F)c(Cl)c(-c2cn3cc(NC(=O)Cc4cccnc4)nc3cn2)c2cn[nH]c12] | ¹H NMR (400 MHz, DMSO-d₆) δ 13.70 − 12.41 (m, 1H), 11.42 (s, 1H), 9.04 (s, 1H), 8.91 (s, 1H), 8.73 (s, 1H), 8.66 (br d, J = 4.9 Hz, 1H), 8.33 (s, 1H), 8.14 (br d, J = 8.8 Hz, 1H), 7.97 (s, 1H), 7.70 (br t, J = 6.4 Hz, 1H), 4.23 − 4.03 (m, 2H), 3.96 (s, 3H), 1.23 (d, J = 6.3 Hz, 6H); LCMS (electrospray) m/z 479.2 (M + H)⁺. | C |
| | N-(6-(5-chloro-6-fluoro-7- | | |
| | (isopropylamino)-1H-indazol-4- | | |
| | yl)imidazo[1,2-a]pyrazin-2-yl)-2- | | |
| | (pyridin-3-yl)acetamide | | |
| 90 | 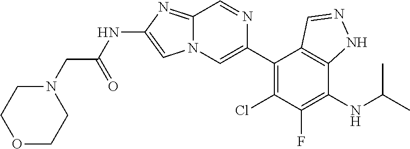 [SMILES: CC(C)Nc1c(F)c(Cl)c(-c2cn3cc(NC(=O)CN4CCOCC4)nc3cn2)c2cn[nH]c12] | ¹H NMR (400 MHz, DMSO-d₆) δ 13.19 (s, 1H), 9.05 (s, 1H), 8.94 (s, 1H), 8.41 (s, 1H), 7.95 (s, 1H), 5.35 − 5.01 (m, 1H), 4.04 (br d, J = 1.5 Hz, 1H), 3.70 (br s, 6H), 3.05 − 2.53 (m, 4H), 1.24 (s, 3H), 1.23 (s, 3H); LCMS (electrospray) m/z 487.1 (M + H)⁺. | C |
| | N-(6-(5-chloro-6-fluoro-7- | | |
| | (isopropylamino)-1H-indazol-4- | | |
| | yl)imidazo[1,2-a]pyrazin-2-yl)-2- | | |
| | morpholinoacetamide | | |
| 91 | 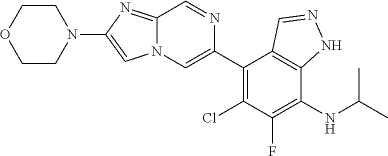 [SMILES: CC(C)Nc1c(F)c(Cl)c(-c2cn3cc(N4CCOCC4)nc3cn2)c2cn[nH]c12] | ¹H NMR (400 MHz, DMSO-d₆) δ 13.15 (s, 1H), 8.85 (s, 1H), 8.72 (s, 1H), 7.92 (s, 1H), 7.57 (s, 1H), 5.21 − 5.10 (m, 1H), 4.10 − 3.98 (m, 1H), 3.80 − 3.72 (m, 4H), 3.31 − 3.24 (m, 4H), 1.23 (d, J = 6.3 Hz, 6H); LCMS (electrospray) m/z 430.1 (M + H)⁺. | H |
| | 5-chloro-6-fluoro-N-isopropyl-4-(2- | | |
| | morpholinoimidazo[1,2-a]pyrazin-6-yl)- | | |
| | 1H-indazol-7-amine | | |
| 92 | 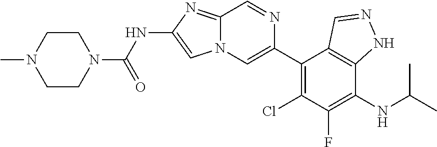 [SMILES: CC(C)Nc1c(F)c(Cl)c(-c2cn3cc(NC(=O)N4CCN(C)CC4)nc3cn2)c2cn[nH]c12] | ¹H NMR (400 MHz, DMSO-d₆) δ 9.76 (s, 1H), 8.94 (s, 1H), 8.86 (d, J = 1.0 Hz, 1H), 8.20 (s, 1H), 8.15 (s, 1H), 8.01 − 7.90 (m, 1H), 5.22 − 5.15 (m, 1H), 4.13 − 3.96 (m, 1H), 3.56 − 3.47 (m, 4H), 2.32 (br t, J = 4.8 Hz, 4H), 2.20 (s, 3H), 1.23 (d, J = 6.2 Hz, 6H); LCMS (electrospray) m/z 486.2 (M + H)⁺. | C |
| | N-(6-(5-chloro-6-fluoro-7- | | |
| | (isopropylamino)-1H-indazol-4- | | |
| | yl)imidazo[1,2-a]pyrazin-2-yl)-4- | | |
| | methylpiperazine-1-carboxamide formate | | |
| 93 | 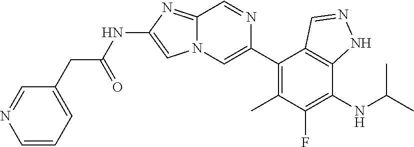 [SMILES: Cc1c(F)c(NC(C)C)c2[nH]ncc2c1-c1cn2cc(NC(=O)Cc3cccnc3)nc2cn1] | ¹H NMR (400 MHz, DMSO-d₆) δ 12.96 − 12.86 (m, 1H), 11.34 (s, 1H), 9.01 (s, 1H), 8.74 (d, J = 0.8 Hz, 1H), 8.55 (d, J = 1.8 Hz, 1H), 8.47 (dd, J = 1.6, 4.8 Hz, 1H), 8.30 (s, 1H), 7.86 (s, 1H), 7.76 (td, J = 1.9, 7.8 Hz, 1H), 7.37 (dd, J = 4.8, 7.8 Hz, 1H), 4.72 (dd, J = 2.5, 9.9 Hz, 1H), 4.05 − 3.92 (m, 1H), 3.81 (s, 2H), 2.25 (d, J = 3.4 Hz, 3H), 1.20 (d, J = 6.3 Hz, 6H); LCMS (electrospray) m/z 458.9 (M + H)⁺. | B |
| | N-(6-(6-fluoro-7-(isopropylamino)-5- | | |
| | methyl-1H-indazol-4-yl)imidazo[1,2- | | |
| | a]pyrazin-2-yl)-2-(pyridin-3-yl)acetamide | | |
| 94 | 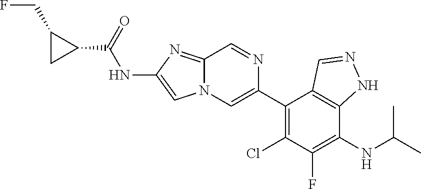 [SMILES: CC(C)Nc1c(F)c(Cl)c(-c2cn3cc(NC(=O)[C@@H]4C[C@@H]4CF)nc3cn2)c2cn[nH]c12] | ¹H NMR (400 MHz, DMSO-d₆) δ 13.17 (br s, 1H), 11.39 (s, 1H), 9.02 (s, 1H), 8.87 (s, 1H), 8.35 (s, 1H), 8.18 (s, 1H), 7.95 (br s, 1H), 5.20 (br d, J = 8.0 Hz, 1H), 4.86 − 4.65 (m, 1H), 4.63 − 4.43 (m, 1H), 4.04 (br d, J = 2.8 Hz, 1H), 2.21 (q, J = 6.8 Hz, 1H), 1.87 − 1.69 (m, 1H), 1.23 (d, J = 6.3 Hz, 6H), 1.21 − 1.14 (m, 1H), 1.07 (br d, J = 5.6 Hz, 1H); LCMS (electrospray) m/z 460.2 (M + H)⁺. | C |
| | (1R,2S)-N-(6-(5-chloro-6-fluoro-7- | | |
| | (isopropylamino)-1H-indazol-4- | | |
| | yl)imidazo[1,2-a]pyrazin-2-yl)-2- | | |
| | (fluoromethyl)cyclopropane-1- | | |
| | carboxamide | | |
| 95 | 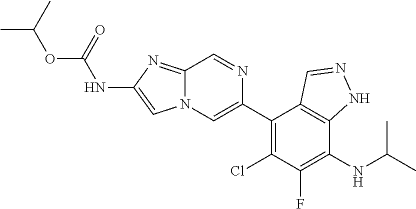 [SMILES: CC(C)Nc1c(F)c(Cl)c(-c2cn3cc(NC(=O)OC(C)C)nc3cn2)c2cn[nH]c12] | ¹H NMR (400 MHz, DMSO-d₆) δ 13.16 − 13.15 (br d, J = 1.5 Hz, 1H), 10.55 − 10.37 (m, 1H), 8.98 (s, 1H), 8.89 (s, 1H), 8.15 (s, 1H), 7.95 (s, 1H), 5.28 − 5.10 (m, 1H), 5.00 − 4.86 (m, 1H), 4.28 − 3.90 (m, 1H), 1.29 (br d, J = 6.1 Hz, 6H), 1.24 (br d, J = 6.0 Hz, 6H); LCMS (electrospray) m/z 446.1 (M + H)⁺. | C |
| | isopropyl (6-(5-chloro-6-fluoro-7- | | |
| | (isopropylamino)-1H-indazol-4- | | |
| | yl)imidazo[1,2-a]pyrazin-2-yl)carbamate | | |
| 96 | 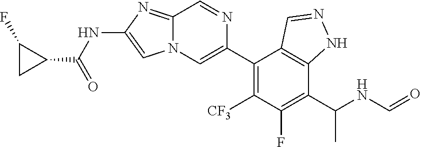 [SMILES: CC(NC=O)c1c(F)c(C(F)(F)F)c(-c2cn3cc(NC(=O)[C@@H]4C[C@@H]4F)nc3cn2)c2cn[nH]c12] | ¹H NMR (400 MHz, DMSO-d₆) δ 13.82 (br s, 1H), 11.41 (s, 1H), 9.01 (s, 1H), 8.89 (d, J = 1.0 Hz, 1H), 8.74 (br d, J = 6.7 Hz, 1H), 8.34 (s, 1H), 8.09 (br d, J = 7.3 Hz, 2H), 5.66 − 5.54 (m, 1H), 5.08 − 4.86 (m, 1H), 2.20 (td, J = 7.0, 13.6 Hz, 1H), 1.72 (dt, J = 3.9, 6.7 Hz, 1H), 1.69 − 1.64 (m, 1H), 1.60 (d, J = 7.2 Hz, 3H), 1.25 − 1.18 (m, 1H); LCMS (electrospray) m/z 494.1 (M + H)⁺. | F |
| | (1S,2S)-2-fluoro-N-(6-(6-fluoro-7-(1- | | |
| | formamidoethyl)-5-(trifluoromethyl)-1H- | | |
| | indazol-4-yl)imidazo[1,2-a]pyrazin-2- | | |
| | yl)cyclopropane-1-carboxamide | | |
| 97 | 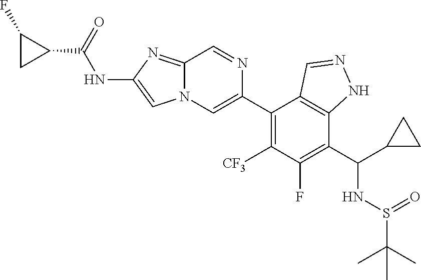 [SMILES: CC(C)(C)S(=O)NC(c1c(F)c(C(F)(F)F)c(-c2cn3cc(NC(=O)[C@@H]4C[C@@H]4F)nc3cn2)c2cn[nH]c12)C1CC1] | ¹H NMR (400 MHz, DMSO-d₆) δ 13.75 (s, 1H), 11.42 (s, 1H), 9.01 (s, 1H), 8.94 (s, 1H), 8.34 (s, 1H), 8.09 (s, 1H), 5.49 (br d, J = 3.6 Hz, 1H), 5.10 − 4.84 (m, 1H), 4.22 (br dd, J = 3.5, 9.6 Hz, 1H), 2.20 (td, J = 6.6, 13.6 Hz, 1H), 1.74 − 1.64 (m, 2H), 1.25 − 1.16 (m, 1H), 1.07 (s, 9H), 0.76 (br d, J = 7.4 Hz, 1H), 0.64 − 0.50 (m, 2H), 0.43 − 0.34 (m, 1H); LCMS (electrospray) m/z 596.1 (M + H)⁺. | F |
| | (1S,2S)-N-(6-(7-(((tert- | | |
| | butylsulfinyl)amino)(cyclopropyl)methyl) | | |
| | -6-fluoro-5-(trifluoromethyl)-1H-indazol- | | |
| | 4-yl)imidazo[1,2-a]pyrazin-2-yl)-2- | | |
| | fluorocyclopropane-1-carboxamide | | |
| 98 | 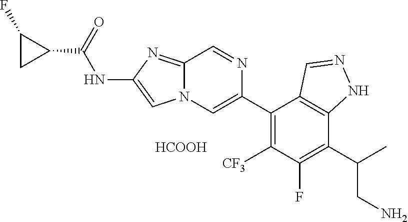 [SMILES: CC(CN)c1c(F)c(C(F)(F)F)c(-c2cn3cc(NC(=O)[C@@H]4C[C@@H]4F)nc3cn2)c2cn[nH]c12 ; "HCOOH"] | ¹H NMR (400 MHz, DMSO-d₆) δ 11.41 (s, 1H), 9.01 (s, 1H), 8.85 (s, 1H), 8.37 (s, 1H), 8.35 (s, 1H), 8.07 (s, 1H), 5.07 − 4.86 (m, 1H), 3.18 (br d, J = 3.9 Hz, 1H), 2.24 − 2.16 (m, 1H), 1.73 − 1.65 (m, 1H), 1.46 (brd, J = 6.9 Hz, 3H), 1.27 − 1.18 (m, 1H); LCMS (electrospray) m/z 480.2 (M + H)⁺. | F |
| | (1S,2S)-N-(6-(7-(1-aminopropan-2-yl)-6- | | |
| | fluoro-5-(trifluoromethyl)-1H-indazol-4- | | |
| | yl)imidazo[1,2-a]pyrazin-2-yl)-2- | | |
| | fluorocyclopropane-1-carboxamide | | |
| | formate | | |
| 99 | 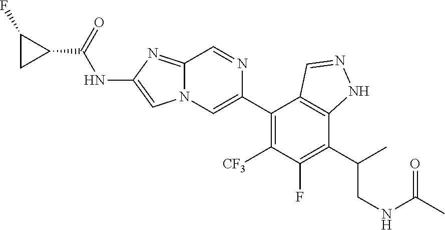 [SMILES: CC(=O)NCC(C)c1c(F)c(C(F)(F)F)c(-c2cn3cc(NC(=O)[C@@H]4C[C@@H]4F)nc3cn2)c2cn[nH]c12] | ¹H NMR (400 MHz, DMSO-d₆) δ 13.76 (br s, 1H), 11.40 (s, 1H), 9.00 (s, 1H), 8.85 (s, 1H), 8.36 (s, 1H), 8.07 (br s, 1H), 8.01 (brt, J = 5.9 Hz, 1H), 5.08 − 4.85 (m, 1H), 3.70 − 3.64 (m, 1H), 3.53 − 3.48 (m, 2H), 2.24 − 2.15 (m, 1H), 1.74 (s, 3H), 1.72 − 1.63 (m, 1H), 1.43 (d, J = 7.1 Hz, 3H), 1.25 − 1.20 (m, 1H); LCMS (electrospray) m/z 522.2 (M + H)⁺. | F |
| | (1S,2S)-N-(6-(7-(1-acetamidopropan-2- | | |
| | yl)-6-fluoro-5-(trifluoromethyl)-1H- | | |
| | indazol-4-yl)imidazo[1,2-a]pyrazin-2-yl)- | | |
| | 2-fluorocyclopropane-1-carboxamide | | |
| 100 | 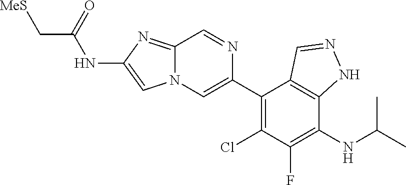 [SMILES: CSCC(=O)Nc1cn2cc(-c3c(Cl)c(F)c(NC(C)C)c4[nH]ncc34)ncc2n1] | ¹H NMR (400 MHz, DMSO-d₆) δ 11.14 (s, 1H), 9.03 (s, 1H), 8.91 (s, 1H), 8.39 (s, 1H), 7.98 (s, 1H), 4.16 − 4.10 (m, 1H), 3.37 (s, 2H), 2.17 (s, 3H), 1.24 (d, J = 6.3 Hz, 6H); LCMS (electrospray) m/z 448.2 (M + H)⁺. | C |
| | N-(6-(5-chloro-6-fluoro-7- | | |
| | (isopropylamino)-1H-indazol-4- | | |
| | yl)imidazo[1,2-a]pyrazin-2-yl)-2- | | |
| | (methylthio)acetamide | | |
| 101 | 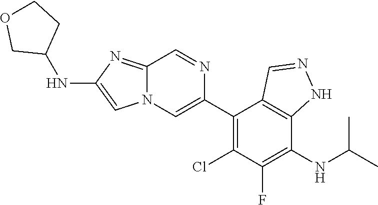 [SMILES: CC(C)Nc1c(F)c(Cl)c(-c2cn3cc(NC4CCOC4)nc3cn2)c2cn[nH]c12] | ¹H NMR (400 MHz, DMSO-d₆) δ 13.29 − 12.99 (m, 1H), 8.72 (s, 1H), 8.64 (s, 1H), 7.93 (br d, J = 1.9 Hz, 1H), 7.33 (s, 1H), 6.47 (d, J = 6.6 Hz, 1H), 5.15 (br d, J = 9.0 Hz, 1H), 4.21 − 4.13 (m, 1H), 3.93 − 3.81 (m, 2H), 3.78 − 3.70 (m, 1H), 3.60 (dd, J = 3.8, 8.7 Hz, 1H), 2.24 − 2.14 (m, 1H), 1.91 − 1.80 (m, 1H), 1.23 (d, J = 6.3 Hz, 6H); LCMS (electrospray) m/z 430.2 (M + H)⁺. | I |
| | 6-(5-chloro-6-fluoro-7-(isopropylamino)- | | |
| | 1H-indazol-4-yl)-N-(tetrahydrofuran-3- | | |
| | yl)imidazo[1,2-a]pyrazin-2-amine | | |
| 102 | 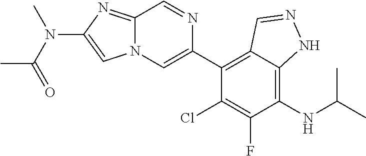 [SMILES: CC(=O)N(C)c1cn2cc(-c3c(Cl)c(F)c(NC(C)C)c4[nH]ncc34)ncc2n1] | ¹H NMR (400 MHz, DMSO-d₆) δ 13.52 − 12.74 (m, 1H), 9.10 ( s, 1H), 8.92 (s, 1H), 8.71 − 8.35 (m, 1H), 7.98 (s, 1H), 5.53 − 4.90 (m, 1H), 4.43 − 3.90 (m, 1H), 3.63 (s, 3H), 2.37 − 2.25 (m, 3H), 1.24 (d, J = 6.3 Hz, 6H); LCMS (electrospray) m/z 416.1 (M + H)⁺. | F |
| | N-(6-(5-chloro-6-fluoro-7- | | |
| | (isopropylamino)-1H-indazol-4- | | |
| | yl)imidazo[1,2-a]pyrazin-2-yl)-N- | | |
| | methylacetamide | | |
| 103 | 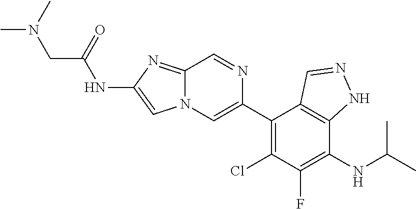 [SMILES: CC(C)Nc1c(F)c(Cl)c(-c2cn3cc(NC(=O)CN(C)C)nc3cn2)c2cn[nH]c12] | ¹H NMR (400 MHz, DMSO-d₆) δ 13.33 − 13.12 (m, 1H), 10.62 (s, 1H), 9.02 (s, 1H), 8.91 (d, J = 1.2 Hz, 1H), 8.41 (s, 1H), 8.23 (s, 1H), 7.95 (br s, 1H), 5.21 (br d, J = 7.2 Hz, 1H), 4.20 − 3.91 (m, 1H), 3.49 (br s, 2H), 2.30 (s, 6H), 1.24 (d, J = 6.2 Hz, 6H); LCMS (electrospray) m/z 445.1 (M + H)⁺. | C |
| | N-(6-(5-chloro-6-fluoro-7- | | |
| | (isopropylamino)-1H-indazol-4- | | |
| | yl)imidazo[1,2-a]pyrazin-2-yl)-2- | | |
| | (dimethylamino)acetamide | | |
| 104 | 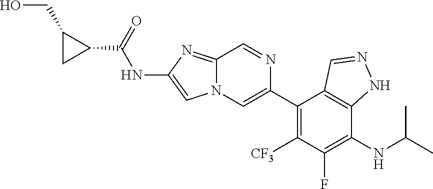 [SMILES: CC(C)Nc1c(F)c(C(F)(F)F)c(-c2cn3cc(NC(=O)[C@@H]4C[C@@H]4CO)nc3cn2)c2cn[nH]c12] | ¹H NMR (400 MHz, DMSO-d₆) δ 13.47 − 13.35 (m, 1H), 11.23 (s, 1H), 8.95 (s, 1H), 8.77 (s, 1H), 8.31 (s, 1H), 7.91 (br s, 1H), 5.24 (brd, J = 6.8 Hz, 1H), 4.48 (t, J = 5.2 Hz, 1H), 4.15 − 4.01 (m, 1H), 3.67 − 3.63 (m, 1H), 3.50 − 3.48 (m, 1H), 2.11 − 2.03 (m, 1H), 1.54 − 1.44 (m, 1H), 1.24 (d, J = 6.4 Hz, 6H), 1.03 (dt, J = 4.0, 8.0 Hz, 1H), 0.98 − 0.93 (m, 1H); LCMS (electrospray) m/z 492.2 (M + H)⁺. | C |
| | (1R,2S)-N-(6-(6-fluoro-7- | | |
| | (isopropylamino)-5-(trifluoromethyl)-1H- | | |
| | indazol-4-yl)imidazo[1,2-a]pyrazin-2-yl)- | | |
| | 2-(hydroxymethyl)cyclopropane-1- | | |
| | carboxamide | | |
| 105 | 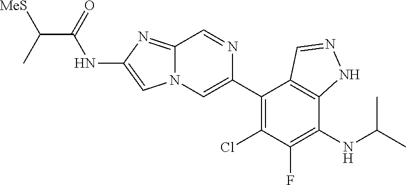 [SMILES: CSC(C)C(=O)Nc1cn2cc(-c3c(Cl)c(F)c(NC(C)C)c4[nH]ncc34)ncc2n1] | ¹H NMR (400 MHz, DMSO-d₆) δ 13.17 (br s, 1H), 11.12 (s, 1H), 9.03 (s, 1H), 8.91 (s, 1H), 8.39 (s, 1H), 7.95 (s, 1H), 5.20 (br d, J = 9.3 Hz, 1H), 4.09 − 4.00 (m, 1H), 3.70 (q, J = 6.8 Hz, 1H), 2.10 (s, 3H), 1.40 (d, J = 7.0 Hz, 3H), 1.23 (d, J = 6.1 Hz, 6H); LCMS (electrospray) m/z 462.1 (M + H)+ | C |
| | N-(6-(5-chloro-6-fluoro-7- | | |
| | (isopropylamino)-1H-indazol-4- | | |
| | yl)imidazo[1,2-a]pyrazin-2-yl)-2- | | |
| | (methylthio)propanamide | | |
| 106 | 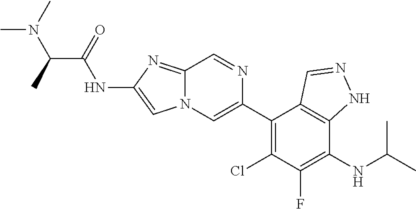 [SMILES: CC(C)Nc1c(F)c(Cl)c(-c2cn3cc(NC(=O)[C@@H](C)N(C)C)nc3cn2)c2cn[nH]c12] | ¹H NMR (400 MHz, DMSO-d₆) δ 13.32 − 13.06 (m, 1H), 10.76 − 10.58 (m, 1H), 9.02 (s, 1H), 8.90 (d, J = 1.4 Hz, 1H), 8.40 (s, 1H), 8.17 (s, 1H), 8.06 − 7.87 (m, 1H), 5.21 (br dd, J = 2.2, 9.3 Hz, 1H), 4.17 − 3.98 (m, 1H), 2.26 (s, 6H), 1.23 (d, J = 6.3 Hz, 6H), 1.19 (d, J = 6.9 Hz, 3H); LCMS (electrospray) m/z 459.1 (M + H)⁺. | C |
| | (R)-N-(6-(5-chloro-6-fluoro-7- | | |
| | (isopropylamino)-1H-indazol-4- | | |
| | yl)imidazo[1,2-a]pyrazin-2-yl)-2- | | |
| | (dimethylamino)propanamide | | |
| 107 | 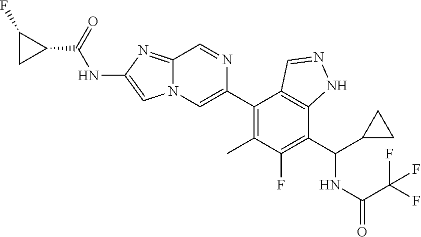 [SMILES: Cc1c(F)c(C(NC(=O)C(F)(F)F)C2CC2)c2[nH]ncc2c1-c1cn2cc(NC(=O)[C@@H]3C[C@@H]3F)nc2cn1] | ¹H NMR (400 MHz, DMSO-d₆) δ 13.19 (s, 1H), 11.37 (s, 1H), 10.14 (s, 1H), 9.05 (s, 1H), 8.87 (d, J = 1.5 Hz, 1H), 8.33 (s, 1H), 7.98 (s, 1H), 5.09 − 4.81 (m, 1H), 4.66 (s, 1H), 2.27 (d, J = 2.8 Hz, 3H), 2.19 (s, 1H), 1.71 (s, 2H), 1.24 − 1.13 (m, 1H), 0.71 (s, 1H), 0.46 (d, J = 38.8 Hz, 3H); LCMS (electrospray) m/z 534.10 (M + H)⁺. | E |
| | (1S,2S)-N-(6-(7-(cyclopropyl(2,2,2- | | |
| | trifluoroacetamido)methyl)-6-fluoro-5- | | |
| | methyl-1H-indazol-4-yl)imidazo[1,2- | | |
| | a]pyrazin-2-yl)-2-fluorocyclopropane-1- | | |
| | carboxamide | | |
| 108 | 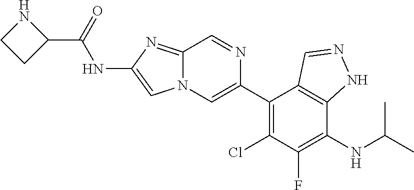 [SMILES: CC(C)Nc1c(F)c(Cl)c(-c2cn3cc(NC(=O)C4CCN4)nc3cn2)c2cn[nH]c12] | ¹H NMR (400 MHz, DMSO-d₆) δ 13.17 (s, 1H), 10.52 (s, 1H), 9.04 (s, 1H), 8.93 (d, J = 1.5 Hz, 1H), 8.43 (s, 1H), 7.96 (s, 1H), 5.20 (d, J = 9.2 Hz, 1H), 4.37 (dd, J = 9.0, 7.3 Hz, 1H), 4.07 (d, J = 11.7 Hz, 1H), 3.59 (q, J = 7.9 Hz, 1H), 2.60 − 2.53 (m, 1H), 2.34 − 2.25 (m, 1H), 1.96 (d, J = 12.2 Hz, 1H), 1.24 (d, J = 6.2 Hz, 6H); LCMS (electrospray) m/z 443.10 (M + H)⁺. | C |
| | N-(6-(5-chloro-6-fluoro-7- | | |
| | (isopropylamino)-1H-indazol-4- | | |
| | yl)imidazo[1,2-a]pyrazin-2-yl)azetidine- | | |
| | 2-carboxamide | | |
| 109 | 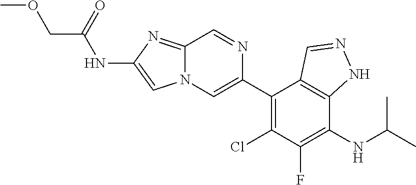 [SMILES: COCC(=O)Nc1cn2cc(-c3c(Cl)c(F)c(NC(C)C)c4[nH]ncc34)ncc2n1] | ¹H-NMR (500 MHz, DMSO-d₆) δ 13.18 (s, 1H), 10.84 (s, 1H), 9.03 (s, 1H), 8.92 (s, 1H), 8.41 (s, 1H), 7.95 (s, 1H), 5.20 (d, J = 9.2 Hz, 1H), 4.12 (s, 2H), 4.01 (m, 1H), 3.35 (s, 3H), 1.23 (d, J = 6.4 Hz, 6H); LCMS (electrospray) m/z 432.10 (M + H)⁺. | C |
| | N-(6-(5-chloro-6-fluoro-7- | | |
| | (isopropylamino)-1H-indazol-4- | | |
| | yl)imidazo[1,2-a]pyrazin-2-yl)-2- | | |
| | methoxyacetamide | | |
| 110 | 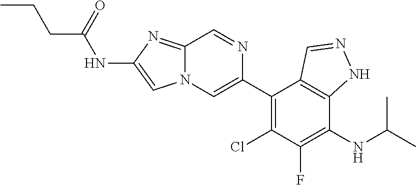 [SMILES: CCCC(=O)Nc1cn2cc(-c3c(Cl)c(F)c(NC(C)C)c4[nH]ncc34)ncc2n1] | ¹H NMR (500 MHz, DMSO-d₆) δ 13.17 (s, 1H), 10.99 (s, 1H), 9.00 (s, 1H), 8.90 (s, 1H), 8.38 (s, 1H), 7.94 (s, 1H), 5.20 (d, J = 9.5 Hz, 1H), 4.10 − 3.98 (m, 1H), 2.39 (t, J = 7.3 Hz, 2H), 1.63 (h, J = 7.4 Hz, 2H), 1.23 (d, J = 6.4 Hz, 6H), 0.92 (t, J = 7.4 Hz, 3H); LCMS (electrospray) m/z 430.10 (M + H)⁺. | C |
| | N-(6-(5-chloro-6-fluoro-7- | | |
| | (isopropylamino)-1H-indazol-4- | | |
| | yl)imidazo[1,2-a]pyrazin-2-yl)butyramide | | |
| 111 | 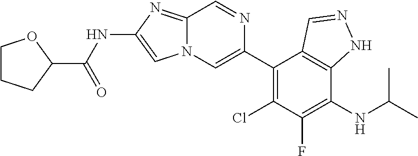 [SMILES: CC(C)Nc1c(F)c(Cl)c(-c2cn3cc(NC(=O)C4CCCO4)nc3cn2)c2cn[nH]c12] | ¹H NMR (500 MHz, DMSO-d₆) δ 13.18 (s, 1H), 10.69 (s, 1H), 9.04 (s, 1H), 8.92 (s, 1H), 8.41 (s, 1H), 7.95 (s, 1H), 5.21 (d, J = 9.4 Hz, 1H), 4.52 (dd, J = 8.2, 5.4 Hz, 1H), 4.01 (dt, J = 14.0, 8.8 Hz, 2H), 3.82 (q, J = 7.1 Hz, 1H), 2.27 − 2.17 (m, 1H), 1.92 (dtq, J = 37.7, 12.1, 6.6 Hz, 3H), 1.24 (d, J = 6.3 Hz, 6H); LCMS (electrospray) m/z 458.20 (M + H)⁺. | C |
| | N-(6-(5-chloro-6-fluoro-7- | | |
| | (isopropylamino)-1H-indazol-4- | | |
| | yl)imidazo[1,2-a]pyrazin-2- | | |
| | yl)tetrahydrofuran-2-carboxamide | | |
| 112 | 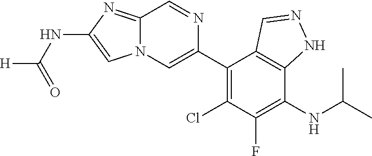 [SMILES: CC(C)Nc1c(F)c(Cl)c(-c2cn3cc(NC=O)nc3cn2)c2cn[nH]c12] | ¹H NMR (500 MHz, DMSO-d₆) δ 13.16 (s, 1H), 11.19 (s, 1H), 9.04 (s, 1H), 8.91 (s, 1H), 8.40 (s, 1H), 8.39 (s, 1H), 7.95 (s, 1H), 5.19 (dd, J = 9.7, 2.8 Hz, 1H), 4.09 − 4.03 (m, 1H), 1.24 (d, J = 6.3 Hz, 6H); LCMS (electrospray) m/z 388.10 (M + H)⁺. | C |
| | N-(6-(5-chloro-6-fluoro-7- | | |
| | (isopropylamino)-1H-indazol-4- | | |
| | yl)imidazo[1,2-a]pyrazin-2-yl)formamide | | |
| 113 | 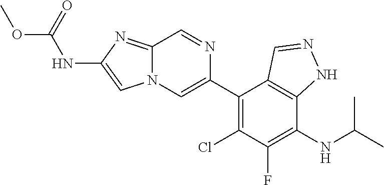 [SMILES: COC(=O)Nc1cn2cc(-c3c(Cl)c(F)c(NC(C)C)c4[nH]ncc34)ncc2n1] | ¹H NMR (400 MHz, DMSO-d₆) δ 13.54 − 12.94 (m, 1H), 10.67 (s, 1H), 8.97 (s, 1H), 8.89 (s, 1H), 8.16 (s, 1H), 7.97 (s, 1H), 5.20 (d, J = 9.2 Hz, 1H), 4.35 − 3.99 (m, 1H), 3.72 (s, 3H), 1.23 (d, J = 6.4 Hz, 9H); LCMS (electrospray) m/z 418.0 (M + H)⁺. | C |
| | methyl (6-(5-chloro-6-fluoro-7- | | |
| | (isopropylamino)-1H-indazol-4- | | |
| | yl)imidazo[1,2-a]pyrazin-2-yl)carbamate | | |
| 114 | 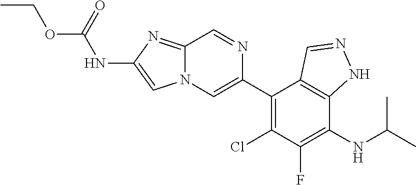 [SMILES: CCOC(=O)Nc1cn2cc(-c3c(Cl)c(F)c(NC(C)C)c4[nH]ncc34)ncc2n1] | ¹H NMR (400 MHz, DMSO-d₆) δ 13.17 (br s, 1H), 10.62 (br s, 1H), 8.97 (s, 1H), 8.89 (s, 1H), 8.15 (s, 1H), 7.94 (s, 1H), 5.20 (br d, J = 7.9 Hz, 1H), 4.18 (q, J = 7.1 Hz, 2H), 4.12 − 3.96 (m, 1H), 1.28 − 1.21 (m, 9H); LCMS (electrospray) m/z 432.0 (M + H)⁺. | C |
| | ethyl (6-(5-chloro-6-fluoro-7- | | |
| | (isopropylamino)-1H-indazol-4- | | |
| | yl)imidazo[1,2-a]pyrazin-2-yl)carbamate | | |
| 115 | 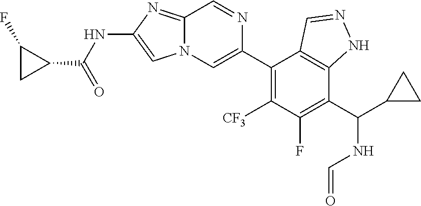 [SMILES: O=CNC(c1c(F)c(C(F)(F)F)c(-c2cn3cc(NC(=O)[C@@H]4C[C@@H]4F)nc3cn2)c2cn[nH]c12)C1CC1] | ¹H NMR (400 MHz, DMSO-d₆) δ 13.83 (br s, 1H), 11.41 (s, 1H), 9.04 − 8.98 (m, 1H), 8.92 (d, J = 1.3 Hz, 1H), 8.80 − 8.75 (m, 1H), 8.72 (s, 1H), 8.33 (s, 1H), 8.09 (s, 1H), 8.07 (s, 1H), 5.85 − 5.67 (m, 1H), 5.08 − 4.94 (m, 1H), 4.92 − 4.74 (m, 1H), 2.23 − 2.17 (m, 1H), 1.67 (brd, J = 3.8 Hz, 1H), 1.25 − 1.18 (m, 1H), 0.74 − 0.40 (m, 4H); LCMS (electrospray) m/z 520.2 (M + H)⁺. | F |
| | (1S,2S)-N-(6-(7- | | |
| | (cyclopropyl(formamido)methyl)-6- | | |
| | fluoro-5-(trifluoromethyl)-1H-indazol-4- | | |
| | yl)imidazo[1,2-a]pyrazin-2-yl)-2- | | |
| | fluorocyclopropane-1-carboxamide | | |
| 116 | 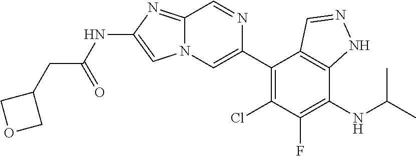 [SMILES: CC(C)Nc1c(F)c(Cl)c(-c2cn3cc(NC(=O)CC4COC4)nc3cn2)c2cn[nH]c12] | ¹H NMR (400 MHz, DMSO-d₆) δ 13.16 (br s, 1H), 11.10 (s, 1H), 9.01 (s, 1H), 8.88 (s, 1H), 8.33 (s, 1H), 7.94 (br s, 1H), 5.19 (br d, J = 8.8 Hz, 1H), 4.69 (dd, J = 6.0, 7.9 Hz, 2H), 4.36 (t, J = 6.2 Hz, 2H), 4.11 − 3.97 (m, 1H), 3.38 − 3.34 (m, 1H), 2.84 (d, J = 7.8 Hz, 2H), 1.23 (d, J = 6.3 Hz, 6H); LCMS (electrospray) m/z 458.1 (M + H)⁺. | C |
| | N-(6-(5-chloro-6-fluoro-7- | | |
| | (isopropylamino)-1H-indazol-4- | | |
| | yl)imidazo[1,2-a]pyrazin-2-yl)-2-(oxetan- | | |
| | 3-yl)acetamide | | |
| 117 | 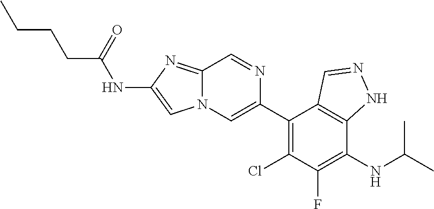 [SMILES: CCCCC(=O)Nc1cn2cc(-c3c(Cl)c(F)c(NC(C)C)c4[nH]ncc34)ncc2n1] | ¹H NMR (500 MHz, DMSO-d₆) δ 13.17 (s, 1H), 10.99 (s, 1H), 9.01 (s, 1H), 8.90 (d, J = 1.5 Hz, 1H), 8.37 (s, 1H), 7.94 (d, J = 1.5 Hz, 1H), 5.20 (dd, J = 9.6, 2.7 Hz, 1H), 4.08 − 3.99 (m, 1H), 2.41 (t, J = 7.4 Hz, 2H), 1.60 (p, J = 7.5 Hz, 2H), 1.32 (h, J = 7.4 Hz, 2H), 1.23 (d, J = 6.3 Hz, 6H), 0.90 (t, J = 7.4 Hz, 3H); LCMS (electrospray) m/z 444.10 (M + H)⁺. | C |
| | N-(6-(5-chloro-6-fluoro-7- | | |
| | (isopropylamino)-1H-indazol-4- | | |
| | yl)imidazo[1,2-a]pyrazin-2- | | |
| | yl)pentanamide | | |
| 118 | 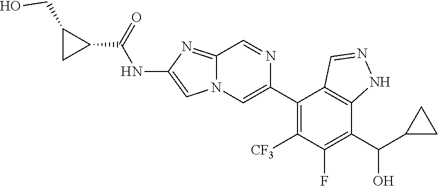 [SMILES: O=C(Nc1cn2cc(-c3c(C(F)(F)F)c(F)c(C(O)C4CC4)c4[nH]ncc34)ncc2n1)[C@@H]1C[C@@H]1CO] | ¹H NMR (400 MHz, DMSO-d₆) δ 13.50 (s, 1H), 11.27 (s, 1H), 8.99 (s, 1H), 8.88 (d, J = 1.1 Hz, 1H), 8.34 (s, 1H), 8.01 (s, 1H), 5.93 (s, 1H), 4.68 (br d, J = 7.9 Hz, 1H), 4.48 (t, J = 5.3 Hz, 1H), 3.66 (td, J = 5.4, 10.9 Hz, 1H), 3.53 − 3.46 (m, 1H), 2.11 − 2.04 (m, 1H), 1.52 − 1.46 (m, 1H), 1.28 (td, J = 7.3, 14.7 Hz, 1H), 1.04 (dt, J = 3.6, 8.0 Hz, 1H), 0.99 − 0.92 (m, 1H), 0.66 − 0.54 (m, 2H), 0.48 − 0.32 (m, 2H); LCMS (electrospray) m/z 505.2 (M + H)⁺. | F |
| | (1R,2S)-N-(6-(7- | | |
| | (cyclopropyl(hydroxy)methyl)-6-fluoro- | | |
| | 5-(trifluoromethyl)-1H-indazol-4- | | |
| | yl)imidazo[1,2-a]pyrazin-2-yl)-2- | | |
| | (hydroxymethyl)cyclopropane-1- | | |
| | carboxamide | | |
| 119 | 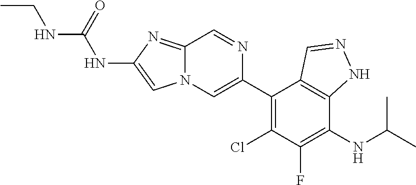 [SMILES: CCNC(=O)Nc1cn2cc(-c3c(Cl)c(F)c(NC(C)C)c4[nH]ncc34)ncc2n1] | ¹H NMR (400 MHz, DMSO-d₆) δ 13.26 − 13.08 (m, 1H), 9.25 (s, 1H), 8.91 (s, 1H), 8.85 (d, J = 1.3 Hz, 1H), 8.23 (s, 1H), 8.07 (s, 1H), 7.94 (br s, 1H), 6.57 (br t, J = 5.4 Hz, 1H), 5.18 (dd, J = 2.4, 9.4 Hz, 1H), 4.13 − 3.96 (m, 1H), 3.17 (dd, J = 5.8, 7.1 Hz, 2H), 1.23 (d, J = 6.4 Hz, 6H), 1.08 (t, J = 7.2 Hz, 3H); LCMS (electrospray) m/z 431.0 (M + H)⁺. | C |
| | 1-(6-(5-chloro-6-fluoro-7- | | |
| | (isopropylamino)-1H-indazol-4- | | |
| | yl)imidazo[1,2-a]pyrazin-2-yl)-3- | | |
| | ethylurea | | |
| 120 | 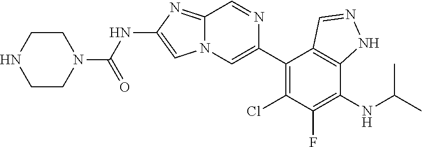 [SMILES: CC(C)Nc1c(F)c(Cl)c(-c2cn3cc(NC(=O)N4CCNCC4)nc3cn2)c2cn[nH]c12] | ¹H NMR (400 MHz, DMSO-d₆) δ 13.40 − 13.05 (m, 1H), 9.89 (s, 1H), 8.95 (s, 1H), 8.88 (d, J = 1.0 Hz, 1H), 8.20 (s, 1H), 8.15 (s, 1H), 8.02 − 7.91 (m, 1H), 5.19 (dd, J = 2.4, 9.3 Hz, 1H), 4.20 − 3.99 (m, 1H), 3.65 − 3.60 (m, 4H), 3.02 − 2.97 (m, 4H), 1.23 (d, J = 6.3 Hz, 6H); LCMS (electrospray) m/z 472.1 (M + H)⁺. | C |
| | N-(6-(5-chloro-6-fluoro-7- | | |
| | (isopropylamino)-1H-indazol-4- | | |
| | yl)imidazo[1,2-a]pyrazin-2-yl)piperazine- | | |
| | 1-carboxamide | | |
| 121 | 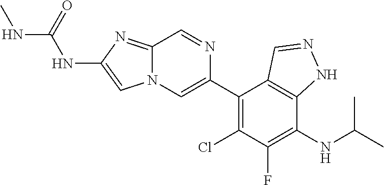 [SMILES: CNC(=O)Nc1cn2cc(-c3c(Cl)c(F)c(NC(C)C)c4[nH]ncc34)ncc2n1] | ¹H NMR (400 MHz, DMSO-d₆) δ 13.36 − 13.00 (m, 1H), 9.35 (s, 1H), 8.91 (s, 1H), 8.85 (s, 1H), 8.09 − 8.02 (m, 1H), 8.00 − 7.88 (m, 1H), 6.57 − 6.39 (m, 1H), 5.19 (br d, J = 8.9 Hz, 1H), 4.18 − 3.90 (m, 1H), 2.71 (d, J = 4.4 Hz, 3H), 1.24 −1.22 (br d, J = 6.2 Hz, 6H); LCMS (electrospray) m/z 417.0 (M + H)⁺. | C |
| | 1-(6-(5-chloro-6-fluoro-7- | | |
| | (isopropylamino)-1H-indazol-4- | | |
| | yl)imidazo[1,2-a]pyrazin-2-yl)-3- | | |
| | methylurea | | |
| 122 | 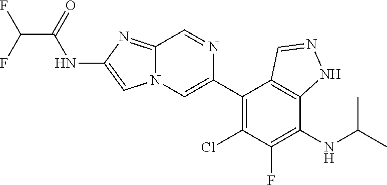 [SMILES: CC(C)Nc1c(F)c(Cl)c(-c2cn3cc(NC(=O)C(F)F)nc3cn2)c2cn[nH]c12] | ¹H-NMR (500 MHz, DMSO-d₆) δ 13.18 (s, 1H), 12.04 (s, 1H), 9.11 (s, 1H), 8.96 (s, 1H), 8.48 (s, 1H), 7.97 (s, 1H), 6.47 (t, J = 44 Hz, 1H) 5.22 (m, 1H), 4.04 (s, J = 4.0 Hz, 1H), 1.24 (d, J = 6.2 Hz, 6H); LCMS (electrospray) m/z 438.10 (M + H)⁺. | C |
| | N-(6-(5-chloro-6-fluoro-7- | | |
| | (isopropylamino)-1H-indazol-4- | | |
| | yl)imidazo[1,2-a]pyrazin-2-yl)-2,2- | | |
| | difluoroacetamide | | |
| 123 | 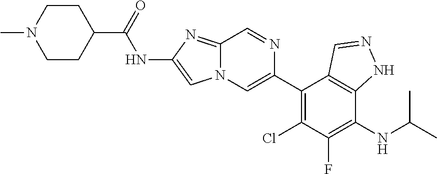 [SMILES: CC(C)Nc1c(F)c(Cl)c(-c2cn3cc(NC(=O)C4CCN(C)CC4)nc3cn2)c2cn[nH]c12] | ¹H NMR (500 MHz, DMSO-d₆) δ 13.19 (s, 1H), 10.98 (s, 1H), 9.00 (s, 1H), 8.90 (s, 1H), 8.36 (s, 1H), 7.94 (s, 1H), 5.21 (d, J = 9.5 Hz, 1H), 4.04 (s, 1H), 2.81 (d, J = 10.9 Hz, 2H), 2.16 (s, 3H), 1.95 (d, J = 18.1 Hz, 2H), 1.87 (d, J = 11.1 Hz, 1H), 1.76 (d, J = 12.7 Hz, 2H), 1.66 (q, J = 11.9 Hz, 2H), 1.23 (d, J = 6.1 Hz, 6H); LCMS (electrospray) m/z 485.20 (M + H)⁺. | C |
| | N-(6-(5-chloro-6-fluoro-7- | | |
| | (isopropylamino)-1H-indazol-4- | | |
| | yl)imidazo[1,2-a]pyrazin-2-yl)-1- | | |
| | methylpiperidine-4-carboxamide | | |
| 124 | 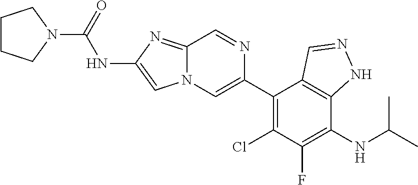 [SMILES: CC(C)Nc1c(F)c(Cl)c(-c2cn3cc(NC(=O)N4CCCC4)nc3cn2)c2cn[nH]c12] | ¹H NMR (500 MHz, DMSO-d₆) δ 13.17 (s, 1H), 9.39 (s, 1H), 8.93 (s, 1H), 8.88 (s, 1H), 8.20 (s, 1H), 7.95 (d, J = 1.5 Hz, 1H), 5.18 (dd, J = 9.6, 2.8 Hz, 1H), 4.04 (d, J = 10.7 Hz, 1H), 3.42 (d, J = 5.7 Hz, 4H), 1.86 (s, 4H), 1.24 (d, J = 6.3 Hz, 6H); LCMS (electrospray) m/z 457.15 (M + H)⁺. | C |
| | N-(6-(5-chloro-6-fluoro-7- | | |
| | (isopropylamino)-1H-indazol-4- | | |
| | yl)imidazo[1,2-a]pyrazin-2- | | |
| | yl)pyrrolidine-1-carboxamide | | |
| 125 | 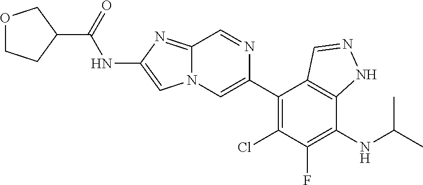 [SMILES: CC(C)Nc1c(F)c(Cl)c(-c2cn3cc(NC(=O)C4CCOC4)nc3cn2)c2cn[nH]c12] | ¹H NMR (400 MHz, DMSO-d₆) δ 13.18 (s, 1H), 11.18 (s, 1H), 9.03 (s, 1H), 8.92 (d, J = 1.4 Hz, 1H), 8.38 (s, 1H), 7.95 (d, J = 1.5 Hz, 1H), 5.20 (dd, J = 9.5, 2.7 Hz, 1H), 4.09 − 4.01 (m, 1H), 3.96 (t, J = 8.2 Hz, 1H), 3.81 (td, J = 7.9, 5.9 Hz, 1H), 3.78 − 3.69 (m, 2H), 3.31 (d, J = 7.6 Hz, 1H), 2.17 − 2.05 (m, 2H), 1.24 (d, J = 6.3 Hz, 6H); LCMS (electrospray) m/z 458.15 (M + H)⁺. | C |
| | N-(6-(5-chloro-6-fluoro-7- | | |
| | (isopropylamino)-1H-indazol-4- | | |
| | yl)imidazo[1,2-a]pyrazin-2- | | |
| | yl)tetrahydrofuran-3-carboxamide | | |
| 126 | 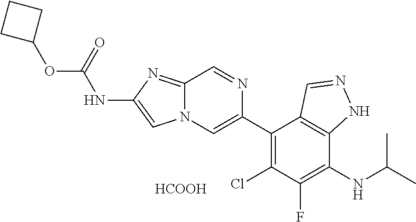 [SMILES: CC(C)Nc1c(F)c(Cl)c(-c2cn3cc(NC(=O)OC4CCC4)nc3cn2)c2cn[nH]c12 ; "HCOOH"] | ¹H NMR (400 MHz, DMSO-d₆) δ 13.25 (br s, 1H), 10.60 (br s, 1H), 8.97 (s, 1H), 8.87 (d, J = 1.1 Hz, 1H), 8.49 (s, 1H), 8.13 (s, 1H), 7.96 (br s, 1H), 5.28 − 5.20 (m, 1H), 4.98 (quin, J = 7.4 Hz, 1H), 2.14 − 2.01 (m, 3H), 1.83 − 1.69 (m, 2H), 1.68 − 1.53 (m, 2H), 1.23 (d, J = 6.3 Hz, 6H); LCMS (electrospray) m/z 458.2 (M + H)⁺. | C |
| | cyclobutyl (6-(5-chloro-6-fluoro-7- | | |
| | (isopropylamino)-1H-indazol-4- | | |
| | yl)imidazo[1,2-a]pyrazin-2-yl)carbamate | | |
| | formate | | |
| 127 | 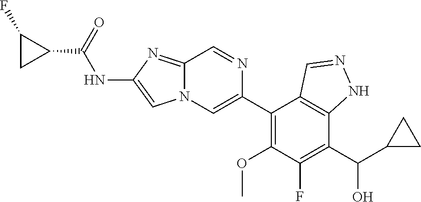 [SMILES: COc1c(F)c(C(O)C2CC2)c2[nH]ncc2c1-c1cn2cc(NC(=O)[C@@H]3C[C@@H]3F)nc2cn1] | ¹H NMR (400 MHz, DMSO-d₆) δ 12.99 (br s, 1H), 11.37 (s, 1H), 9.09 (s, 1H), 9.07 (s, 1H), 8.43 (s, 1H), 8.21 (s, 1H), 5.06 − 4.88 (m, 1H), 4.61 (brd, J = 8.1 Hz, 1H), 3.84 (br s, 1H), 3.76 (s, 3H), 2.20 (td, J = 6.9, 13.8 Hz, 1H), 1.75 − 1.64 (m, 1H), 1.46 (br dd, J = 5.3, 11.1 Hz, 1H), 1.25 − 1.18 (m, 1H), 0.63 − 0.51 (m, 2H), 0.44 − 0.30 (m, 2H); LCMS (electrospray) m/z 455.2 (M + H)⁺. | F |
| | (1S,2S)-N-(6-(7- | | |
| | (cyclopropyl(hydroxy)methyl)-6-fluoro- | | |
| | 5-methoxy-1H-indazol-4-yl)imidazo[1,2- | | |
| | a]pyrazin-2-yl)-2-fluorocyclopropane-1- | | |
| | carboxamide | | |
| 128 | 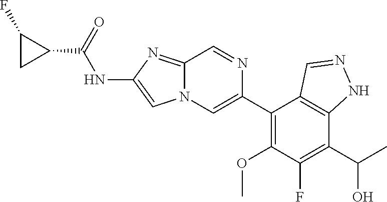 [SMILES: COc1c(F)c(C(C)O)c2[nH]ncc2c1-c1cn2cc(NC(=O)[C@@H]3C[C@@H]3F)nc2cn1] | ¹H NMR (400 MHz, DMSO-d₆) δ 12.94 (br s, 1H), 11.36 (s, 1H), 9.08 (s, 1H), 9.06 (s, 1H), 8.43 (s, 1H), 8.22 (s, 1H), 5.74 (br d, J = 0.9 Hz, 1H), 5.48 − 5.35 (m, 1H), 5.10 − 4.85 (m, 1H), 3.76 (s, 3H), 2.25 − 2.14 (m, 1H), 1.77 − 1.65 (m, 1H), 1.55 (d, J = 6.5 Hz, 3H), 1.21 (tdd, J = 6.1, 8.9, 12.3 Hz, 1H); LCMS (electrospray) m/z 429 (M + H)⁺. | F |
| | (1S,2S)-2-fluoro-N-(6-(6-fluoro-7-(1- | | |
| | hydroxyethyl)-5-methoxy-1H-indazol-4- | | |
| | yl)imidazo[1,2-a]pyrazin-2- | | |
| | yl)cyclopropane-1-carboxamide | | |
| 129 | 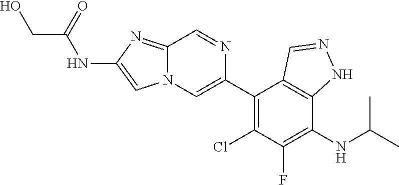 [SMILES: CC(C)Nc1c(F)c(Cl)c(-c2cn3cc(NC(=O)CO)nc3cn2)c2cn[nH]c12] | ¹H NMR (500 MHz, DMSO-d₆) δ 13.17 (s, 1H), 10.49 (s, 1H), 9.04 (s, 1H), 8.93 (s, 1H), 8.42 (s, 1H), 7.95 (s, 1H), 5.56 (t, J = 6.2 Hz, 1H), 5.24 − 5.15 (m, 1H), 4.11 (d, J = 6.4 Hz, 2H), 4.06 − 4.00 (m, 1H), 1.24 (d, J = 6.2 Hz, 7H); LCMS (electrospray) m/z 418.10 (M + H)⁺. | C |
| | N-(6-(5-chloro-6-fluoro-7- | | |
| | (isopropylamino)-1H-indazol-4- | | |
| | yl)imidazo[1,2-a]pyrazin-2-yl)-2- | | |
| | hydroxyacetamide | | |
| 130 | 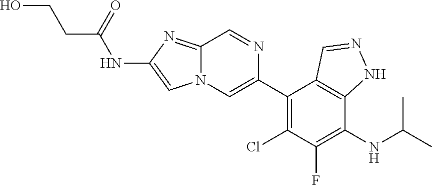 [SMILES: CC(C)Nc1c(F)c(Cl)c(-c2cn3cc(NC(=O)CCO)nc3cn2)c2cn[nH]c12] | ¹H NMR (500 MHz, DMSO-d₆) δ 13.17 (s, 1H), 10.98 (s, 1H), 9.01 (s, 1H), 8.92 (s, 1H), 8.38 (s, 1H), 7.95 (s, 1H), 5.20 (d, J = 8.9 Hz, 1H), 4.71 (t, J = 5.1 Hz, 1H), 4.09 − 3.99 (m, 1H), 3.73 (q, J = 6.1 Hz, 2H), 2.57 (t, J = 6.4 Hz, 2H), 1.23 (d, J = 6.2 Hz, 7H); LCMS (electrospray) m/z 432.10 (M + H)⁺. | C |
| | N-(6-(5-chloro-6-fluoro-7- | | |
| | (isopropylamino)-1H-indazol-4- | | |
| | yl)imidazo[1,2-a]pyrazin-2-yl)-3- | | |
| | hydroxypropanamide | | |
| 131 | 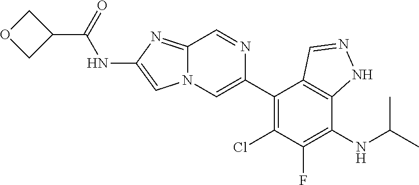 [SMILES: CC(C)Nc1c(F)c(Cl)c(-c2cn3cc(NC(=O)C4COC4)nc3cn2)c2cn[nH]c12] | ¹H NMR (500 MHz, DMSO-d₆) δ 13.18 (s, 1H), 11.22 (s, 1H), 9.04 (s, 1H), 8.93 (s, 1H), 8.41 (s, 1H), 7.96 (s, 1H), 5.21 (d, J = 9.6 Hz, 1H), 5.04 (t, J = 5.3 Hz, 1H), 4.05 (s, 1H), 3.87 − 3.80 (m, 2H), 3.71 (dt, J = 11.3, 5.9 Hz, 1H), 3.61 (dt, J = 11.0, 5.9 Hz, 1H), 3.16 (p, J = 6.5 Hz, 1H), 1.24 (d, J = 6.3 Hz, 6H).; LCMS (electrospray) m/z 444.10 (M + H)⁺. | C |
| | N-(6-(5-chloro-6-fluoro-7- | | |
| | (isopropylamino)-1H-indazol-4- | | |
| | yl)imidazo[1,2-a]pyrazin-2-yl)oxetane-3- | | |
| | carboxamide | | |
| 132 | 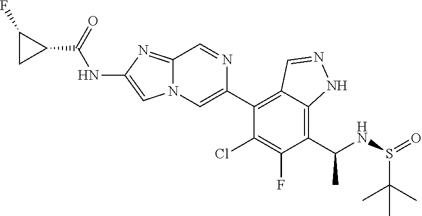 [SMILES: C[C@H](N[S@@](=O)C(C)(C)C)c1c(F)c(Cl)c(-c2cn3cc(NC(=O)[C@@H]4C[C@@H]4F)nc3cn2)c2cn[nH]c12] | ¹H NMR (400 MHz, DMSO-d₆) δ 13.40 (br s, 1H), 11.47 − 11.35 (m, 1H), 9.07 (br s, 1H), 9.02 (s, 1H), 8.44 − 8.36 (m, 1H), 8.12 − 8.02 (m, 1H), 5.62 − 5.47 (m, 1H), 5.24 − 5.12 (m, 1H), 5.10 − 4.85 (m, 1H), 2.21 (br d, J = 4.3 Hz, 1H), 1.78 − 1.74 (m, 1H), 1.71 (br d, J = 6.7 Hz, 3H), 1.24 − 1.20 (m, 1H), 1.06 (br s, 9H); LCMS (electrospray) m/z 536.1 (M + H)⁺. | G |
| | (1S,2S)-N-(6-(7-((S)-1-(((S)-tert- | | |
| | butylsulfinyl)amino)ethyl)-5-chloro-6- | | |
| | fluoro-1H-indazol-4-yl)imidazo[1,2- | | |
| | a]pyrazin-2-yl)-2-fluorocyclopropane-1- | | |
| | carboxamide | | |
| 133 | 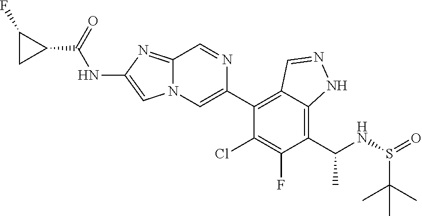 [SMILES: C[C@@H](N[S@](=O)C(C)(C)C)c1c(F)c(Cl)c(-c2cn3cc(NC(=O)[C@@H]4C[C@@H]4F)nc3cn2)c2cn[nH]c12] | ¹H NMR (400 MHz, DMSO-d₆) δ 13.51 − 13.33 (m, 1H), 11.41 (s, 1H), 9.08 (s, 1H), 9.03 (s, 1H), 8.39 (s, 1H), 8.06 (s, 1H), 5.50 (br d, J = 3.5 Hz, 1H), 5.16 (br dd, J = 3.7, 8.1 Hz, 1H), 5.07 − 4.86 (m, 1H), 2.23 − 2.19 (m, 1H), 1.71 (brd, J = 6.8 Hz, 3H), 1.24 − 1.18 (m, 1H), 1.07 (s, 9H); LCMS (electrospray) m/z 536.1 (M + H)⁺. | G |
| | (1S,2S)-N-(6-(7-((R)-1-(((R)-tert- | | |
| | butylsulfinyl)amino)ethyl)-5-chloro-6- | | |
| | fluoro-1H-indazol-4-yl)imidazo[1,2- | | |
| | a]pyrazin-2-yl)-2-fluorocyclopropane-1- | | |
| | carboxamide | | |
| 134 | 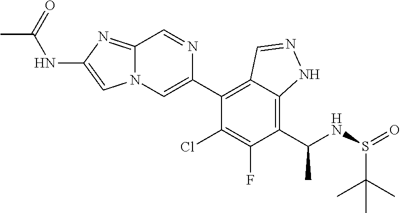 [SMILES: CC(=O)Nc1cn2cc(-c3c(Cl)c(F)c([C@H](C)N[S@@](=O)C(C)(C)C)c4[nH]ncc34)ncc2n1] | ¹H NMR (400 MHz, DMSO-d₆) δ 13.39 (br s, 1H), 11.07 (s, 1H), 9.06 (s, 1H), 9.02 (d, J = 1.2 Hz, 1H), 8.40 (s, 1H), 8.05 (s, 1H), 5.51 (br d, J = 3.1 Hz, 1H), 5.16 (br dd, J = 3.7, 6.7 Hz, 1H), 2.14 (s, 3H), 1.71 (d, J = 6.8 Hz, 3H), 1.07 (s, 9H); LCMS (electrospray) m/z 492.1 (M + H)⁺. | G |
| | N-(6-(7-((S)-1-(((S)-tert- | | |
| | butylsulfinyl)amino)ethyl)-5-chloro-6- | | |
| | fluoro-1H-indazol-4-yl)imidazo[1,2- | | |
| | a]pyrazin-2-yl)acetamide | | |
| 135 | 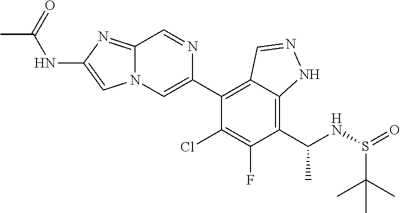 [SMILES: CC(=O)Nc1cn2cc(-c3c(Cl)c(F)c([C@@H](C)N[S@](=O)C(C)(C)C)c4[nH]ncc34)ncc2n1] | ¹H NMR (400 MHz, DMSO-d₆) δ 13.40 (br s, 1H), 11.07 (s, 1H), 9.06 (s, 1H), 9.02 (d, J = 1.2 Hz, 1H), 8.40 (s, 1H), 8.06 (s, 1H), 5.50 (d, J = 3.4 Hz, 1H), 5.17 (br dd, J = 4.0, 6.6 Hz, 1H), 2.14 (s, 3H), 1.71 (brd, J = 6.7 Hz, 3H), 1.07 (s, 9H); LCMS (electrospray) m/z 492.2 (M + H)⁺. | G |
| | N-(6-(7-((R)-1-(((R)-tert- | | |
| | butylsulfinyl)amino)ethyl)-5-chloro-6- | | |
| | fluoro-1H-indazol-4-yl)imidazo[1,2- | | |
| | a]pyrazin-2-yl)acetamide | | |
| 136 | 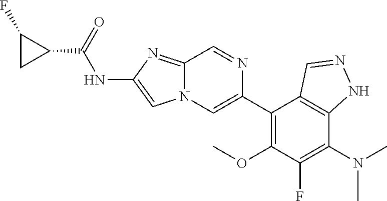 [SMILES: COc1c(F)c(N(C)C)c2[nH]ncc2c1-c1cn2cc(NC(=O)[C@@H]3C[C@@H]3F)nc2cn1] | ¹H NMR (400 MHz, DMSO-d₆) δ 13.18 (br s, 1H), 11.34 (s, 1H), 9.06 (s, 1H), 9.02 (d, J = 1.3 Hz, 1H), 8.41 (s, 1H), 8.23 (s, 1H), 5.08 − 4.85 (m, 1H), 3.76 (s, 3H), 3.00 (br s, 6H), 2.24 − 2.15 (m, 1H), 1.77 − 1.64 (m, 1H), 1.27 − 1.18 (m, 1H); LCMS (electrospray) m/z 428.2 (M + H)⁺. | F |
| | (1S,2S)-N-(6-(7-(dimethylamino)-6- | | |
| | fluoro-5-methoxy-1H-indazol-4- | | |
| | yl)imidazo[1,2-a]pyrazin-2-yl)-2- | | |
| | fluorocyclopropane-1-carboxamide | | |
| 137 | 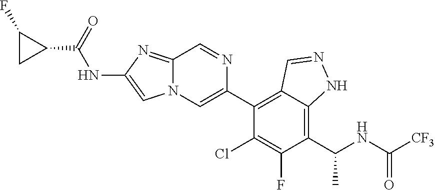 [SMILES: C[C@@H](NC(=O)C(F)(F)F)c1c(F)c(Cl)c(-c2cn3cc(NC(=O)[C@@H]4C[C@@H]4F)nc3cn2)c2cn[nH]c12] | ¹H NMR (400 MHz, DMSO-d₆) δ 13.53 (br s, 1H), 11.41 (s, 1H), 9.99 (br d, J = 5.7 Hz, 1H), 9.08 (s, 1H), 9.02 (d, J = 1.3 Hz, 1H), 8.38 (s, 1H), 8.09 (s, 1H), 5.56 (br d, J = 5.6 Hz, 1H), 5.10 − 4.80 (m, 1H), 2.25 − 2.15 (m, 1H), 1.70 (brd, J = 7.3 Hz, 3H), 1.68 − 1.60 (m, 1H), 1.32 − 1.14 (m, 1H); LCMS (electrospray) m/z 528.1 (M + H)+ | G |
| | (1S,2S)-N-(6-(5-chloro-6-fluoro-7-((R)- | | |
| | 1-(2,2,2-trifluoroacetamido)ethyl)-1H- | | |
| | indazol-4-yl)imidazo[1,2-a]pyrazin-2-yl)- | | |
| | 2-fluorocyclopropane-1-carboxamide | | |
| 138 | 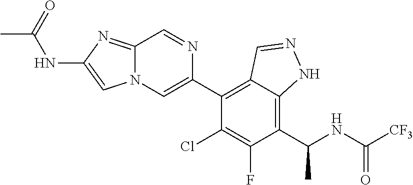 [SMILES: CC(=O)Nc1cn2cc(-c3c(Cl)c(F)c([C@H](C)NC(=O)C(F)(F)F)c4[nH]ncc34)ncc2n1] | ¹H NMR (400 MHz, DMSO-d₆) δ 14.15 − 13.15 (m, 1H), 11.07 (s, 1H), 9.06 (s, 1H), 9.01 (s, 1H), 8.40 (s, 1H), 8.10 (s, 1H), 5.62 − 5.51 (m, 1H), 2.14 (s, 3H), 1.70 (br d, J = 7.1 Hz, 3H); LCMS (electrospray) m/z 483.9 (M + H)⁺. | G |
| | (S)-N-(1-(4-(2-acetamidoimidazo[1,2- | | |
| | a]pyrazin-6-yl)-5-chloro-6-fluoro-1H- | | |
| | indazol-7-yl)ethyl)-2,2,2- | | |
| | trifluoroacetamide | | |
| 139 | 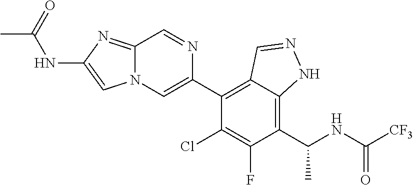 [SMILES: CC(=O)Nc1cn2cc(-c3c(Cl)c(F)c([C@@H](C)NC(=O)C(F)(F)F)c4[nH]ncc34)ncc2n1] | ¹H NMR (400 MHz, DMSO-d₆) δ 13.53 (br s, 1H), 11.07 (s, 1H), 9.99 (br d, J = 5.4 Hz, 1H), 9.06 (s, 1H), 9.01 (s, 1H), 8.40 (s, 1H), 8.09 (br s, 1H), 5.56 (br d, J = 6.2 Hz, 1H), 2.14 (s, 3H), 1.70 (br d, J = 7.1 Hz, 3H); LCMS (electrospray) m/z 483.9 (M + H)+ | G |
| | (R)-N-(1-(4-(2-acetamidoimidazo[1,2- | | |
| | a]pyrazin-6-yl)-5-chloro-6-fluoro-1H- | | |
| | indazol-7-yl)ethyl)-2,2,2- | | |
| | trifluoroacetamide | | |
| 140 | 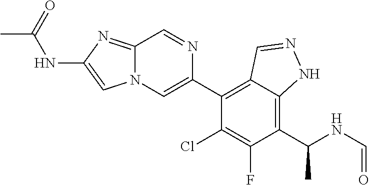 [SMILES: CC(=O)Nc1cn2cc(-c3c(Cl)c(F)c([C@H](C)NC=O)c4[nH]ncc34)ncc2n1] | ¹H NMR (400 MHz, DMSO-d₆) δ 13.50 (br s, 1H), 11.07 (s, 1H), 9.06 (s, 1H), 8.99 (d, J = 1.3 Hz, 1H), 8.67 (br d, J = 7.0 Hz, 1H), 8.40 (s, 1H), 8.07 (s, 2H), 5.65 − 5.50 (m, 1H), 2.14 (s, 3H), 1.59 (d, J = 7.2 Hz, 3H); LCMS (electrospray) m/z 416.1 (M + H)⁺. | G |
| | (S)-N-(6-(5-chloro-6-fluoro-7-(1- | | |
| | formamidoethyl)-1H-indazol-4- | | |
| | yl)imidazo[1,2-a]pyrazin-2-yl)acetamide | | |
| 141 | 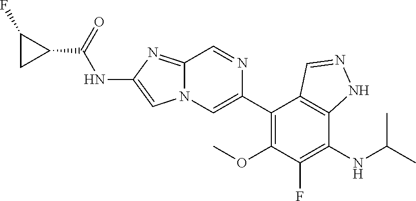 [SMILES: COc1c(F)c(NC(C)C)c2[nH]ncc2c1-c1cn2cc(NC(=O)[C@@H]3C[C@@H]3F)nc2cn1] | ¹H NMR (400 MHz, DMSO-d₆) δ 12.94 (br s, 1H), 11.31 (s, 1H), 9.03 (s, 1H), 8.99 (d, J = 1.2 Hz, 1H), 8.38 (s, 1H), 8.26 (br s, 1H), 5.11 − 4.82 (m, 2H), 4.13 − 3.95 (m, 1H), 3.76 (s, 3H), 2.24 − 2.13 (m, 1H), 1.75 − 1.64 (m, 1H), 1.23 (d, J = 6.2 Hz, 6H), 1.21 − 1.15 (m, 1H); LCMS (electrospray) m/z 442.3 (M + H)⁺. | F |
| | (1S,2S)-2-fluoro-N-(6-(6-fluoro-7- | | |
| | (isopropylamino)-5-methoxy-1H-indazol- | | |
| | 4-yl)imidazo[1,2-a]pyrazin-2- | | |
| | yl)cyclopropane-1-carboxamide | | |
| 142 | 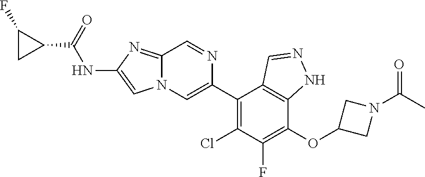 [SMILES: CC(=O)N1CC(Oc2c(F)c(Cl)c(-c3cn4cc(NC(=O)[C@@H]5C[C@@H]5F)nc4cn3)c3cn[nH]c23)C1] | ¹H NMR (400 MHz, DMSO-d₆) δ 14.29 − 13.66 (m, 1H), 11.40 (s, 1H), 9.06 (s, 1H), 8.97 (s, 1H), 8.39 (s, 1H), 8.11 (br s, 1H), 5.35 (br s, 1H), 5.13 − 4.81 (m, 1H), 4.59 − 4.42 (m, 1H), 4.39 − 4.19 (m, 2H), 4.12 − 3.99 (m, 1H), 2.27 − 2.19 (m, 1H), 1.83 (s, 3H), 1.73 − 1.63 (m, 1H), 1.21 (br dd, J = 9.0, 11.9 Hz, 1H); LCMS (electrospray) m/z 502.1 (M + H)⁺. | E |
| | (1S,2S)-N-(6-(7-((1-acetylazetidin-3- | | |
| | yl)oxy)-5-chloro-6-fluoro-1H-indazol-4- | | |
| | yl)imidazo[1,2-a]pyrazin-2-yl)-2- | | |
| | fluorocyclopropane-1-carboxamide | | |
| 143 | 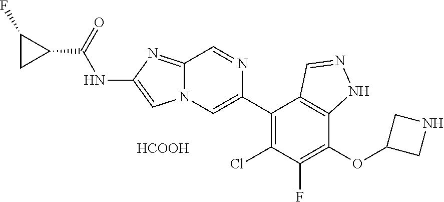 [SMILES: O=C(Nc1cn2cc(-c3c(Cl)c(F)c(OC4CNC4)c4[nH]ncc34)ncc2n1)[C@@H]1C[C@@H]1F ; "HCOOH"] | ¹H NMR (400 MHz, DMSO-d₆) δ 11.05 (s, 1H), 9.04 (s, 1H), 8.95 (s, 1H), 8.40 (s, 1H), 8.23 (s, 1H), 8.12 (s, 1H), 5.35 (br d, J = 4.6 Hz, 1H), 4.32 − 4.22 (m, 2H), 4.17 − 4.12 (m, 2H), 2.13 (s, 3H); LCMS (electrospray) m/z 416.0 (M + H)⁺. | E |
| | (1S,2S)-N-(6-(7-(azetidin-3-yloxy)-5- | | |
| | chloro-6-fluoro-1H-indazol-4- | | |
| | yl)imidazo[1,2-a]pyrazin-2-yl)-2- | | |
| | fluorocyclopropane-1-carboxamide | | |
| | formate | | |
| 144 | 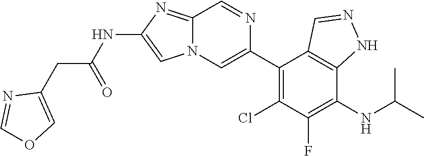 [SMILES: CC(C)Nc1c(F)c(Cl)c(-c2cn3cc(NC(=O)Cc4cocn4)nc3cn2)c2cn[nH]c12] | ¹H NMR (400 MHz, DMSO-d₆) δ 11.25 (s, 1H), 9.04 (s, 1H), 8.92 (s, 1H), 8.37 (s, 1H), 8.33 (s, 1H), 8.00 (br d, J = 5.7 Hz, 2H), 4.21 − 4.08 (m, 1H), 3.75 (s, 2H), 1.24 (d, J = 6.2 Hz, 6H); LCMS (electrospray) m/z 469.2 (M + H)⁺. | C |
| | N-(6-(5-chloro-6-fluoro-7- | | |
| | (isopropylamino)-1H-indazol-4- | | |
| | yl)imidazo[1,2-a]pyrazin-2-yl)-2-(oxazol- | | |
| | 4-yl)acetamide | | |
| 145 | 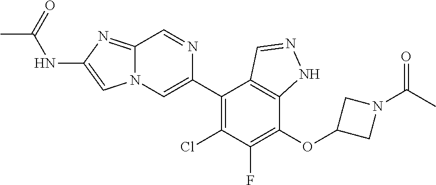 [SMILES: CC(=O)Nc1cn2cc(-c3c(Cl)c(F)c(OC4CN(C(C)=O)C4)c4[nH]ncc34)ncc2n1] | ¹H NMR (400 MHz, DMSO-d₆) δ 14.04 − 13.73 (m, 1H), 11.12 − 11.01 (m, 1H), 9.11 − 9.02 (m, 1H), 8.96 (d, J = 1.3 Hz, 1H), 8.40 (s, 1H), 8.09 (br d, J = 10.0 Hz, 1H), 5.43 − 5.25 (m, 1H), 4.52 (dd, J = 7.0, 9.6 Hz, 1H), 4.37 − 4.19 (m, 2H), 4.03 (br d, J = 9.2 Hz, 1H), 2.13 (s, 3H), 1.85 − 1.80 (m, 3H); LCMS (electrospray) m/z 458.1 (M + H)⁺. | E |
| | N-(6-(7-((1-acetylazetidin-3-yl)oxy)-5- | | |
| | chloro-6-fluoro-1H-indazol-4- | | |
| | yl)imidazo[1,2-a]pyrazin-2-yl)acetamide | | |
| 146 | 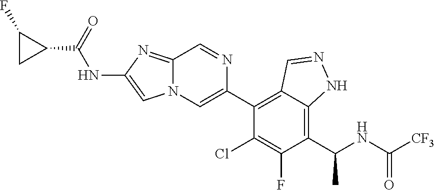 [SMILES: C[C@H](NC(=O)C(F)(F)F)c1c(F)c(Cl)c(-c2cn3cc(NC(=O)[C@@H]4C[C@@H]4F)nc3cn2)c2cn[nH]c12] | ¹H NMR (400 MHz, DMSO-d₆) δ 13.52 (s, 1H), 11.41 (s, 1H), 9.98 (br d, J = 6.2 Hz, 1H), 9.08 (d, J = 0.6 Hz, 1H), 9.02 (d, J = 1.3 Hz, 1H), 8.39 (s, 1H), 8.10 (s, 1H), 5.64 − 5.52 (m, 1H), 5.09 − 4.86 (m, 1H), 2.25 − 2.15 (m, 1H), 1.70 (d, J = 7.2 Hz, 3H), 1.69 − 1.64 (m, 1H), 1.26 − 1.16 (m, 1H); LCMS (electrospray) m/z 528.2 (M + H)⁺. | G |
| | (1S,2S)-N-(6-(5-chloro-6-fluoro-7-((S)-1- | | |
| | (2,2,2-trifluoroacetamido)ethyl)-1H- | | |
| | indazol-4-yl)imidazo[1,2-a]pyrazin-2-yl)- | | |
| | 2-fluorocyclopropane-1-carboxamide | | |
| 147 | 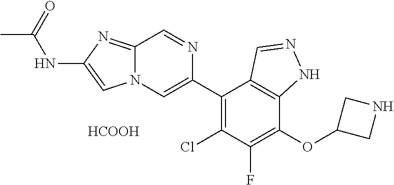 [SMILES: CC(=O)Nc1cn2cc(-c3c(Cl)c(F)c(OC4CNC4)c4[nH]ncc34)ncc2n1 ; "HCOOH"] | ¹H NMR (400 MHz, DMSO-d₆) δ 11.05 (s, 1H), 9.04 (s, 1H), 8.95 (s, 1H), 8.40 (s, 1H), 8.23 (s, 1H), 8.12 (s, 1H), 5.35 (br d, J = 4.6 Hz, 1H), 4.32 − 4.22 (m, 2H), 4.17 − 4.12 (m, 2H), 2.13 (s, 3H); LCMS (electrospray) m/z 416.0 (M + H)⁺. | E |
| | N-(6-(7-(azetidin-3-yloxy)-5-chloro-6- | | |
| | fluoro-1H-indazol-4-yl)imidazo[1,2- | | |
| | a]pyrazin-2-yl)acetamide formate | | |
| 148 | 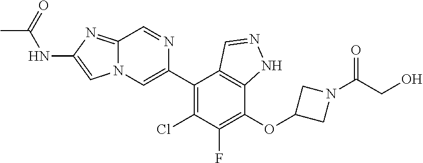 [SMILES: CC(=O)Nc1cn2cc(-c3c(Cl)c(F)c(OC4CN(C(=O)CO)C4)c4[nH]ncc34)ncc2n1] | ¹H NMR (400 MHz, DMSO-d₆) δ 13.86 (s, 1H), 11.05 (s, 1H), 9.04 (s, 1H), 8.96 (s, 1H), 8.40 (s, 1H), 8.07 (s, 1H), 5.33 (s, 1H), 5.06-5.03 (brt, J = 5.7 Hz, 1H), 4.64 − 4.55 (m, 1H), 4.41-4.38 (m, 1H), 4.36- 4.29 (br dd, J = 6.8, 10.4 Hz, 1H), 4.28- 4.11 (br d, J = 10.9 Hz, 1H), 3.98-3.96 (br d, J = 5.6 Hz, 2H), 2.13 (s, 3H); LCMS (electrospray) m/z 474.1 (M + H)⁺. | E |
| | N-(6-(5-chloro-6-fluoro-7-((1-(2- | | |
| | hydroxyacetyl)azetidin-3-yl)oxy)-1H- | | |
| | indazol-4-yl)imidazo[1,2-a]pyrazin-2- | | |
| | yl)acetamide | | |
| 149 | 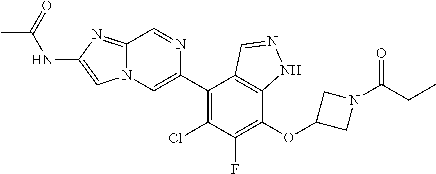 [SMILES: CCC(=O)N1CC(Oc2c(F)c(Cl)c(-c3cn4cc(NC(C)=O)nc4cn3)c3cn[nH]c23)C1] | ¹H NMR (400 MHz, DMSO-d₆) δ 13.86 (s, 1H), 11.05 (s, 1H), 9.04 (s, 1H), 8.96 (d, J = 1.1 Hz, 1H), 8.40 (s, 1H), 8.13 (s, 1H), 8.06 (s, 1H), 5.31 (br dd, J = 3.8, 5.5 Hz, 1H), 4.56 − 4.48 (m, 1H), 4.32 − 4.21 (m, 2H), 4.09 − 4.00 (m, 1H), 2.15 − 2.11 (m, 5H), 0.99 (t, J = 7.5 Hz, 3H); LCMS (electrospray) m/z 472.2 (M + H)⁺. | E |
| | N-(6-(5-chloro-6-fluoro-7-((1- | | |
| | propionylazetidin-3-yl)oxy)-1H-indazol- | | |
| | 4-yl)imidazo[1,2-a]pyrazin-2- | | |
| | yl)acetamide | | |
| 150 | 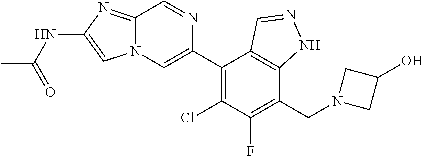 [SMILES: CC(=O)Nc1cn2cc(-c3c(Cl)c(F)c(CN4CC(O)C4)c4[nH]ncc34)ncc2n1] | ¹H NMR (400 MHz, DMSO-d₆) δ 13.46 (br dd, J = 4.8, 8.1 Hz, 1H), 11.06 (s, 1H), 9.05 (s, 1H), 9.00 (s, 1H), 8.40 (s, 1H), 8.14 (s, 1H), 8.02 (s, 1H), 5.33 (br d, J = 6.2 Hz, 1H), 4.25 − 4.12 (m, 1H), 3.98 (s, 2H), 3.54 − 3.50 (m, 2H), 2.96 − 2.88 (m, 2H), 2.13 (s, 3H); LCMS (electrospray) m/z 430.2 (M + H)⁺. | E |
| | N-(6-(5-chloro-6-fluoro-7-((3- | | |
| | hydroxyazetidin-1-yl)methyl)-1H- | | |
| | indazol-4-yl)imidazo[1,2-a]pyrazin-2- | | |
| | yl)acetamide | | |
| 151 | 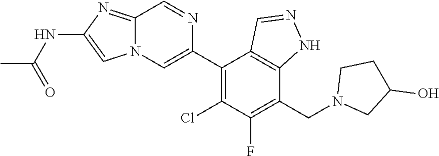 [SMILES: CC(=O)Nc1cn2cc(-c3c(Cl)c(F)c(CN4CCC(O)C4)c4[nH]ncc34)ncc2n1] | ¹H NMR (400 MHz, DMSO-d₆) δ 13.70 − 12.98 (m, 1H), 11.07 (s, 1H), 9.06 (s, 1H), 9.01 (s, 1H), 8.41 (s, 1H), 8.04 (s, 1H), 4.77 (br d, J = 4.2 Hz, 1H), 4.24 − 4.16 (m, 1H), 4.02 (br s, 2H), 2.78 − 2.70 (m, 2H), 2.68 (br s, 1H), 2.14 (s, 3H), 2.06 − 1.96 (m, 2H), 1.61 − 1.52 (m, 1H); LCMS (electrospray) m/z 444.2 (M + H)⁺. | E |
| | N-(6-(5-chloro-6-fluoro-7-((3- | | |
| | hydroxypyrrolidin-1-yl)methyl)-1H- | | |
| | indazol-4-yl)imidazo[1,2-a]pyrazin-2- | | |
| | yl)acetamide | | |
| 152 | 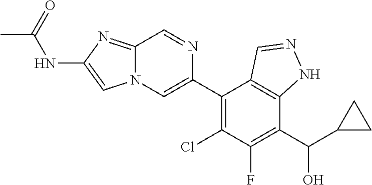 [SMILES: CC(=O)Nc1cn2cc(-c3c(Cl)c(F)c(C(O)C4CC4)c4[nH]ncc34)ncc2n1] | ¹H NMR (500 MHz, DMSO-d₆) δ 13.27 (s, 1H), 11.08 (s, 1H), 9.07 (s, 1H), 9.00 (d, J = 1.5 Hz, 1H), 8.40 (s, 1H), 8.01 (d, J = 1.6 Hz, 1H), 5.89 (d, J = 3.8 Hz, 1H), 4.62 (dd, J = 8.0, 3.8 Hz, 1H), 2.14 (s, 3H), 1.50 − 1.41 (m, 1H), 0.60 (dq, J = 13.5, 6.5 Hz, 2H), 0.38 (ddd, J = 39.0, 9.4, 4.5 Hz, 2H); LCMS (electrospray) m/z 415.10 (M + H)⁺. | E |
| | N-(6-(5-chloro-7- | | |
| | (cyclopropyl(hydroxy)methyl)-6-fluoro- | | |
| | 1H-indazol-4-yl)imidazo[1,2-a]pyrazin-2- | | |
| | yl)acetamide | | |
| 153 | 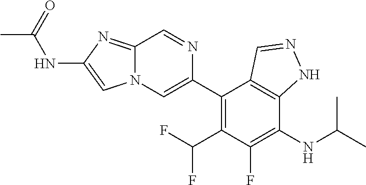 [SMILES: CC(=O)Nc1cn2cc(-c3c(C(F)F)c(F)c(NC(C)C)c4[nH]ncc34)ncc2n1] | ¹H NMR (500 MHz, DMSO-d₆) δ 13.29 (s, 1H), 11.04 (s, 1H), 8.98 (s, 1H), 8.85 (s, 1H), 8.36 (s, 1H), 8.09 (s, 1H), 7.07 (t, J = 52.7 Hz, 2H), 5.06 (dd, J = 9.6, 2.7 Hz, 1H), 4.03 (q, J = 6.3 Hz, 1H), 2.13 (s, 3H), 1.22 (d, J = 6.2 Hz, 7H); LCMS (electrospray) m/z 418.10 (M + H)⁺. | C |
| | N-(6-(5-(difluoromethyl)-6-fluoro-7- | | |
| | (isopropylamino)-1H-indazol-4- | | |
| | yl)imidazo[1,2-a]pyrazin-2-yl)acetamide | | |
| 154 | 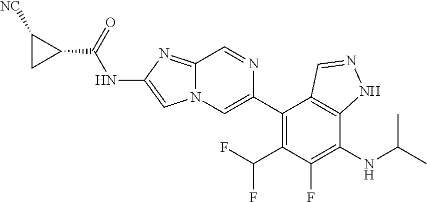 [SMILES: CC(C)Nc1c(F)c(C(F)F)c(-c2cn3cc(NC(=O)[C@@H]4C[C@@H]4C#N)nc3cn2)c2cn[nH]c12] | ¹H NMR (500 MHz, DMSO-d₆) δ 13.30 (s, 1H), 11.65 (s, 1H), 9.02 (s, 1H), 8.87 (s, 1H), 8.41 (s, 1H), 8.10 (s, 1H), 7.07 (t, J = 52.7 Hz, 2H), 5.07 (d, J = 9.7 Hz, 1H), 4.07 − 3.99 (m, 1H), 2.33 − 2.26 (m, 1H), 1.50 (ddq, J = 21.0, 8.4, 4.4 Hz, 2H), 1.22 (d, J = 6.3 Hz, 6H), 1.19 − 1.16 (m, 1H); LCMS (electrospray) m/z 469.10 (M + H)⁺. | C |
| | (1R,2S)-2-cyano-N-(6-(5- | | |
| | (difluoromethyl)-6-fluoro-7- | | |
| | (isopropylamino)-1H-indazol-4- | | |
| | yl)imidazo[1,2-a]pyrazin-2- | | |
| | yl)cyclopropane-1-carboxamide | | |
| 155 | 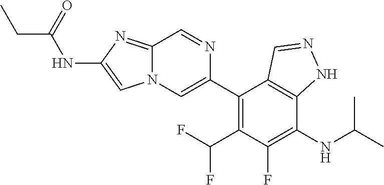 [SMILES: CCC(=O)Nc1cn2cc(-c3c(C(F)F)c(F)c(NC(C)C)c4[nH]ncc34)ncc2n1] | ¹H NMR (500 MHz, DMSO-d₆) δ 13.28 (s, 1H), 10.98 (s, 1H), 8.98 (s, 1H), 8.87 (s, 1H), 8.38 (s, 1H), 8.09 (s, 1H), 7.08 (t, J = 52.7 Hz, 1H), 5.07 (d, J = 9.5 Hz, 1H), 4.04 (s, 1H), 2.43 (q, J = 7.5 Hz, 2H), 1.23 (d, J = 6.3 Hz, 7H), 1.11 (t, J = 7.5 Hz, 3H); LCMS (electrospray) m/z 432.10 (M + H)⁺. | C |
| | N-(6-(5-(difluoromethyl)-6-fluoro-7- | | |
| | (isopropylamino)-1H-indazol-4- | | |
| | yl)imidazo[1,2-a]pyrazin-2- | | |
| | yl)propionamide | | |
| 156 | 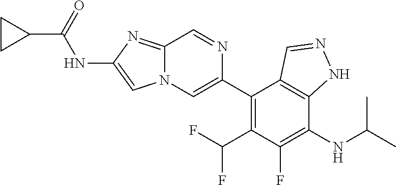 [SMILES: CC(C)Nc1c(F)c(C(F)F)c(-c2cn3cc(NC(=O)C4CC4)nc3cn2)c2cn[nH]c12] | ¹H NMR (500 MHz, DMSO-d₆) δ 13.28 (s, 1H), 11.32 (s, 1H), 8.99 (s, 1H), 8.86 (d, J = 1.5 Hz, 1H), 8.33 (s, 1H), 8.09 (d, J = 1.6 Hz, 1H), 7.08 (t, J = 52.7 Hz, 1H), 5.07 (dd, J = 9.9, 2.8 Hz, 1H), 4.03 (d, J = 4.0 Hz, 1H), 1.98 (p, J = 6.8 Hz, 1H), 1.23 (d, J = 6.3 Hz, 6H), 0.88 − 0.83 (m, 5H); LCMS (electrospray) m/z 444.20 (M + H)⁺. | C |
| | N-(6-(5-(difluoromethyl)-6-fluoro-7- | | |
| | (isopropylamino)-1H-indazol-4- | | |
| | yl)imidazo[1,2-a]pyrazin-2- | | |
| | yl)cyclopropanecarboxamide | | |
| 157 | 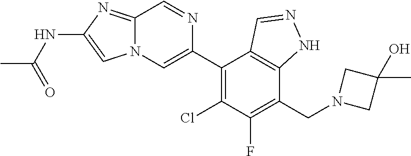 [SMILES: CC(=O)Nc1cn2cc(-c3c(Cl)c(F)c(CN4CC(C)(O)C4)c4[nH]ncc34)ncc2n1] | ¹H NMR (400 MHz, DMSO-d₆) δ 13.43 (br s, 1H), 11.07 (s, 1H), 9.06 (s, 1H), 9.01 (s, 1H), 8.40 (s, 1H), 8.03 (s, 1H), 5.23 (s, 1H), 4.01 (s, 2H), 3.20 (br d, J = 7.5 Hz, 2H), 3.05 (br d, J = 6.4 Hz, 2H), 2.14 (s, 3H), 1.34 (s, 3H); LCMS (electrospray) m/z 444.2 (M + H)⁺. | E |
| | N-(6-(5-chloro-6-fluoro-7-((3-hydroxy-3- | | |
| | methylazetidin-1-yl)methyl)-1H-indazol- | | |
| | 4-yl)imidazo[1,2-a]pyrazin-2- | | |
| | yl)acetamide | | |
| 158 | 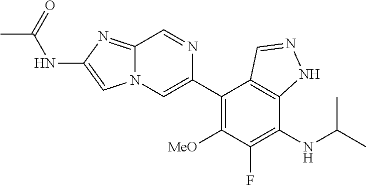 [SMILES: COc1c(F)c(NC(C)C)c2[nH]ncc2c1-c1cn2cc(NC(C)=O)nc2cn1] | ¹H NMR (400 MHz, DMSO-d₆) δ 12.94 (br s, 1H), 11.05 − 10.93 (m, 1H), 9.05 − 9.01 (m, 1H), 8.99 (s, 1H), 8.40 (s, 1H), 8.23 (s, 1H), 5.07 − 4.99 (m, 1H), 4.06 − 4.00 (m, 1H), 3.76 (s, 3H), 2.13 (s, 3H), 1.23 (d, J = 6.2 Hz, 6H); LCMS (electrospray) m/z 398.2 (M + H)⁺. | F |
| | N-(6-(6-fluoro-7-(isopropylamino)-5- | | |
| | methoxy-1H-indazol-4-yl)imidazo[1,2- | | |
| | a]pyrazin-2-yl)acetamide | | |
| 159 | 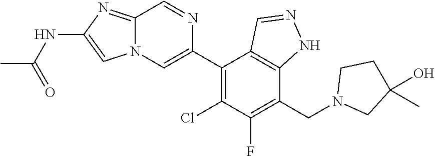 [SMILES: CC(=O)Nc1cn2cc(-c3c(Cl)c(F)c(CN4CCC(C)(O)C4)c4[nH]ncc34)ncc2n1] | ¹H NMR (400 MHz, DMSO-d₆) δ 13.35 (br s, 1H), 11.07 (s, 1H), 9.06 (s, 1H), 9.01 (s, 1H), 8.40 (s, 1H), 8.04 (s, 1H), 4.79 − 4.62 (m, 1H), 4.02 (s, 2H), 2.78 (q, J = 7.7 Hz, 1H), 2.62 − 2.54 (m, 2H), 2.46 (br d, J = 9.2 Hz, 1H), 2.14 (s, 3H), 1.76 (brt, J = 6.6 Hz, 2H), 1.23 (s, 3H); LCMS (electrospray) m/z 458.2 (M + H)⁺. | E |
| | N-(6-(5-chloro-6-fluoro-7-((3-hydroxy-3- | | |
| | methylpyrrolidin-1-yl)methyl)-1H- | | |
| | indazol-4-yl)imidazo[1,2-a]pyrazin-2- | | |
| | yl)acetamide | | |
| 160 | 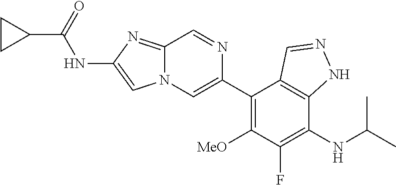 [SMILES: COc1c(F)c(NC(C)C)c2[nH]ncc2c1-c1cn2cc(NC(=O)C3CC3)nc2cn1] | ¹H NMR (400 MHz, DMSO-d₆) δ 12.94 (br s, 1H), 11.26 (s, 1H), 9.02 (s, 1H), 8.98 (d, J = 1.4 Hz, 1H), 8.35 (s, 1H), 8.24 (br s, 1H), 5.02 (dd, J = 2.0, 9.5 Hz, 1H), 4.19 − 3.92 (m, 1H), 3.76 (s, 3H), 2.04 − 1.93 (m, 1H), 1.23 (d, J = 6.3 Hz, 6H), 0.89 − 0.82 (m, 4H); LCMS (electrospray) m/z 424.3 (M + H)⁺. | F |
| | N-(6-(6-fluoro-7-(isopropylamino)-5- | | |
| | methoxy-1H-indazol-4-yl)imidazo[1,2- | | |
| | a]pyrazin-2-yl)cyclopropanecarboxamide | | |
| 161 | 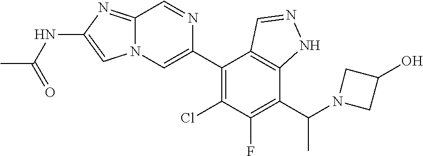 [SMILES: CC(=O)Nc1cn2cc(-c3c(Cl)c(F)c(C(C)N4CC(O)C4)c4[nH]ncc34)ncc2n1] | ¹H NMR (400 MHz, DMSO-d₆) δ 11.07 (s, 1H), 9.04 (s, 1H), 9.01 − 8.96 (m, 1H), 8.42 − 8.37 (m, 1H), 8.13 (s, 1H), 8.10 − 7.98 (m, 1H), 7.96 − 7.88 (m, 1H), 5.60 − 5.38 (m, 1H), 4.36 − 4.15 (m, 2H), 4.08 − 3.92 (m, 1H), 3.71 (br s, 2H), 2.14 − 2.14 (m, 1H), 2.13 (s, 2H), 1.70 − 1.52 (m, 1H), 1.43 − 1.25 (m, 3H); LCMS (electrospray) m/z 444.3 (M + H)⁺. | E |
| | N-(6-(5-chloro-6-fluoro-7-(1-(3- | | |
| | hydroxyazetidin-1-yl)ethyl)-1H-indazol- | | |
| | 4-yl)imidazo[1,2-a]pyrazin-2- | | |
| | yl)acetamide | | |
| 162 | 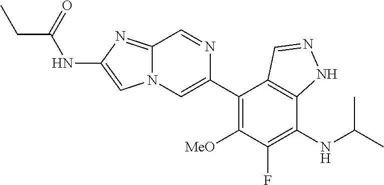 [SMILES: CCC(=O)Nc1cn2cc(-c3c(OC)c(F)c(NC(C)C)c4[nH]ncc34)ncc2n1] | ¹H NMR (400 MHz, DMSO-d₆) δ 12.94 (br s, 1H), 10.93 (s, 1H), 9.01 (s, 1H), 8.99 (s, 1H), 8.40 (s, 1H), 8.24 (br s, 1H), 5.02 (br d, J = 8.0 Hz, 1H), 4.03 (br dd, J = 2.8, 9.2 Hz, 1H), 3.76 (s, 3H), 2.43 (q, J = 7.5 Hz, 2H), 1.24 (d, J = 6.3 Hz, 6H), 1.11 (t, J = 7.6 Hz, 3H); LCMS (electrospray) m/z 412.3 (M + H)⁺. | F |
| | N-(6-(6-fluoro-7-(isopropylamino)-5- | | |
| | methoxy-1H-indazol-4-yl)imidazo[1,2- | | |
| | a]pyrazin-2-yl)propionamide | | |
| 163 | 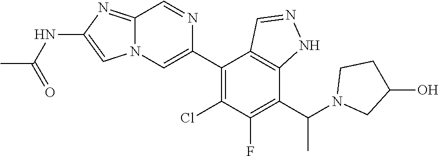 [SMILES: CC(=O)Nc1cn2cc(-c3c(Cl)c(F)c(C(C)N4CCC(O)C4)c4[nH]ncc34)ncc2n1] | ¹H NMR (400 MHz, DMSO-d₆) δ 13.13 (br s, 1H), 11.07 (s, 1H), 9.06 (s, 1H), 9.00 (s, 1H), 8.40 (s, 1H), 8.03 (br d, J = 6.2 Hz, 1H), 4.85 (m, 1H), 4.20 (m, 1H), 4.00 (m, 1H), 2.72 (br d, J = 9.0 Hz, 2H), 2.40 (m, 1H), 2.27 (m, 1H), 2.14 (s, 3H), 2.05 (m, 1H), 1.60 (m, 1H), 1.48 (br d, J = 4.6 Hz, 3H); LCMS (electrospray) m/z 458.2 (M + H)⁺. | E |
| | N-(6-(5-chloro-6-fluoro-7-(1-(3- | | |
| | hydroxypyrrolidin-1-yl)ethyl)-1H- | | |
| | indazol-4-yl)imidazo[1,2-a]pyrazin-2- | | |
| | yl)acetamide | | |
| 164 | 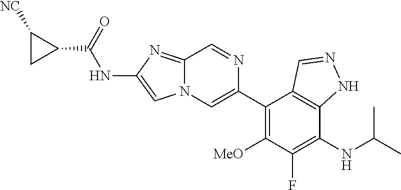 [SMILES: COc1c(F)c(NC(C)C)c2[nH]ncc2c1-c1cn2cc(NC(=O)[C@@H]3C[C@@H]3C#N)nc2cn1] | ¹H NMR (400 MHz, DMSO-d₆) δ 12.93 (br s, 1H), 11.59 (s, 1H), 9.06 (s, 1H), 9.01 (s, 1H), 8.45 (s, 1H), 8.25 (br s, 1H), 5.14 − 4.92 (m, 1H), 4.12 − 3.97 (m, 1H), 3.77 (s, 3H), 2.33 − 2.24 (m, 1H), 1.54 − 1.46 (m, 2H), 1.24 (d, J = 6.2 Hz, 6H), 1.16 (t, J = 7.3 Hz, 1H); LCMS (electrospray) m/z 449.1 (M + H)⁺. | C |
| | (1R,2S)-2-cyano-N-(6-(6-fluoro-7- | | |
| | (isopropylamino)-5-methoxy-1H-indazol- | | |
| | 4-yl)imidazo[1,2-a]pyrazin-2- | | |
| | yl)cyclopropane-1-carboxamide | | |
| 165 | 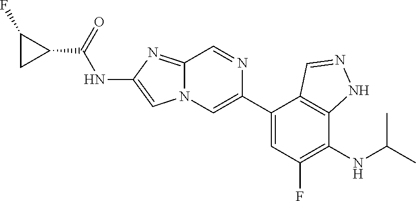 [SMILES: CC(C)Nc1c(F)cc(-c2cn3cc(NC(=O)[C@@H]4C[C@@H]4F)nc3cn2)c2cn[nH]c12] | ¹H NMR (500 MHz, DMSO-d₆) δ 13.07 (s, 1H), 11.33 (s, 1H), 9.17 (s, 1H), 9.01 (s, 1H), 8.61 (s, 1H), 8.35 (s, 1H), 7.55 (d, J = 14.2 Hz, 2H), 5.11 − 4.76 (m, 2H), 4.00 (d, J = 11.3 Hz, 1H), 2.19 (t, J = 7.2 Hz, 1H), 1.70 (dd, J = 23.2, 3.8 Hz, 1H), 1.22 (d, J = 6.3 Hz, 7H); LCMS (electrospray) m/z 412.15 (M + H)⁺. | J |
| | (1S,2S)-2-fluoro-N-(6-(6-fluoro-7- | | |
| | (isopropylamino)-1H-indazol-4- | | |
| | yl)imidazo[1,2-a]pyrazin-2- | | |
| | yl)cyclopropane-1-carboxamide | | |

### Evaluation of Compounds

### HPK1 Kinase Assay

HPK1 kinase activity was measured by Promega's ADP-Glo™ kinase assay. In this assay, 5 ng of recombinant human HPK1 (signalchem) is incubated with 5 μL of compounds (0.5% DMSO), 5 μL of MBP (0.5 μg/μl) and 5 μL of ATP (25 μM) in buffer (40 mM Tris, 7.5; 20 mM MgCl₂; 0.1 mg/ml BSA; 50 μM DTT.). The assay was started by incubating the reaction mixture in a 96-well plate at 30° C. for 40 minutes. After the incubation, 25 μL ADP-Glo reagent was added and the reaction was incubated at room temperature for 40-min to stop the reaction and degrade residual ATP. The ADP product was then converted to ATP by adding 50 μL per well of detection reagent. Luminescence was detected after 30-min room temperature incubation with the Molecular device I3X plate reader. The IC₅₀ values were calculated from a series of percent inhibition values determined at a range of inhibitor concentration using software routines as implemented in the GraphPad Prism 7 software and SigmaPlot13.0.

Table 2 shows IC₅₀ values of the invented compounds which represent + for >1000 nM, ++ for 501-1000 nM, +++ for 101-500 nM, ++++ for <100 nM.

TABLE 2
In vitro activity against HPK1 data

| | HPK1 IC₅₀ |
|---|---|
| Example | (nM) |
| 1 | ++++ |
| 2 | ++++ |
| 3 | +++ |
| 4 | ++++ |
| 5 | ++++ |
| 6 | ++++ |
| 7 | +++ |
| 8 | ++++ |
| 9 | ++++ |
| 10 | +++ |
| 11 | ++++ |
| 12 | ++ |
| 13 | +++ |
| 14 | ++++ |
| 15 | ++++ |
| 16 | ++++ |
| 17 | ++++ |
| 18 | +++ |
| 19 | ++++ |
| 20 | ++++ |
| 21 | ++++ |
| 22 | ++++ |
| 23 | ++++ |
| 24 | ++++ |
| 25 | +++ |
| 26 | ++++ |
| 27 | ++++ |
| 28 | ++++ |
| 29 | ++++ |
| 30 | ++++ |
| 31 | ++++ |
| 32 | ++++ |
| 33 | ++++ |
| 34 | ++++ |
| 35 | ++++ |
| 36 | ++++ |
| 37 | ++++ |
| 38 | ++++ |
| 39 | ++++ |
| 40 | +++ |
| 41 | ++++ |
| 42 | ++++ |
| 43 | ++++ |
| 44 | ++++ |
| 45 | ++ |
| 46 | ++++ |
| 47 | ++ |
| 48 | ++++ |
| 49 | ++++ |
| 50 | ++++ |
| 51 | ++++ |
| 52 | ++++ |
| 53 | ++++ |
| 54 | ++++ |
| 55 | +++ |
| 56 | ++++ |
| 57 | ++++ |
| 58 | +++ |
| 59 | ++++ |
| 60 | ++++ |
| 61 | ++++ |
| 66 | ++++ |
| 67 | ++++ |
| 68 | +++ |
| 69 | ++++ |
| 70 | ++++ |
| 71 | ++++ |
| 72 | ++ |
| 73 | ++ |
| 74 | ++++ |
| 75 | ++++ |
| 76 | ++++ |
| 77 | ++++ |
| 78 | ++++ |
| 79 | ++++ |
| 80 | ++++ |
| 81 | ++++ |
| 82 | +++ |
| 83 | ++++ |
| 84 | ++++ |
| 85 | ++++ |
| 86 | ++++ |
| 87 | ++++ |
| 88 | ++++ |
| 89 | ++++ |
| 90 | ++++ |
| 91 | ++ |
| 92 | ++++ |
| 93 | ++++ |
| 94 | ++++ |
| 95 | ++++ |
| 96 | ++++ |
| 97 | ++++ |
| 98 | ++++ |
| 99 | ++++ |
| 100 | ++++ |
| 101 | +++ |
| 102 | ++ |
| 103 | ++++ |
| 104 | ++++ |
| 105 | +++ |
| 106 | ++ |
| 107 | ++++ |
| 108 | +++ |
| 109 | ++++ |
| 110 | ++++ |
| 111 | +++ |
| 112 | ++++ |
| 113 | ++++ |
| 114 | ++++ |
| 115 | ++++ |
| 116 | ++++ |
| 117 | ++++ |
| 118 | ++++ |
| 119 | ++++ |
| 120 | ++++ |
| 121 | ++ |
| 122 | ++++ |
| 123 | ++++ |
| 124 | ++++ |
| 125 | ++++ |
| 126 | ++++ |
| 127 | ++++ |
| 128 | ++++ |
| 129 | ++++ |
| 130 | ++++ |
| 131 | ++++ |
| 132 | ++++ |
| 133 | ++++ |
| 134 | +++ |
| 135 | ++++ |
| 136 | ++++ |
| 137 | ++++ |
| 138 | ++++ |
| 139 | ++++ |
| 140 | ++++ |
| 141 | ++++ |
| 142 | +++ |
| 143 | ++ |
| 144 | ++++ |
| 145 | ++ |
| 146 | ++++ |
| 147 | ++ |
| 148 | ++ |
| 149 | ++ |
| 150 | ++ |
| 151 | ++ |
| 152 | ++++ |
| 153 | ++++ |
| 154 | ++++ |
| 155 | ++++ |
| 156 | ++++ |
| 157 | ++ |
| 158 | ++++ |
| 159 | ++ |
| 160 | ++++ |
| 161 | ++ |
| 162 | ++++ |
| 163 | ++ |
| 164 | ++++ |
| 165 | |

1. A compound of Formula (I): 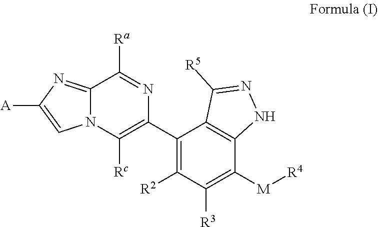
or a pharmaceutically acceptable salt, hydrate, or solvate thereof, wherein:
A is —NH—C(O)-L-R¹, —NH—R¹, or —R¹;
L is a bond, —NH—, —O—, or —CH₂—;
R¹ is —H, -D, —CD₃, alkyl, alkenyl, alkynyl, cycloalkyl, aryl, heteroaryl, heterocyclyl, halo, hydroxyl, —CD₂OH, —CN, —NO₂, haloalkyl, —C(O)R⁹, —C(O)OR⁹, —C(O)NR¹⁰R¹¹, —OR⁹, —OC(O)R⁹, —OC(O)NR¹⁰R¹¹, —SR⁹, —S(O)R⁹, —S(O)₂R⁹, —S(O)(═NH)R¹⁰, —S(O)₂NR¹⁰R¹¹, —NR¹⁰R¹¹, —N(R⁶) NR¹⁰R¹¹, —N(R⁶) OR⁹, —N(R⁶)C(O)R⁹, —N(R⁶)C(O)R⁹, —N(R⁶)C(O)OR⁹, —N(R⁶)C(O)NR¹⁰R¹¹, —N(R⁶)S(O)₂R⁹, —N(R⁶)S(O)₂NR¹⁰R¹¹, or —P(O)R¹²R¹³, wherein the alkyl, alkenyl, alkynyl, cycloalkyl, aryl, heteroaryl, or heterocyclyl is optionally substituted with one or more substituents independently selected from R⁷;
R² is —H, -D, —CD₃, alkyl, alkenyl, alkynyl, cycloalkyl, aryl, heteroaryl, heterocyclyl, halo, hydroxyl, —CD₂OH, —CN, —NO₂, haloalkyl, —C(O)R⁹, —C(O)OR⁹, —C(O)NR¹⁰R¹¹, —OR⁹, —OC(O)R⁹, —OC(O)NR¹⁰R¹¹, —SR⁹, —S(O)R⁹, —S(O)₂R⁹, —S(O)(═NH)R¹⁰, —S(O)₂NR¹⁰R¹¹, —NR¹⁰R¹¹, —N(R⁶) NR¹⁰R¹¹, —N(R⁶) OR⁹, —N(R⁶)C(O)R⁹, —N(R⁶)C(O)R⁹, —N(R⁶)C(O)OR⁹, —N(R⁶)C(O)NR¹⁰R¹¹, —N(R⁶)S(O)₂R⁹, —N(R⁶)S(O)₂NR¹⁰R¹¹, or —P(O)R¹²R¹³, wherein the alkyl, alkenyl, alkynyl, cycloalkyl, aryl, heteroaryl, or heterocyclyl is optionally substituted with one or more groups selected from the group consisting of halo, hydroxyl, alkyl, alkenyl, alkynyl, haloalkyl, hydroxyalkyl, —CN, —NO₂, —NR¹⁰R¹¹, —NR¹⁰C(═O)R⁹, —NR¹⁰C(═O)NR¹⁰R¹¹, —NR¹⁰C(═O)OR⁹, —OR⁹, —C(═O)R⁹, —C(═O)OR⁹, —C(═O)NR¹⁰R¹¹, —OC(═O)R⁹, —OC(═O)OR⁹, and —OC(═O)NR¹⁰R¹¹;
R³ is —H, -D, —CD₃, alkyl, alkenyl, alkynyl, cycloalkyl, aryl, heteroaryl, heterocyclyl, halo, cyano, hydroxy, —CH₂OH, —CD₂OH, —OH, —CN, —NO₂, haloalkyl, —C(O)R⁹, —C(O)OR⁹, —C(O)NR¹⁰R¹¹, —OR⁹, —OC(O)R⁹, —OC(O)NR¹⁰R¹¹, —SR⁹, —S(O)R⁹, —S(O)₂R⁹, —S(O)(═NH)R¹⁰, —S(O)₂NR¹⁰R¹¹, —NR¹⁰R¹¹, —N(R⁶) NR¹⁰R¹¹, —N(R⁶) OR⁹, —N(R⁶)C(O)R⁹, —N(R⁶)C(O)OR⁹, —N(R⁶)C(O)NR¹⁰R¹¹, —N(R⁶)S(O)₂R⁹, —N(R⁶)S(O)₂NR¹⁰R¹¹, or —P(O)R¹²R¹³;
M is a bond, —O—, —S—, —CHR⁶—, or —NR⁶—;
R⁴ is —H, -D, alkyl, alkenyl, alkynyl, cycloalkyl, aryl, heteroaryl, heterocyclyl, halo, cyano, hydroxy, —C(O)R⁹, —C(O)OR⁹, —C(O)NR¹⁰R¹¹, —S(O)₂R⁹, —S(O)(═NH)R¹⁰, —S(O)₂NR¹⁰R¹¹, or —P(O)R¹²R¹³, wherein the alkyl, alkenyl, alkynyl, cycloalkyl, aryl, heteroaryl, or heterocyclyl is optionally substituted with one or more groups selected from the group consisting of halo, hydroxyl, alkyl, alkenyl, alkynyl, haloalkyl, hydroxyalkyl, —CN, —CD₃, —NO₂, —NR¹⁰R¹¹, —NR¹⁰C(═O)R⁹, —NR¹⁰C(═O)NR¹⁰R¹¹, —NR¹⁰C(═O)OR⁹, —NR¹⁰S(O)₂R⁹, —OR⁹, —C(═O)R⁹, —C(═O)OR⁹, —C(═O)NR¹⁰R¹¹, —OC(═O)R⁹, —OC(═O)OR⁹, and —OC(═O)NR¹⁰R¹¹;
R⁵ is —H, -D, —CD₃, alkyl, alkenyl, alkynyl, cycloalkyl, halo, hydroxyl, —CH₂OH, —CD₂OH, —CN, or haloalkyl;
R⁶ is —H, C₁₋₆ alkyl, or cycloalkyl;
R⁷ is alkyl, alkenyl, alkynyl, cycloalkyl, aryl, heteroaryl, heterocyclyl, halo, oxo, cyano, hydroxy, —C(O)R⁹, —C(O)OR⁹, —C(O)NR¹⁰R¹¹, —OR⁹, —OC(O)R⁹, —OC(O)NR¹⁰R¹¹, —SR⁹, —S(O)R⁹, —S(O)₂R⁹, —S(O)(═NH)R¹⁰, —S(O)₂NR¹⁰R¹¹, —NR¹⁰R¹¹, —N(R⁶) NR¹⁰R¹¹, —N(R⁶) OR⁹, —N(R⁶)C(O)R⁹, —N(R⁶)C(O)OR⁹, —N(R⁶)C(O)NR¹⁰R¹¹, —N(R⁶)S(O)₂R⁹, —N(R⁶)S(O)₂NR¹⁰R¹¹, or —P(O)R¹²R¹³;
R⁹ is —H, alkyl, haloalkyl, alkenyl, alkynyl, cycloalkyl, aryl, heteroaryl, or heterocyclyl;
Each R¹⁰ and R¹¹ is independently —H, alkyl, alkenyl, alkynyl, cycloalkyl, aryl, heteroaryl, or heterocyclyl, or R¹⁰ and R¹¹ are taken together with the nitrogen atom to which they are attached to form a 4- to 12-membered heterocyclyl optionally substituted with one or more groups selected from the group consisting of halo, hydroxyl, alkyl, alkenyl, alkynyl, haloalkyl, hydroxyalkyl, —CN, —NO₂, —NR¹⁰R¹¹, —NR¹⁰C(═O)R⁹, —NR¹⁰C(═O)NR¹⁰R¹¹, —NR¹⁰C(═O)OR⁹, —OR⁹, —C(═O)R⁹, —C(═O)OR⁹, —C(═O)NR¹⁰R¹¹, —OC(═O)R⁹, —OC(═O)OR⁹, and —OC(═O)NR¹⁰R¹¹;
Each R¹² and R₁₃ is independently alkyl, alkoxyl, cycloalkyl, aryl, heteroaryl, or heterocyclyl, or R¹² and R₁₃ are taken together with the phosphorus atom to which they are attached to form a 4- to 8-membered heterocyclyl optionally substituted with one or more groups selected from the group consisting of halo, hydroxyl, alkyl, alkenyl, alkynyl, haloalkyl, hydroxyalkyl, —CN, —NO₂, —NR¹⁰R¹¹, —NR¹⁰C(═O)R⁹, —NR¹⁰C(═O)NR¹⁰R¹¹, —NR¹⁰C(═O)OR⁹, —OR⁹, —C(═O)R⁹, —C(═O)OR⁹, —C(═O)NR¹⁰R¹¹, —OC(═O)R⁹, —OC(═O)OR⁹, and —OC(═O)NR¹⁰R¹¹; and
Each of R^{a} and R^{c} is independently —H, -D, halo, haloalkyl, —CN, —OR⁹ or —NR¹⁰R¹¹.

2. The compound of claim 1 or a pharmaceutically acceptable salt thereof, wherein
each of R² and R³ is independently —H, -D, halo, alkoxy, alkylthio, haloalkyl, alkyl, hydroxyalkyl, —COOH, —C(O) OCH₃, or —CN;
R⁵ is —H, halo, or alkyl;
each of R^{a} and R^{c} is independently —H, halo, or alkyl;
M is a bond, —O—, —CHR⁶—, or —NR⁶—; and
R⁴ is —H, -D, C₁₋₆ alkyl, C₂₋₆ alkenyl, C₂₋₆ alkynyl, cycloalkyl, aryl, heteroaryl, heterocyclyl, halo, cyano, hydroxy, —C(O)R⁹, —NHC(O)R⁹, —C(O)NR¹⁰R¹¹, —S(O)₂R⁹, —S(O)(═NH)R¹⁰, or —S(O)₂NR¹⁰R¹¹, wherein the C₁₋₆ alkyl, C₂₋₆ alkenyl, C₂₋₆ alkynyl, cycloalkyl, aryl, heteroaryl, or heterocyclyl is optionally substituted with one or more groups selected from the group consisting of halo, hydroxy, alkyl, alkenyl, alkynyl, haloalkyl, hydroxyalkyl, —C(O)CH₃, —CN, —CD₃, —NR¹⁰R¹¹, —NR¹⁰S(O)₂R⁹, and —NR¹⁰C(═O)R⁹.

3. The compound of claim 1 or a pharmaceutically acceptable salt thereof, wherein R² is alkyl, haloalkyl, hydroxyalkyl, alkoxy, halo, —COOH, —C(O) OCH₃, or —CN; R³ is halo or alkyl; and M-R⁴ is selected from the group consisting of:

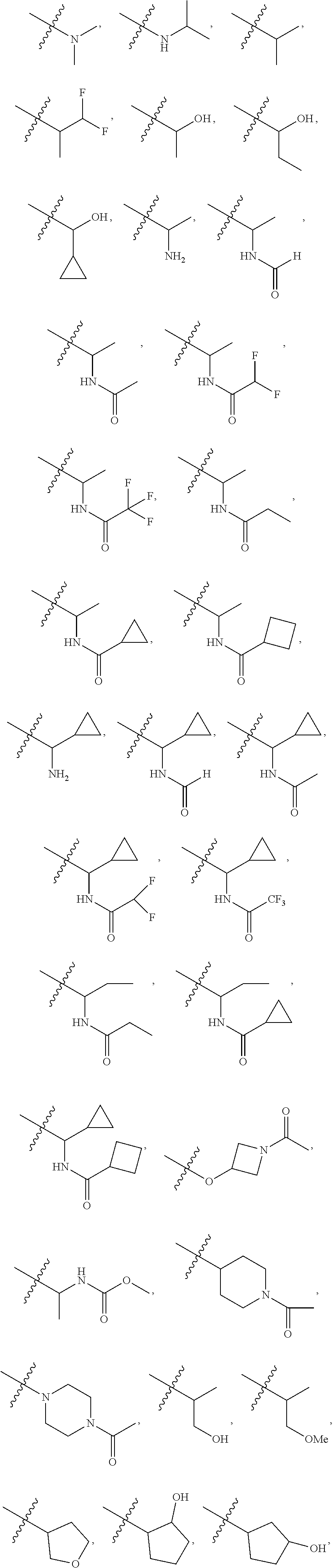

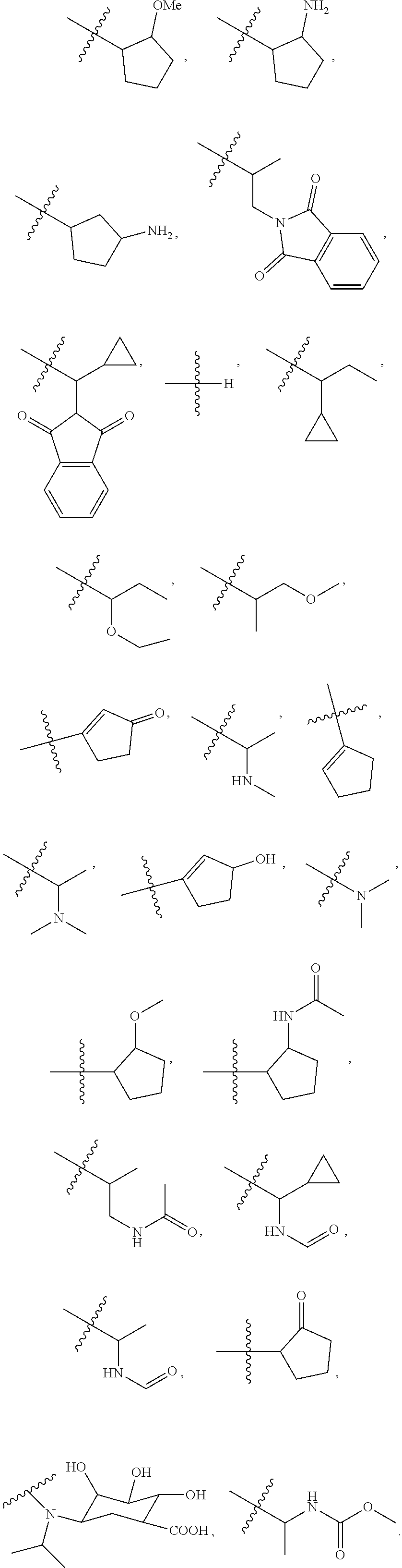

4. The compound of claim 1 or a pharmaceutically acceptable salt thereof, which is a compound of Formula (II): 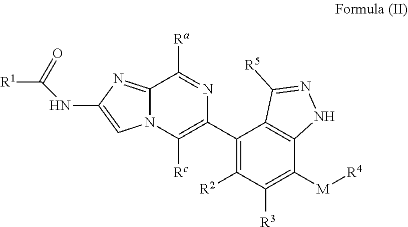
wherein R¹ is cyclopropyl which is optionally substituted with one or two R⁷.

5. The compound of claim 4 or a pharmaceutically acceptable salt thereof, wherein R¹ is F

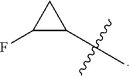 [SMILES: *C1CC1F]

6. The compound of claim 5 or a pharmaceutically acceptable salt thereof, which is selected from the group consisting of:
(1S,2S)—N-(6-(7-(cyclopropyl(hydroxy)methyl)-6-fluoro-5-methyl-1H-indazol-4-yl)imidazo[1,2-a]pyrazin-2-yl)-2-fluorocyclopropane-1-carboxamide;
(1S,2S)—N-(6-(7-(cyclopropyl(methoxy)methyl)-6-fluoro-5-methyl-1H-indazol-4-yl)imidazo[1,2-a]pyrazin-2-yl)-2-fluorocyclopropane-1-carboxamide;
(1S,2S)—N-(6-(7-(1-ethoxyethyl)-6-fluoro-5-methyl-1H-indazol-4-yl)imidazo[1,2-a]pyrazin-2-yl)-2-fluorocyclopropane-1-carboxamide;
(1S,2S)-2-fluoro-N-(6-(6-fluoro-7-(1-methoxypropan-2-yl)-5-methyl-1H-indazol-4-yl)imidazo[1,2-a]pyrazin-2-yl)cyclopropane-1-carboxamide;
(1S,2S)—N-(6-(7-(1,1-difluoropropan-2-yl)-6-fluoro-5-methyl-1H-indazol-4-yl)imidazo[1,2-a]pyrazin-2-yl)-2-fluorocyclopropane-1-carboxamide;
(1S,2S)-2-fluoro-N-(6-(6-fluoro-5-methyl-7-(tetrahydrofuran-3-yl)-1H-indazol-4-yl)imidazo[1,2-a]pyrazin-2-yl)cyclopropane-1-carboxamide;
(1S,2S)-2-fluoro-N-(6-(6-fluoro-5-methyl-7-(3-oxocyclopent-1-en-1-yl)-1H-indazol-4-yl)imidazo[1,2-a]pyrazin-2-yl)cyclopropane-1-carboxamide;
(1S,2S)-2-fluoro-N-(6-(6-fluoro-7-(1-hydroxypropan-2-yl)-5-methyl-1H-indazol-4-yl)imidazo[1,2-a]pyrazin-2-yl)cyclopropane-1-carboxamide;
(1S,2S)-2-fluoro-N-(6-(6-fluoro-7-(2-hydroxycyclopentyl)-5-methyl-1H-indazol-4-yl)imidazo[1,2-a]pyrazin-2-yl)cyclopropane-1-carboxamide;
(1S,2S)-2-fluoro-N-(6-(6-fluoro-5-methyl-7-(1-(methylamino)ethyl)-1H-indazol-4-yl)imidazo[1,2-a]pyrazin-2-yl)cyclopropane-1-carboxamide;
(1S,2S)—N-(6-(7-(cyclopent-1-en-1-yl)-6-fluoro-5-methyl-1H-indazol-4-yl)imidazo[1,2-a]pyrazin-2-yl)-2-fluorocyclopropane-1-carboxamide;
(1S,2S)—N-(6-(7-(1-(dimethylamino)ethyl)-6-fluoro-5-methyl-1H-indazol-4-yl)imidazo[1,2-a]pyrazin-2-yl)-2-fluorocyclopropane-1-carboxamide;
(1S,2S)—N-(6-(7-(1-(dimethylamino)propan-2-yl)-6-fluoro-5-methyl-1H-indazol-4-yl)imidazo[1,2-a]pyrazin-2-yl)-2-fluorocyclopropane-1-carboxamide;
6-((5-chloro-6-fluoro-4-(2-((1S,2S)-2-fluorocyclopropane-1-carboxamido)imidazo[1,2-a]pyrazin-6-yl)-1H-indazol-7-yl)(isopropyl)amino)-3,4,5-tihydroxytetrahydro-2H-pyran-2-carboxylic acid;
(1S,2S)-2-fluoro-N-(6-(6-fluoro-7-(3-hydroxycyclopent-1-en-1-yl)-5-methyl-1H-indazol-4-yl)imidazo[1,2-a]pyrazin-2-yl)cyclopropane-1-carboxamide;
(1S,2S)-2-fluoro-N-(6-(6-fluoro-7-(2-methoxycyclopentyl)-5-methyl-1H-indazol-4-yl)imidazo[1,2-a]pyrazin-2-yl)cyclopropane-1-carboxamide;
(1S,2S)—N-(6-(5-(difluoromethyl)-7-(dimethylamino)-6-fluoro-1H-indazol-4-yl)imidazo[1,2-a]pyrazin-2-yl)-2-fluorocyclopropane-1-carboxamide;
2-((5-chloro-6-fluoro-4-(2-((1S,2S)-2-fluorocyclopropane-1-carboxamido)imidazo[1,2-a]pyrazin-6-yl)-1H-indazol-7-yl)(isopropyl)amino)-6-(methoxycarbonyl)tetrahydro-2H-pyran-3,4,5-triyl triacetate;
(1S,2S)—N-(6-(7-(cyclopropyl(hydroxy)methyl)-5-(difluoromethyl)-6-fluoro-1H-indazol-4-yl)imidazo[1,2-a]pyrazin-2-yl)-2-fluorocyclopropane-1-carboxamide;
(1S,2S)—N-(6-(7-(cyclopropyl(1,3-dioxoisoindolin-2-yl)methyl)-6-fluoro-5-methyl-1H-indazol-4-yl)imidazo[1,2-a]pyrazin-2-yl)-2-fluorocyclopropane-1-carboxamid;
(1S,2S)—N-(6-(7-(1-(1,3-dioxoisoindolin-2-yl)propan-2-yl)-6-fluoro-5-methyl-1H-indazol-4-yl)imidazo[1,2-a]pyrazin-2-yl)-2-fluorocyclopropane-1-carboxamide;
(1S,2S)—N-(6-(7-(amino(cyclopropyl)methyl)-6-fluoro-5-methyl-1H-indazol-4-yl)imidazo[1,2-a]pyrazin-2-yl)-2-fluorocyclopropane-1-carboxamide;
(1S,2S)—N-(6-(7-(1-aminopropan-2-yl)-6-fluoro-5-methyl-1H-indazol-4-yl)imidazo[1,2-a]pyrazin-2-yl)-2-fluorocyclopropane-1-carboxamide;
(1S,2S)—N-(6-(7-(1-aminoethyl)-6-fluoro-5-methyl-1H-indazol-4-yl)imidazo[1,2-a]pyrazin-2-yl)-2-fluorocyclopropane-1-carboxamide;
(1R,2R)—N-(6-(7-(dimethylamino)-6-fluoro-5-(trifluoromethyl)-1H-indazol-4-yl)imidazo[1,2-a]pyrazin-2-yl)-2-fluorocyclopropane-1-carboxamide;
(1S,2S)—N-(6-(5-(difluoromethyl)-6-fluoro-7-(isopropylamino)-1H-indazol-4-yl)imidazo[1,2-a]pyrazin-2-yl)-2-fluorocyclopropane-1-carboxamide;
(1S,2R)—N-(6-(7-(dimethylamino)-6-fluoro-5-(trifluoromethyl)-1H-indazol-4-yl)imidazo[1,2-a]pyrazin-2-yl)-2-fluorocyclopropane-1-carboxamide;
(1R,2S)—N-(6-(7-(dimethylamino)-6-fluoro-5-(trifluoromethyl)-1H-indazol-4-yl)imidazo[1,2-a]pyrazin-2-yl)-2-fluorocyclopropane-1-carboxamide;
(1R,2R)-2-fluoro-N-(6-(6-fluoro-7-(isopropylamino)-5-methyl-1H-indazol-4-yl)imidazo[1,2-a]pyrazin-2-yl)cyclopropane-1-carboxamide;
(1S,2R)-2-fluoro-N-(6-(6-fluoro-7-(isopropylamino)-5-methyl-1H-indazol-4-yl)imidazo[1,2-a]pyrazin-2-yl)cyclopropane-1-carboxamide;
(1R,2S)-2-fluoro-N-(6-(6-fluoro-7-(isopropylamino)-5-methyl-1H-indazol-4-yl)imidazo[1,2-a]pyrazin-2-yl)cyclopropane-1-carboxamide;
(1S,2S)—N-(6-(7-(cyclopropyl(hydroxy)methyl)-6-fluoro-5-(trifluoromethyl)-1H-indazol-4-yl)imidazo[1,2-a]pyrazin-2-yl)-2-fluorocyclopropane-1-carboxamide;
(1S,2S)-2-fluoro-N-(6-(6-fluoro-5-(hydroxymethyl)-7-(isopropylamino)-1H-indazol-4-yl)imidazo[1,2-a]pyrazin-2-yl)cyclopropane-1-carboxamide;
(1S,2S)—N-(6-(7-(amino(cyclopropyl)methyl)-5-(difluoromethyl)-6-fluoro-1H-indazol-4-yl)imidazo[1,2-a]pyrazin-2-yl)-2-fluorocyclopropane-1-carboxamide;
(1S,2S)—N-(6-(7-(acetamido(cyclopropyl)methyl)-6-fluoro-5-methyl-1H-indazol-4-yl)imidazo[1,2-a]pyrazin-2-yl)-2-fluorocyclopropane-1-carboxamide;
(1S,2S)—N-(6-(7-(amino(cyclopropyl)methyl)-6-fluoro-5-(trifluoromethyl)-1H-indazol-4-yl)imidazo[1,2-a]pyrazin-2-yl)-2-fluorocyclopropane-1-carboxamide;
(1S,2S)—N-(6-(7-(1-aminoethyl)-6-fluoro-5-(trifluoromethyl)-1H-indazol-4-yl)imidazo[1,2-a]pyrazin-2-yl)-2-fluorocyclopropane-1-carboxamide;
(1S,2S)—N-(6-(7-(cyclopropyl(1,3-dioxoisoindolin-2-yl)methyl)-5-(difluoromethyl)-6-fluoro-1H-indazol-4-yl)imidazo[1,2-a]pyrazin-2-yl)-2-fluorocyclopropane-1-carboxamide;
(1S,2S)—N-(6-(5-(difluoromethyl)-6-fluoro-7-(1-hydroxyethyl)-1H-indazol-4-yl)imidazo[1,2-a]pyrazin-2-yl)-2-fluorocyclopropane-1-carboxamide;
(1S,2S)—N-(6-(5-(difluoromethyl)-7-(1-(1,3-dioxoisoindolin-2-yl)ethyl)-6-fluoro-1H-indazol-4-yl)imidazo[1,2-a]pyrazin-2-yl)-2-fluorocyclopropane-1-carboxamide;
(1S,2S)—N-(6-(7-(1-acetamidoethyl)-5-(difluoromethyl)-6-fluoro-1H-indazol-4-yl)imidazo[1,2-a]pyrazin-2-yl)-2-fluorocyclopropane-1-carboxamide;
(1S,2S)—N-(6-(7-(acetamido(cyclopropyl)methyl)-5-(difluoromethyl)-6-fluoro-1H-indazol-4-yl)imidazo[1,2-a]pyrazin-2-yl)-2-fluorocyclopropane-1-carboxamide;
(1S,2S)—N-(6-(7-(1-aminoethyl)-5-(difluoromethyl)-6-fluoro-1H-indazol-4-yl)imidazo[1,2-a]pyrazin-2-yl)-2-fluorocyclopropane-1-carboxamide;
(1S,2S)—N-(6-(7-(acetamido(cyclopropyl)methyl)-6-fluoro-5-(trifluoromethyl)-1H-indazol-4-yl)imidazo[1,2-a]pyrazin-2-yl)-2-fluorocyclopropane-1-carboxamide formate;
methyl 6-fluoro-4-(2-((1S,2S)-2-fluorocyclopropane-1-carboxamido)imidazo[1,2-a]pyrazin-6-yl)-7-(isopropylamino)-1H-indazole-5-carboxylate;
(1S,2S)—N-(6-(7-(1-acetamidoethyl)-6-fluoro-5-(trifluoromethyl)-1H-indazol-4-yl)imidazo[1,2-a]pyrazin-2-yl)-2-fluorocyclopropanecarboxamide formate;
(1S,2S)—N-(6-(7-(1-(1,3-dioxoisoindolin-2-yl)ethyl)-6-fluoro-5-(trifluoromethyl)-1H-indazol-4-yl)imidazo[1,2-a]pyrazin-2-yl)-2-fluorocyclopropanecarboxamide formate;
(1S,2S)—N-(6-(7-(cyclopropanecarboxamido(cyclopropyl)methyl)-6-fluoro-5-methyl-1H-indazol-4-yl)imidazo[1,2-a]pyrazin-2-yl)-2-fluorocyclopropane-1-carboxamide;
(1S,2S)—N-(6-(7-(1-(1,3-dioxoisoindolin-2-yl)ethyl)-6-fluoro-5-methyl-1H-indazol-4-yl)imidazo[1,2-a]pyrazin-2-yl)-2-fluorocyclopropane-1-carboxamide;
(1S,2S)—N-(6-(7-(1-(cyclopropanecarboxamido)ethyl)-6-fluoro-5-methyl-1H-indazol-4-yl)imidazo[1,2-a]pyrazin-2-yl)-2-fluorocyclopropane-1-carboxamide;
(1S,2S)—N-(6-(7-(1-acetamidoethyl)-6-fluoro-5-methyl-1H-indazol-4-yl)imidazo[1,2-a]pyrazin-2-yl)-2-fluorocyclopropane-1-carboxamide;
(1S,2S)-2-fluoro-N-(6-(6-fluoro-5-methyl-7-(1-(2,2,2-trifluoroacetamido)ethyl)-1H-indazol-4-yl)imidazo[1,2-a]pyrazin-2-yl)cyclopropane-1-carboxamide;
(1S,2S)—N-(6-(5-cyano-6-fluoro-1H-indazol-4-yl)imidazo[1,2-a]pyrazin-2-yl)-2-fluorocyclopropane-1-carboxamide;
6-fluoro-4-(2-((1S,2S)-2-fluorocyclopropane-1-carboxamido)imidazo[1,2-a]pyrazin-6-yl)-7-(isopropylamino)-1H-indazole-5-carboxylic acid;
(1S,2S)—N-(6-(5-(difluoromethyl)-6-fluoro-7-(1-formamidoethyl)-1H-indazol-4-yl)imidazo[1,2-a]pyrazin-2-yl)-2-fluorocyclopropane-1-carboxamide;
(1S,2S)—N-(6-(7-(1-(cyclopropanecarboxamido)ethyl)-5-(difluoromethyl)-6-fluoro-1H-indazol-4-yl)imidazo[1,2-a]pyrazin-2-yl)-2-fluorocyclopropane-1-carboxamide;
(1S,2S)—N-(6-(7-(cyclopropanecarboxamido(cyclopropyl)methyl)-5-(difluoromethyl)-6-fluoro-1H-indazol-4-yl)imidazo[1,2-a]pyrazin-2-yl)-2-fluorocyclopropane-1-carboxamide;
(1S,2S)-2-fluoro-N-(6-(6-fluoro-7-(1-hydroxypropan-2-yl)-5-(trifluoromethyl)-1H-indazol-4-yl)imidazo[1,2-a]pyrazin-2-yl)cyclopropane-1-carboxamide;
(1S,2S)—N-(6-(7-(1-((tert-butylsulfinyl)amino)ethyl)-5-chloro-6-fluoro-1H-indazol-4-yl)imidazo[1,2-a]pyrazin-2-yl)-2-fluorocyclopropane-1-carboxamide;
(1S,2S)—N-(6-(7-(1-((tert-butylsulfinyl)amino)ethyl)-6-fluoro-5-(trifluoromethyl)-1H-indazol-4-yl)imidazo[1,2-a]pyrazin-2-yl)-2-fluorocyclopropane-1-carboxamide;
(1S,2S)-2-fluoro-N-(6-(6-fluoro-7-(1-formamidoethyl)-5-(trifluoromethyl)-1H-indazol-4-yl)imidazo[1,2-a]pyrazin-2-yl)cyclopropane-1-carboxamide;
(1S,2S)—N-(6-(7-(((tert-butylsulfinyl)amino)(cyclopropyl)methyl)-6-fluoro-5-(trifluoromethyl)-1H-indazol-4-yl)imidazo[1,2-a]pyrazin-2-yl)-2-fluorocyclopropane-1-carboxamide;
(1S,2S)—N-(6-(7-(1-aminopropan-2-yl)-6-fluoro-5-(trifluoromethyl)-1H-indazol-4-yl)imidazo[1,2-a]pyrazin-2-yl)-2-fluorocyclopropane-1-carboxamide formate;
(1S,2S)—N-(6-(7-(1-acetamidopropan-2-yl)-6-fluoro-5-(trifluoromethyl)-1H-indazol-4-yl)imidazo[1,2-a]pyrazin-2-yl)-2-fluorocyclopropane-1-carboxamide;
(1S,2S)—N-(6-(7-(cyclopropyl(2,2,2-trifluoroacetamido)methyl)-6-fluoro-5-methyl-1H-indazol-4-yl)imidazo[1,2-a]pyrazin-2-yl)-2-fluorocyclopropane-1-carboxamide;
(1S,2S)—N-(6-(7-(cyclopropyl(formamido)methyl)-6-fluoro-5-(trifluoromethyl)-1H-indazol-4-yl)imidazo[1,2-a]pyrazin-2-yl)-2-fluorocyclopropane-1-carboxamide;
(1S,2S)—N-(6-(7-(cyclopropyl(hydroxy)methyl)-6-fluoro-5-methoxy-1H-indazol-4-yl)imidazo[1,2-a]pyrazin-2-yl)-2-fluorocyclopropane-1-carboxamide;
(1S,2S)-2-fluoro-N-(6-(6-fluoro-7-(1-hydroxyethyl)-5-methoxy-1H-indazol-4-yl)imidazo[1,2-a]pyrazin-2-yl)cyclopropane-1-carboxamide;
(1S,2S)—N-(6-(7-((S)-1-(((S)-tert-butylsulfinyl)amino)ethyl)-5-chloro-6-fluoro-1H-indazol-4-yl)imidazo[1,2-a]pyrazin-2-yl)-2-fluorocyclopropane-1-carboxamide;
(1S,2S)—N-(6-(7-((R)-1-(((R)-tert-butylsulfinyl)amino)ethyl)-5-chloro-6-fluoro-1H-indazol-4-yl)imidazo[1,2-a]pyrazin-2-yl)-2-fluorocyclopropane-1-carboxamide;
(1S,2S)—N-(6-(7-(dimethylamino)-6-fluoro-5-methoxy-1H-indazol-4-yl)imidazo[1,2-a]pyrazin-2-yl)-2-fluorocyclopropane-1-carboxamide;
(1S,2S)—N-(6-(5-chloro-6-fluoro-7-((R)-1-(2,2,2-trifluoroacetamido)ethyl)-1H-indazol-4-yl)imidazo[1,2-a]pyrazin-2-yl)-2-fluorocyclopropane-1-carboxamide;
(1S,2S)-2-fluoro-N-(6-(6-fluoro-7-(isopropylamino)-5-methoxy-1H-indazol-4-yl)imidazo[1,2-a]pyrazin-2-yl)cyclopropane-1-carboxamide;
(1S,2S)—N-(6-(7-((1-acetylazetidin-3-yl)oxy)-5-chloro-6-fluoro-1H-indazol-4-yl)imidazo[1,2-a]pyrazin-2-yl)-2-fluorocyclopropane-1-carboxamide;
(1S,2S)—N-(6-(7-(azetidin-3-yloxy)-5-chloro-6-fluoro-1H-indazol-4-yl)imidazo[1,2-a]pyrazin-2-yl)-2-fluorocyclopropane-1-carboxamide formate;
(1S,2S)—N-(6-(5-chloro-6-fluoro-7-((S)-1-(2,2,2-trifluoroacetamido)ethyl)-1H-indazol-4-yl)imidazo[1,2-a]pyrazin-2-yl)-2-fluorocyclopropane-1-carboxamide; and
(1S,2S)-2-fluoro-N-(6-(6-fluoro-7-(isopropylamino)-1H-indazol-4-yl)imidazo[1,2-a]pyrazin-2-yl)cyclopropane-1-carboxamide.

7. The compound of claim 4 or a pharmaceutically acceptable salt thereof, wherein R¹ is selected from the group consisting of:

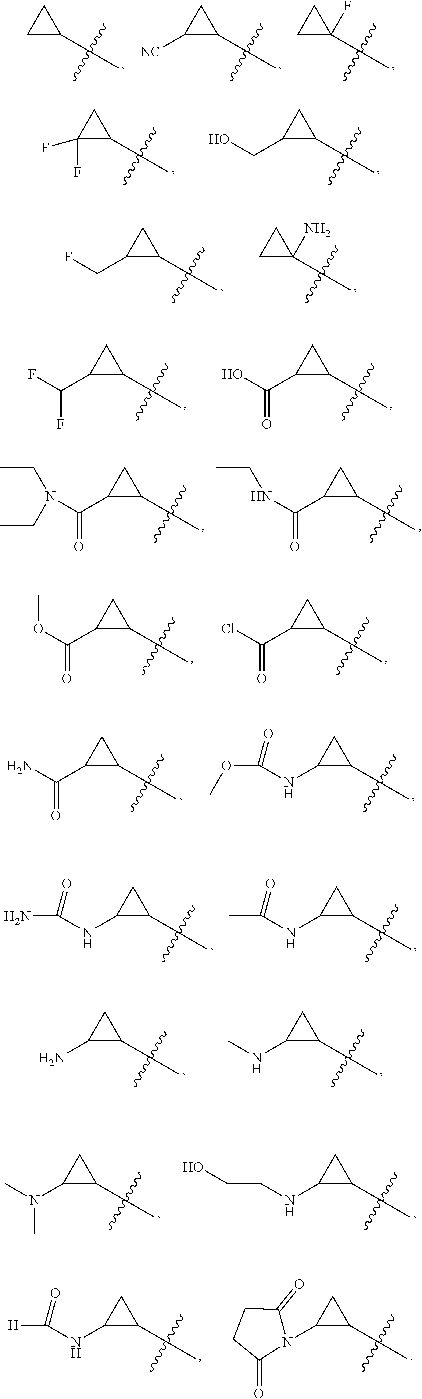

8. The compound of claim 7 or a pharmaceutically acceptable salt thereof, which is selected from the group consisting of:
(1S,2R)-2-((6-(5-chloro-6-fluoro-7-(isopropylamino)-1H-indazol-4-yl)imidazo[1,2-a]pyrazin-2-yl)carbamoyl)cyclopropane-1-carboxylic acid;
(1R,2S)—N1-(6-(5-chloro-6-fluoro-7-(isopropylamino)-1H-indazol-4-yl)imidazo[1,2-a]pyrazin-2-yl)-N2,N2-diethylcyclopropane-1,2-dicarboxamide;
(1R,2S)—N1-(6-(5-chloro-6-fluoro-7-(isopropylamino)-1H-indazol-4-yl)imidazo[1,2-a]pyrazin-2-yl)-N2-ethylcyclopropane-1,2-dicarboxamide;
methyl (1S,2R)-2-((6-(5-chloro-6-fluoro-7-(isopropylamino)-1H-indazol-4-yl)imidazo[1,2-a]pyrazin-2-yl)carbamoyl)cyclopropane-1-carboxylate;
(1R,2S)—N-(6-(5-chloro-6-fluoro-7-(isopropylamino)-1H-indazol-4-yl)imidazo[1,2-a]pyrazin-2-yl)-2-(chloromethyl)cyclopropane-1-carboxamide;
(1R,2S)—N-(6-(5-chloro-6-fluoro-7-(isopropylamino)-1H-indazol-4-yl)imidazo[1,2-a]pyrazin-2-yl)-2-(hydroxymethyl)cyclopropane-1-carboxamide;
(1R,2S)—N1-(6-(5-chloro-6-fluoro-7-(isopropylamino)-1H-indazol-4-yl)imidazo[1,2-a]pyrazin-2-yl)cyclopropane-1,2-dicarboxamide;
N-(6-(5-chloro-6-fluoro-7-(isopropylamino)-1H-indazol-4-yl)imidazo[1,2-a]pyrazin-2-yl)cyclopropanecarboxamide;
methyl (1-((6-(5-chloro-6-fluoro-7-(isopropylamino)-1H-indazol-4-yl)imidazo[1,2-a]pyrazin-2-yl)carbamoyl)cyclopropyl)carbamate;
(1R,2S)—N-(6-(5-chloro-6-fluoro-7-(isopropylamino)-1H-indazol-4-yl)imidazo[1,2-a]pyrazin-2-yl)-2-cyanocyclopropane-1-carboxamide;
N-(6-(5-chloro-6-fluoro-7-(isopropylamino)-1H-indazol-4-yl)imidazo[1,2-a]pyrazin-2-yl)-1-fluorocyclopropane-1-carboxamide;
1-amino-N-(6-(5-chloro-6-fluoro-7-(isopropylamino)-1H-indazol-4-yl)imidazo[1,2-a]pyrazin-2-yl)cyclopropane-1-carboxamide;
(1R,2S)—N-(6-(5-chloro-6-fluoro-7-(isopropylamino)-1H-indazol-4-yl)imidazo[1,2-a]pyrazin-2-yl)-2-(fluoromethyl)cyclopropane-1-carboxamide;
(1R,2S)—N-(6-(6-fluoro-7-(isopropylamino)-5-(trifluoromethyl)-1H-indazol-4-yl)imidazo[1,2-a]pyrazin-2-yl)-2-(hydroxymethyl)cyclopropane-1-carboxamide;
(1R,2S)—N-(6-(7-(cyclopropyl(hydroxy)methyl)-6-fluoro-5-(trifluoromethyl)-1H-indazol-4-yl)imidazo[1,2-a]pyrazin-2-yl)-2-(hydroxymethyl)cyclopropane-1-carboxamide;
(1R,2S)-2-cyano-N-(6-(5-(difluoromethyl)-6-fluoro-7-(isopropylamino)-1H-indazol-4-yl)imidazo[1,2-a]pyrazin-2-yl)cyclopropane-1-carboxamide;
N-(6-(5-(difluoromethyl)-6-fluoro-7-(isopropylamino)-1H-indazol-4-yl)imidazo[1,2-a]pyrazin-2-yl)cyclopropanecarboxamide;
N-(6-(6-fluoro-7-(isopropylamino)-5-methoxy-1H-indazol-4-yl)imidazo[1,2-a]pyrazin-2-yl)cyclopropanecarboxamide; and
(1R,2S)-2-cyano-N-(6-(6-fluoro-7-(isopropylamino)-5-methoxy-1H-indazol-4-yl)imidazo[1,2-a]pyrazin-2-yl)cyclopropane-1-carboxamide.

9. The compound of claim 1 or a pharmaceutically acceptable salt thereof, which is a compound of Formula (III): 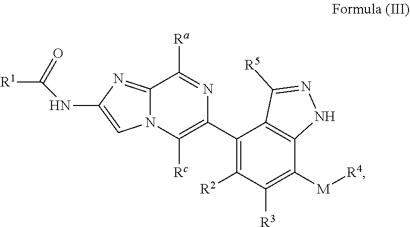
wherein R¹ is alkyl, alkenyl, alkynyl, halo, hydroxyl, —CD₂OH, —CN, —NO₂, haloalkyl, —C(O)R⁹, —C(O)OR⁹, —C(O)NR¹⁰R¹¹, —OR⁹, —OC(O)R⁹, —OC(O)NR¹⁰R¹¹, —SR⁹, —S(O)R⁹, —S(O)₂R⁹, —S(O)(=NH)R¹⁰, —S(O)₂NR¹⁰OR¹¹, —NR¹⁰R¹¹, —N(R⁶)NR¹⁰R¹¹, —N(R⁶)OR⁹, —N(R⁶)C(O)R⁹, —N(R⁶)C(O)R⁹, —N(R⁶)C(O)OR⁹, —N(R⁶)C(O)NR¹⁰R¹¹, —N(R⁶)S(O)₂R⁹, —N(R⁶)S(O)₂NR¹⁰R¹¹, or —P(O)R¹²R¹³, wherein the alkyl, alkenyl, or alkynyl is optionally substituted with one or more substituents independently selected from R⁷.

10. The compound of claim 9 or a pharmaceutically acceptable salt thereof, wherein R¹ is selected from the group consisting of fluoromethyl, difluoromethyl, trifluoromethyl, methoxymethyl, hydroxyethyl, hydroxypropyl, methyl, ethyl, ethyl, butyl, isopropyl, isobutyl, hydroxymethyl, hydroxyethyl, aminomethyl, aminoethyl, dimethylaminomethyl, methiomethyl, methylamino, ethylamino, isopropylamino, methoxy, ethoxy, isopropyloxy,

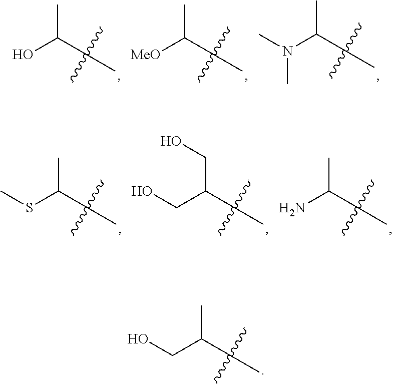

11. The compound of claim 10 or a pharmaceutically acceptable salt thereof, which is selected from the group consisting of:
N-(6-(5-chloro-6-fluoro-7-(isopropylamino)-1H-indazol-4-yl)imidazo[1,2-a]pyrazin-2-yl)acetamide;
N-(6-(5-chloro-6-fluoro-7-(isopropylamino)-1H-indazol-4-yl)imidazo[1,2-a]pyrazin-2-yl)propionamide;
N-(6-(7-(cyclopropyl(hydroxy)methyl)-6-fluoro-5-(trifluoromethyl)-2H-indazol-4-yl)imidazo[1,2-a]pyrazin-2-yl)acetamide;
N-(6-(5-chloro-6-fluoro-7-(isopropylamino)-1H-indazol-4-yl)imidazo[1,2-a]pyrazin-2-yl)-2,2,2-trifluoroacetamide;
(S)-2-amino-N-(6-(5-chloro-6-fluoro-7-(isopropylamino)-1H-indazol-4-yl)imidazo[1,2-a]pyrazin-2-yl)propenamide;
2-amino-N-(6-(5-chloro-6-fluoro-7-(isopropylamino)-1H-indazol-4-yl)imidazo[1,2-a]pyrazin-2-yl)acetamide;
N-(6-(5-chloro-6-fluoro-7-(isopropylamino)-1H-indazol-4-yl)imidazo[1,2-a]pyrazin-2-yl)isobutyramide;
N-(6-(5-chloro-6-fluoro-7-(isopropylamino)-1H-indazol-4-yl)imidazo[1,2-a]pyrazin-2-yl)pivalamide;
N-(6-(6-fluoro-7-(isopropylamino)-5-(trifluoromethyl)-1H-indazol-4-yl)imidazo[1,2-a]pyrazin-2-yl)acetamide;
isopropyl (6-(5-chloro-6-fluoro-7-(isopropylamino)-1H-indazol-4-yl)imidazo[1,2-a]pyrazin-2-yl)carbamate;
N-(6-(5-chloro-6-fluoro-7-(isopropylamino)-1H-indazol-4-yl)imidazo[1,2-a]pyrazin-2-yl)-2-(methylthio)acetamide;
N-(6-(5-chloro-6-fluoro-7-(isopropylamino)-1H-indazol-4-yl)imidazo[1,2-a]pyrazin-2-yl)-N-methylacetamide;
N-(6-(5-chloro-6-fluoro-7-(isopropylamino)-1H-indazol-4-yl)imidazo[1,2-a]pyrazin-2-yl)-2-(dimethylamino)acetamide;
N-(6-(5-chloro-6-fluoro-7-(isopropylamino)-1H-indazol-4-yl)imidazo[1,2-a]pyrazin-2-yl)-2-(methylthio)propenamide;
(R)—N-(6-(5-chloro-6-fluoro-7-(isopropylamino)-1H-indazol-4-yl)imidazo[1,2-a]pyrazin-2-yl)-2-(dimethylamino)propenamide;
N-(6-(5-chloro-6-fluoro-7-(isopropylamino)-1H-indazol-4-yl)imidazo[1,2-a]pyrazin-2-yl)-2-methoxyacetamide;
N-(6-(5-chloro-6-fluoro-7-(isopropylamino)-1H-indazol-4-yl)imidazo[1,2-a]pyrazin-2-yl)butyramide;
N-(6-(5-chloro-6-fluoro-7-(isopropylamino)-1H-indazol-4-yl)imidazo[1,2-a]pyrazin-2-yl)formamide;
methyl (6-(5-chloro-6-fluoro-7-(isopropylamino)-1H-indazol-4-yl)imidazo[1,2-a]pyrazin-2-yl)carbamate;
ethyl (6-(5-chloro-6-fluoro-7-(isopropylamino)-1H-indazol-4-yl)imidazo[1,2-a]pyrazin-2-yl)carbamate;
N-(6-(5-chloro-6-fluoro-7-(isopropylamino)-1H-indazol-4-yl)imidazo[1,2-a]pyrazin-2-yl)pentanamide;
1-(6-(5-chloro-6-fluoro-7-(isopropylamino)-1H-indazol-4-yl)imidazo[1,2-a]pyrazin-2-yl)-3-ethylurea;
1-(6-(5-chloro-6-fluoro-7-(isopropylamino)-1H-indazol-4-yl)imidazo[1,2-a]pyrazin-2-yl)-3-methylurea;
N-(6-(5-chloro-6-fluoro-7-(isopropylamino)-1H-indazol-4-yl)imidazo[1,2-a]pyrazin-2-yl)-2,2-difluoroacetamide;
N-(6-(5-chloro-6-fluoro-7-(isopropylamino)-1H-indazol-4-yl)imidazo[1,2-a]pyrazin-2-yl)-2-hydroxyacetamide;
N-(6-(5-chloro-6-fluoro-7-(isopropylamino)-1H-indazol-4-yl)imidazo[1,2-a]pyrazin-2-yl)-3-hydroxypropanamide;
N-(6-(7-((S)-1-(((S)-tert-butylsulfinyl)amino)ethyl)-5-chloro-6-fluoro-1H-indazol-4-yl)imidazo[1,2-a]pyrazin-2-yl)acetamide;
N-(6-(7-((R)-1-(((R)-tert-butylsulfinyl)amino)ethyl)-5-chloro-6-fluoro-1H-indazol-4-yl)imidazo[1,2-a]pyrazin-2-yl)acetamide;
(S)—N-(1-(4-(2-acetamidoimidazo[1,2-a]pyrazin-6-yl)-5-chloro-6-fluoro-1H-indazol-7-yl)ethyl)-2,2,2-trifluoroacetamide;
(R)—N-(1-(4-(2-acetamidoimidazo[1,2-a]pyrazin-6-yl)-5-chloro-6-fluoro-1H-indazol-7-yl)ethyl)-2,2,2-trifluoroacetamide;
(S)—N-(6-(5-chloro-6-fluoro-7-(1-formamidoethyl)-1H-indazol-4-yl)imidazo[1,2-a]pyrazin-2-yl)acetamide;
N-(6-(7-((1-acetylazetidin-3-yl)oxy)-5-chloro-6-fluoro-1H-indazol-4-yl)imidazo[1,2-a]pyrazin-2-yl)acetamide;
N-(6-(7-(azetidin-3-yloxy)-5-chloro-6-fluoro-1H-indazol-4-yl)imidazo[1,2-a]pyrazin-2-yl)acetamide formate;
N-(6-(5-chloro-6-fluoro-7-((1-(2-hydroxyacetyl)azetidin-3-yl)oxy)-1H-indazol-4-yl)imidazo[1,2-a]pyrazin-2-yl)acetamide;
N-(6-(5-chloro-6-fluoro-7-((1-propionylazetidin-3-yl)oxy)-1H-indazol-4-yl)imidazo[1,2-a]pyrazin-2-yl)acetamide;
N-(6-(5-chloro-6-fluoro-7-((3-hydroxyazetidin-1-yl)methyl)-1H-indazol-4-yl)imidazo[1,2-a]pyrazin-2-yl)acetamide;
N-(6-(5-chloro-6-fluoro-7-((3-hydroxypyrrolidin-1-yl)methyl)-1H-indazol-4-yl)imidazo[1,2-a]pyrazin-2-yl)acetamide;
N-(6-(5-chloro-7-(cyclopropyl(hydroxy)methyl)-6-fluoro-1H-indazol-4-yl)imidazo[1,2-a]pyrazin-2-yl)acetamide;
N-(6-(5-(difluoromethyl)-6-fluoro-7-(isopropylamino)-1H-indazol-4-yl)imidazo[1,2-a]pyrazin-2-yl)acetamide;
N-(6-(5-(difluoromethyl)-6-fluoro-7-(isopropylamino)-1H-indazol-4-yl)imidazo[1,2-a]pyrazin-2-yl)propionamide;
N-(6-(5-chloro-6-fluoro-7-((3-hydroxy-3-methylazetidin-1-yl)methyl)-1H-indazol-4-yl)imidazo[1,2-a]pyrazin-2-yl)acetamide;
N-(6-(6-fluoro-7-(isopropylamino)-5-methoxy-1H-indazol-4-yl)imidazo[1,2-a]pyrazin-2-yl)acetamide;
N-(6-(5-chloro-6-fluoro-7-((3-hydroxy-3-methylpyrrolidin-1-yl)methyl)-1H-indazol-4-yl)imidazo[1,2-a]pyrazin-2-yl)acetamide;
N-(6-(5-chloro-6-fluoro-7-(1-(3-hydroxyazetidin-1-yl)ethyl)-1H-indazol-4-yl)imidazo[1,2-a]pyrazin-2-yl)acetamide;
N-(6-(6-fluoro-7-(isopropylamino)-5-methoxy-1H-indazol-4-yl)imidazo[1,2-a]pyrazin-2-yl)propionamide; and
N-(6-(5-chloro-6-fluoro-7-(1-(3-hydroxypyrrolidin-1-yl)ethyl)-1H-indazol-4-yl)imidazo[1,2-a]pyrazin-2-yl)acetamide.

12. The compound of claim 1 or a pharmaceutically acceptable salt thereof, which is a compound of Formula (IV): 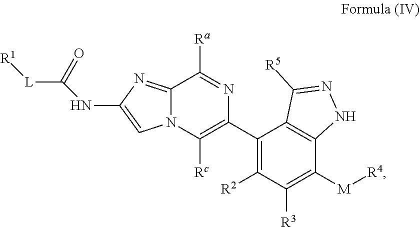
wherein R¹ is cycloalkyl, aryl, heteroaryl, or heterocyclyl, wherein the cycloalkyl, aryl, heteroaryl, or heterocyclyl is optionally substituted with one or more substituents independently selected from R⁷.

13. The compound of claim 12 or a pharmaceutically acceptable salt thereof, wherein R¹ is selected from the group consisting of:

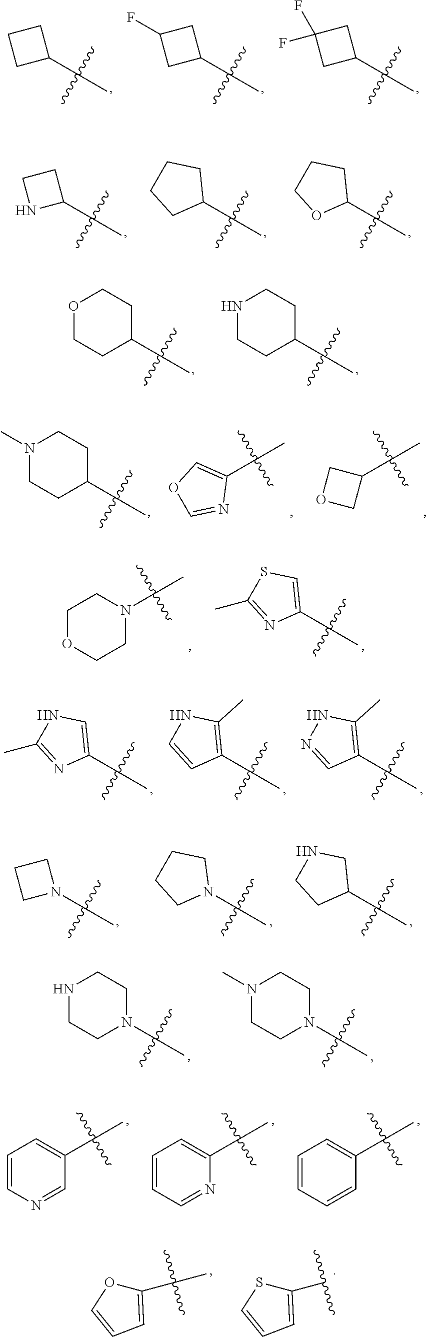

14. The compound of claim 13 or a pharmaceutically acceptable salt thereof, which is selected from the group consisting of:
N-(6-(5-chloro-6-fluoro-7-(isopropylamino)-1H-indazol-4-yl)imidazo[1,2-a]pyrazin-2-yl)benzamide;
N-(6-(5-chloro-6-fluoro-7-(isopropylamino)-1H-indazol-4-yl)imidazo[1,2-a]pyrazin-2-yl)picolinamide;
N-(6-(5-chloro-6-fluoro-7-(isopropylamino)-1H-indazol-4-yl)imidazo[1,2-a]pyrazin-2-yl)cyclobutanecarboxamide;
N-(6-(5-chloro-6-fluoro-7-(isopropylamino)-1H-indazol-4-yl)imidazo[1,2-a]pyrazin-2-yl)pyrrolidine-3-carboxamide;
N-(6-(5-chloro-6-fluoro-7-(isopropylamino)-1H-indazol-4-yl)imidazo[1,2-a]pyrazin-2-yl)morpholine-4-carboxamide;
N-(6-(5-chloro-6-fluoro-7-(isopropylamino)-1H-indazol-4-yl)imidazo[1,2-a]pyrazin-2-yl)-2-(pyridin-3-yl)acetamide;
N-(6-(5-chloro-6-fluoro-7-(isopropylamino)-1H-indazol-4-yl)imidazo[1,2-a]pyrazin-2-yl)-2-morpholinoacetamide;
N-(6-(5-chloro-6-fluoro-7-(isopropylamino)-1H-indazol-4-yl)imidazo[1,2-a]pyrazin-2-yl)-4-methylpiperazine-1-carboxamide formate;
N-(6-(6-fluoro-7-(isopropylamino)-5-methyl-1H-indazol-4-yl)imidazo[1,2-a]pyrazin-2-yl)-2-(pyridin-3-yl)acetamide;
N-(6-(5-chloro-6-fluoro-7-(isopropylamino)-1H-indazol-4-yl)imidazo[1,2-a]pyrazin-2-yl)azetidine-2-carboxamide;
N-(6-(5-chloro-6-fluoro-7-(isopropylamino)-1H-indazol-4-yl)imidazo[1,2-a]pyrazin-2-yl)tetrahydrofuran-2-carboxamide;
N-(6-(5-chloro-6-fluoro-7-(isopropylamino)-1H-indazol-4-yl)imidazo[1,2-a]pyrazin-2-yl)-2-(oxetan-3-yl)acetamide;
N-(6-(5-chloro-6-fluoro-7-(isopropylamino)-1H-indazol-4-yl)imidazo[1,2-a]pyrazin-2-yl)piperazine-1-carboxamide;
N-(6-(5-chloro-6-fluoro-7-(isopropylamino)-1H-indazol-4-yl)imidazo[1,2-a]pyrazin-2-yl)-1-methylpiperidine-4-carboxamide;
N-(6-(5-chloro-6-fluoro-7-(isopropylamino)-1H-indazol-4-yl)imidazo[1,2-a]pyrazin-2-yl)pyrrolidine-1-carboxamide;
N-(6-(5-chloro-6-fluoro-7-(isopropylamino)-1H-indazol-4-yl)imidazo[1,2-a]pyrazin-2-yl)tetrahydrofuran-3-carboxamide;
cyclobutyl (6-(5-chloro-6-fluoro-7-(isopropylamino)-1H-indazol-4-yl)imidazo[1,2-a]pyrazin-2-yl)carbamate formate; and
N-(6-(5-chloro-6-fluoro-7-(isopropylamino)-1H-indazol-4-yl)imidazo[1,2-a]pyrazin-2-yl)oxetane-3-carboxamide.

15. The compound of claim 1 or a pharmaceutically acceptable salt thereof, which is a compound of Formula (V):

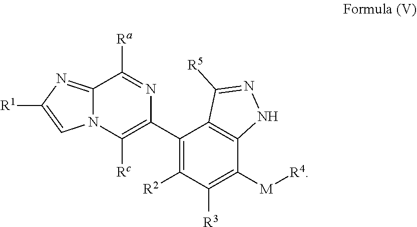

16. The compound of claim 15 or a pharmaceutically acceptable salt thereof, wherein R¹ is selected from the group consisting of:

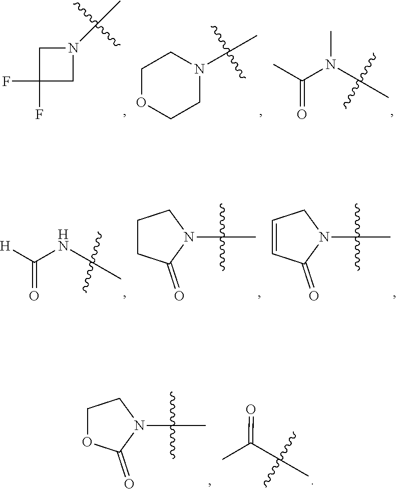

17. The compound of claim 16 or a pharmaceutically acceptable salt thereof, which is selected from the group consisting of:
1-(6-(5-chloro-6-fluoro-7-(isopropylamino)-1H-indazol-4-yl)imidazo[1,2-a]pyrazin-2-yl)-1,5-dihydro-2H-pyrrol-2-one; and
5-chloro-6-fluoro-N-isopropyl-4-(2-morpholinoimidazo[1,2-a]pyrazin-6-yl)-1H-indazol-7-amine.

18. The compound of claim 1 or a pharmaceutically acceptable salt thereof, which is a compound of Formula (VI):

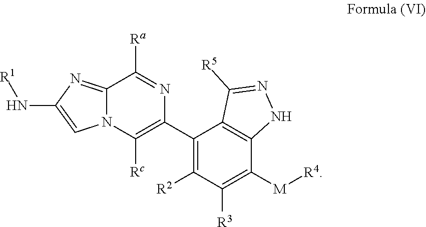

19. The compound of claim 19 or a pharmaceutically acceptable salt thereof, wherein R¹ is selected from the group consisting of:

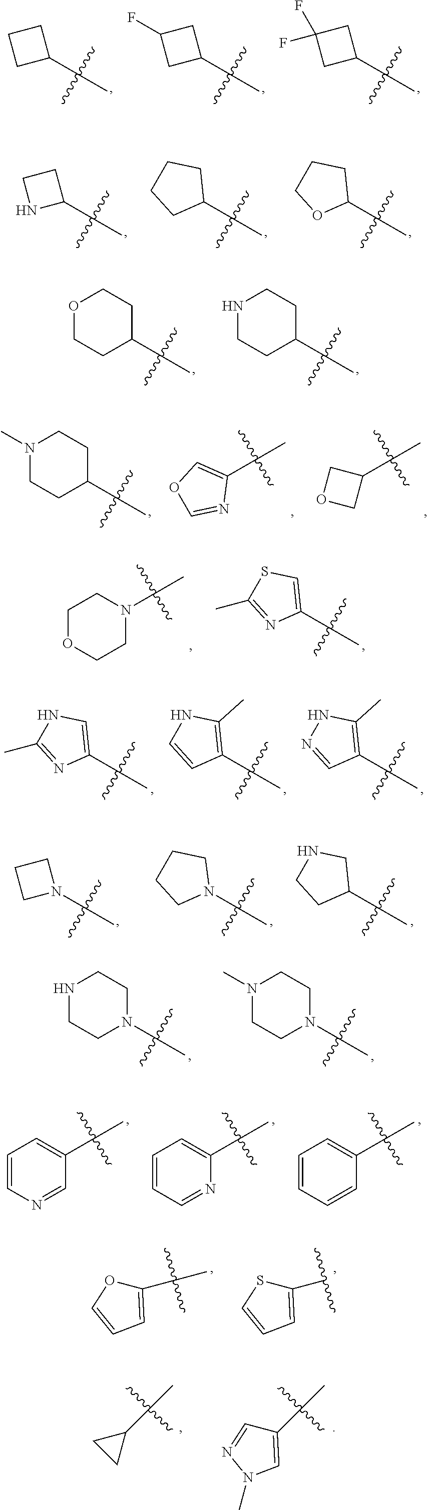

20. The compound of claim 19 or a pharmaceutically acceptable salt thereof, which is 6-(5-chloro-6-fluoro-7-(isopropylamino)-1H-indazol-4-yl)-N-(tetrahydrofuran-3-yl)imidazo[1,2-a]pyrazin-2-amine.

21. A pharmaceutical composition comprising the compound of claim 1 or a pharmaceutically acceptable salt thereof and a pharmaceutically acceptable carrier or diluent.

22. A method of treating a subject with a disease or disorder associated with modulation of HPK1 comprising:
administering to the subject in need thereof a therapeutically effective amount of the compound of claim 1 or a pharmaceutically acceptable salt thereof.

23. The method of claim 22, wherein the disease is cancer, and wherein the compound is administered with anti-PD-1 agent, anti-PD-L1 agent, or anti-CTLA4 agent.

24. The method of claim 22, wherein the disease is cancer, metastasis, inflammation or auto-immune disease.

25. The method of claim 24, wherein the cancer is selected from the group consisting of carcinomas, melanomas, blastomas, sarcomas, lymphomas and leukemias, including without limitation, bladder carcinoma, brain tumors, breast cancer, cervical cancer, colorectal cancer, esophageal cancer, endometrial cancer, hepatocellular carcinoma, laryngeal cancer, lung cancer, osteosarcoma, ovarian cancer, pancreatic cancer, prostate cancer, renal carcinoma and thyroid cancer, acute lymphocytic leukemia, acute myeloid leukemia, ependymoma, Ewing's sarcoma, glioblastoma, medulloblastoma, neuroblastoma, osteosarcoma, rhabdomyosarcoma, rhabdoid cancer, and nephroblastoma (Wilm's tumor).
